(19) United States
(12) Patent Application Publication
(10) Pub. No.: US 2026/0337482 A1
(43) Pub. Date: Oct. 29, 2026

(54) ORGANIC COMPOUND AND LIGHT-EMITTING DEVICE

(71) Applicant: SEMICONDUCTOR ENERGY LABORATORY CO., LTD., Atsugi-shi (JP)

(72) Inventors: Kyoko TAKEDA, Atsugi (JP); Yui YOSHIYASU, Atsugi (JP); Tomohiro KUBOTA, Atsugi (JP); Takeyoshi WATABE, Atsugi (JP); Nobuharu OHSAWA, Zama (JP); Keito TOSU, Atsugi (JP)

(21) Appl. No.: 19/571,295

(22) Filed: Mar. 18, 2026

(30) Foreign Application Priority Data
Mar. 21, 2025 (JP) 2025-046525

(51) Int. Cl. C09K 11/06; C07F 7/08; H10K 50/16; H10K 85/40

(52) U.S. Cl. CPC C09K 11/06; C07F 7/0812; H10K 50/166; H10K 85/40; C09K 2211/1059

(57) ABSTRACT

An organic compound having a low refractive index of a film is provided. An organic compound represented by General Formula (G1) is provided. In General Formula (G1), each of Q¹, Q², and Q³ independently represents N or CH, and at least any two of Q¹, Q², and Q³ represent N; X represents C, Si, or Ge; each of R^{a}, R^{b}, and R^{c} independently represents a cycloalkyl group or an aryl group; Ar¹ represents an aryl group; each of α and β independently represents an arylene group; R^{d} and R^{e} represent bonds that are bonded to each other to form a ring, or each of R^{d} and R^{e} independently represents hydrogen, an alkyl group, a cycloalkyl group, a trisubstituted silyl group, an aryl group, or a heteroaryl group; each of R¹ to R¹⁵ independently represents hydrogen, an alkyl group, a cycloalkyl group, a trisubstituted silyl group, an aryl group, or a heteroaryl group, and at least any one of R¹ to R¹⁵ represents an alkyl group; and each of n and m independently represents an integer greater than or equal to 0 and less than or equal to 3.

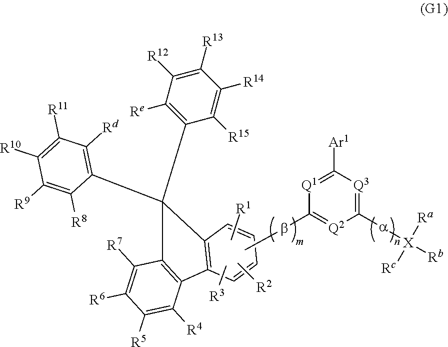

### BACKGROUND OF THE INVENTION

### 1. Field of the Invention

One embodiment of the present invention relates to an organic compound, an organic semiconductor device, a light-emitting device, a light-receiving device, a light-emitting apparatus, a light-receiving apparatus, a display device, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor apparatus, a display device, a liquid crystal display device, a light-emitting apparatus, a lighting device, a power storage device, a memory device, an imaging device, a driving method thereof, and a manufacturing method thereof.

### 2. Description of the Related Art

Light-emitting devices (organic EL devices) that include organic compounds and utilize electroluminescence (EL) have been put to practical use. In the basic structure of such light-emitting devices, an organic compound layer (EL layer) including a light-emitting material is sandwiched between a pair of electrodes. Carriers are injected by application of voltage to the device, and recombination energy of the carriers is used, whereby light emission can be obtained from the light-emitting material.

Such light-emitting devices are of self-luminous type and thus have advantages over liquid crystal devices such as high visibility and no need for backlight when used in pixels of a display, and are suitable as devices used in flat panel displays. Displays that include such light-emitting devices are also highly advantageous in that they can be thin and lightweight. Another feature of such light-emitting devices is that they have an extremely fast response speed. Since light-emitting layers of such light-emitting devices can be formed two-dimensionally and continuously, planar light emission can be achieved. This feature is difficult to realize with point light sources typified by incandescent lamps or LEDs or linear light sources typified by fluorescent lamps; thus, such light-emitting devices also have great potential as planar light sources, which can be applied to lightings and the like.

Displays or lighting devices including light-emitting devices can be used for a variety of electronic appliances as described above, and in pursuit of improving the characteristics of light-emitting devices, progress has been made in research and development of organic compounds that can be suitably used for the light-emitting devices.

For example, Non-Patent Document 1 discloses an organic compound with a low refractive index of a film. Non-Patent Document 2 discloses that a slope of a giant surface potential (GSP) of an evaporated film of an organic compound significantly changes depending on the substituent introduced into the organic compound.

### REFERENCES

[Non-Patent Document 1] Jaeho Lee et al., “Synergetic electrode architecture for efficient graphene-based flexible organic light-emitting diodes”, Nature COMMUNICATIONS, Jun. 2, 2016, DOI: 10.1038/ncomms 11791.
[Non-Patent Document 2] Masaki Tanaka et al., “Spontaneous formation of metastable orientation with well-organized permanent dipole moment in organic glassy films”, Nature Materials, 2022, Vol. 21, pp. 819-825.
[Non-Patent Document 3] Yutaka Noguchi et al., “Charge accumulation at organic semiconductor interfaces due to a permanent dipole moment and its orientational order in bilayer devices”, Journal of Applied Physics, 2012, 111, 114508.
[Non-Patent Document 4]Y. Noguchi et al., “Spontaneous Orientation Polarization of Polar Molecules and Interface Properties of Organic Electronic Devices”, Journal of the Vacuum Society of Japan, 2015, Vol. 58, No. 3.
[Non-Patent Document 5] Connie B. Roth, “Polymers under nanoconfinement: where are we now in understanding local property changes?”, Chemical Society Reviews, 2021, 50, 8050-8066.

### SUMMARY OF THE INVENTION

An object of one embodiment of the present invention is to provide an organic compound having a low refractive index of a film. Another object is to provide an organic compound having a small GSP_slope of an evaporated film. Another object is to provide an organic compound having high T_{g} of a film and powder. Another object is to provide an organic compound having a high carrier-transport property. Another object is to provide a novel organic compound.

Another object is to provide a light-emitting device having a low driving voltage. Another object is to provide a light-emitting device having high reliability. Another object is to provide a light-emitting device, a light-emitting apparatus, an electronic appliance, a display device, and an electronic device each having low power consumption.

Note that the description of these objects does not preclude the existence of other objects. One embodiment of the present invention does not need to achieve all of these objects. Other objects will be apparent from and can be derived from the description of the specification, the drawings, the claims, and the like.

One embodiment of the present invention is an organic compound represented by General Formula (G1).

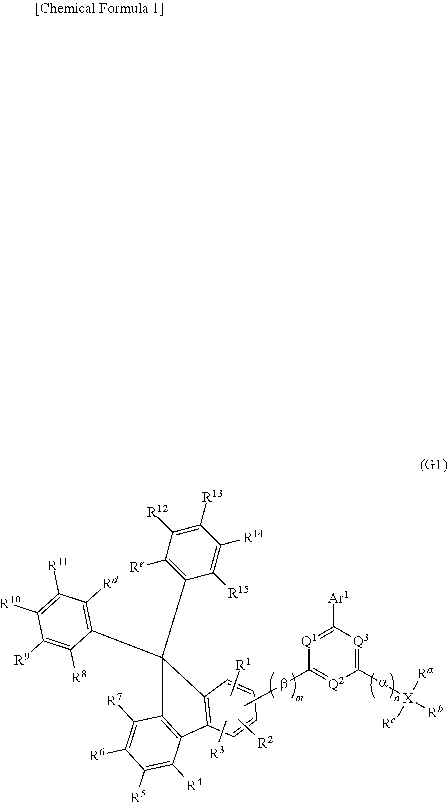

In General Formula (G1), each of Q¹, Q², and Q³ independently represents N or CH (including CD), and at least any two of Q¹, Q², and Q³ represent N; X represents C, Si, or Ge; each of R^{a}, R^{b}, and R^{c} independently represents a cycloalkyl group having 3 to 10 carbon atoms or a substituted or unsubstituted aryl group having 6 to 12 carbon atoms; Ar¹ represents a substituted or unsubstituted aryl group having 6 to 30 carbon atoms; each of α and β independently represents a substituted or unsubstituted arylene group having 6 to 30 carbon atoms; R^{d} and R^{e} represent bonds that are bonded to each other to form a ring, or each of R^{d} and R^{e} independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; each of R¹ to R¹⁵ independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms; and each of n and m independently represents an integer greater than or equal to 0 and less than or equal to 3.

Another embodiment of the present invention is an organic compound with the above structure in which each of α and β independently represents a substituted or unsubstituted arylene group having 6 to 10 carbon atoms.

Another embodiment of the present invention is an organic compound with the above structure in which each of n and m independently represents an integer greater than or equal to 0 and less than or equal to 2.

Another embodiment of the present invention is an organic compound with the above structure in which X is Si.

Another embodiment of the present invention is an organic compound with the above structure in which R^{d} and R^{e} represent bonds that are bonded to each other to form a ring.

Another embodiment of the present invention is an organic compound represented by General Formula (G4).

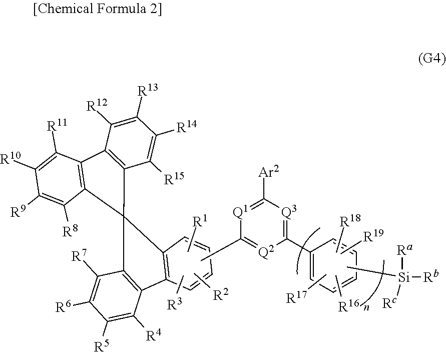

In General Formula (G4), each of Q¹, Q², and Q³ independently represents N or CH (including CD), and at least any two of Q¹, Q², and Q³ represent N; each of R^{a}, R^{b}, and R^{c} independently represents an alkyl group having 1 to 4 carbon atoms, a cycloalkyl group having 3 to 10 carbon atoms, or a substituted or unsubstituted aryl group having 6 to 12 carbon atoms; Ar² represents a substituted or unsubstituted aryl group having 6 to 10 carbon atoms; each of R¹ to R¹⁹ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; and n is an integer greater than or equal to 0 and less than or equal to 3.

Another embodiment of the present invention is an organic compound with the above structure in which Ar² represents a substituted or unsubstituted phenyl group.

Another embodiment of the present invention is an organic compound with the above structure in which n is 1.

Another embodiment of the present invention is an organic compound represented by General Formula (G6).

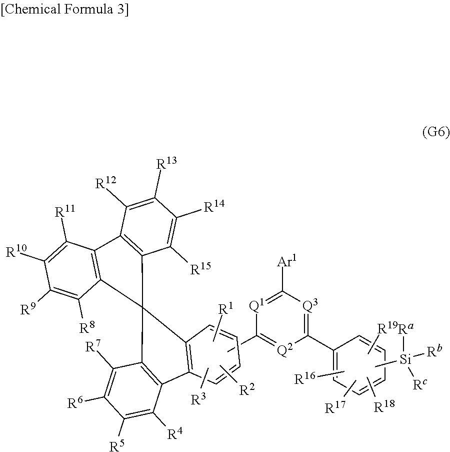

In General Formula (G6), each of Q¹, Q², and Q³ independently represents N or CH (including CD), and at least any two of Q¹, Q², and Q³ represent N; each of R^{a}, R^{b}, and R^{c} independently represents a cycloalkyl group having 3 to 10 carbon atoms or a substituted or unsubstituted aryl group having 6 to 12 carbon atoms; Ar¹ represents a substituted or unsubstituted aryl group having 6 to 30 carbon atoms; and each of R¹ to R¹⁹ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms.

Another embodiment of the present invention is an organic compound with the above structure in which Ar¹ represents a substituted or unsubstituted aryl group having 6 to 10 carbon atoms.

Another embodiment of the present invention is an organic compound with the above structure in which each of R^{a}, R^{b}, and R^{c} independently represents a substituted or unsubstituted aryl group having 6 to 12 carbon atoms.

Another embodiment of the present invention is an organic compound represented by General Formula (G7).

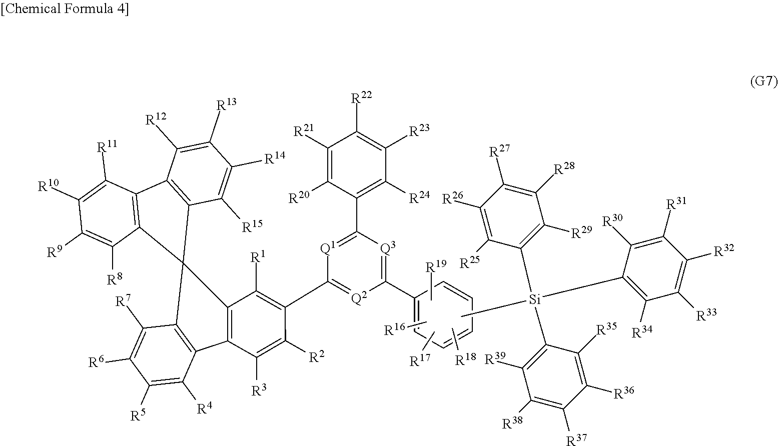

In General Formula (G7), each of Q¹, Q², and Q³ independently represents N or CH (including CD), and at least any two of Q¹, Q², and Q³ represent N; each of R¹ to R²⁴ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms; and each of R²⁵ to R³⁹ independently represents hydrogen (including deuterium) or an alkyl group having 1 to 10 carbon atoms.

Another embodiment of the present invention is an organic compound represented by any one of Structural Formula (122), Structural Formula (128), Structural Formula (129), Structural Formula (132), and Structural Formula (151).

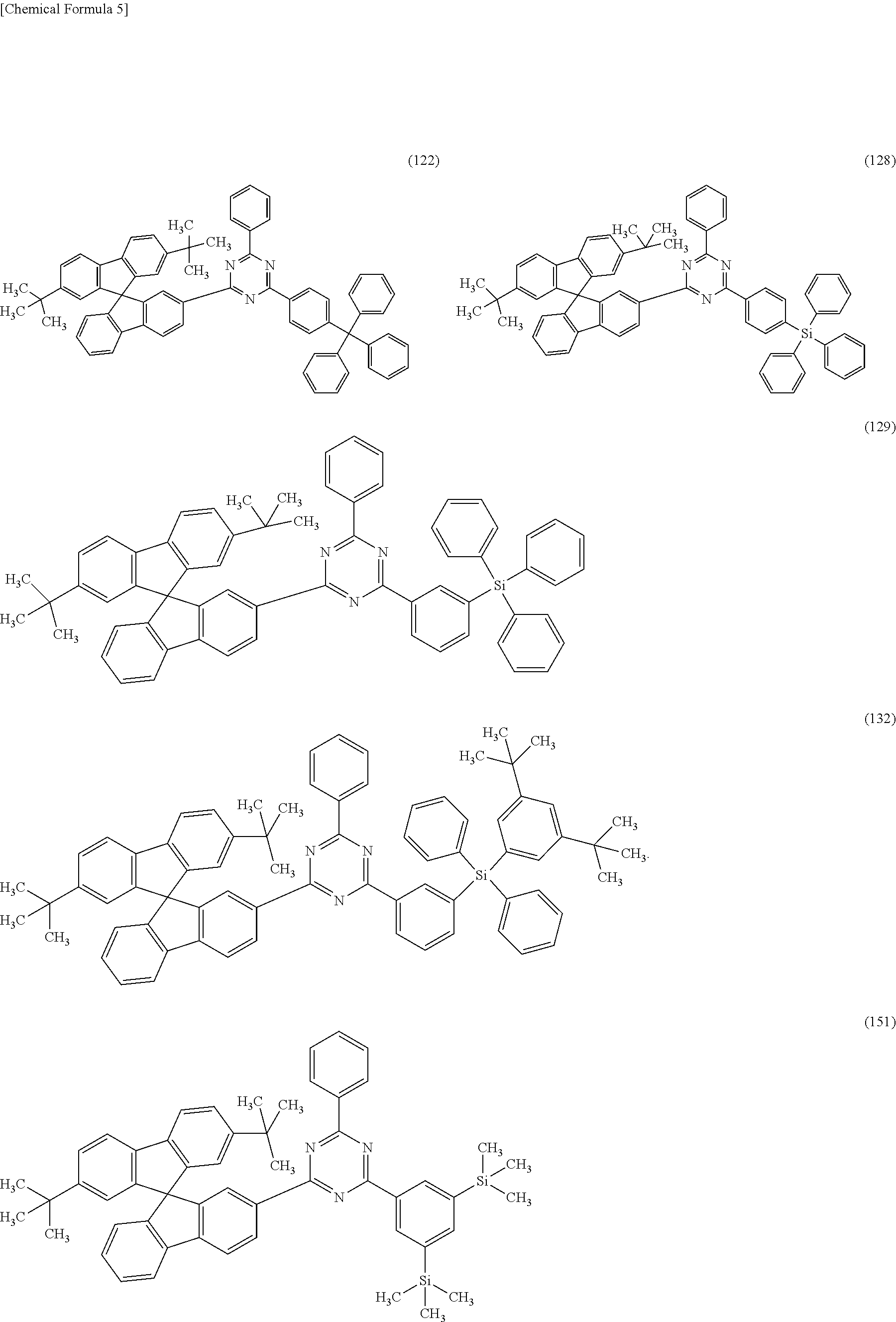

Another embodiment of the present invention is a light-emitting device including the organic compound having any of the above structures.

One embodiment of the present invention can provide an organic compound having a low refractive index of a film. Another embodiment of the present invention can provide an organic compound having a small GSP_slope of an evaporated film. Another embodiment of the present invention can provide an organic compound having high T_{g} of a film and powder. Another embodiment of the present invention can provide an organic compound having a high carrier-transport property. Another embodiment of the present invention can provide a novel organic compound.

Another embodiment of the present invention can provide a light-emitting device having a low driving voltage. Another embodiment of the present invention can provide a light-emitting device having high reliability. Another embodiment of the present invention can provide a light-emitting device, a light-emitting apparatus, an electronic appliance, a display device, and an electronic device each having low power consumption.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIGS. 1A to 1F each illustrate a structure of a light-emitting device according to an embodiment.

FIGS. 2A and 2B are perspective views illustrating a structure example of a display module.

FIGS. 3A and 3B are cross-sectional views illustrating structure examples of a display device.

FIG. 4 is a perspective view illustrating a structure example of a display device.

FIG. 5 is a cross-sectional view illustrating a structure example of a display device.

FIGS. 6A and 6B illustrate examples of electronic appliances.

FIGS. 7A to 7F illustrate examples of electronic appliances.

FIGS. 8A to 8E illustrate a method for forming an organic compound layer.

FIGS. 9A to 9D illustrate a method for forming the organic compound layer.

FIG. 10 illustrates a structure of a measurement device 1 and a measurement device 2.

FIGS. 11A and 11B illustrate structures of light-emitting devices according to examples.

FIG. 12 shows capacity-voltage characteristics of the measurement device 1 and the measurement device 2.

FIG. 13 shows current density-voltage characteristics of the measurement device 1 and the measurement device 2.

FIG. 14 is a ¹H-NMR chart of tBu-PSi-SFTzn.

FIG. 15 is an enlarged view of the ¹H-NMR chart of tBu-PSi-SFTzn.

FIG. 16 shows an emission spectrum and an absorption spectrum of tBu-PSi-SFTzn in a toluene solution.

FIG. 17 shows an emission spectrum and an absorption spectrum of a thin film of tBu-PSi-SFTzn.

FIG. 18 shows a measurement result of refractive indices of a thin film of tBu-PSi-SFTzn.

FIG. 19 is a ¹H-NMR chart of tBu-mPSi-SFTzn.

FIG. 20 is an enlarged view of the ¹H-NMR chart of tBu-mPSi-SFTzn.

FIG. 21 shows an emission spectrum and an absorption spectrum of tBu-mPSi-SFTzn in a toluene solution.

FIG. 22 shows an emission spectrum and an absorption spectrum of a thin film of tBu-mPSi-SFTzn.

FIG. 23 shows a measurement result of refractive indices of a thin film of tBu-mPSi-SFTzn.

FIG. 24 shows luminance-current density characteristics of the device 1, the device 2, a comparative device 3, and a comparative device 4.

FIG. 25 shows luminance-voltage characteristics of the device 1, the device 2, the comparative device 3, and the comparative device 4.

FIG. 26 shows current efficiency-luminance characteristics of the device 1, the device 2, the comparative device 3, and the comparative device 4.

FIG. 27 shows current density-voltage characteristics of the device 1, the device 2, the comparative device 3, and the comparative device 4.

FIG. 28 shows electroluminescence spectra of the device 1, the device 2, the comparative device 3, and the comparative device 4.

FIG. 29 shows a luminance change over driving time of each of the device 1, the comparative device 3, and the comparative device 4.

FIG. 30 shows luminance-current density characteristics of a device 5, a device 6, and a comparative device 7.

FIG. 31 shows luminance-voltage characteristics of the device 5, the device 6, and the comparative device 7.

FIG. 32 shows current efficiency-luminance characteristics of the device 5, the device 6, and the comparative device 7.

FIG. 33 shows current density-voltage characteristics of the device 5, the device 6, and the comparative device 7.

FIG. 34 shows electroluminescence spectra of the device 5, the device 6, and the comparative device 7.

FIG. 35 shows a luminance change over driving time of each of the device 5, the device 6, and the comparative device 7.

FIG. 36 shows luminance-current density characteristics of a device 8, a device 9, a comparative device 10, a comparative device 11, and a comparative device 12.

FIG. 37 shows luminance-voltage characteristics of the device 8, the device 9, the comparative device 10, the comparative device 11, and the comparative device 12.

FIG. 38 shows current efficiency-luminance characteristics of the device 8, the device 9, the comparative device 10, the comparative device 11, and the comparative device 12.

FIG. 39 shows current density-voltage characteristics of the device 8, the device 9, the comparative device 10, the comparative device 11, and the comparative device 12.

FIG. 40 shows external quantum efficiency-luminance characteristics of the device 8, the device 9, the comparative device 10, the comparative device 11, and the comparative device 12.

FIG. 41 shows electroluminescence spectra of the device 8, the device 9, the comparative device 10, the comparative device 11, and the comparative device 12.

FIG. 42 shows a luminance change over driving time of each of the device 8, the device 9, the comparative device 10, the comparative device 11, and the comparative device 12.

FIG. 43 is a ¹H-NMR chart of an intermediate 1.

FIG. 44 is a ¹H-NMR chart of an intermediate 2.

FIG. 45 is a ¹H-NMR chart of an intermediate 3.

FIG. 46 is a ¹H-NMR chart of tBu-mPSi-SFTzn-02.

FIG. 47 is an enlarged view of the ¹H-NMR chart of tBu-mPSi-SFTzn-02.

FIG. 48 shows an emission spectrum and an absorption spectrum of tBu-mPSi-SFTzn-02 in a toluene solution.

FIG. 49 shows an emission spectrum and an absorption spectrum of a thin film of tBu-mPSi-SFTzn-02.

FIG. 50 shows a measurement result of refractive indices of a thin film of tBu-mPSi-SFTzn-02.

FIG. 51 is a ¹H-NMR chart of tBu-mmTMSP-SFTzn.

FIG. 52 is an enlarged view of the ¹H-NMR chart of tBu-mmTMSP-SFTzn.

FIG. 53 shows an emission spectrum and an absorption spectrum of tBu-mmTMSP-SFTzn in a toluene solution.

FIG. 54 shows an emission spectrum and an absorption spectrum of a thin film of tBu-mmTMSP-SFTzn.

FIG. 55 shows a measurement result of refractive indices of a thin film of tBu-mmTMSP-SFTzn.

FIG. 56 is a ¹H-NMR chart of tBu-TrP-SFTzn.

FIG. 57 is an enlarged view of the ¹H-NMR chart of tBu-TrP-SFTzn.

FIG. 58 shows an emission spectrum and an absorption spectrum of tBu-TrP-SFTzn in a toluene solution.

FIG. 59 shows an emission spectrum and an absorption spectrum of a thin film of tBu-TrP-SFTzn.

FIG. 60 shows a measurement result of refractive indices of a thin film of tBu-TrP-SFTzn.

FIG. 61 shows luminance-current density characteristics of a device 13, a device 14, and a comparative device 15.

FIG. 62 shows luminance-voltage characteristics of the device 13, the device 14, and the comparative device 15.

FIG. 63 shows current efficiency-luminance characteristics of the device 13, the device 14, and the comparative device 15.

FIG. 64 shows current density-voltage characteristics of the device 13, the device 14, and the comparative device 15.

FIG. 65 shows external quantum efficiency-luminance characteristics of the device 13, the device 14, and the comparative device 15.

FIG. 66 shows electroluminescence spectra of the device 13, the device 14, and the comparative device 15.

FIG. 67 shows a luminance change over driving time of each of the device 13, the device 14, and the comparative device 15.

FIG. 68 shows luminance-current density characteristics of a device 16, a comparative device 17, and a comparative device 18.

FIG. 69 shows luminance-voltage characteristics of the device 16, the comparative device 17, and the comparative device 18.

FIG. 70 shows current efficiency-luminance characteristics of the device 16, the comparative device 17, and the comparative device 18.

FIG. 71 shows current density-voltage characteristics of the device 16, the comparative device 17, and the comparative device 18.

FIG. 72 shows external quantum efficiency-luminance characteristics of the device 16, the comparative device 17, and the comparative device 18.

FIG. 73 shows electroluminescence spectra of the device 16, the comparative device 17, and the comparative device 18.

FIG. 74 shows a luminance change over driving time of each of the device 16, the comparative device 17, and the comparative device 18.

FIG. 75 shows luminance-current density characteristics of a device 19, a comparative device 20, and a comparative device 21.

FIG. 76 shows luminance-voltage characteristics of the device 19, the comparative device 20, and the comparative device 21.

FIG. 77 shows current efficiency-luminance characteristics of the device 19, the comparative device 20, and the comparative device 21.

FIG. 78 shows current density-voltage characteristics of the device 19, the comparative device 20, and the comparative device 21.

FIG. 79 shows external quantum efficiency-luminance characteristics of the device 19, the comparative device 20, and the comparative device 21.

FIG. 80 shows electroluminescence spectra of the device 19, the comparative device 20, and the comparative device 21.

FIG. 81 shows a luminance change over driving time of each of the device 19, the comparative device 20, and the comparative device 21.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Thus, the present invention should not be construed as being limited to the description in the following embodiments.

Note that the position, size, range, or the like of each component illustrated in drawings and the like is not accurately represented in some cases for easy understanding. Thus, the disclosed invention is not necessarily limited to the position, size, range, or the like disclosed in the drawings and the like.

Ordinal numbers such as “first” and “second” in this specification and the like are used in order to avoid confusion among components and do not denote the priority or the order such as the order of steps, the stacking order, or the order of placement. A term without an ordinal number in this specification might be provided with an ordinal number in a claim in order to avoid confusion among components. A term with an ordinal number in this specification might be provided with no ordinal number in a claim. A term with an ordinal number in this specification might be provided with a different ordinal number in a claim.

In the description of structures of the present invention in this specification and the like with reference to the drawings, the same components in different drawings are denoted by the same reference numeral in some cases.

In this specification and the like, the terms “film” and “layer” can be interchanged with each other. For example, the term “conductive layer” can be changed to the term “conductive film” in some cases. For another example, the term “insulating film” can be changed into the term “insulating layer” in some cases.

Note that in this specification and the like, hydrogen (H) includes protium (¹H) and deuterium (²H or D). Protium is a stable hydrogen isotope having a mass number of 1. Deuterium is a stable hydrogen isotope having a mass number of 2.

### Embodiment 1

In this embodiment, an organic compound of one embodiment of the present invention will be described.

The organic compound of one embodiment of the present invention includes an electron-transport skeleton having a pyrimidine ring or a triazine ring, which is a π-electron deficient heteroaromatic ring (hereinafter, simply referred to as an electron-transport skeleton in some cases). The organic compound of one embodiment of the present invention includes at least any one of a trisubstituted methyl group (—CR₃), a trisubstituted silyl group (—SiR₃), and a trisubstituted germyl group (—GeR₃) that are each bonded to the electron-transport skeleton directly or through a linker. In this specification and the like, a trisubstituted methyl group (—CR₃) refers to a monovalent group having a structure in which three substituents are bonded to a carbon atom (C). A trisubstituted silyl group (—SiR₃) refers to a monovalent group having a structure in which three substituents are bonded to a silicon atom (Si). A trisubstituted germyl group (—GeR₃) refers to a monovalent group having a structure in which three substituents are bonded to a germanium atom (Ge). Specific examples of the three substituents in a trisubstituted methyl group, a trisubstituted silyl group, and a trisubstituted germyl group include an alkyl group and an aryl group. Note that the three substituents may be different or the same. The organic compound of one embodiment of the present invention includes a 9,9′-spirobifluorene ring or a 9,9-diphenylfluorene ring that is bonded to the electron-transport skeleton directly or through a linker.

Such a structure can increase the electron-transport property of the organic compound. Such a structure can reduce the refractive index of the film formed using the organic compound. Such a structure can reduce the slope of the giant surface potential (GSP) of the evaporated film formed using the organic compound. Such a structure can increase the glass transition temperature (T_{g}) of the film and powder formed using the organic compound.

Note that GSP is a phenomenon due to spontaneous orientation polarization (SOP) caused by deviation of permanent electric dipole moment orientation of an evaporated film to the thickness direction.

The surface potential of an evaporated film with GSP changes linearly with increasing thickness without saturation. For example, the surface potential of an evaporated film of tris(8-quinolinolato)aluminum(III) (abbreviation: Alq₃) reaches approximately 28 V at a thickness of 560 nm. The electric field strength reaches 5×10⁵ V/cm, which is approximately the same level as electric field strength during driving of a general light-emitting device.

The slope of a GSP (GSP_slope) is represented by AV/Ad, where AV is the amount of change in surface potential (mV) and Δd is the amount of change in the thickness (nm) of a film whose GSP changes in proportion to the thickness. Note that a GSP_slope of a film whose surface potential increases with increasing thickness is a positive GSP_slope, and a GSP_slope of a film whose surface potential decreases with increasing thickness is a negative GSP_slope. It can be said that Alq₃ described above is a material that has a positive GSP_slope. The potential of a layer with a positive GSP_slope is lower on a substrate side, and the potential of a layer with a negative GSP_slope is higher on the substrate side.

As described above, GSP is a phenomenon due to SOP caused by deviation in the thickness direction of permanent electric dipole moment orientation. That is, the following phenomena can be regarded as occurring: in a layer with a positive GSP_slope, negative polarization charge is induced on the side where evaporation starts (the substrate side) and positive polarization charge is induced on the side where evaporation ends (a second electrode side); in a similar manner, in a layer with a negative GSP_slope, positive polarization charge is induced on the side where evaporation starts (the substrate side) and negative polarization charge is induced on the side where evaporation ends (the second electrode side). Thus, GSP originates in such induction of polarization charge.

Evaporated films of most organic compounds have a positive GSP_slope; thus, in the case where a first layer is formed on and in contact with a second layer, for example, a GSP_slope of the first layer and a GSP_slope of the second layer are denoted by the same positive sign. In this case, a polarization charge of the second layer on the first layer side is canceled out by a polarization charge of the first layer on the second layer side, and only a remaining charge can be an interface charge (fixed charge) at the interface between the first layer and the second layer.

Such interface charge at the interface between evaporated films might cause adverse effects on the characteristics of a light-emitting device. It is effective to design an organic compound with a controllable GSP_slope of an evaporated film.

It is known that the use of a material with a low refractive index for an EL layer of a light-emitting device can increase the light extraction efficiency of the light-emitting device. Thus, for example, in improving the characteristics of a light-emitting device, it is useful to use an organic compound having a high carrier-transport property, a low refractive index of a film, and a small GSP_slope of an evaporated film in a carrier-transport layer of the light-emitting device. However, such an organic compound has not been easy to develop.

That is because a low refractive index of a film has a trade-off relationship with a high carrier-transport property or high reliability in a light-emitting device. This problem arises because the carrier-transport property, reliability, and the like of an organic compound are largely dependent on the presence of an unsaturated bond, and an organic compound having many unsaturated bonds tends to have a high refractive index of a film. When an organic compound has many saturated hydrocarbon groups to have a low refractive index of a film, it tends to suffer from a poor carrier-transport property, low reliability, or the like.

Saturated hydrocarbon groups, which do not have conjugation, cause smaller intermolecular dispersion force and van der Waals forces than unsaturated hydrocarbon groups. Because of this, when a vacuum-evaporated film is formed using a compound having saturated and unsaturated hydrocarbon groups, the molecules tend to orient so that the unsaturated hydrocarbon group sites face the substrate or already deposited film and the saturated hydrocarbon group sites face the film surface. Additionally, since a saturated hydrocarbon group is an electron-donating group, the dipole moment of the molecule tends to be such that the saturated hydrocarbon group side is positively polar. Thus, a film formed using a compound having a saturated hydrocarbon group tends to exhibit a large positive GSP_slope.

By contrast, the organic compound of one embodiment of the present invention has a low refractive index of a film and a small GSP_slope of an evaporated film. When being provided with the organic compound of one embodiment of the present invention, the light-emitting device can have high light extraction efficiency and a low driving voltage.

As a method for forming an organic compound layer used for a light-emitting device in a predetermined shape, a vacuum evaporation method with a metal mask (mask vapor deposition) is widely used. However, in these days of higher density and higher resolution, mask vapor deposition has come close to the limit of increasing the resolution for various reasons such as the alignment accuracy and the distance between the mask and the substrate. By contrast, a finer pattern can be formed by shape processing of an organic compound layer by a lithography method. Moreover, because of the ease of large-area processing in this method, the processing of an organic compound layer by a lithography method is being researched.

At the time of processing an organic compound layer by a lithography method, if the water solubility of an organic compound used for the organic compound layer is high, the organic compound layer is dissolved when exposed to water or a chemical solution containing water as a solvent, which might cause degraded characteristics, a shape defect, or the like. In addition, if the T_{g} of a film and powder of the organic compound used for the organic compound layer is low, the organic compound layer is crystallized in a heating step for removing water adsorbed onto the organic compound layer after being exposed to water or a chemical solution containing water as a solvent, which might reduce film quality.

By contrast, the organic compound of one embodiment of the present invention has a feature of high T_{g} of a film and powder in addition to the above features. Thus, the organic compound of one embodiment of the present invention can be suitably used also for a light-emitting device fabricated through a step of processing its organic compound layer by a lithography method.

One embodiment of the present invention is the organic compound represented by General Formula (G1).

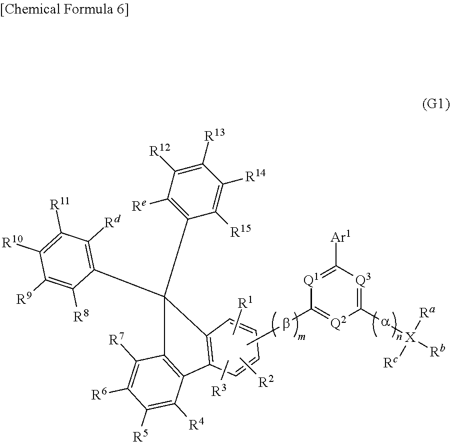

In General Formula (G1), each of Q¹, Q², and Q³ independently represents N or CH (including CD), and at least any two of Q¹, Q², and Q³ represent N; X represents C, Si, or Ge; each of R^{a}, R^{b}, and R^{c} independently represents a cycloalkyl group having 3 to 10 carbon atoms or a substituted or unsubstituted aryl group having 6 to 12 carbon atoms; Ar¹ represents a substituted or unsubstituted aryl group having 6 to 30 carbon atoms; each of α and β independently represents a substituted or unsubstituted arylene group having 6 to 30 carbon atoms; R^{d} and R^{e} represent bonds that are bonded to each other to form a ring, or each of R^{d} and R^{e} independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; each of R¹ to R¹⁵ independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms; and each of n and m independently represents an integer greater than or equal to 0 and less than or equal to 3.

The organic compound represented by General Formula (G1) includes an electron-transport skeleton having a pyrimidine ring or a triazine ring, which is a π-electron deficient heteroaromatic ring. The organic compound represented by General Formula (G1) includes at least any one of a trisubstituted silyl group, a trisubstituted methyl group, and a trisubstituted germanium group that are each bonded to the electron-transport skeleton directly or through a linker. The organic compound represented by General Formula (G1) includes a 9,9′-spirobifluorene ring or a 9,9-diphenylfluorene ring that is bonded to the electron-transport skeleton directly or through a linker. Thus, the organic compound represented by General Formula (G1) has a high electron-transport property. The organic compound represented by General Formula (G1) has a low refractive index of a film. The organic compound represented by General Formula (G1) has a small GSP_slope of an evaporated film. The organic compound represented by General Formula (G1) has high glass transition temperature (T_{g}) of a film and powder. The 9,9′-spirobifluorene ring or the 9,9-diphenylfluorene ring of the organic compound represented by General Formula (G1) includes an alkyl group having 1 to 10 carbon atoms. This can intensify the effect of increasing the T_{g} of a film and powder of the organic compound.

In the organic compound represented by General Formula (G1), it is preferable that X be Si. A trisubstituted silyl group has an electron-donating property lower than that of a trisubstituted methyl group; thus, when X is Si, the molecule can have a small permanent electric dipole moment. Such a molecule can be formed into a film with a small spontaneous orientation polarization (SOP) by a vacuum evaporation method. Accordingly, when X is Si, the GSP_slope of the evaporated film of the organic compound can be reduced. An organic compound having a trisubstituted silyl group is preferable because it can be synthesized at a cost lower than that of an organic compound having a trisubstituted germyl group.

Accordingly, one embodiment of the present invention is an organic compound represented by General Formula (G2).

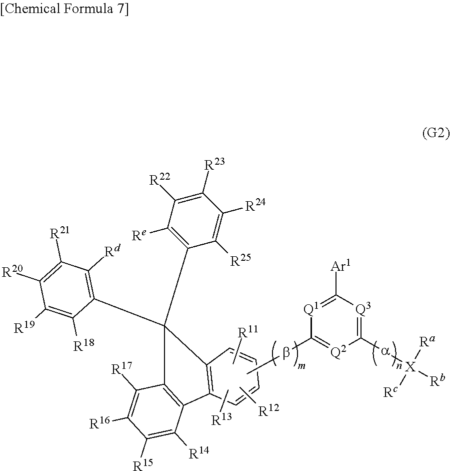

In General Formula (G2), each of Q¹, Q², and Q³ independently represents N or CH (including CD), and at least any two of Q¹, Q², and Q³ represent N; each of R^{a}, R^{b}, and R^{c} independently represents a cycloalkyl group having 3 to 10 carbon atoms or a substituted or unsubstituted aryl group having 6 to 12 carbon atoms; Ar¹ represents a substituted or unsubstituted aryl group having 6 to 30 carbon atoms; each of α and β independently represents a substituted or unsubstituted arylene group having 6 to 30 carbon atoms; R^{d} and R^{e} represent bonds that are bonded to each other to form a ring, or each of R^{d} and R^{e} independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; each of R¹¹ to R²⁵ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹¹ to R²⁵ represents an alkyl group having 1 to 10 carbon atoms; and each of n and m independently represents an integer greater than or equal to 0 and less than or equal to 3.

The organic compound represented by General Formula (G2) is different from the organic compound represented by General Formula (G1) in that X in General Formula (G1) is limited to Si. As described above, such a limitation can reduce the GSP_slope of the evaporated film of the organic compound. Furthermore, the cost of synthesis can be low.

In each of the organic compounds represented by General Formula (G1) and General Formula (G2), R^{d} and R^{e} preferably represent bonds that are bonded to each other to form a ring. That is, each of the organic compounds preferably has a 9,9′-spirobifluorene ring. This can improve the rigidity of the molecule, so that the T_{g} of a film and powder of the organic compound can be further increased.

Accordingly, one embodiment of the present invention is an organic compound represented by General Formula (G3).

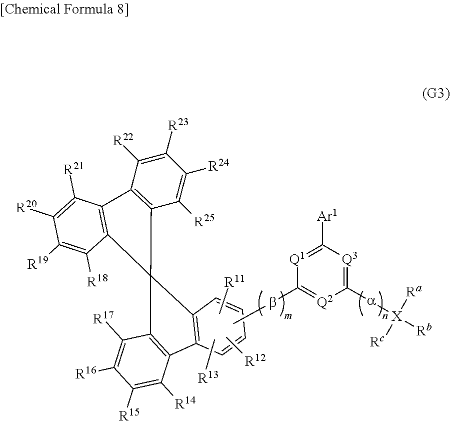

In General Formula (G3), each of Q¹, Q², and Q³ independently represents N or CH (including CD), and at least any two of Q¹, Q², and Q³ represent N; each of R^{a}, R^{b}, and R^{c} independently represents a cycloalkyl group having 3 to 10 carbon atoms or a substituted or unsubstituted aryl group having 6 to 12 carbon atoms; Ar¹ represents a substituted or unsubstituted aryl group having 6 to 30 carbon atoms; each of α and β independently represents a substituted or unsubstituted arylene group having 6 to 30 carbon atoms; each of R¹¹ to R²⁵ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹¹ to R²⁵ represents an alkyl group having 1 to 10 carbon atoms; and each of n and m independently represents an integer greater than or equal to 0 and less than or equal to 3.

The organic compound represented by General Formula (G3) is different from the organic compound represented by General Formula (G2) in that R^{d} and R^{e} in General Formula (G2) are limited to a bond that is bonded to each other to form a ring. As described above, such a limitation can further increase the T_{g} of a film and powder of the organic compound.

In each of the organic compounds represented by General Formula (G1) to General Formula (G3), it is preferable that each of α and β independently represent a substituted or unsubstituted arylene group having 6 to 10 carbon atoms, in which case raw materials are easy to obtain.

In each of the organic compounds represented by General Formula (G1) to General Formula (G3), it is preferable that each of n and m independently represent an integer greater than or equal to 0 and less than or equal to 2. This enables both the degree of the electron-transport property and the degree of the T_{g} of a film and powder of the organic compound to be adjustable, so that a device having desired characteristics can be fabricated.

Another embodiment of the present invention is an organic compound represented by General Formula (G4).

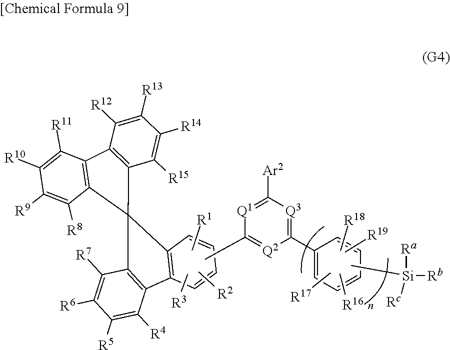

In General Formula (G4), each of Q¹, Q², and Q³ independently represents N or CH (including CD), and at least any two of Q, Q², and Q³ represent N; each of R^{a}, R^{b}, and R^{c} independently represents an alkyl group having 1 to 4 carbon atoms, a cycloalkyl group having 3 to 10 carbon atoms, or a substituted or unsubstituted aryl group having 6 to 12 carbon atoms; Ar² represents a substituted or unsubstituted aryl group having 6 to 10 carbon atoms; each of R¹ to R¹⁹ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; and n is an integer greater than or equal to 0 and less than or equal to 3.

The organic compound represented by General Formula (G4) includes an electron-transport skeleton having a pyrimidine ring or a triazine ring, which is a it-electron deficient heteroaromatic ring. The organic compound represented by General Formula (G4) includes a trisubstituted silyl group that are each bonded to the electron-transport skeleton directly or through a linker. The organic compound represented by General Formula (G4) includes a 9,9′-spirobifluorene ring that is bonded to the electron-transport skeleton directly or through a linker. Thus, the organic compound represented by General Formula (G4) has a high electron-transport property. The organic compound represented by General Formula (G4) has a low refractive index of a film. The organic compound represented by General Formula (G4) has a small GSP_slope of an evaporated film. The organic compound represented by General Formula (G4) has high T_{g} of a film and powder.

Here, in order to examine the effects of introducing a trisubstituted silyl group into an organic compound, the permanent electric dipole moments of the stable structures in the singlet ground states of trimethylsilylbenzene, which is an organic compound including a trisubstituted silyl group, and tert-butylbenzene, which is a comparative organic compound, were analyzed and compared, and the result of the analysis and comparison will be described. Since a trimethylsilyl group and a tert-butyl group include saturated hydrocarbon groups, introduction of a trimethylsilyl group or a tert-butyl group into an aromatic ring of an organic compound is expected to reduce the refractive index of the film of the organic compound.

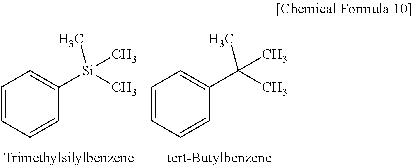

A density functional theory (DFT) method was used as the calculation method. As a functional, B3LYP was used, and as a basis function, 6-311G(d,p) was used. Gaussian 16 was used as a computational program.

As a result of the calculation, the magnitude of the permanent electric dipole moment of tert-butylbenzene is 0.3110 Debye. This permanent electric dipole moment results from donation of an electron to the benzene ring from a tert-butyl group, which is an electron-donating group. By contrast, the magnitude of the permanent electric dipole moment of trimethylsilylbenzene is 0.0345 Debye, which is less than that of the tert-butylbenzene and close to zero. This indicates that the trimethylsilyl group hardly exhibits an electron-donating property with respect to the benzene ring. Therefore, it can be said that a trisubstituted silyl group has a poorer electron-donating property and is accordingly more effective in reducing the permanent electric dipole moment of a molecule than a group having a structure in which the same three substituents as those in the trisubstituted silyl group are bonded to a carbon atom.

By this effect, a molecule for an organic semiconductor that includes a trisubstituted silyl group is capable of maintaining a small permanent electric dipole moment. Accordingly, such a molecule can be formed into a film with a small SOP by a vacuum evaporation method. Thus, like the organic compound represented by General Formula (G4), an organic compound into which a trisubstituted silyl group is introduced can have a smaller GSP_slope of an evaporated film than an organic compound into which a trisubstituted methyl group is introduced.

In the organic compound represented by General Formula (G4), it is preferable that Ar² be a substituted or unsubstituted phenyl group. With Ar² being a phenyl group, the chemical reactivity of the electron-transport skeleton can be lowered, which can contribute to inhibiting the deterioration.

In the organic compound represented by General Formula (G4), n is preferably greater than or equal to 1 and less than or equal to 3, further preferably 1. An arylene group with an appropriate size is preferably present between the electron-transport skeleton and the trisubstituted silyl group, in which case the organic compound can have a high electron-transport property. Furthermore, when being provided with the organic compound of one embodiment of the present invention, the light-emitting device is expected to have improved light extraction efficiency due to the low refractive index of the film of the organic compound. Thus, n is further preferably 1. When n is greater than or equal to 2, the n phenylene groups are preferably 1,3-substitution phenylene rather than 1,4-substitution phenylene, in which case the refractive index of a film can be reduced and the T_{g} of a film and powder can be sufficiently increased for the usage environment.

Accordingly, one embodiment of the present invention is an organic compound represented by General Formula (G5).

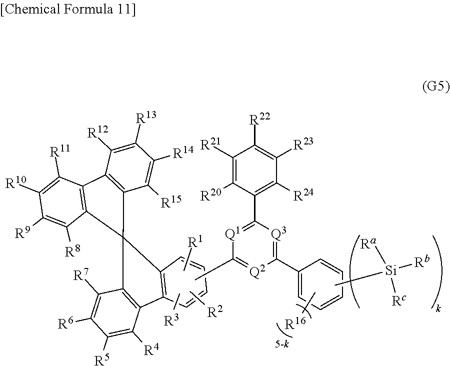

In General Formula (G5), each of Q¹, Q², and Q³ independently represents N or CH (including CD), and at least any two of Q¹, Q², and Q³ represent N; each of R^{a}, R^{b}, and R^{c} independently represents an alkyl group having 1 to 4 carbon atoms, a cycloalkyl group having 3 to 10 carbon atoms, or a substituted or unsubstituted aryl group having 6 to 12 carbon atoms; each of R¹ to R²⁴ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; and k is an integer greater than or equal to 1 and less than or equal to 5. In the case where k is greater than or equal to 2, the plurality of R^{a}s may be the same or different from each other, the plurality of R^{b}s may be the same or different from each other, and the plurality of R^{c}s may be the same or different from each other. In the case where 5-k is greater than or equal to 2, that is, k is greater than or equal to 1 and less than or equal to 3, the plurality of R¹⁶s may be the same or different from each other.

An organic compound represented by General Formula (G5) is different from the organic compound represented by General Formula (G4) in that Ar² in General Formula (G4) is limited to a substituted or unsubstituted phenyl group and n in General Formula (G4) is limited to 1. As described above, such a limitation can further increase the electron-transport property of the organic compound.

In each of the organic compounds represented by General Formula (G4) and General Formula (G5), it is preferable that k be greater than or equal to 2 when each of R^{a}, R^{b} and R^{c} independently represents an alkyl group having 1 to 4 carbon atoms. This can further reduce the refractive index of the film of the organic compound and the GSP_slope of the evaporated film of the organic compound.

Another embodiment of the present invention is the organic compound represented by General Formula (G6).

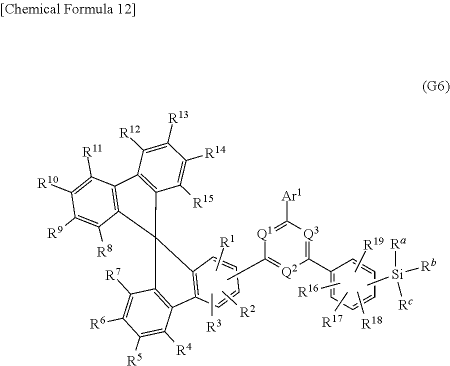

In General Formula (G6), each of Q¹, Q², and Q³ independently represents N or CH (including CD), and at least any two of Q¹, Q², and Q³ represent N; each of R^{a}, R^{b}, and R^{c} independently represents a cycloalkyl group having 3 to 10 carbon atoms or a substituted or unsubstituted aryl group having 6 to 12 carbon atoms; Ar¹ represents a substituted or unsubstituted aryl group having 6 to 30 carbon atoms; and each of R¹ to R¹⁹ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms.

The organic compound represented by General Formula (G6) includes an electron-transport skeleton having a pyrimidine ring or a triazine ring, which is a π-electron deficient heteroaromatic ring. The organic compound represented by General Formula (G6) includes a trisubstituted silyl group that are each bonded to the electron-transport skeleton directly or through a linker. The organic compound represented by General Formula (G6) includes a 9,9′-spirobifluorene ring that is bonded to the electron-transport skeleton directly or through a linker. Thus, the film formed using the organic compound represented by General Formula (G6) can have both a high electron-transport property and a low refractive index of a film. The organic compound represented by General Formula (G6) has a small GSP_slope of an evaporated film. The organic compound represented by General Formula (G6) has high T_{g} of a film and powder. The 9,9′-spirobifluorene ring of the organic compound represented by General Formula (G6) includes an alkyl group having 1 to 10 carbon atoms. This can intensify the effect of increasing the T_{g} of a film and powder of the organic compound.

Another embodiment of the present invention is an organic compound with the above structure in which Ar¹ preferably represents a substituted or unsubstituted aryl group having 6 to 10 carbon atoms, further preferably represents a substituted or unsubstituted phenyl group. With Ar¹ being a phenyl group, the chemical reactivity of the electron-transport skeleton can be lowered, which can contribute to inhibiting the deterioration.

Another embodiment of the present invention is an organic compound with the above structure in which each of R^{a}, R^{b}, and R^{c} independently preferably represents a substituted or unsubstituted aryl group having 6 to 12 carbon atoms, further preferably represents a substituted or unsubstituted phenyl group. This enables the evaporated film formed using the organic compound to have a small GSP_slope and high T_{g}.

Accordingly, one embodiment of the present invention is an organic compound represented by General Formula (G7).

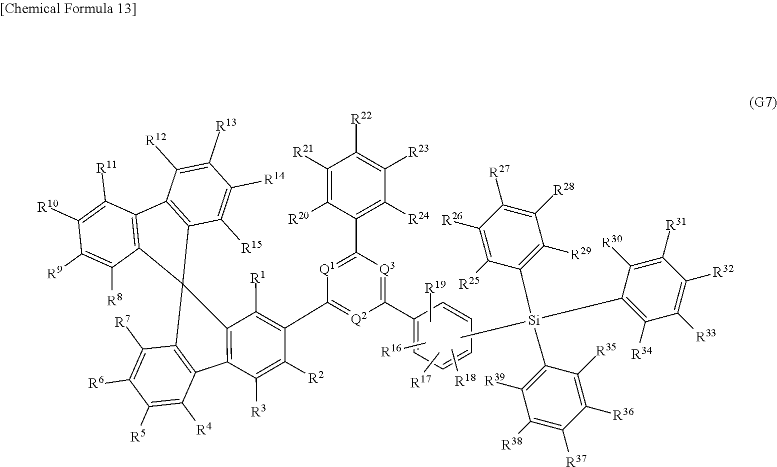

In General Formula (G7), each of Q¹, Q², and Q³ independently represents N or CHI (including CD), and at least any two of Q, Q², and Q³ represent N; each of R¹ to R²⁴ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms; and each of R²⁵ to R³⁹ independently represents hydrogen (including deuterium) or an alkyl group having 1 to 10 carbon atoms.

An organic compound represented by General Formula (G7) is different from the organic compound represented by General Formula (G6) in that Ar¹ in General Formula (G6) is limited to a substituted or unsubstituted phenyl group and each of R^{a}, R^{b} and R^{c} in General Formula (G6) is limited to a substituted or unsubstituted phenyl group. By such a limitation, the chemical reactivity of the electron-transport skeleton can be lowered, which can contribute to inhibiting the deterioration. Furthermore, the film formed using the organic compound can have a small GSP_slope and high T_{g}.

In the organic compound represented by any of the general formulae above, each of Q¹ to Q³ preferably represents N. This can lower the LUMO level which determines the electron-transport property of the organic compound, so that the electron-transport property can be further increased.

Next, specific examples of substituents that can be used for the organic compounds represented by the general formulae above will be described. Note that groups that can be used in the general formulae above are not limited to the following specific examples. In addition, in the specific examples described below, some or all of hydrogen atoms may be deuterium.

### <<Alkyl Group Having 1 to 10 Carbon Atoms>>

An alkyl group having 1 to 10 carbon atoms is a monovalent group obtained by removing one hydrogen from an alkane having 1 to 10 carbon atoms. Specific examples include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, and a 2,3-dimethylbutyl group. Among the above-described alkyl groups, a methyl group is preferably used because there is less steric hindrance due to the proximity of the substituents (alkyl groups), contributing to the higher stability of the molecule. A methyl group is preferably used also because it is sterically small among alkyl groups and therefore less likely to impede the carrier-transport property. Among the above-described alkyl groups, using the alkyl group having two or more carbon atoms can further reduce the refractive index of the film of the organic compound. In order to further reduce the refractive index of a film, using a branched-chain alkyl group is preferable. In other words, by selecting an alkyl group to be used, the balance between the refractive index and the electron-transport property can be adjusted.

### <<Cycloalkyl Group Having 3 to 10 Carbon Atoms>>

A cycloalkyl group having 3 to 10 carbon atoms is a monovalent group obtained by removing one hydrogen from a monocyclic or polycyclic cycloalkane having 3 to 10 carbon atoms. Specific examples include a cyclopropyl group, a cyclobutyl group, a cyclopentyl group, a cyclohexyl group, a 4-methylcyclohexyl group, a cycloheptyl group, a cyclooctyl group, a cyclononyl group, a cyclodecyl group, a norbornyl group, a bicyclo[2,2,2]octyl group, a decahydronaphthyl group, and an adamantyl group. Among the cycloalkyl groups, a cycloalkyl group having 6 or more carbon atoms is preferably used, in which case the refractive index of the film of the organic compound can be lower and the glass transition temperature (T_{g}) of the organic compound can be higher than in the case where a cycloalkyl group having 5 or less carbon atoms is used. In particular, a cyclohexyl group is preferable because it is chemically stable and easily manufactured at low cost.

### <<Aryl Group Having 6 to 30 Carbon Atoms and Aryl Group Having 6 to 12 Carbon Atoms>>

An aryl group having 6 to 30 carbon atoms is a monovalent group obtained by removing one hydrogen from one of carbon atoms forming a ring of a monocyclic or polycyclic aromatic compound having 6 to 30 carbon atoms. Specific examples include a phenyl group, a biphenyl-2-yl group (o-biphenyl group), a biphenyl-3-yl group (m-biphenyl group), a biphenyl-4-yl group (p-biphenyl group), a 1-naphthyl group, a 2-naphthyl group, a phenylnaphthyl group, a naphthylphenyl group, a terphenyl group, a fluorenyl group, a 9,9-dimethylfluorenyl group, a quaterphenyl group, a spirobifluorenyl group, a phenanthryl group, an anthryl group, a binaphthylphenyl group, and a fluoranthenyl group. Among the above-described aryl groups having 6 to 30 carbon atoms, any of aryl groups having 6 to 12 carbon atoms can be selected and used as the aryl group having 6 to 12 carbon atoms. In the case where the aryl group having 6 to 30 carbon atoms or the aryl group having 6 to 12 carbon atoms includes a substituent, specific examples of the substituent include an alkyl group having 1 to 6 carbon atoms, a cycloalkyl group having 3 to 10 carbon atoms, and a phenyl group. Among aryl groups, an aromatic compound composed of a six-membered ring is preferable because of its high stability and high reliability when being used for a light-emitting device. An aromatic ring compound having a large number of carbon atoms can improve the electron-transport property of the organic compound.

### <<Heteroaryl Group Having 3 to 30 Carbon Atoms>>

A heteroaryl group having 3 to 30 carbon atoms is a monovalent group obtained by removing one hydrogen from one of carbon atoms forming a ring of a monocyclic or polycyclic heterocyclic aromatic compound having 3 to 30 carbon atoms. Specific examples include a carbazolyl group, a dibenzothiophenyl group, a dibenzofuranyl group, a benzocarbazolyl group, a naphthobenzothiophenyl group, a naphthobenzofuranyl group, a dibenzocarbazolyl group, a dinaphthothiophenyl group, a dinaphthofuranyl group, a triazinyl group, a pyrimidinyl group, a pyrazinyl group, a triazolyl group, a pyridinyl group, a benzofuropyrimidinyl group, a benzothiopyrimidinyl group, a benzofuropyrazinyl group, a benzothiopyrazinyl group, a benzofuropyridinyl group, a benzothiopyridinyl group, and a bicarbazolyl group. In the case where the heteroaryl group having 3 to 30 carbon atoms includes a substituent, examples of the substituent include an alkyl group having 1 to 6 carbon atoms, a cycloalkyl group having 3 to 10 carbon atoms, and a phenyl group. Among heteroaryl groups, a heteroaryl group having a six-membered ring is preferable because of its high stability and high reliability. A heteroaryl group having two or more nitrogen is preferable because it improves the electron-transport property. A heteroaryl group having one nitrogen is preferable because it reduces the refractive index of a film. A heteroaromatic ring compound having a large number of carbon atoms can improve the electron-transport property of the organic compound.

### <<Trisubstituted Silyl Group Having 3 to 36 Carbon Atoms>>

The trisubstituted silyl group having 3 to 36 carbon atoms is a group having a structure in which three substituents having 3 to 36 carbon atoms in total are bonded to silicon (Si). Examples of the substituent include an alkyl group and an aryl group. Specific examples of the trisubstituted silyl group having 3 to 36 carbon atoms include a trimethylsilyl group, a triethylsilyl group, a tert-butyldimethylsilyl group, and a triphenylsilyl group. A trialkylsilyl group having 3 to 18 carbon atoms, such as a trimethylsilyl group, a triethylsilyl group, or a tert-butyl dimethylsilyl group, is preferable because it can further reduce the refractive index of the film formed using the organic compound. A triarylsilyl group having 18 to 36 carbon atoms, such as a triphenylsilyl group, is preferable because it can increase the T_{g} of the film formed using the organic compound. The triarylsilyl group may further include a substituent such as an alkyl group. Part of the three substituents of the trisubstituted silyl group having 3 to 36 carbon atoms can be an alkyl group and another part can be an aryl group. For example, reagents and the like for introducing diarylmonoalkylsilyl groups are commercially available and are easy to obtain. Since having substituents freely set as described above, a trisubstituted silyl group is a highly versatile group whose substituents can be selected appropriately in consideration of the refractive index and T_{g} of the film.

### <<Trisubstituted Methyl Group Having 4 to 37 Carbon Atoms>>

The trisubstituted methyl group having 4 to 37 carbon atoms is a group having a structure in which three substituents having 3 to 36 carbon atoms in total are bonded to carbon (C). Examples of the substituent include an alkyl group and an aryl group. The aryl group may include a substituent. Specific examples of the trisubstituted methyl group having 4 to 37 carbon atoms include a triphenylmethyl group, a bis(4-fluorophenyl)(phenyl)methyl group, a tris(4-fluorophenyl)methyl group, a tris(4-methylphenyl)methyl group, and a tris(4-methoxyphenyl)methyl group. A triarylmethyl group having 18 to 36 carbon atoms, such as a triphenylmethyl group, is preferable because it can increase the T_{g} of the film formed using the organic compound. The triarylmethyl group may further include a substituent such as an alkyl group. A triphenylmethyl group is a substituent generally used as a protecting group in a chemical reaction and reagents are easy to obtain. It is also easy to obtain reagents in which a variety of substituents are introduced into a phenyl group.

The above substituents are specific examples of the substituent that can be used for the organic compounds represented by the general formulae.

Specific examples of the organic compounds of embodiments of the present invention represented by the general formulae above include organic compounds represented by Structural Formula (100) to Structural Formula (151) below. Note that the organic compound of one embodiment of the present invention is not limited to the organic compounds represented by the following structural formulae.

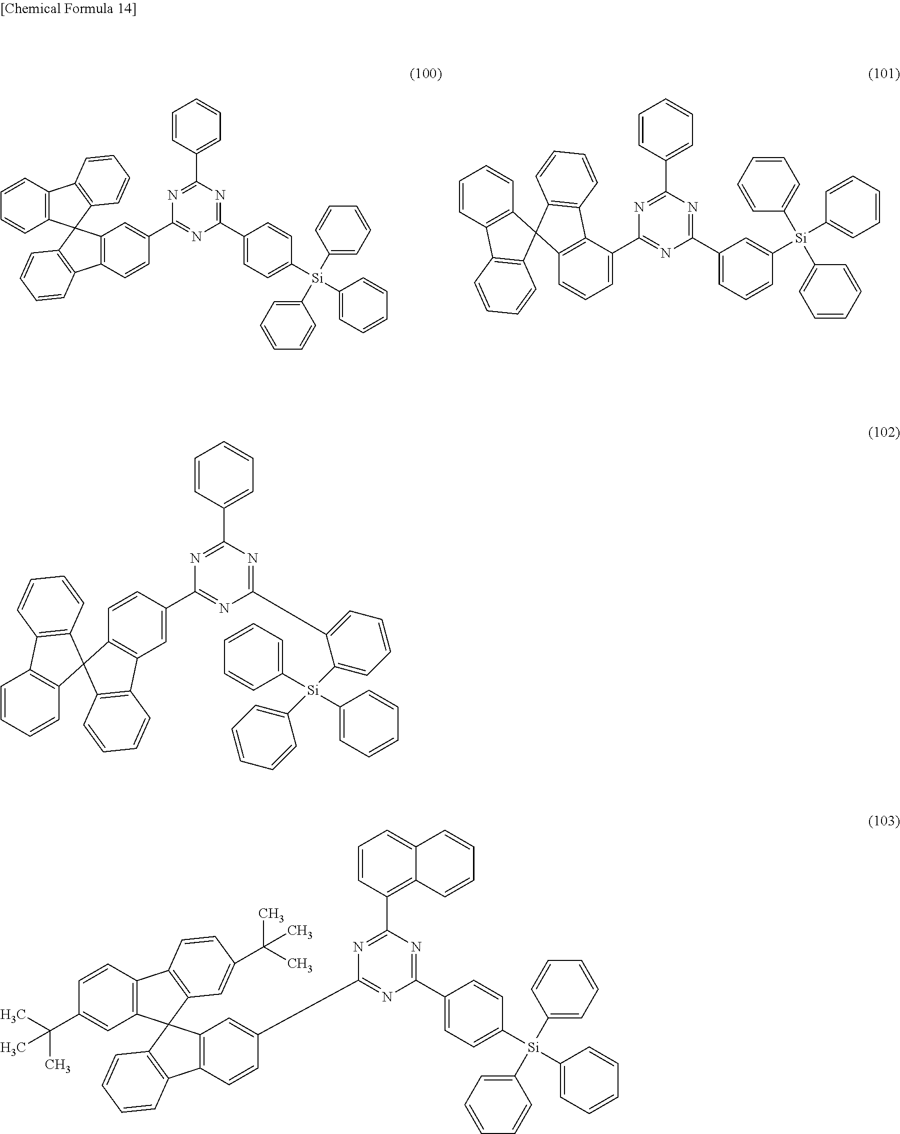

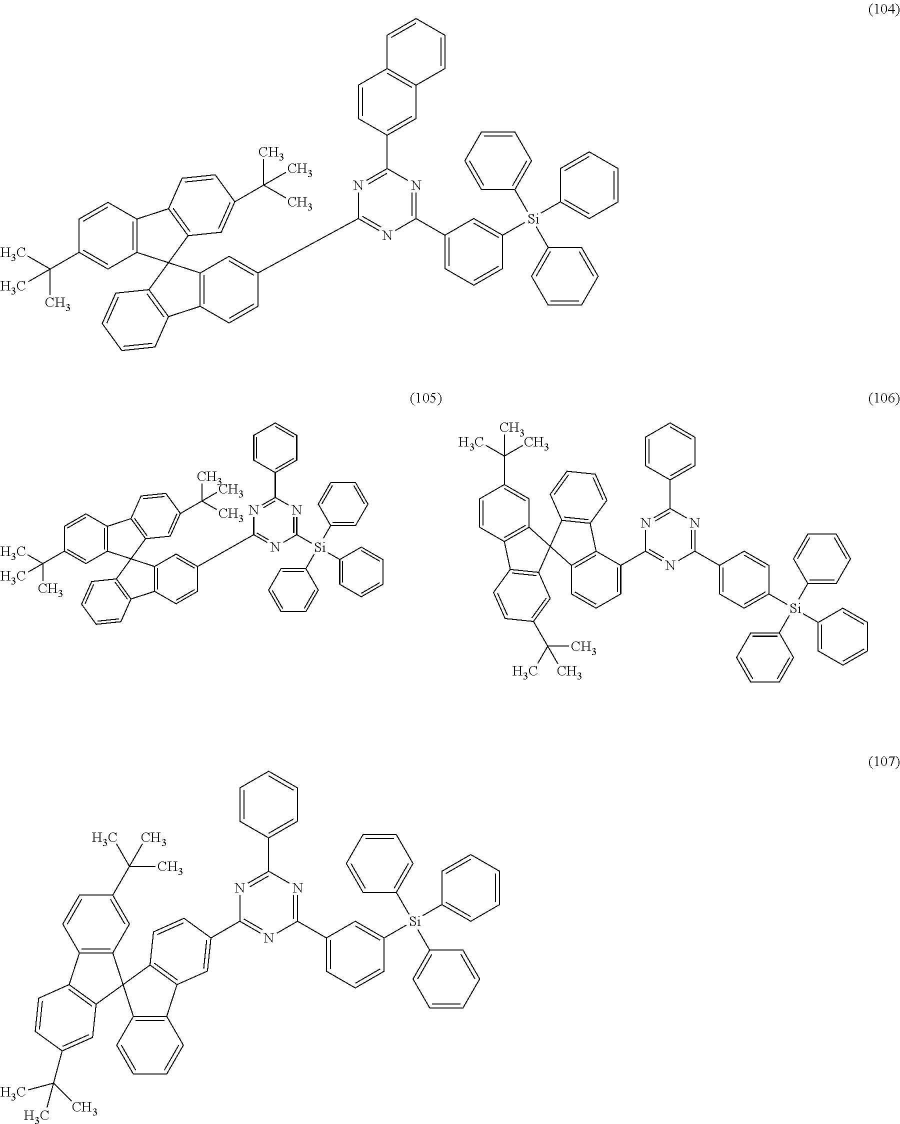

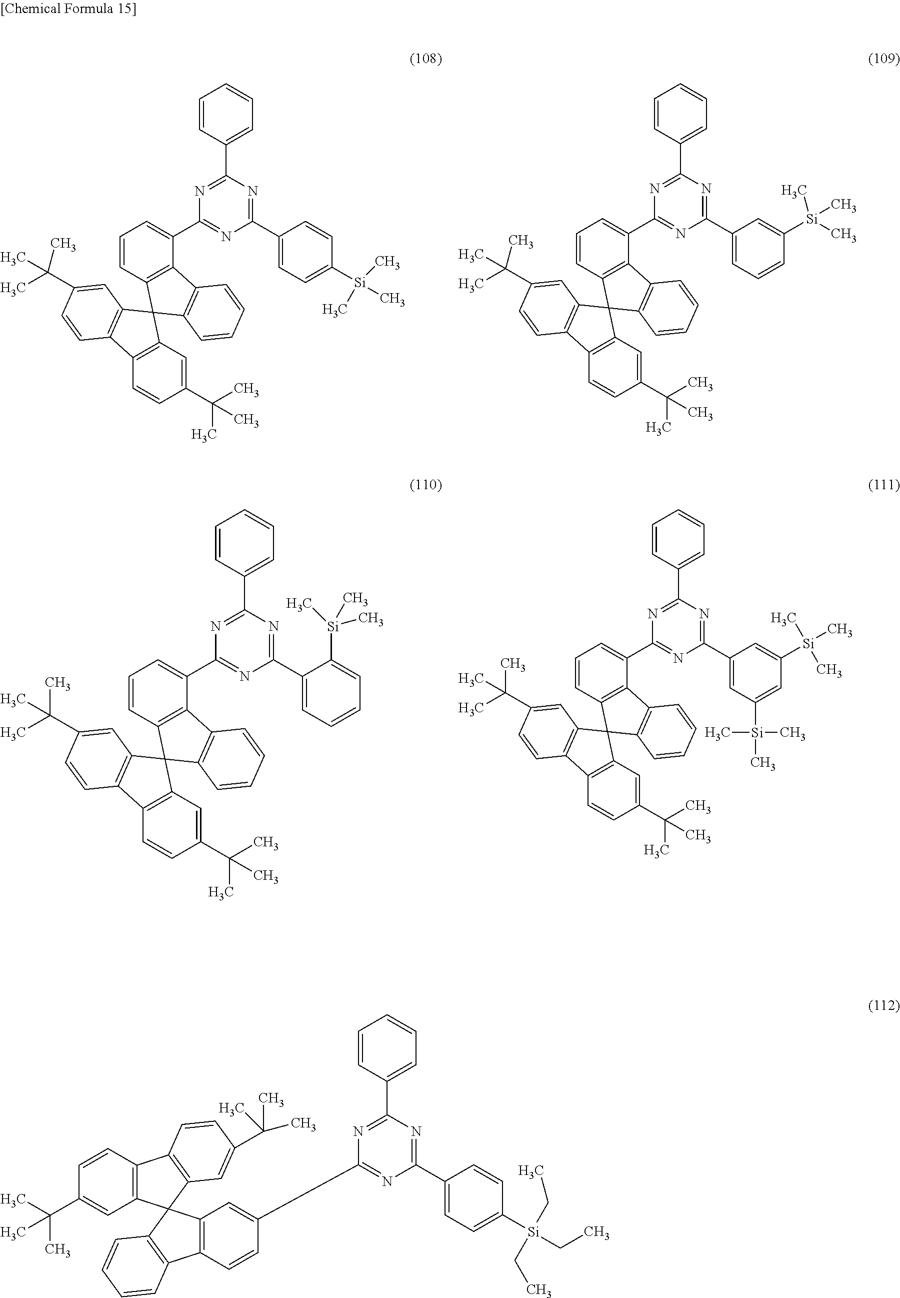

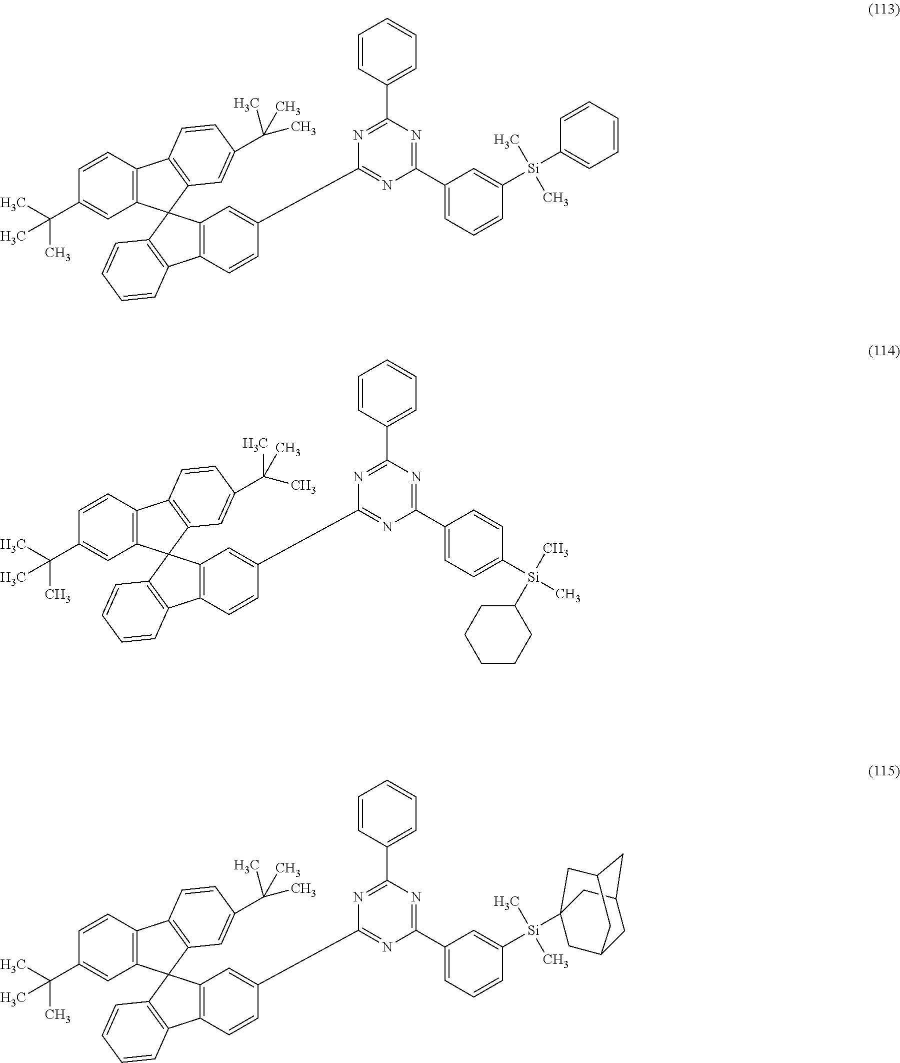

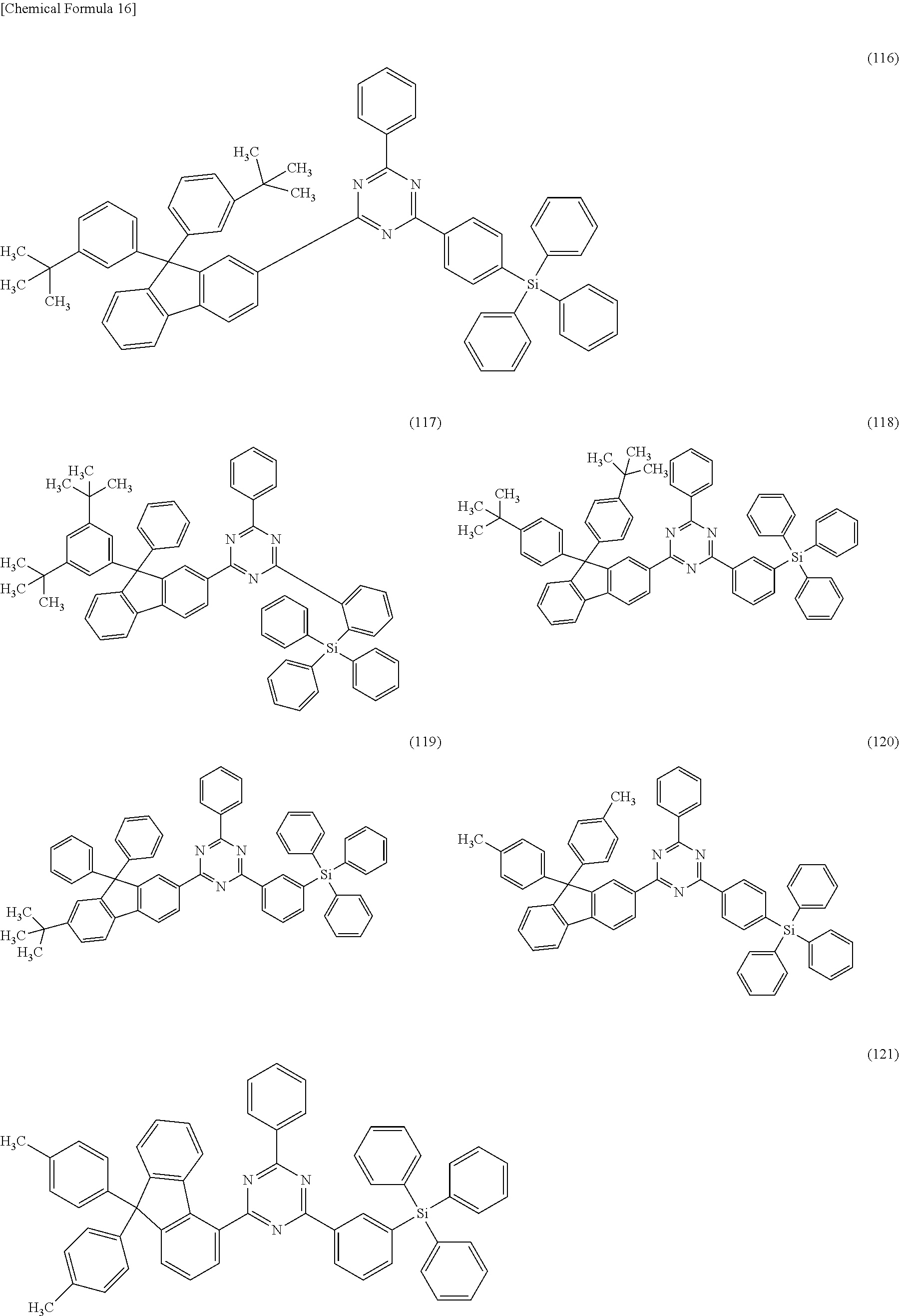

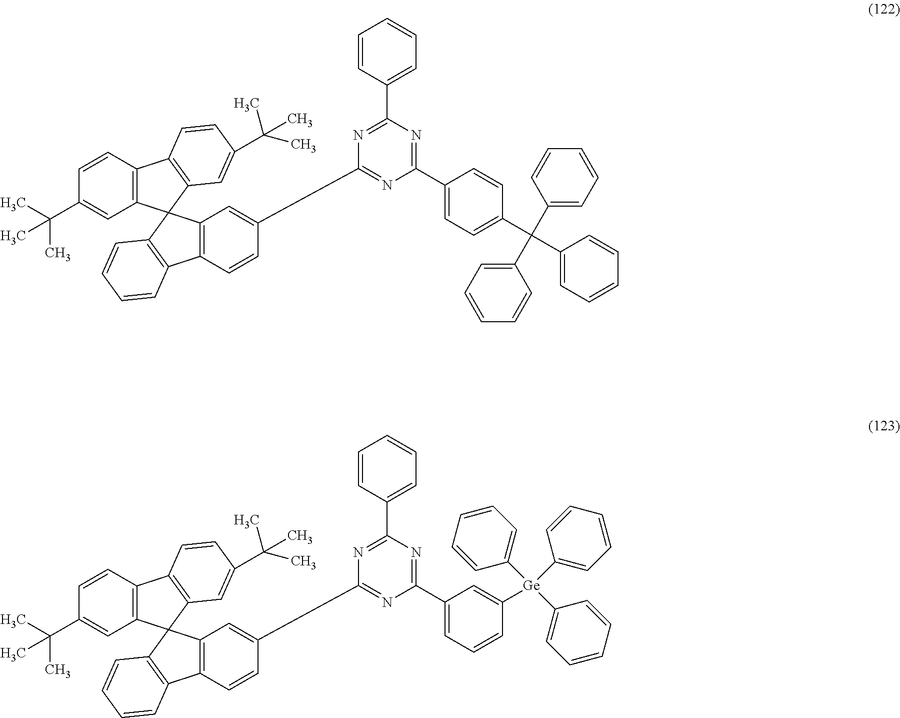

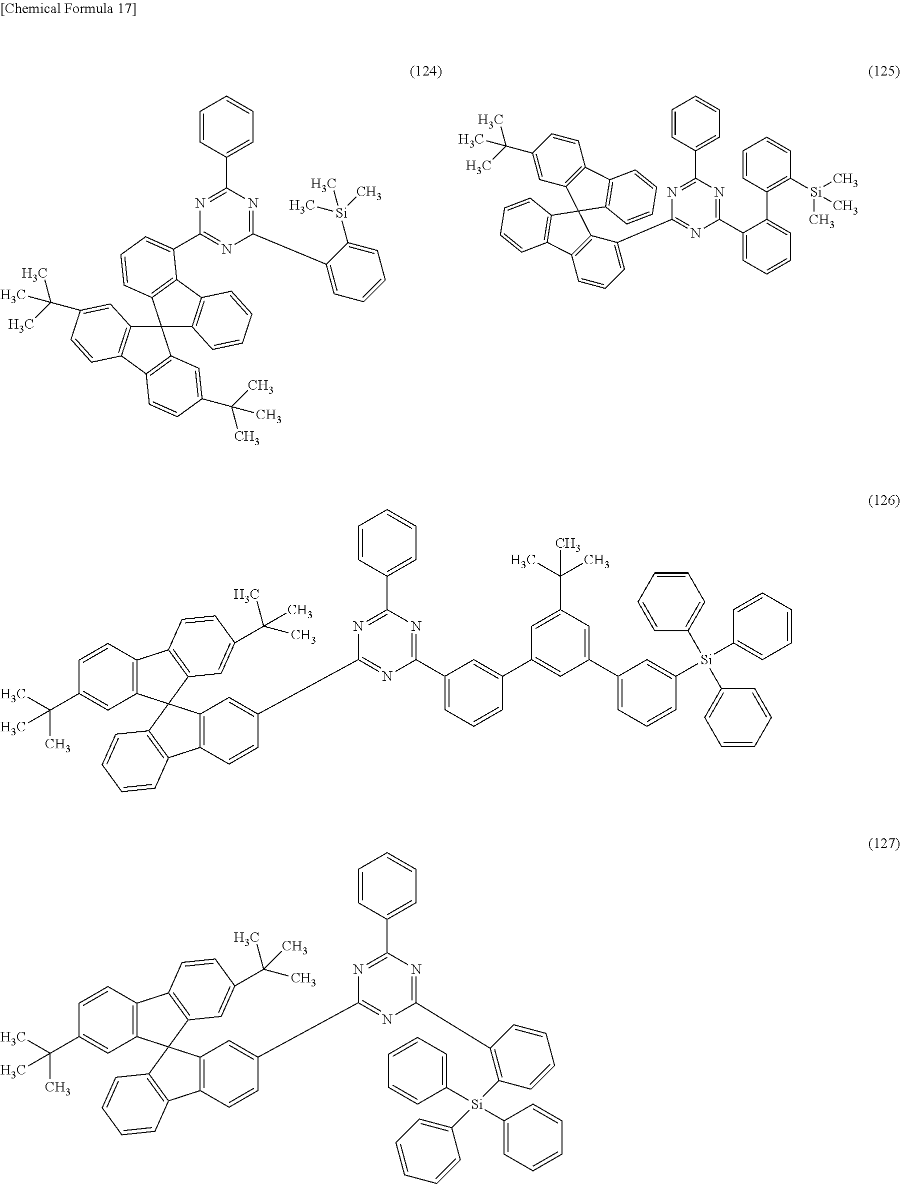

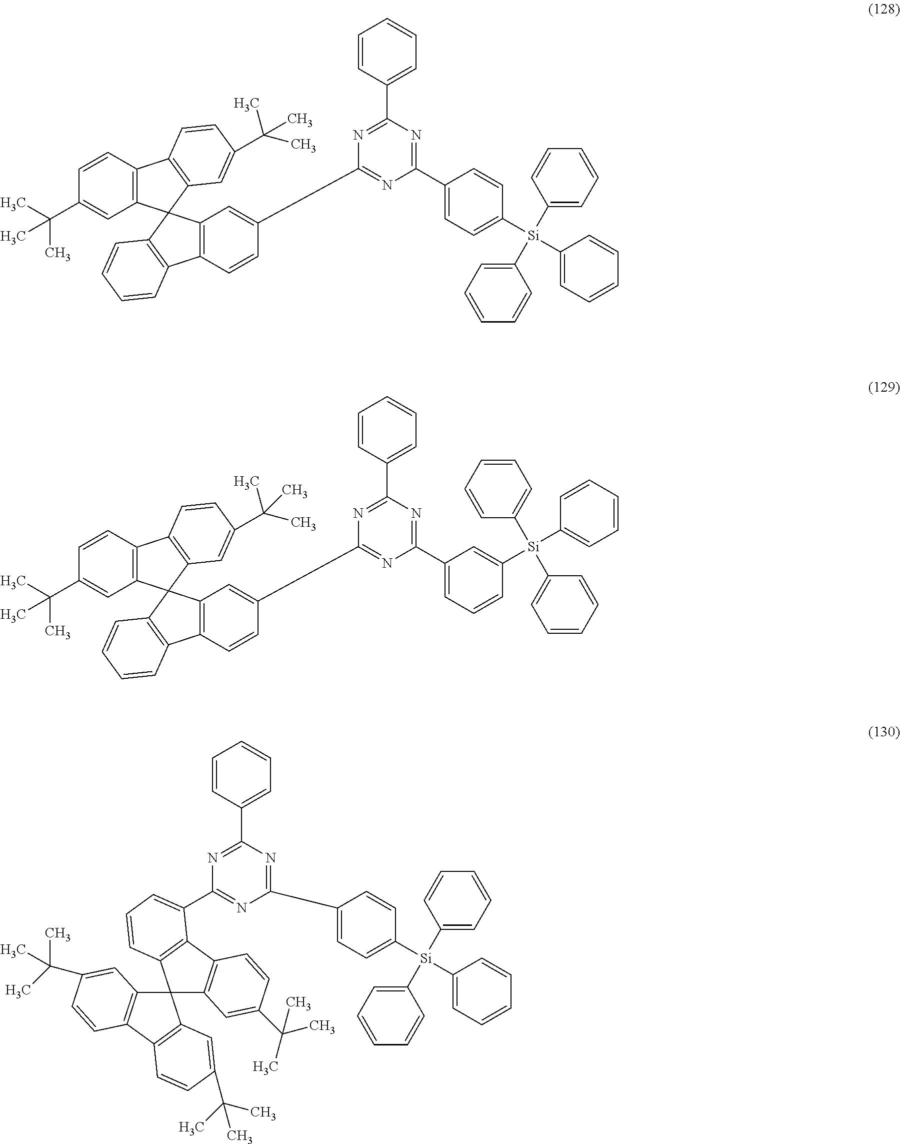

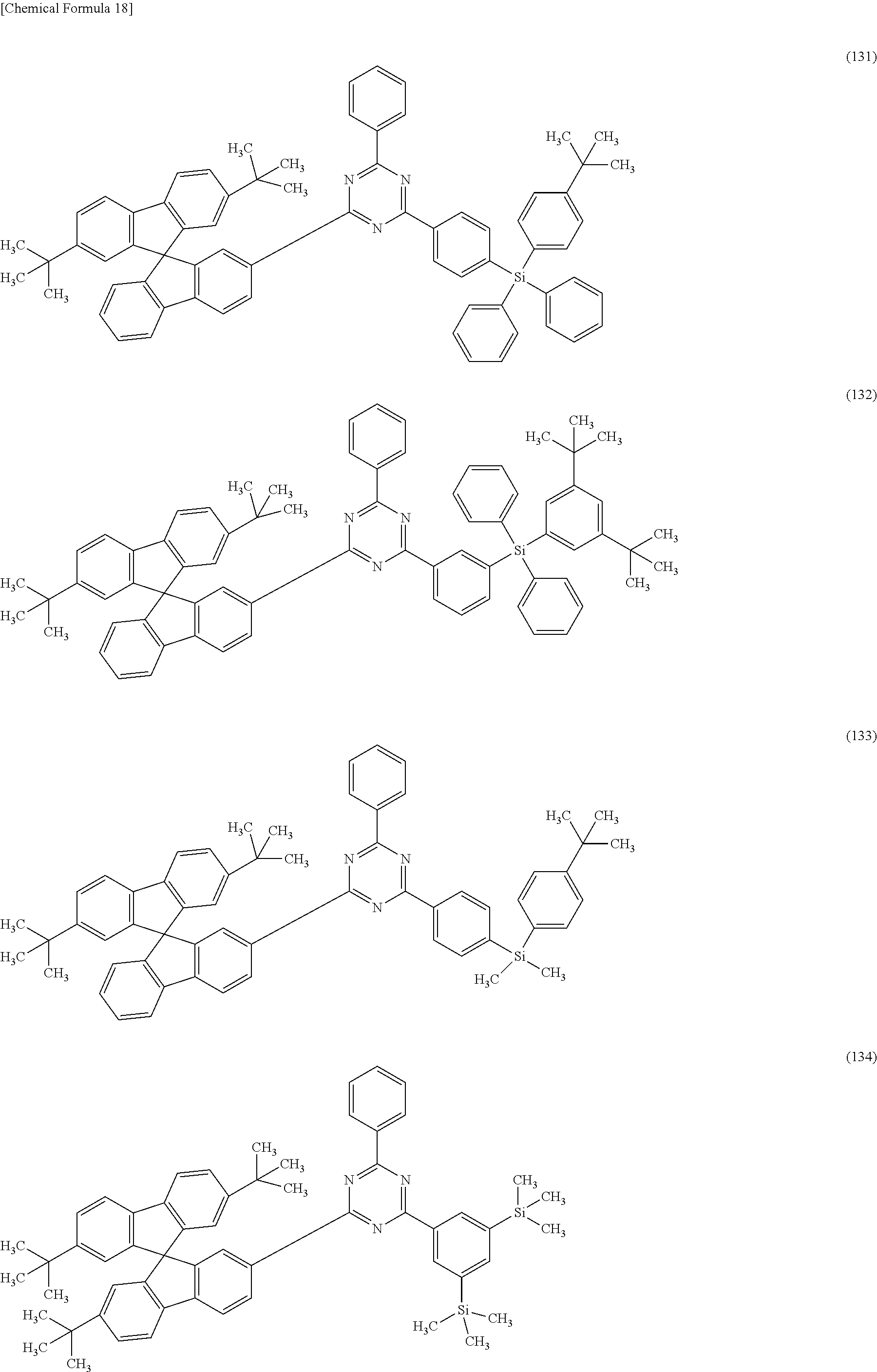

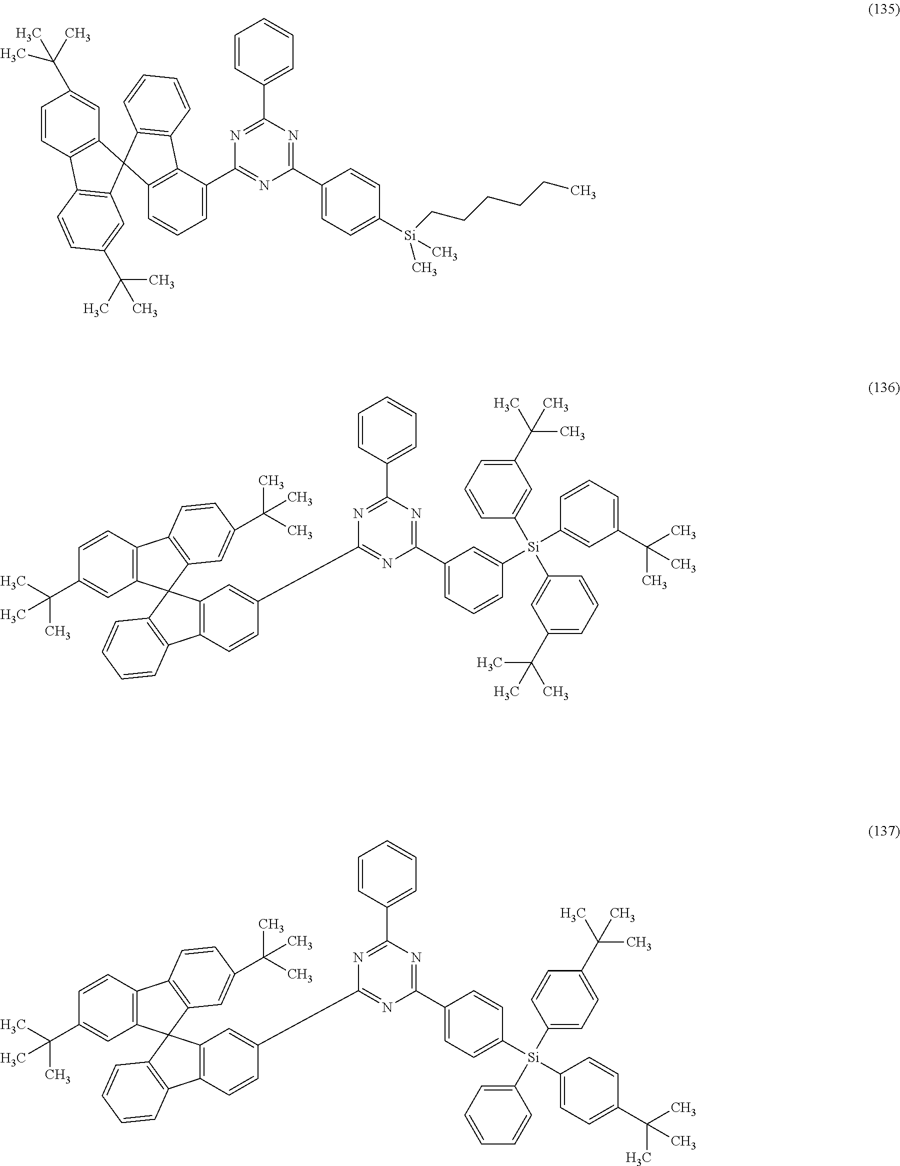

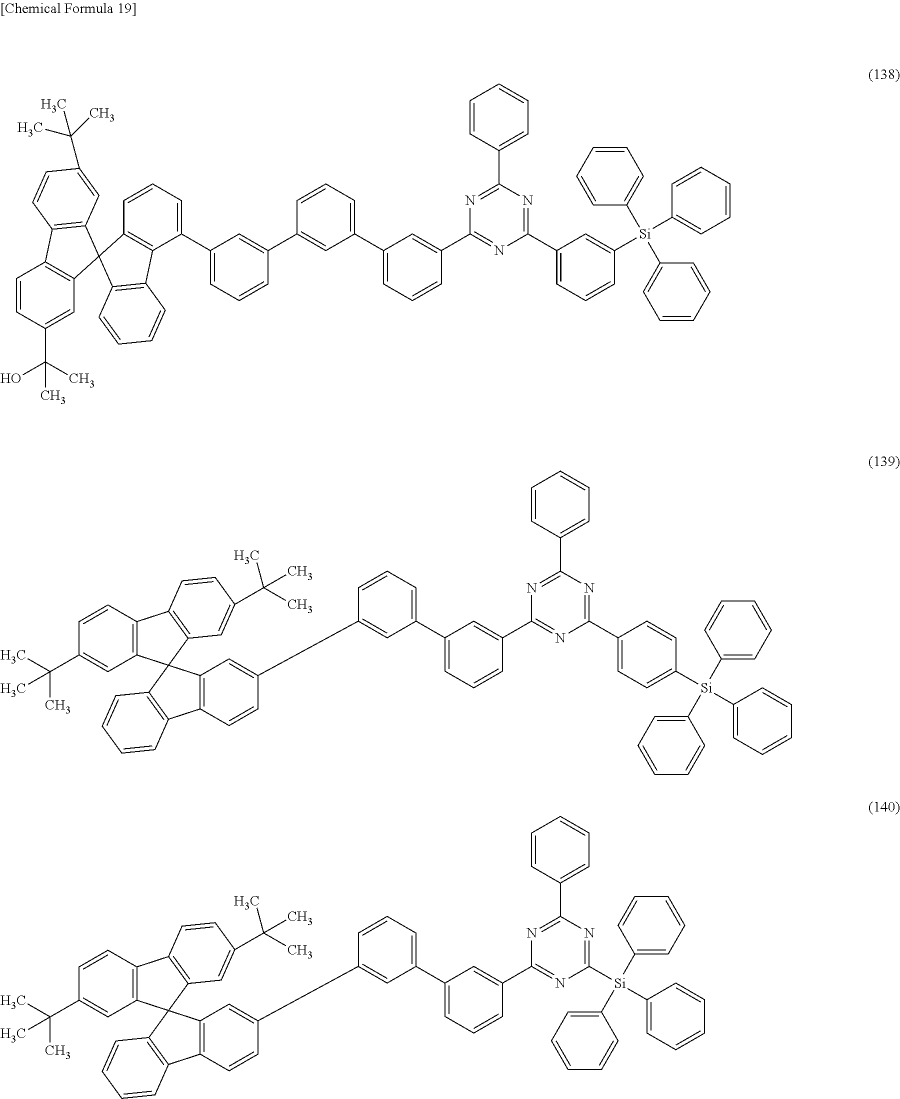

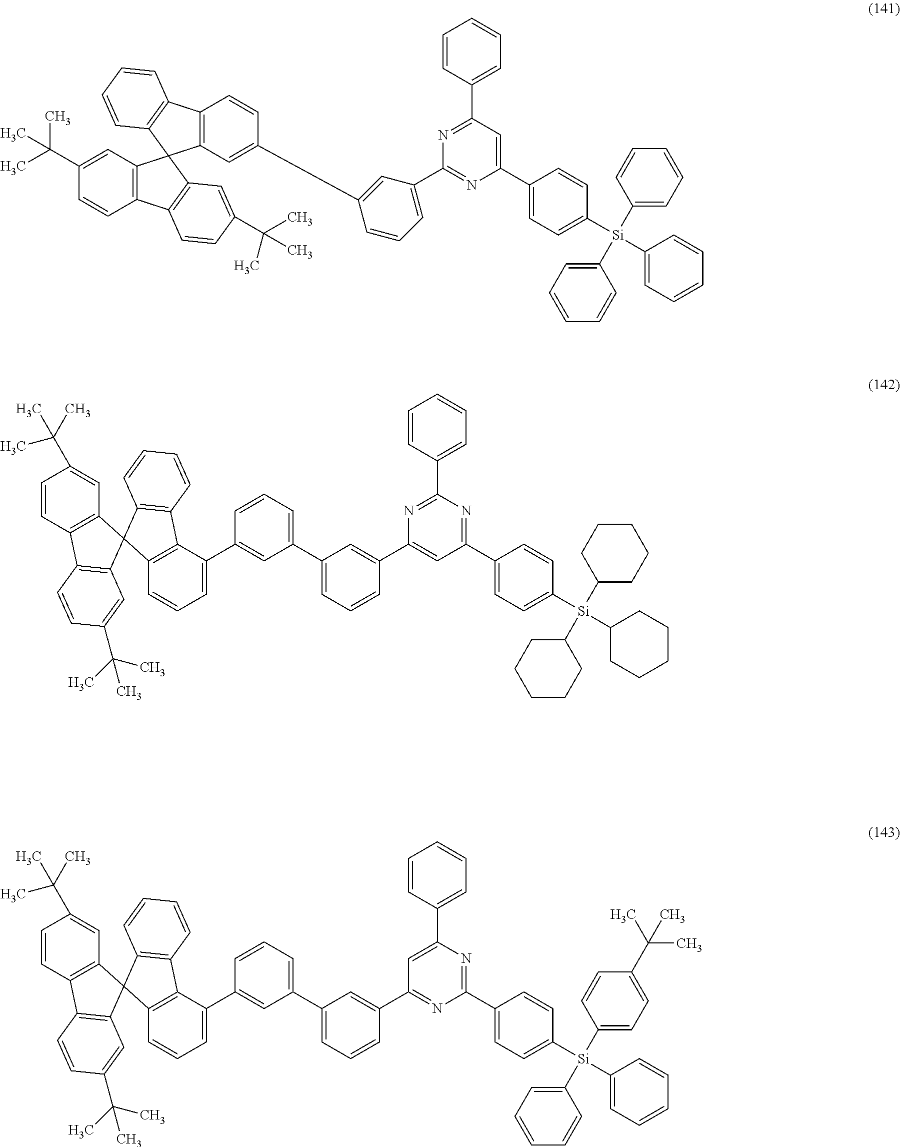

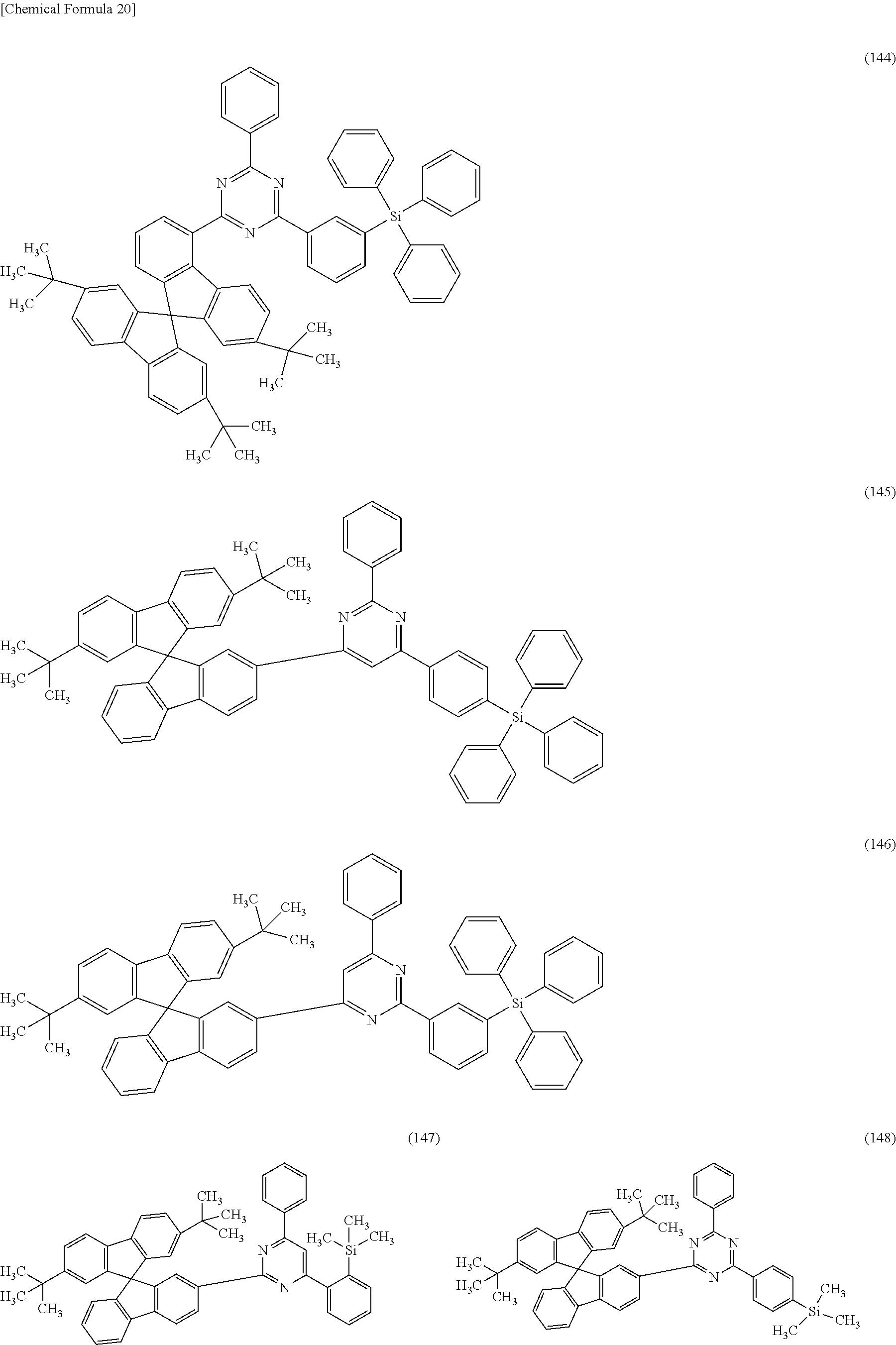

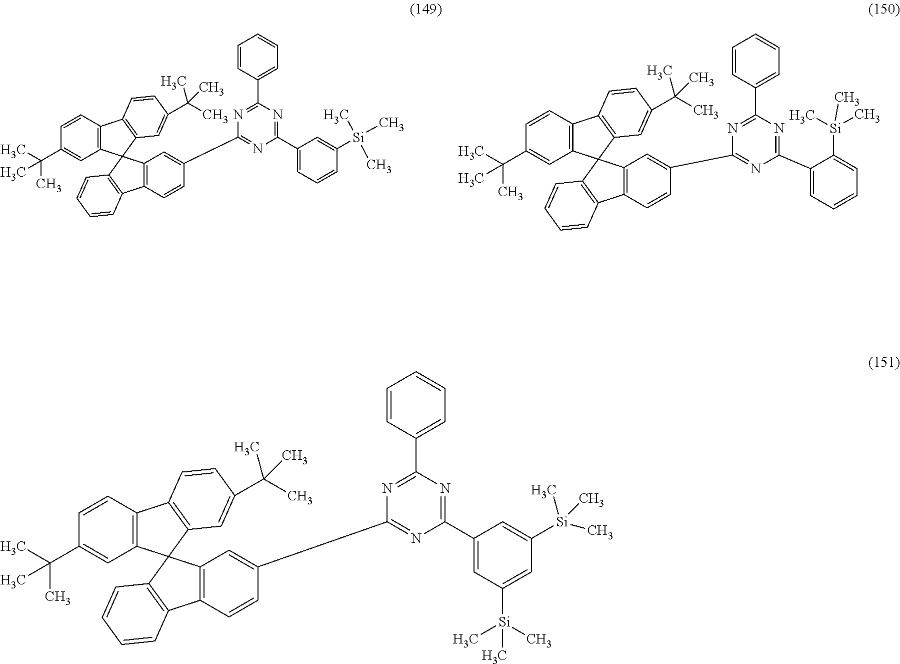

Next, as an example of a method of synthesizing the organic compound of one embodiment of the present invention, methods of synthesizing the organic compounds represented by General Formula (G1) and General Formula (G4) are described.

The organic compound represented by General Formula (G1) above can be obtained by reacting a halogen compound (B1) with a boronic acid compound (B2), as shown in Synthesis Scheme (A-1) below.

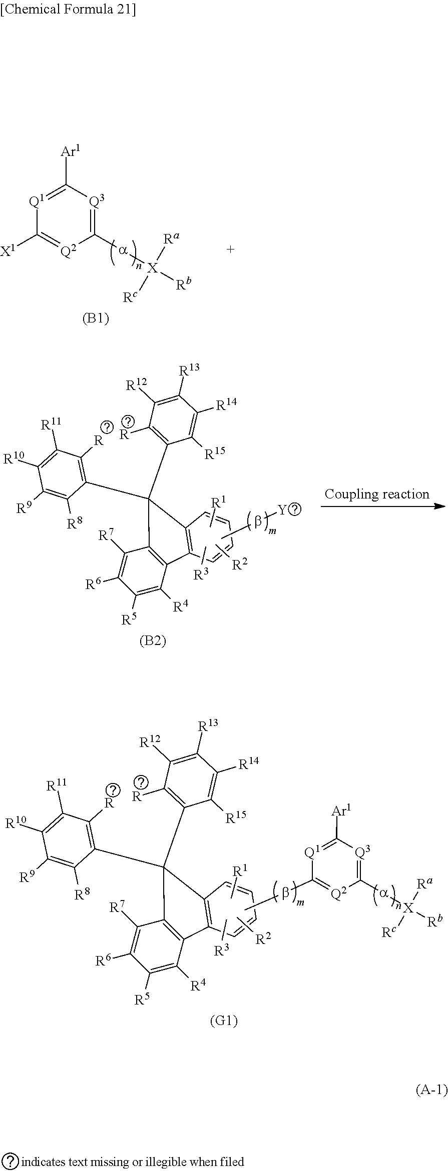

In Synthesis Scheme (A-1) above, X¹ represents a halogen. Each of Q¹, Q², and Q³ independently represents N or CH (including CD), and at least any two of Q¹, Q², and Q³ represent N; X represents C, Si, or Ge; each of R^{a}, R^{b}, and R^{c} independently represents a cycloalkyl group having 3 to 10 carbon atoms or a substituted or unsubstituted aryl group having 6 to 12 carbon atoms; Ar¹ represents a substituted or unsubstituted aryl group having 6 to 30 carbon atoms; each of α and β independently represents a substituted or unsubstituted arylene group having 6 to 30 carbon atoms; and R^{d} and R^{e} represent bonds that are bonded to each other to form a ring, or each of R^{d} and R^{e} independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms. Each of R¹ to R¹⁵ independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms; each of n and m independently represents an integer greater than or equal to 0 and less than or equal to 3; and Y¹ represents a boronic acid, a boronic ester, a cyclic-triolborate salt, or the like. As the cyclic-triolborate salt, a lithium salt, a potassium salt, or a sodium salt may be used.

The organic compound represented by General Formula (G4) above can be obtained by reacting a halogen compound (B3) with a boronic acid compound (B4), as shown in Synthesis Scheme (A-2) below.

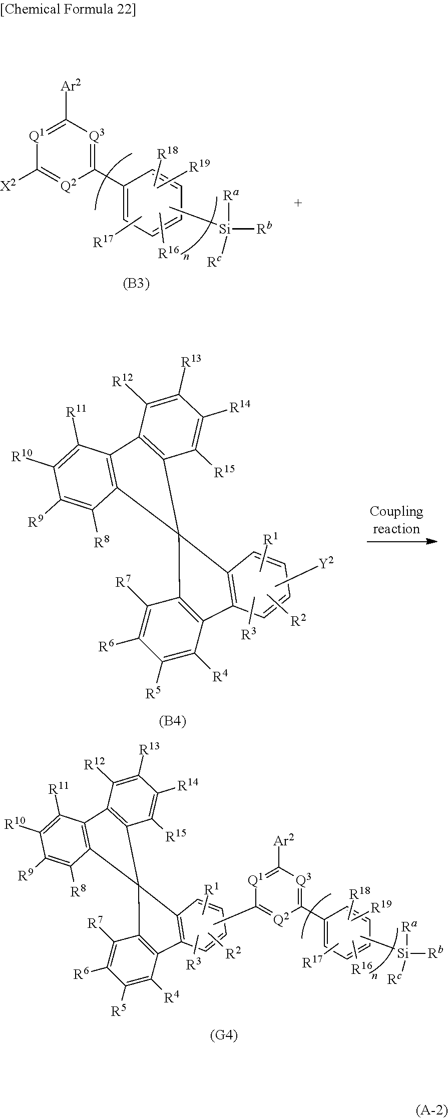

In Synthesis Scheme (A-2), X² represents a halogen. Each of Q¹, Q², and Q³ independently represents N or CH (including CD), and at least any two of Q¹, Q², and Q³ represent N; each of R^{a}, R^{b}, and R^{c} independently represents an alkyl group having 1 to 4 carbon atoms, a cycloalkyl group having 3 to 10 carbon atoms, or a substituted or unsubstituted aryl group having 6 to 12 carbon atoms; Ar² represents a substituted or unsubstituted aryl group having 6 to 10 carbon atoms. Each of R¹ to R¹⁹ independently represents hydrogen (including deuterium), an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; n represents an integer greater than or equal to 0 and less than or equal to 3; Y² represents a boronic acid, a boronic ester, a cyclic-triolborate salt, or the like. As the cyclic-triolborate salt, a lithium salt, a potassium salt, or a sodium salt may be used.

Examples of a palladium catalyst that can be used in the coupling reaction represented by the synthesis scheme above include palladium(II) acetate, tetrakis(triphenylphosphine)palladium(0), bis(triphenylphosphine)palladium(II) dichloride, and allylpalladium(II) chloride dimer.

Examples of a ligand of the above palladium catalyst include di(1-adamantyl)-n-butylphosphine, (±)-2,2′-bis(diphenylphosphino)-1,1′-binaphthyl, tri(ortho-tolyl)phosphine, triphenylphosphine, tricyclohexylphosphine, and di-tert-butyl(2,2-diphenyl-1-methyl-1-cyclopropyl)phosphine.

Examples of a base that can be used in the coupling reaction represented by the synthesis scheme above include an organic base such as potassium tert-butoxide and an inorganic base such as potassium carbonate, sodium carbonate, or tripotassium phosphate.

Examples of a solvent that can be used in the coupling reaction represented by the synthesis scheme above include toluene, xylene, mesitylene, benzene, tetrahydrofuran, dioxane, and diethylene glycol dimethyl ether. However, the solvent that can be used is not limited to these solvents.

The reaction employed in the synthesis scheme above is not limited to the Suzuki-Miyaura reaction. A Migita-Kosugi-Stille coupling reaction using an organotin compound, a coupling reaction using a Grignard reagent, an Ullmann reaction using copper or a copper compound, a nucleophilic substitution reaction or the like can be employed.

A variety of kinds of the compound (B1) to the compound (B4) above are commercially available or can be synthesized.

The organic compound of one embodiment of the present invention can be synthesized in the above manner, but the present invention is not limited thereto, and any other synthesis method may be employed.

### <Method for Obtaining GSP_Slope>

Here, a method for obtaining the GSP_slope of an organic compound film formed by a vacuum evaporation method is described.

A phenomenon in which the surface potential of an evaporated film increases in proportion to the thickness of the film is called the giant surface potential as described above. In general, a slope of a plot of the surface potential of an evaporated film in the thickness direction by Kelvin probe measurement is assumed as the level of the giant surface potential, that is, GSP_slope (mV/nm). Meanwhile, in the case where two different layers are stacked, a change in the density of charges (mC/m²) accumulated at the interface, which is in association with GSP_slope, can be utilized to estimate the value of the GSP_slope. The density of the charges accumulated at the interface can be calculated by C-V (capacity-voltage) measurement on an element structure in which charges are accumulated on one of the layers.

When a voltage is applied to a stack of organic thin films with different SOPs (a thin film 1 on the anode side and a thin film 2 on the cathode side; the anode is closer to the substrate than the cathode) and carriers accumulated at the interface (accumulated charges) are electrons, the following equations hold.

$$\begin{matrix}[ Equation 1 ] & \\ {σ}_{acc} = ( {V}_{th} - {V}_{inj} ) \frac{{ε}_{1}}{{d}_{1}} = {σ}_{int} & ( 1 )\end{matrix}$$

$$\begin{matrix}[ Equation 2 ] & \\ {σ}_{int} = {P}_{1} - {P}_{2} = \frac{{ε}_{1} {V}_{1}}{{d}_{1}} - \frac{{ε}_{2} {V}_{2}}{{d}_{2}} & ( 2 )\end{matrix}$$

In Equation (1), σ_{acc} is an accumulated charge density, σᵢₙₜ is an interface charge density, Vᵢₙⱼ is an electron-injection voltage, Vₜₕ is a threshold voltage, d₁ is the thickness of the thin film 1, and ε₁ is the dielectric constant of the thin film 1. Note that Vᵢₙⱼ and Vₜₕ can be estimated from the capacity-voltage characteristics of a device. As the dielectric constant, it is possible to use a value obtained by multiplying the vacuum permittivity and the relative permittivity, which is assumed to be the square of an ordinary refractive index nₒ (the value at a wavelength of 633 nm). As described above, according to Equation (1), the interface charge density σᵢₙₜ can be calculated using Vᵢₙⱼ and Vₜₕ estimated from the capacity-voltage characteristics, the dielectric constant ε₁ of the thin film 1 calculated from the ordinary refractive index, and a thickness d₁ of the thin film 1.

Next, in Equation (2), P₁ and P₂ are the degree of SOP of the thin film 1 and the degree of SOP of the thin film 2, respectively, in the direction perpendicular to the substrate surface, ε₂ is the dielectric constant of the thin film 2, and d₂ is the thickness of the thin film 2. Since the interface charge density σᵢₙₜ can be obtained from Equation (1) above, the use of a substance with a known GSP_slope for the thin film 1 enables the GSP_slope of the thin film 2 to be estimated.

Each of the measurement device 1 and the measurement device 2 is fabricated using tris(8-quinolinolato)aluminum(III) (abbreviation: Alq₃) whose GSP_slope is known, for the thin film 1 and 2-(2′,7′-di-tert-butyl-9,9′-spirobi[9H-fluoren]-2-yl)-4-phenyl-6-[4-(triphenylsilyl)phenyl]-1,3,5-triazine (abbreviation: tBu-PSi-SFTzn), which is the organic compound of one embodiment of the present invention, for the thin film 2, and an example of calculating the GSP_slope of an evaporated film of tBu-PSi-SFTzn is shown below. Note that the GSP_slope of Alq₃ measured using a Kelvin probe is 48 mV/nm according to Non-Patent Document 3. Chemical formulae of tBu-PSi-SFTzn and Alq₃ are shown below.

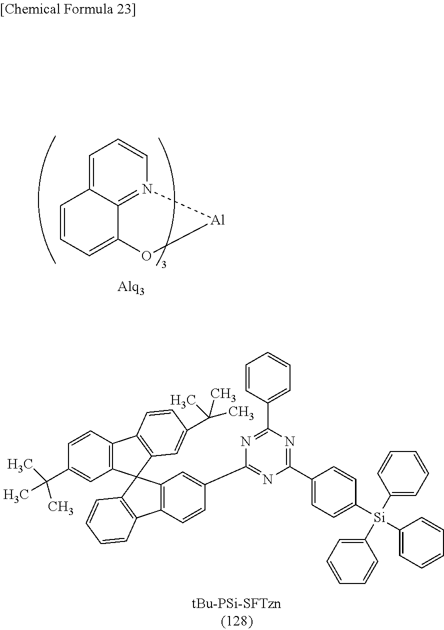

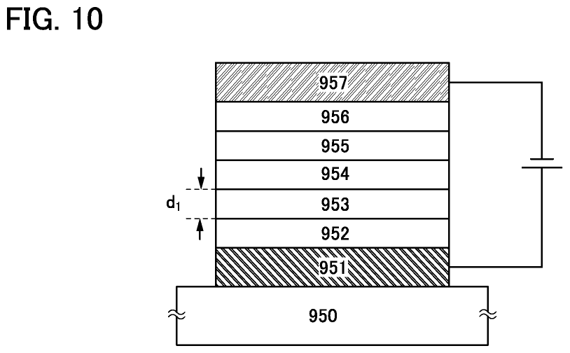

As illustrated in FIG. 10, each of the measurement device 1 and the measurement device 2 has a structure in which a layer 952, a layer 953, a layer 954, a layer 955, and a layer 956 are stacked in this order over an anode 951 formed over a glass substrate 950, and a cathode 957 is stacked over the layer 956. Table 1 shows the device structure of each of the measurement device 1 and the measurement device 2.

The layer 953 and the layer 954 in each of the measurement device 1 and the measurement device 2 correspond to the thin film 1 and the thin film 2, respectively. The layer 952 was formed by co-evaporation of N-(biphenyl-4-yl)-N-[4-(9-phenyl-9H-carbazol-3-yl)phenyl]-9,9-dimethyl-9H-fluoren-2-amine (abbreviation: PCBBiF) and OCHD-003, which is an organic compound having an electron-acceptor property, to a thickness of 10 nm at the weight ratio of 1:0.1 (PCBBiF:OCHD-003). The layer 953 was formed by evaporation of Alq₃ to a thickness of 80 nm (the measurement device 1) or 100 nm (the measurement device 2). The layer 954 was formed by evaporation of tBu-PSi-SFTzn to a thickness of 100 nm. The layer 955 was formed by evaporation of 4,7-di-1-pyrrolidinyl-1,10-phenanthroline (abbreviation: Pyrrd-Phen) to a thickness of 1 nm.

TABLE 1

| | | |
|---|---|---|
| Film | Measurement | Measurement |
| thickness | device 1 | device 2 |

| | | | |
|---|---|---|---|
| Cathode 957 | 200 | nm | Al |
| Layer 956 | 1 | nm | LiF |
| Layer 955 | 1 | nm | Pyrrd-Phen |
| Layer 954 | 100 | nm | tBu-PSi-SFTzn |

| | | | |
|---|---|---|---|
| Layer 953 | — | Alq₃ (80 nm) | Alq₃ (100 nm) |

| | | | |
|---|---|---|---|
| Layer 952 | 10 | nm | PCBBiF:OCHD-003 (1:0.1) |
| Anode 951 | 110 | nm | ITSO |

Note that the layers 952 to 956 and the cathode in each measurement device were formed from the anode side by a vacuum evaporation method under the conditions where the substrate temperature was set to room temperature and the deposition rate ranged from 0.2 nm/s to 0.6 nm/s. In fabrication of the measurement device, the deposition rate of each layer is preferably within the range of 3 nm/min to 600 nm/min. The thickness of each layer in the measurement device is preferably greater than or equal to 3 nm and less than or equal to 500 nm, further preferably greater than or equal to 50 nm and less than or equal to 300 nm.

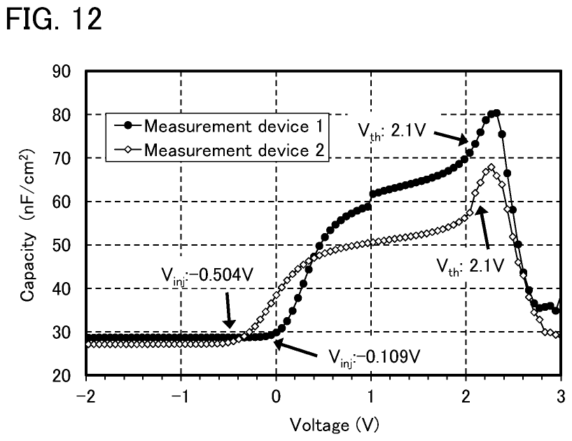

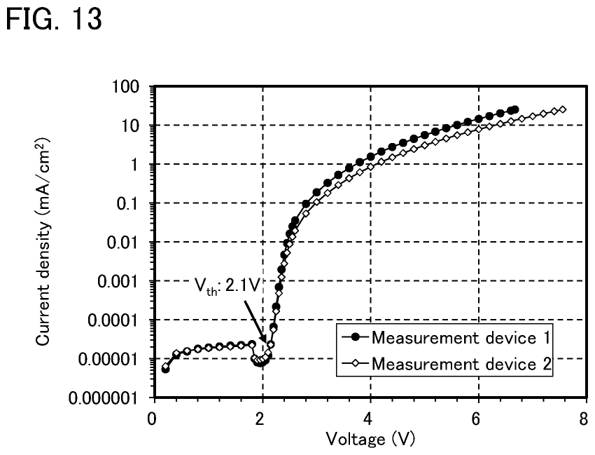

FIG. 12 shows capacity-voltage characteristics of each of the measurement device 1 and the measurement device 2 and FIG. 13 shows current density-voltage characteristics thereof.

Table 2 shows the electron-injection voltage Vᵢₙⱼ, the interface charge density σᵢₙₜ, and the GSP_slope of each of the measurement device 1 and the measurement device 2, which were obtained from FIG. 12, Equation (1), and Equation (2), and the refractive index nₒ of a film of Alq₃, the refractive index nₒ of a film of tBu-PSi-SFTzn, the actually measured thickness d₁ of the layer 953, and the threshold voltage Vₜₕ obtained from FIG. 13, which were used in the calculation. Note that the threshold voltage Vₜₕ can also be determined from the current density-voltage characteristics. As the actually measured thickness d₁ of the layer 953, a highly accurate value of an actually formed film calculated by a spectroscopic ellipsometry method was used.

TABLE 2

| | Measure- | Measure- |
|---|---|---|
| | ment | ment |
| | device 1 | device 2 |
| Electron-injection voltage Vᵢₙⱼ (V) | −0.109 | −0.504 |
| Threshold voltage Vₜₕ(V) | 2.1 | 2.1 |
| Accumulated charge density σᵢₙₜ (mC/m²) | 0.91 | 0.86 |
| Ordinary refractive index nₒ of | 1.71 | |
| Alq₃ (@633 nm) | | |
| Ordinary refractive index nₒ of | 1.68 | |
| tBu-PSi-SFTzn (@633 nm) | | |
| d₁ (nm) | 63.01 | 78.74 |
| GSP_slope (mV/nm) | 13.4 | 15.4 |

| | |
|---|---|
| GSP_slope (mV/nm) (average value) | 14.4 |

The GSP_slope of an evaporated film of tBu-PSi-SFTzn was estimated to be 14.4 mV/nm by averaging the GSP_slopes of the layers 954 obtained using the measurement results of the measurement device 1 and the measurement device 2 shown in Table 2.

In this manner, a device in which Alq₃ with a known GSP_slope of a film and a film formed using an organic compound whose GSP_slope is to be obtained are stacked is fabricated and the capacity-voltage characteristics are measured, so that the GSP_slope of the organic compound can be estimated.

In the above, the method for calculating the GSP_slope of the organic compound used for the electron-transport layer in which electrons serve as carriers is described. In the case of using the GSP_slope of an organic compound used for a hole-transport layer in which holes serve as carriers, the GSP_slope can be calculated in a similar manner using Equation (3) shown below, as described in Non-Patent Document 4.

$$[ Equation 3 ]$$

$${σ}_{a c c} = ( {V}_{t h} - {V}_{inj} ) \frac{{ε}_{2}}{{d}_{2}} = - {σ}_{int} \qquad ( 3 )$$

Organic compounds used for layers of a light-emitting device are preferably selected in consideration of the GSP_slopes of the evaporated films of the organic compounds, which are measured in advance by the above measurement method.

The structures described in this embodiment can be used in appropriate combination with any of the structures described in the other embodiments.

### Embodiment 2

In this embodiment, a light-emitting device of one embodiment of the present invention is described.

The light-emitting device of one embodiment of the present invention is preferably formed using an organic compound including an electron-transport skeleton having a pyrimidine ring or a triazine ring, which is a π-electron deficient heteroaromatic ring. The organic compound includes at least any one of a trisubstituted methyl group (—CR₃), a trisubstituted silyl group (—SiR₃), and a trisubstituted germyl group (—GeR₃) that are each bonded to the electron-transport skeleton directly or through a linker. The organic compound includes a 9,9′-spirobifluorene ring or a 9,9-diphenylfluorene ring that is bonded to the electron-transport skeleton directly or through a linker.

An organic compound represented by General Formula (G0) below can be given as an example of the organic compound and can be used in the light-emitting device of one embodiment of the present invention.

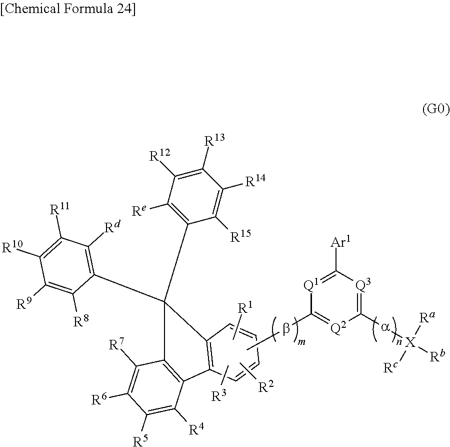

In General Formula (G0), each of Q¹, Q², and Q³ independently represents N or CH (including CD), and at least any two of Q¹, Q², and Q³ represent N; X represents C, Si, or Ge; each of R^{a}, R^{b}, and R^{c} independently represents an alkyl group having 1 to 4 carbon atoms, a cycloalkyl group having 3 to 10 carbon atoms, or a substituted or unsubstituted aryl group having 6 to 12 carbon atoms; Ar¹ represents a substituted or unsubstituted aryl group having 6 to 30 carbon atoms; each of α and β independently represents a substituted or unsubstituted arylene group having 6 to 30 carbon atoms; R^{d} and R^{e} represent bonds that are bonded to each other to form a ring, or each of R^{d} and R^{e} independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; each of R¹ to R¹⁵ independently represents hydrogen (including deuterium), an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; and each of n and m independently represents an integer greater than or equal to 0 and less than or equal to 3.

The organic compound represented by General Formula (G0) has an excellent carrier-transport property. The organic compound with such a structure has a low refractive index of a film. The organic compound with such a structure has a small GSP_slope of an evaporated film. The organic compound with such a structure has high T_{g} of a film and powder. When being provided with the organic compound represented by General Formula (G0), the light-emitting device can have high light extraction efficiency and a low driving voltage. The organic compound with such a structure can be suitably used also for a light-emitting device fabricated through a step of processing its organic compound layer by a lithography method.

Specific examples of the substituent that can be used in the organic compound represented by General Formula (G0) are similar to those described in Embodiment 1 and thus are not described here.

The organic compound represented by any of General Formula (G1) to General Formula (G7) described in Embodiment 1 can be used in the light-emitting device of one embodiment of the present invention.

In the light-emitting device of one embodiment of the present invention, the organic compound represented by any of General Formula (G0) to General Formula (G7) can also be used for a mixed layer in combination with a metal complex. In that case, the metal complex is particularly preferably an organic complex including an alkali metal. Examples of the organic complex including an alkali metal include 8-quinolinolato-lithium (abbreviation: Liq), 8-quinolinolato-sodium (abbreviation: Naq), and 8-quinolinolato-potassium (abbreviation: Kq), which are represented by structural formulae below, and derivatives thereof. It is preferable to use a mixed layer including the metal complex and the organic compound represented by any of General Formula (G0) to General Formula (G7) as the electron-transport layer in the light-emitting device of one embodiment of the present invention, in which case electron injection from the cathode can be facilitated and the electron-transport property of the electron-transport layer can be controlled.

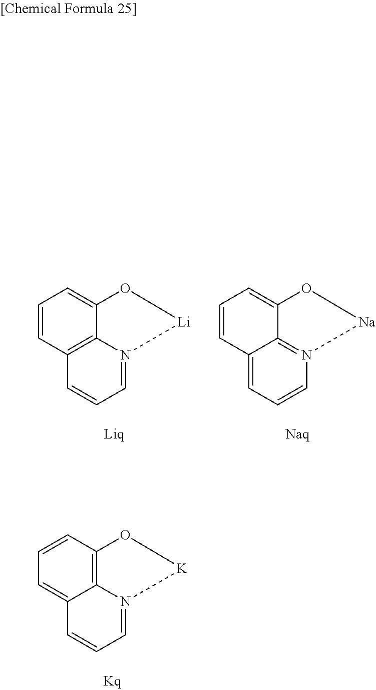

In the case where a mixed layer including the metal complex and the organic compound represented by any of General Formula (G0) to General Formula (G7) is used as an electron-transport layer, the electron-transport layer is preferably in contact with the cathode. Alternatively, an electron-injection layer preferably has a thickness less than or equal to 5 nm when provided between the electron-transport layer and the cathode. Thus, the effect of the electron-transport property can be enhanced.

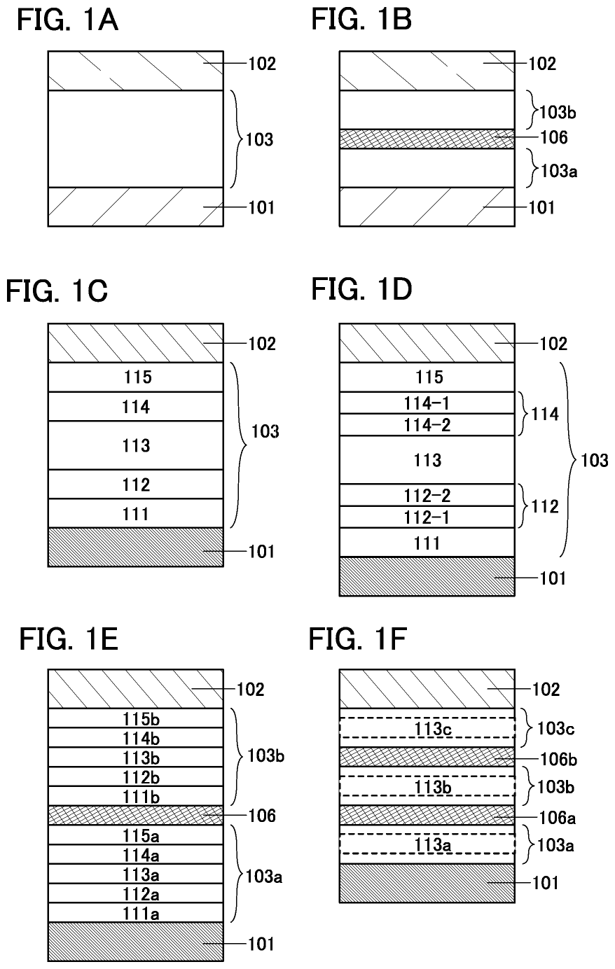

Next, structures of the light-emitting device of one embodiment of the present invention are described with reference to FIGS. 1A to 1F.

### <<Basic Structure of Light-Emitting Device>>

A basic structure of a light-emitting device is described. FIG. 1A illustrates a light-emitting device including, between a pair of electrodes, an EL layer including a light-emitting layer. Specifically, an EL layer 103 is located between a first electrode 101 and a second electrode 102.

FIG. 1B illustrates a light-emitting device that has a stacked-layer structure (tandem structure) in which a plurality of EL layers (two EL layers 103a and 103b in FIG. 1B) are provided between a pair of electrodes and a charge-generation layer 106 is provided between the EL layers. A light-emitting device having a tandem structure enables manufacturing of a display device that has high efficiency without changing the amount of current.

The charge-generation layer 106 has a function of injecting electrons into one of the EL layers (the EL layers 103a and 103b) and injecting holes into the other of the EL layers (the EL layers 103a and 103b) when a potential difference is caused between the first electrode 101 and the second electrode 102. Thus, when voltage is applied in FIG. 1B such that the potential of the first electrode 101 can be higher than that of the second electrode 102, electrons are injected into the EL layer 103a from the charge-generation layer 106 and holes are injected into the EL layer 103b from the charge-generation layer 106.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

FIG. 1C illustrates a stacked-layer structure of the EL layer 103 in the light-emitting device of one embodiment of the present invention. In this case, the first electrode 101 is regarded as functioning as an anode, and the second electrode 102 is regarded as functioning as a cathode. The EL layer 103 has a structure in which a hole-injection layer 111, a hole-transport layer 112, the light-emitting layer 113, an electron-transport layer 114, and an electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can extend driving lifetime; in other words, the structure can achieve higher reliability than a single-layer structure in some cases. In the case where a plurality of EL layers are provided as in the tandem structure illustrated in FIG. 1B, the layers in each EL layer are sequentially stacked from the anode side as described above. When the first electrode 101 is the cathode and the second electrode 102 is the anode, the stacking order of the layers in the EL layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the EL layers (103, 103a, and 103b) contains an appropriate combination of a light-emitting substance and a plurality of substances, so that fluorescent light of a desired color or phosphorescent light of a desired color can be obtained. The plurality of EL layers (the EL layers 103a and 103b) in FIG. 1B may exhibit their respective emission colors. In that case, the light-emitting substances and other substances can be different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 1C. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by controlling the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify light obtained from the light-emitting layer 113 at a desired wavelength (λ), it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, the above effect can be sufficiently obtained wherever the reflective regions may be set in the first electrode 101 and the second electrode 102. Furthermore, the optical path length between the first electrode 101 and the light-emitting layer is, to be exact, the optical path length between the reflective region in the first electrode 101 and the light-emitting region in the light-emitting layer. However, it is difficult to precisely determine the reflective region in the first electrode 101 and the light-emitting region in the light-emitting layer; thus, it is assumed that the above effect can be sufficiently obtained wherever the reflective region and the light-emitting region may be set in the first electrode 101 and the light-emitting layer, respectively.

FIG. 1D illustrates a modification example of the stacked-layer structure illustrated in FIG. 1C. Also in this case, the first electrode 101 is regarded as functioning as an anode, and the second electrode 102 is regarded as functioning as a cathode. In this modification example, the hole-transport layer 112 and the electron-transport layer 114 each have a stacked-layer structure of two layers. In other words, the EL layer 103 has a structure in which the hole-injection layer 111, a first hole-transport layer 112-1, a second hole-transport layer 112-2, a light-emitting layer 113, a second electron-transport layer 114-2, a first electron-transport layer 114-1, and the electron-injection layer 115 are stacked in this order over the first electrode 101. Note that the light-emitting layer 113 is positioned between the first electrode 101 and the second electrode 102. The first hole-transport layer 112-1 is positioned between the first electrode 101 and the light-emitting layer 113. The first electron-transport layer 114-1 is positioned between the light-emitting layer 113 and the second electrode 102. The hole-injection layer 111 is positioned between the first electrode 101 and the hole-transport layer 112. The electron-injection layer 115 is positioned between the electron-transport layer 114 and the second electrode 102. The second hole-transport layer 112-2 is positioned between the first hole-transport layer 112-1 and the light-emitting layer 113. In other words, the second electron-transport layer 114-2 is positioned between the light-emitting layer 113 and the first electron-transport layer 114-1. In the case where the EL layer 103 has such a stacked-layer structure, one or more of the hole-injection layer 111, the hole-transport layer 112, and the light-emitting layer 113 preferably contains the organic compound represented by any of General Formula (G4) to General Formula (G9).

The second hole-transport layer 112-2 is provided to prevent passing of electrons from the light-emitting layer 113 to the first electrode 101 side, for example. Accordingly, the second hole-transport layer 112-2 can also be referred to as an electron-blocking layer. It is particularly preferable to use the organic compound represented by any of General Formula (G4) to General Formula (G9) for the second hole-transport layer 112-2 in contact with the light-emitting layer 113. The second electron-transport layer 114-2 is provided to prevent passing of holes from the light-emitting layer 113 to the second electrode 102 side, for example. Accordingly, the second electron-transport layer 114-2 can also be referred to as a hole-blocking layer.

The light-emitting device illustrated in FIG. 1E is a light-emitting device having a tandem structure. Owing to a microcavity structure of the light-emitting device, light (monochromatic light) with different wavelengths from the EL layers (the EL layers 103a and 103b) can be extracted. It is therefore unnecessary to separately form EL layers for obtaining a plurality of emission colors (e.g., R, G, and B). Thus, high resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 1F is an example of the light-emitting device having the tandem structure illustrated in FIG. 1B, and includes three EL layers (103a, 103b, and 103c) stacked with charge-generation layers (106a and 106b) positioned therebetween, as illustrated in FIG. 1F. The three EL layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

### <<Specific Structure of Light-Emitting Device>>

Next, a specific structure of the light-emitting device of one embodiment of the present invention will be described. Here, the description is made using FIG. 1E illustrating the tandem structure. Note that the structure of the EL layer applies also to the structure of the light-emitting devices having a single structure in FIGS. 1A and 1C. When the light-emitting device in FIG. 1E has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device in FIG. 1E, when the first electrode 101 is the anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

### <Hole-Injection Layer>

The hole-injection layers (111, 111a, and 111b) inject holes from the first electrode 101 serving as the anode and the charge-generation layers (106, 106a, and 106b) to the EL layers (103, 103a, and 103b) and contain an organic acceptor material and a material having a high hole-injection property.

The organic acceptor material allows holes to be generated in another organic compound whose HOMO level is close to the LUMO level of the organic acceptor material when charge separation is caused between the organic acceptor material and the organic compound. Thus, as the organic acceptor material, a compound including an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly suitable because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 of the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among the above oxides, molybdenum oxide is preferable because it is stable in atmospheric air, has a low hygroscopic property, and is easily handled. Other examples include a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI); (C₆₀-Ih)[5,6]fullerene (abbreviation: C₆₀); (C₇₀-D5h)[5,6]fullerene (abbreviation: C₇₀); an organic compound such as phthalocyanine (abbreviation: H₂Pc); and a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). A phthalocyanine-based metal complex such as CuPc or ZnPc and 2,3,8,9,14,15-hexafluorodiquinoxalino[2,3-a:2′,3′-c]phenazine are especially preferable. Among these materials, CuPc and ZnPc are preferable because they are inexpensive and have favorable characteristics. Using ZnPc, which has a low diffusion coefficient with respect to silicon, reduces the probability that metal diffusion to a semiconductor adversely affects the semiconductor characteristics; accordingly, ZnPc is particularly suitable for a display device manufactured using a silicon semiconductor.

Other examples are aromatic amine compounds, which are low-molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), 4,4′-bis[N-(4-diphenylaminophenyl)-N-phenylamino]biphenyl (abbreviation: DPAB), N,N′-bis[4-bis(3-methylphenyl)aminophenyl]-N,N′-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide](abbreviation: PTPDMA), and poly[N,N′-bis(4-butylphenyl)-N,N′-bis(phenyl)benzidine](abbreviation: Poly-TPD). Alternatively, it is possible to use a high-molecular compound to which acid is added, such as poly(3,4-ethylenedioxythiophene)/polystyrenesulfonic acid (abbreviation: PEDOT/PSS) or polyaniline/polystyrenesulfonic acid (abbreviation: PAni/PSS), for example.

As the material having a high hole-injection property, a mixed material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material is preferably a substance having a hole mobility higher than or equal to 1×10⁻⁶ cm²/Vs in the case where the square root of the electric field strength [V/cm] is 600. Note that any other substance can also be used as long as the substance has a hole-transport property higher than an electron-transport property.

As the hole-transport material, materials having a high hole-transport property, such as a compound including a π-electron rich heteroaromatic ring (e.g., a carbazole derivative, a furan derivative, and a thiophene derivative) and an aromatic amine (an organic compound including an aromatic amine skeleton), are preferable.

Examples of the carbazole derivative (an organic compound including a carbazole ring) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-3,3′-bi-9H-carbazole (abbreviation: QNCCP).

Specific examples of the aromatic amine having a carbazolyl group include 4-phenyl-4′-(9-phenyl-9H-carbazol-3-yl)triphenylamine (abbreviation: PCBA1BP), N-(biphenyl-4-yl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-(1,1′:3′,1″-terphenyl-4-yl)-9,9-dimethyl-9H-fluoren-2-amine, N-[4-(9-phenyl-9H-carbazol-3-yl)phenyl]-N-(1,1′:4′,1″-terphenyl-4-yl)-9,9-dimethyl-9H-fluoren-2-amine, N-[4-(9-phenyl-9H-carbazol-3-yl)phenyl]-N-(1,1:3′,1″-terphenyl-4-yl)-9,9-dimethyl-9H-fluoren-4-amine, N-[4-(9-phenyl-9H-carbazol-3-yl)phenyl]-N-(1,1′:4′,1″-terphenyl-4-yl)-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), PCzPCA1, PCzPCA2, PCzPCN1, 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), N-(9,9-spirobi[9H-fluoren]-2-yl)-N,9-diphenylcarbazol-3-amine (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 9-[4-(9-phenyl-9H-carbazol-3-yl)phenyl]phenanthrene (abbreviation: PCPPn), 3-[4-(1-naphthyl)phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthryl)phenyl]-9H-carbazole (abbreviation: CzPA).

Specific examples of the furan derivative (an organic compound including a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound including a thiophene ring) include 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), N-(9,9-spirobi[9H-fluoren]-2-yl)-N,N′,N′-triphenyl-1,4-phenylenediamine (abbreviation: DPASF), N,N′-diphenyl-N,N′-bis(4-diphenylaminophenyl)spirobi[9H-fluorene]-2,7-diamine (abbreviation: DPA2SF), 4,4′,4″-tris[N-(1-naphthyl)-N-phenylamino]triphenylamine (abbreviation: 1′-TNATA), TDATA, m-MTDATA, N,N′-di(p-tolyl)-N,N′-diphenyl-p-phenylenediamine (abbreviation: DTDPPA), DPAB, DNTPD, DPA3B, N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), NN-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-([2,1′-binaphthyl]-6-yl)triphenylamine (abbreviation: BBAαNβNB), 4,4′-diphenyl-4″-([2,1′-binaphthyl]-7-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-([2,2′-binaphthyl]-6-yl)triphenylamine (abbreviation: BBA(βN2)B), 4,4′-diphenyl-4″-([2,2′-binaphthyl]-7-yl)triphenylamine (abbreviation: BBA(βN2)B-03), 4,4′-diphenyl-4″-([1,2′-binaphthyl]-4-yl)triphenylamine (abbreviation: BBAβNαNB), 4,4′-diphenyl-4″-([1,2′-binaphthyl]-5-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other than the above, PVK, PVTPA, PTPDMA, Poly-TPD, or the like that is a high-molecular compound (e.g., an oligomer, a dendrimer, or a polymer) can be used as the hole-transport material. Alternatively, a high-molecular compound to which acid is added, such as PEDOT/PSS or PAni/PSS can be used, for example.

Note that the hole-transport material is not limited to the above examples, and any of a variety of known materials may be used alone or in combination as the hole-transport material.

The hole-injection layers (111, 111a, and 111b) can be formed by any of known film formation methods such as a vacuum evaporation method.

### <Hole-Transport Layer>

The hole-transport layers (112, 112a, and 112b) transport the holes, which are injected from the first electrode 101 by the hole-injection layers (111, 111a, and 111b), to the light-emitting layers (113, 113a, and 113b). Note that the hole-transport layers (112, 112a, and 112b) each include a hole-transport material. Thus, the hole-transport layers (112, 112a, and 112b) can be formed using any of the hole-transport materials that can be used for the hole-injection layers (111, 111a, and 111b).

Note that in the light-emitting device of one embodiment of the present invention, the organic compound used for the hole-transport layers (112, 112a, and 112b) can also be used for the light-emitting layers (113, 113a, and 113b). The same organic compound is preferably used for the hole-transport layers (112, 112a, and 112b) and the light-emitting layers (113, 113a, and 113b), in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) include a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables exhibiting different emission colors (e.g., white light emission obtained by a combination of complementary emission colors). When a plurality of light-emitting layers are provided, the light-emitting layers can exhibit the same color. The structure in which a plurality of light-emitting layers that emit light of the same color are stacked can sometimes achieve higher reliability than a single-layer structure. Furthermore, a stacked-layer structure in which one light-emitting layer contains two or more kinds of light-emitting substances may be employed.

The light-emitting layers (113, 113a, and 113b) may each contain one or more kinds of organic compounds (e.g., a host material) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (S1 level) of the second host material is higher than that of the first host material, and the lowest triplet excitation energy level (T1 level) of the second host material is higher than that of the guest material. Preferably, the lowest triplet excitation energy level (T1 level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, low voltage, and a long lifetime can be achieved.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small energy difference between the S1 level and the T1 level and functions as a thermally activated delayed fluorescent (TADF) material capable of converting triplet excitation energy into singlet excitation energy. In an example of a preferable combination of two or more kinds of organic compounds forming an exciplex, one compound of the two or more kinds of organic compounds has a π-electron deficient heteroaromatic ring and the other compound has a π-electron rich heteroaromatic ring. A phosphorescent substance such as an iridium-, rhodium-, or platinum-based organometallic complex or a metal complex may be used as one compound of the combination for forming an exciplex. The organic compound represented by any of General Formula (G0) to General Formula (G7) has an electron-transport property and thus can be used as the host material.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances that emit fluorescent light (fluorescent substances) can be given as examples of the light-emitting substance that converts singlet excitation energy into light emission and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine](abbreviation: 1,6BnfAPrn), N,N′-(pyrene-1,6-diyl)bis[(N-phenylbenzo[b]naphtho[1,2-d]furan)-8-amine](abbreviation: 1,6BnfAPrn-02), and N,N′-(pyrene-1,6-diyl)bis[(6,N-diphenylbenzo[b]naphtho[1,2-d]furan)-8-amine](abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N′,N′-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), or 3,10-bis[N-(dibenzofuran-3-yl)-N-phenylamino]naphtho[2,3-b;6,7-b′]bisbenzofuran (abbreviation: 3,10FrA2Nbf(IV)-02). In particular, a pyrenediamine compound such as 1,6FLPAPrn, 1,6mMemFLPAPrn, or 1,6BnfAPrn-03 can be used, for example.

A fused heteroaromatic compound including nitrogen and boron, especially a compound having a diaza-boranaphtho-anthracene skeleton, emits high color purity blue light with a narrow emission spectrum and can thus be suitably used. Examples of the compound include 5,9-diphenyl-5H,9H-[1,4]benzazaborino[2,3,4-kl]phenazaborine (abbreviation: DABNA-1), 9-(biphenyl-3-yl)-N,N,5,11-tetraphenyl-5H,9H-[1,4]benzazaborino[2,3,4-kl]phenazaborin-3-amine (abbreviation: DABNA-2), 2,12-di(tert-butyl)-5,9-di(4-tert-butylphenyl)-N,N-diphenyl-5H,9H-[1,4]benzazaborino[2,3,4-kl]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-kl]phenazaborine (abbreviation: Me-tBu4DABNA), N7,N7,N13,N13,5,9,11,15-octaphenyl-5H,9H,11H,15H-[1,4]benzazaborino[2,3,4-kl][1,4]benzazaborino[4′,3′,2′:4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, a compound having an indole skeleton, such as 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-G), 9,11-bis[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-Y), or the like can be suitably used.

<<Light-Emitting Substance that Converts Triplet Excitation Energy into Light Emission>>

Examples of the light-emitting substance that converts triplet excitation energy into light and that can be used in the light-emitting layer 113 include substances that emit phosphorescent light (phosphorescent substances) and TADF materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably includes a metal element with large spin-orbit interaction, and can be an organometallic complex, a metal complex (platinum complex), or a rare earth metal complex, for example. Specifically, the phosphorescent substance preferably includes a transition metal element. It is preferable that the phosphorescent substance include a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (Peaking at Wavelength from 450 nm to 570 nm: Blue or Green)>>

As examples of a phosphorescent substance which emits blue or green light and whose emission spectrum has a peak wavelength higher than or equal to 450 nm and lower than or equal to 570 nm, the following substances can be given.

Examples include organometallic complexes having a 4H-triazole ring, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Jr(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN3}-4-cyanophenyl-κC)iridium(III) (abbreviation: CNImIr); organometallic complexes having a benzimidazolidene skeleton, such as tris[(6-tert-butyl-3-phenyl-2H-imidazo[4,5-b]pyrazin-1-yl-κC2)phenyl-κC]iridium(III) (abbreviation: [Ir(cb)₃]); organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C2′]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), bis[2-(4′,6′-difluorophenyl)pyridinato-N,C^{2′}]iridium(III) acetylacetonate (abbreviation: FIr(acac)); and platinum complexes such as (2-{3-[3-(3,5-di-tert-butylphenyl)benzimidazol-1-yl-2-ylidene-κC2]phenoxy-κC2}-9-(4-tert-butyl-2-pyridinyl-κN)carbazole-2,1-diyl-κC1)platinum(II) (abbreviation: PtON-TBBI). Alternatively, a compound obtained by substituting deuterium for part of hydrogen in any of these compounds can also be used.

<<Phosphorescent Substance (Peaking at Wavelength from 495 nm to 590 nm: Green or Yellow)>>

As examples of a phosphorescent substance which emits green or yellow light and whose emission spectrum has a peak wavelength higher than or equal to 495 nm and lower than or equal to 590 nm, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN3]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d3-methyl-8-(2-pyridinyl-κN)benzofuro[2,3-b]pyridine-κC]bis[2-(5-d3-methyl-2-pyridinyl-κN2)phenyl-κC]iridium(III) (abbreviation: Ir(5mppy-d3)₂(mbfpypy-d3)), {2-(methyl-d3)-8-[4-(1-methylethyl-1-d)-2-pyridinyl-κN]benzofuro[2,3-b]pyridin-7-yl-KC}bis{5-(methyl-d3)-2-[5-(methyl-d3)-2-pyridinyl-KN]phenyl-κC}iridium(III) (abbreviation: Ir(5mtpy-d6)₂(mbfpypy-iPr-d4)), [2-(methyl-d3)-8-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)), [2-(4-d3-methyl-5-phenyl-2-pyridinyl-κN2)phenyl-κC]bis[2-(5-d3-methyl-2-pyridinyl-κN2)phenyl-κC]iridium(III) (abbreviation: [Ir(5mppy-d3)₂(mdppy-d3)]), [2-methyl-8-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]), and tris{2-[5-(methyl-d3)-4-phenyl-2-pyridinyl-N]phenyl-κC}iridium(III) (abbreviation: Ir(5m4dppy-d3)₃); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); organometallic platinum complexes, such as (2-{1-(5-tert-butylbiphenyl-2-yl)-4-[3-tert-butyl-5-(4-phenyl-2-pyridinyl-κN)phenyl-κC6]-2-benzimidazolyl-κN3}-4,6-di-tert-butylphenolato-κO)platinum(II) (abbreviation: Pt(tBudppymmtBubiz-tBubp)) and [2-(4-(3,5-di-tert-butylphenyl)-6-{3-[4-(5′-tert-butyl[1,1′:3′,1″-terphenyl]-2′-yl)-2-pyridinyl-κN]phenyl-κC2}-2-pyridinyl-κN)phenolato-κO]platinum(II) (abbreviation: Pt(4tButpppypyp-mmtBup)); and a rare earth metal complex such as tris(acetylacetonato)(monophenanthroline)terbium(III) (abbreviation: [Tb(acac)₃(Phen)]). Alternatively, a compound obtained by substituting deuterium for part of hydrogen in any of these compounds can also be used.

<<Phosphorescent Substance (Peaking at Wavelength from 570 nm to 750 nm: Yellow or Red)>>

As examples of a phosphorescent substance which emits yellow or red light and whose emission spectrum has a peak wavelength higher than or equal to 570 nm and lower than or equal to 750 nm, the following substances can be given.

Examples of the phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-KN]phenyl-κC}(2,2,6,6-tetramethyl-3,5-heptanedionato-κ²O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptanedionato-κ²O,O′)iridium(III) (abbreviation: [Ir(dmdppr-dmp)₂(dpm)]), (acetylacetonato)bis(2-methyl-3-phenylquinoxalinato-N,C^{2′})iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C2′)iridium(III) (abbreviation: [Ir(piq)₃]), bis(1-phenylisoquinolinato-N,C^{2′})iridium(III) acetylacetonate (abbreviation: [Ir(piq)₂(acac)]), bis[4,6-dimethyl-2-(2-quinolinyl-κN)phenyl-κC](2,4-pentanedionato-κ²O,O′)iridium(III) (abbreviation: [Ir(dmpqn)₂(acac)]) (3,7-diethyl-4,6-nonanedionato-κO4,κO6)bis[2,4-dimethyl-6-[7-(1-methylethyl)-1-isoquinolinyl-κN]phenyl-κC]iridium(III) and (3,7-diethyl-4,6-nonanedionato-κO4,κO6)bis[2,4-dimethyl-6-[5-(1-methylethyl)-2-quinolinyl-K]phenyl-κC]iridium(III); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). Alternatively, a compound obtained by substituting deuterium for part of hydrogen in any of these compounds can also be used.

### <<TADF Material>>

Any of materials described below can be used as the TADF material. The TADF material is a material that has a small energy difference between the S1 level and the T1 level (preferably less than or equal to 0.20 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation energy level and the singlet excitation energy level is greater than or equal to 0.00 eV and less than or equal to 0.20 eV, preferably greater than or equal to 0.00 eV and less than or equal to 0.10 eV. Delayed fluorescent light by the TADF material refers to light emission having a spectrum similar to that of normal fluorescent light and an extremely long lifetime. The lifetime is longer than or equal to 1×10⁻⁶ seconds, or longer than or equal to 1×10⁻³ seconds.

Note that the TADF material can be also used as an electron-transport material, a hole-transport material, or a host material.

Examples of the TADF material include fullerene, a derivative thereof, an acridine derivative such as proflavine, and eosin. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

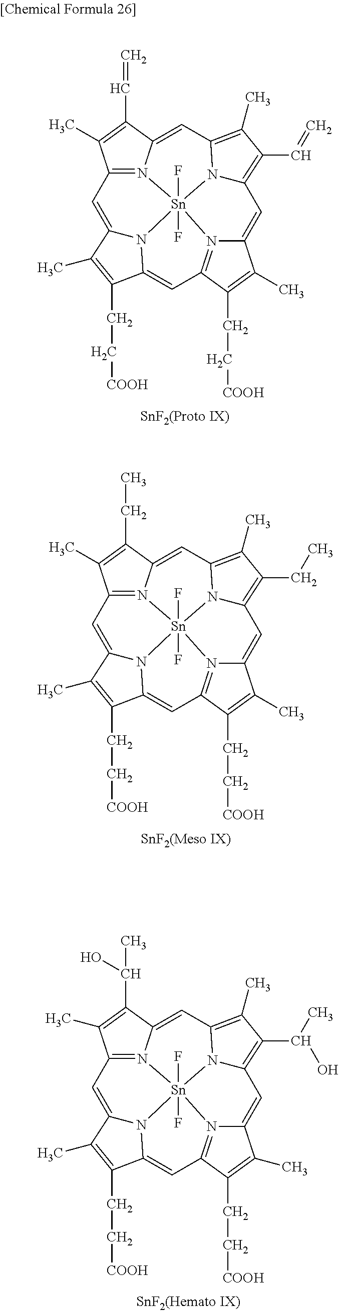

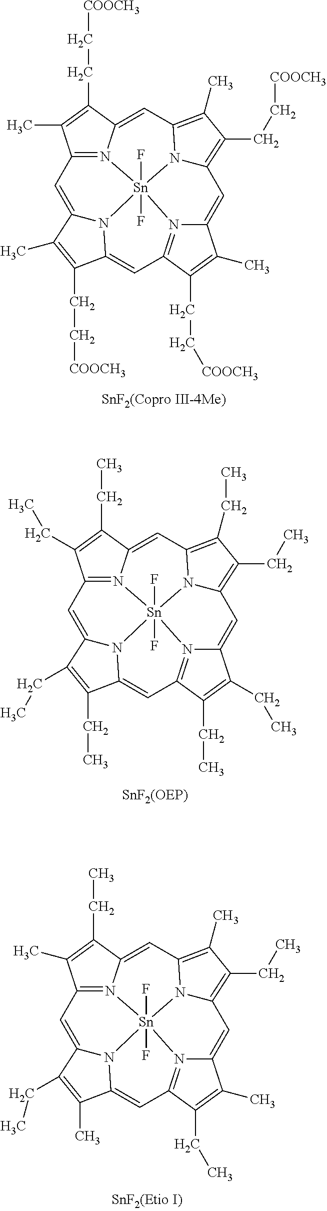

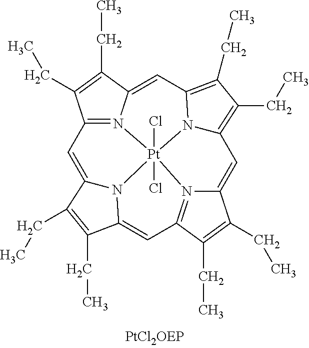

Additionally, a heteroaromatic compound including a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-[3,3′-bi-9H-carbazol]-9-yl)benzofuro[3,2-d]pyrimidine (abbreviation: 4PCCzBfpm), 4-[4-(9′-phenyl-[3,3′-bi-9H-carbazol]-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are enhanced and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

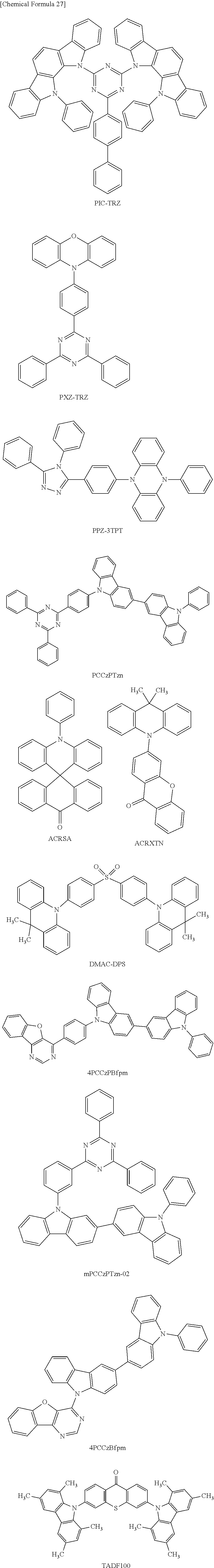

In addition to the above, another example of a material having a function of converting triplet excitation energy into light emission is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, and 113b), one or more kinds selected from substances having a larger energy gap than the light-emitting substance (guest material) can be used.

### <<Host Material for Fluorescence>>

In the case where the light-emitting substance used in the light-emitting layers (113, 113a, and 113b) is a fluorescent substance, an organic compound (a host material) used in combination with the fluorescent substance is preferably an organic compound that has a high energy level in a singlet excited state and has a low energy level in a triplet excited state or an organic compound having a high fluorescence quantum yield. Therefore, the hole-transport material (described above) and the electron-transport material (described below) shown in this embodiment, for example, can be used as long as they are organic compounds that satisfy such a condition. In addition, the organic compound represented by any of General Formula (G0) to General Formula (G7) can be used.

In terms of a preferable combination with the light-emitting substance (fluorescent substance), examples of the organic compound (host material), some of which overlap the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), PCPN, 9,10-diphenylanthracene (abbreviation: DPAnth), NN-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), CzPA, 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthryl)benzo[b]naphtho[2,3-d]furan (abbreviation: Bnf(II)PhA), 9-(1-naphthyl)-10-[4-(2-naphthyl)phenyl]anthracene (abbreviation: αN-βNPAnth), 2,9-di(1-naphthyl)-10-phenylanthracene (abbreviation: 2αN-uNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mβNPAnth), 1-{4-[10-(biphenyl-4-yl)-9-anthryl]phenyl}-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### <<Host Material for Phosphorescence>>

In the case where the light-emitting substance used in the light-emitting layers (113, 113a, and 113b) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance. In addition, the organic compound represented by any of General Formula (G0) to General Formula (G7) can be used.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferable, and it is particularly preferable to combine a compound that easily accepts holes (hole-transport material) and a compound that easily accepts electrons (electron-transport material).

In terms of a preferred combination with the light-emitting substance (phosphorescent substance), examples of the organic compounds (the host material and the assist material), some of which are mentioned in the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes.

Specific examples of the aromatic amine and the carbazole derivative, which are organic compounds having a high hole-transport property among the above-described organic compounds, are the same as the specific examples of the hole-transport materials described above, and these are each preferable as a host material.

Among the above organic compounds, specific examples of the dibenzothiophene derivative and the dibenzofuran derivative, which are organic compounds having a high hole-transport property, are mmDBFFLBi-II, DBF3P-II, DBT3P-II, DBTFLP-III, DBTFLP-IV, and 4-[3-(triphenylen-2-yl)phenyl]dibenzothiophene (abbreviation: mDBTPTp-II), and these are each preferable as a host material.

Other examples of preferable host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property, include an organic compound including a heteroaromatic ring having an azole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound including a heteroaromatic ring having a phenanthroline ring such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), or 2,2′-(1,3-phenylene)bis(9-phenyl-1,10-phenanthroline) (abbreviation: mPPhen2P); and an organic compound including a heteroaromatic ring having a dibenzoquinoxaline ring such as 2-[3-(dibenzothiophen-4-yl)phenyl]dibenzo[f;h]quinoxaline (abbreviation: 2mDBTPDBq-II), 2-[3′-(dibenzothiophen-4-yl)biphenyl-3-yl]dibenzo[f;h]quinoxaline (abbreviation: 2mDBTBPDBq-II), 2-[3′-(9H-carbazol-9-yl)biphenyl-3-yl]dibenzo[f;h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), or 2-[4′-(9-phenyl-9H-carbazol-3-yl)-3,1′-biphenyl-1-yl]dibenzo[f,h]quinoxaline (abbreviation: 2mpPCBPDBq), and these are each preferable as a host material.

Among the above organic compounds, specific examples of the pyridine derivative, the diazine derivative (e.g., the pyrimidine derivative, the pyrazine derivative, and the pyridazine derivative), the triazine derivative, and the furodiazine derivative, which are organic compounds having a high electron-transport property, include organic compounds including a heteroaromatic ring having a diazine ring such as 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), PCCzPTzn, mPCCzPTzn-02, 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 11-[3′-(dibenzothiophen-4-yl)biphenyl-3-yl]phenanthro[9′,10′:4,5]furo[2,3-b]pyrazine (abbreviation: 11mDBtBPPnfpr), 11-[3′-(dibenzothiophen-4-yl)biphenyl-4-yl]phenanthro[9′,10′:4,5]furo[2,3-b]pyrazine, 11-[(3′-9H-carbazol-9-yl)biphenyl-3-yl]phenanthro[9′,10′:4,5]furo[2,3-b]pyrazine, 12-(9′-phenyl-[3,3′-bi-9H-carbazol]-9-yl)phenanthro[9′,10′:4,5]furo[2,3-b]pyrazine (abbreviation: 12PCCzPnfpr), 9-[3′-(9-phenyl-9H-carbazol-3-yl)biphenyl-4-yl]naphtho[1′,2′:4,5]furo[2,3-b]pyrazine (abbreviation: 9pmPCBPNfpr), 9-(9′-phenyl-[3,3′-bi-9H-carbazol]-9-yl)naphtho[1′,2′:4,5]furo[2,3-b]pyrazine (abbreviation: 9PCCzNfpr), 10-(9′-phenyl-[3,3′-bi-9H-carbazol]-9-yl)naphtho[1′,2′:4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′:4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′:4,5]furo[2,3-b]pyrazine (abbreviation: 9mFDBtPNfpr), 9-[3′-(6-phenyldibenzothiophen-4-yl)biphenyl-3-yl]naphtho[1′,2′:4,5]furo[2,3-b]pyrazine (abbreviation: 9mDBtBPNfpr-02), 9-[3-(9′-phenyl-[3,3′-bi-9H-carbazol]-9-yl)phenyl]naphtho[1′,2′:4,5]furo[2,3-b]pyrazine (abbreviation: 9mPCCzPNfpr), 9-[3′-(2,8-diphenyldibenzothiophen-4-yl)biphenyl-3-yl]naphtho[1′,2′:4,5]furo[2,3-b]pyrazine, 11-[3′-(2,8-diphenyldibenzothiophen-4-yl)biphenyl-3-yl]phenanthro[9′,10′:4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-[8-([1,1′:4′,1″-terphenyl]-4-yl)-1-dibenzofuranyl]-1,3,5-triazine (abbreviation: mBP-TPDBfTzn), 6-(biphenyl-3-yl)-4-[3,5-bis(9H-carbazol-9-yl)phenyl]-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm), and these are each preferable as a host material.

Among the above organic compounds, specific examples of metal complexes that are organic compounds having a high electron-transport property include zinc- or aluminum-based metal complexes, such as tris(8-quinolinolato)aluminum(III) (abbreviation: Alq₃), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring, and these are each preferable as a host material.

Moreover, high-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy) are preferable as the host material.

Furthermore, the following organic compounds with a diazine ring, which have a bipolar property, a high hole-transport property, and a high electron-transport property, can be used as the host material: 9-phenyl-9′-(4-phenyl-2-quinazolinyl)-3,3′-bi-9H-carbazole (abbreviation: PCCzQz), 2mpPCBPDBq, mINc(II)PTzn, 11-[4-(biphenyl-4-yl)-6-phenyl-1,3,5-triazin-2-yl]-11,12-dihydro-12-phenyl-indolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### <Electron-Transport Layer>

The electron-transport layers (114, 114a, and 114b) transport electrons, which are injected from the second electrode 102 and the charge-generation layers (106, 106a, and 106b) by electron-injection layers (115, 115a, and 115b) described later, to the light-emitting layers (113, 113a, and 113b). The heat resistance of the light-emitting device of one embodiment of the present invention can be improved by including the stacked electron-transport layers. The electron-transport material used in the electron-transport layers (114, 114a, and 114b) is preferably a substance having an electron mobility higher than or equal to 1×10⁻⁶ cm²/Vs in the case where the square root of the electric field strength [V/cm] is 600. Note that any other substance can also be used as long as the substance has an electron-transport property higher than a hole-transport property. The electron-transport layers (114, 114a, and 114b) can function even with a single-layer structure and may have a stacked-layer structure including two or more layers. When a photolithography process is performed over the electron-transport layer including the above-described mixed material, which has heat resistance, an adverse effect of the thermal process on the device characteristics can be reduced.

### <<Electron-Transport Material>>

As the electron-transport material that can be used for the electron-transport layers (114, 114a, and 114b), an organic compound having a high electron-transport property can be used, and for example, a heteroaromatic compound can be used. The term heteroaromatic compound refers to a cyclic compound including at least two different kinds of elements in a ring. Examples of cyclic structures include a three-membered ring, a four-membered ring, a five-membered ring, a six-membered ring, and the like, among which a five-membered ring and a six-membered ring are particularly preferable. The elements included in the heteroaromatic compound are preferably one or more of nitrogen, oxygen, sulfur, and the like in addition to carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used. In addition, the organic compound represented by any of General Formula (G0) to General Formula (G7) can be used.

Note that the electron-transport material can be different from the materials used in the light-emitting layer. Not all excitons formed by recombination of carriers in the light-emitting layer can contribute to light emission and some excitons are diffused into a layer in contact with the light-emitting layer or a layer in the vicinity of the light-emitting layer. In order to avoid this phenomenon, the energy level (the lowest singlet excitation energy level or the lowest triplet excitation energy level) of a material used for the layer in contact with the light-emitting layer or the layer in the vicinity of the light-emitting layer is preferably higher than that of a material used for the light-emitting layer. Thus, when a material different from the material of the light-emitting layer is used as the electron-transport material, a device having high efficiency can be obtained.

The heteroaromatic compound is an organic compound including at least one heteroaromatic ring.

The heteroaromatic ring includes any one of a pyridine ring, a diazine ring, a triazine ring, an azole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having an azole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, sulfur, and the like, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, sulfur, and the like include a heteroaromatic compound having a heteroaromatic ring, such as a pyridine ring, a diazine ring (a pyrimidine ring, a pyrazine ring, a pyridazine ring, or the like), a triazine ring, or an azole ring. Other examples include a heteroaromatic compound having a bipyridine structure, a heteroaromatic compound having a terpyridine structure, and the like, which are included in examples of a heteroaromatic compound in which pyridine rings are connected.

Examples of the heteroaromatic compound having a fused ring structure partly including the above six-membered ring structure include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to a furan ring of a furodiazine ring), or a benzimidazole ring.

Specific examples of the heteroaromatic compound having a five-membered ring structure (e.g., an azole ring (including an imidazole ring, a triazole ring, and an oxadiazole ring), an oxazole ring, a thiazole ring, or a benzimidazole ring) are PBD, OXD-7, CO11, TAZ, 3-(4-tert-butylphenyl)-4-(4-ethylphenyl)-5-(4-biphenylyl)-1,2,4-triazole (abbreviation: p-EtTAZ), TPBI, mDBTBIm-II, and BzOs.

Specific examples of the above-described heteroaromatic compound having a six-membered ring structure (including a heteroaromatic ring having a pyridine ring, a diazine ring, a triazine ring, or the like) include: a heteroaromatic compound including a heteroaromatic ring having a pyridine ring, such as 35DCzPPy or TmPyPB; a heteroaromatic compound including a heteroaromatic ring having a triazine ring, such as PCCzPTzn, mPCCzPTzn-02, mINc(II)PTzn, mTpBPTzn, BP-SFTzn, 2,4NP-6PyPPm, PCDBfTzn, mBP-TPDBfTzn, 2-{3-[3-(dibenzothiophen-4-yl)phenyl]phenyl}-4,6-diphenyl-1,3,5-triazine (abbreviation: mDBtBPTzn), or mFBPTzn; and a heteroaromatic compound including a heteroaromatic ring having a diazine (pyrimidine) ring, such as 4,6mPnP2Pm, 4,6mDBTP2Pm-II, 4,6mCzP2Pm, 4,6mCzBP2Pm, 6mBP-4Cz2PPm, 6BP-4Cz2PPm, 4-[3-(dibenzothiophen-4-yl)phenyl]-8-(naphthalen-2-yl)-benzofuro[3,2-d]pyrimidine (abbreviation: 8βN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-benzofuro[3,2-d]pyrimidine (abbreviation: 4,8mDBtP2Bfpm), 8-[3′-(dibenzothiophen-4-yl)biphenyl-3-yl]naphtho[1′,2′:4,5]furo[3,2-d]pyrimidine (abbreviation: 8mDBtBPNfpm), or 8-([2,2′-binaphthalen]-6-yl)-4-[3-(dibenzothiophen-4-yl)phenyl]-[1]benzofuro[3,2-d]pyrimidine (abbreviation: 8(PN2)-4mDBtPBfpm). Note that the above aromatic compounds including a heteroaromatic ring include a heteroaromatic compound having a fused heteroaromatic ring.

Other examples include heteroaromatic compounds including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-([2,2′-bipyridine]-6,6′-diyl)bis(4-phenylbenzo[h]quinazoline) (abbreviation: 6,6′(P-Bqn)2BPy), 2,2′-(pyridine-2,6-diyl)bis{4-[4-(2-naphthyl)phenyl]-6-phenylpyrimidine}(abbreviation: 2,6(NP-PPm)2Py), or 6mBP-4Cz2PPm, and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tzn), or 2-[3-(2,6-dimethyl-3-pyridyl)-5-(9-phenanthryl)phenyl]-4,6-diphenyl-1,3,5-triazine (abbreviation: mPn-mDMePyPTzn).

Specific examples of the above-described heteroaromatic compound having a fused ring structure partly including a six-membered ring structure (the heteroaromatic compound having a fused ring structure) include a heteroaromatic compound having a quinoxaline ring, such as Bphen, BCP, NBphen, mPPhen2P, 2,6(P-Bqn)2Py, 2mDBTPDBq-II, 2mDBTBPDBq-II, 2mCzBPDBq, 2CzPDBq-III, 7mDBTPDBq-II, 6mDBTPDBq-II, and 2mpPCBPDBq. In particular, an organic compound having a phenanthroline ring, such as NBphen (T_{g}: 165° C.), mPPhen2P (T_{g}: 135° C.), 2,2′-(biphenyl-4,4′-diyl)bis(9-phenyl-1,10-phenanthroline) (abbreviation: PPhen2BP) (T_{g}: 166° C.), 2,2′-biphenyl-3,3′-diylbis(9-phenyl-1,10-phenanthroline) (abbreviation: mPPhen2BP) (T_{g}: 144° C.), 2,8-bis(phenanthrolin-5-yl)dibenzofuran (abbreviation: 2,8Phen2DBf) (T_{g}: 210° C.), or 5,5′,5″-(benzene-1,3,5-triyl)tri-1,10-phenanthroline (abbreviation: Phen3P) (T_{g}: 257° C.), is preferable because it has high T_{g} of powder, higher than or equal to 100° C.

For the electron-transport layers (114, 114a, and 114b), any of the metal complexes below can be used as well as the heteroaromatic compounds described above. Examples of the metal complexes include a metal complex having a quinoline ring or a benzoquinoline ring, such as Alq₃, Almq₃, 8-quinolinolato-lithium (abbreviation: Liq), BAlq, and Znq, and a metal complex having an oxazole ring or a thiazole ring, such as ZnPBO and ZnBTZ.

It is also possible to use high-molecular compounds such as PPy, PF-Py, and PF-BPy as the electron-transport material.

Each of the electron-transport layers (114, 114a, and 114b) is not limited to a single layer and may be a stack of two or more layers each including any of the above substances.

### <Electron-Injection Layer>

The electron-injection layers (115, 115a, and 115b) include a substance having a high electron-injection property. The electron-injection layers (115, 115a, and 115b) are layers for increasing the efficiency of electron injection from the second electrode 102 and are preferably formed using a material whose value of the LUMO level has a small difference (less than or equal to 0.50 eV) from the work function of a material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal or a compound of a rare earth metal, such as erbium fluoride (ErF₃) or ytterbium (Yb), can also be used. It is also possible to use a compound including a 1,3,4,6,7,8-tetrahydro-2H-pyrimido[1,2-a]pyrimidine skeleton, such as 1-(9,9′-spirobi[9H-fluoren]-2-yl)-1,3,4,6,7,8-hexahydro-2H-pyrimido[1,2-a]pyrimidine (abbreviation: 2hppSF), 1,1′-(9,9′-spirobi[9H-fluorene]-2,7-diyl)bis(1,3,4,6,7,8-hexahydro-2H-pyrimido[1,2-a]pyrimidine) (abbreviation: 2,7hpp2SF), or 1,1′-pyridine-2,6-diyl-bis(1,3,4,6,7,8-hexahydro-2H-pyrimido[1,2-a]pyrimidine) (abbreviation: hpp2Py). To form the electron-injection layers (115, 115a, and 115b), two or more of the above materials may be mixed or stacked. Electride may also be used for the electron-injection layers (115, 115a, and 115b). Examples of an electride include substances in which electrons are added at high concentration to a calcium oxide-aluminum oxide. Any of the substances for forming the electron-transport layers (114, 114a, and 114b), which are given above, can also be used.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound can be used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A mixed material in which an organic compound and a metal are mixed may also be used for the electron-injection layers (115, 115a, and 115b). The organic compound used here preferably has a LUMO level higher than or equal to −3.60 eV and lower than or equal to −2.30 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure partly including a six-membered ring structure (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal that belongs to Group 5, Group 7, Group 9, or Group 11 or a material that belongs to Group 13 in the periodic table is preferably used, and examples thereof include Ag, Cu, Al, and In. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength k of light emitted from the light-emitting layer 113b. In that case, the optical path length can be adjusted by changing the thickness of the electron-transport layer 114b or the electron-injection layer 115b.

### <Charge-Generation Layer>

The charge-generation layer 106 has a function of injecting electrons into the EL layer 103a and injecting holes into the EL layer 103b when voltage is applied between the first electrode (anode) 101 and the second electrode (cathode) 102. The charge-generation layer 106 may be either a p-type layer in which an electron acceptor is added to a hole-transport material or an electron-injection buffer layer in which an electron donor is added to an electron-transport material. Alternatively, both of these structures may be stacked. Furthermore, an electron-relay layer may be provided between the p-type layer and the electron-injection buffer layer. Note that forming the charge-generation layer 106 with the use of any of the above materials can inhibit an increase in driving voltage caused by the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include F₄-TCNQ and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films including the respective materials may be used.

In the case where the charge-generation layer 106 is an electron-injection buffer layer in which an electron donor is added to an electron-transport material, any of the materials described in this embodiment can be used as the electron-transport material. In addition, the organic compound represented by any of General Formula (G0) to General Formula (G7) can be used. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An alkali metal compound such as Liq may be used. An organic compound such as tetrathianaphthacene may be used as the electron donor. An organic compound including a 1,3,4,6,7,8-tetrahydro-2H-pyrimido[1,2-a]pyrimidine skeleton, such as 2hppSF, 2,7hpp2SF, or hpp2Py may be used as the electron donor. When any of these organic compounds is used as the electron donor, the electron-transport material to be combined with the electron donor is preferably an organic compound including a heteroaromatic ring having a phenanthroline ring, such as Bphen, BCP, NBphen, or 2,2′-(1,3-phenylene)bis(9-phenyl-1,10-phenanthroline) (abbreviation: mPPhen2P), in which case driving voltage of the light-emitting device can be reduced.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer can be higher than or equal to −5.00 eV, further preferably higher than or equal to −5.00 eV and lower than or equal to −3.00 eV. Note that as the substance having an electron-transport property in the electron-relay layer, a phthalocyanine-based material or a metal complex having a metal-oxygen bond and an aromatic ligand is preferably used.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

Although FIG. 1E illustrates the structure in which two EL layers 103 are stacked, three or more EL layers may be stacked with charge-generation layers each provided between different EL layers.

### <Cap Layer>

Although not illustrated in FIGS. 1A to 1F, a cap layer may be provided over the second electrode 102 of the light-emitting device. For example, a material with a high refractive index of a film can be used for the cap layer. When the cap layer is provided over the second electrode 102, extraction efficiency of light emitted through the second electrode 102 can be improved.

Specific examples of a material that can be used for the cap layer include 5,5′-diphenyl-2,2′-di-5H-[1]benzothieno[3,2-c]carbazole (abbreviation: BisBTc) and DBT3P-II.

### <Substrate>

The light-emitting device described in this embodiment can be formed over a variety of substrates. Note that the type of the substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, and paper or a base material film including a fibrous material.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as an acrylic resin, polypropylene, polyester, polyvinyl fluoride, polyvinyl chloride, polyamide, polyimide, aramid, an epoxy resin, an inorganic vapor deposition film, and paper.

For fabrication of the light-emitting device in this embodiment, a gas phase method such as an evaporation method or a liquid phase method such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers with various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high-molecular compound (e.g., an oligomer, a dendrimer, or a polymer), a middle-molecular compound (a compound between a low-molecular compound and a high-molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

The structures described in this embodiment can be used in appropriate combination with any of the structures described in the other embodiments.

### Embodiment 3

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head-mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and notebook personal computers, a monitor of a computer and the like, digital signage, and a large game machine such as a pachinko machine.

### [Display Module]

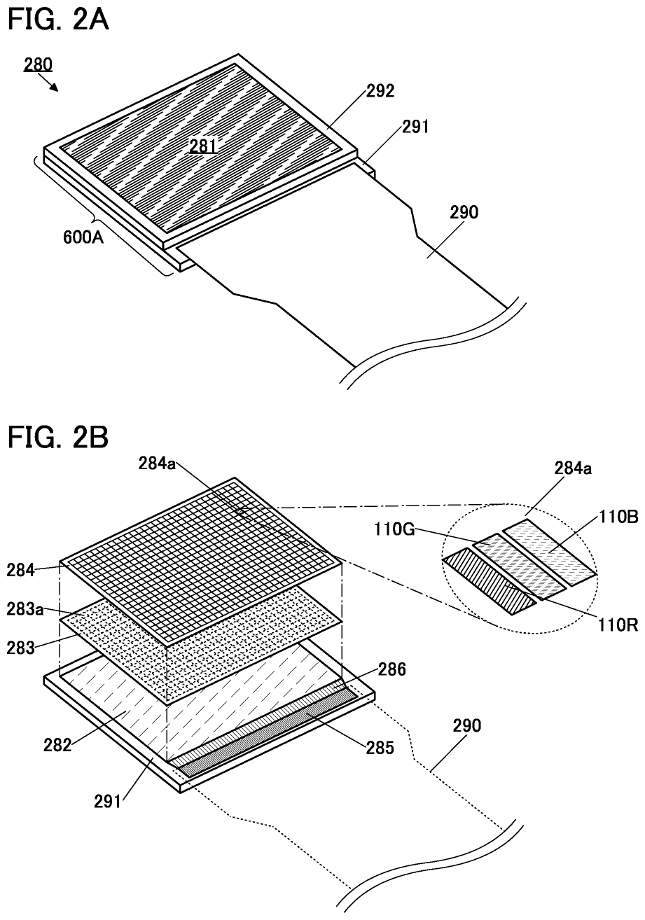

FIG. 2A is a perspective view of a display module 280. The display module 280 includes a display device 600A and an FPC 290.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 2B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion over the substrate 291 that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 2B. The pixels 284a can employ any of the structures described in the above embodiments

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor per light-emitting device. A gate signal is input to a gate of the selection transistor, and a video signal is input to a source or a drain of the selection transistor. Thus, an active-matrix display device is achieved.

The circuit portion 282 includes a circuit for driving the pixel circuits 283a in the pixel circuit portion 283. For example, the circuit portion 282 preferably includes one or both ofa gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%. Furthermore, the pixels 284a can be arranged extremely densely and thus the display portion 281 can have significantly high resolution. For example, the pixels 284a are preferably arranged in the display portion 281 to give a resolution higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

The pixel 284a includes a subpixel 110R, a subpixel 110G, and a subpixel 110B.

In this specification and the like, for example, description common to the subpixel 110R, the subpixel 110G, and the subpixel 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on a pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared light (IR).

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion. For example, the display module 280 can be suitably used in a display portion of a wearable electronic appliance, such as a wrist watch.

### [Display Device 600A]

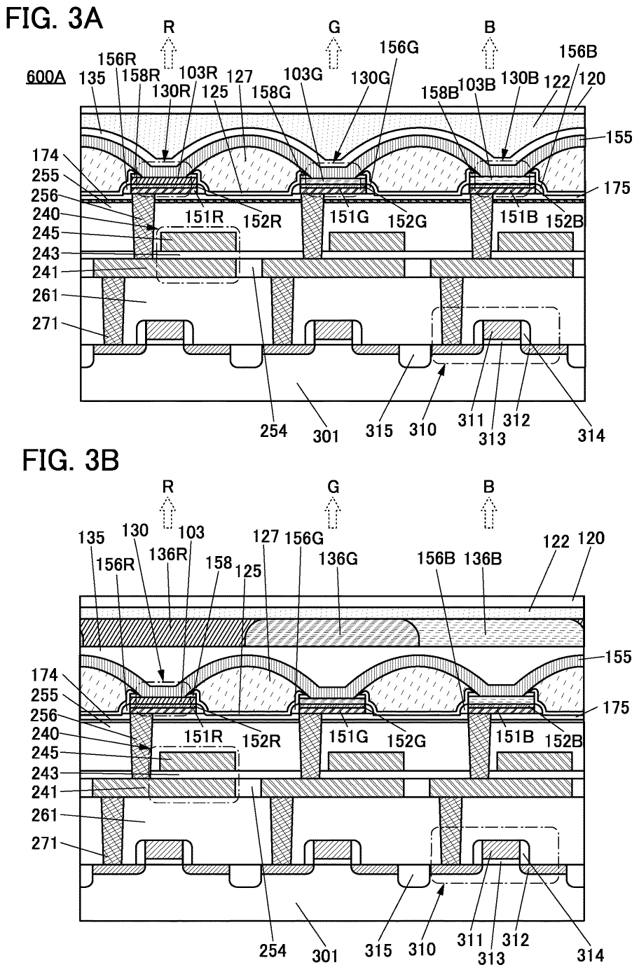

The display device 600A illustrated in FIG. 3A includes a substrate 301, a light-emitting device 130R, a light-emitting device 130G, and a light-emitting device 130B, a capacitor 240, and a transistor 310.

The substrate 301 corresponds to the substrate 291 in FIGS. 2A and 2B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layer 241 and the conductive layer 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. An insulating layer 174 is provided over the insulating layer 255. An insulating layer 175 is provided over the insulating layer 174. The light-emitting device 130R, the light-emitting device 130G, and the light-emitting device 130B are provided over the insulating layer 175. The light-emitting device 130R, the light-emitting device 130G, and the light-emitting device 130B each have a structure as described in Embodiments 1 and 2. An insulator is provided in regions between adjacent light-emitting devices. For example, in FIG. 3A, an inorganic insulating layer 125 and an insulating layer 127 over the inorganic insulating layer 125 are provided in those regions.

Each of the light-emitting devices included in the display device 600A is a light-emitting device in which an EL layer is formed by processing a film to be the EL layer by a lithography method.

The light-emitting device 130R includes the first electrode including a conductive layer 151R and a conductive layer 152R, an EL layer 103R over the first electrode, and a common layer 155 over the EL layer 103R. The light-emitting device 130G includes a first electrode including a conductive layer 151G and a conductive layer 152G, an EL layer 103G over the first electrode, and the common layer 155 over the EL layer 103G. The light-emitting device 130B includes a first electrode including a conductive layer 151B and a conductive layer 152B, an EL layer 103B over the first electrode, and the common layer 155 over the EL layer 103B. The common layer 155 shared by the light-emitting devices includes at least the second electrode. The common layer 155 may include an electron-injection layer positioned between the second electrode and each EL layer. For example, the light-emitting device 130R can emit red light, the light-emitting device 130G can emit green light, and the light-emitting device 130B can emit blue light. In this specification and the like, description common to the light-emitting device 130R, the light-emitting device 130G, and the light-emitting device 130B is sometimes made using the collective term “light-emitting device 130”. In this specification and the like, description common to the conductive layer 151R, the conductive layer 151G, and the conductive layer 151B is sometimes made using the collective term “conductive layer 151”. In this specification and the like, description common to the conductive layer 152R, the conductive layer 152G, and the conductive layer 152B is sometimes made using the collective term “conductive layer 152”.

In the light-emitting device 130, one of the first electrode and the second electrode functions as an anode and the other functions as a cathode. Hereinafter, description is made on the assumption that the first electrode functions as the anode and the second electrode functions as the cathode unless otherwise specified.

The EL layers included in the light-emitting device 130 are island-shaped and independent of each other on a light-emitting device basis or on an emission color basis. Providing the island-shaped EL layer 103 in each of the light-emitting devices 130 can suppress leakage current between the adjacent light-emitting devices 130 even in a high-resolution display device. This can prevent crosstalk, so that a display device with extremely high contrast can be obtained. Specifically, a display device having high current efficiency at low luminance can be obtained.

In the display device of one embodiment of the present invention, the first electrode (pixel electrode) of the light-emitting device preferably has a stacked-layer structure. For example, in the example shown in FIG. 3A, the first electrode of the light-emitting device 130 is a stack of the conductive layer 151 and the conductive layer 152. In the case where the display device 600A is of a top-emission type and the pixel electrode of the light-emitting device 130 functions as the anode, for example, the conductive layer 151 preferably has high visible light reflectance, and the conductive layer 152 preferably has a visible-light-transmitting property and a high work function. In the case where the display device 600A is of a top-emission type, the higher the visible light reflectance of the pixel electrode is, the higher the efficiency of extraction of the light emitted by the EL layer 103 is. In the case where the pixel electrode functions as the anode, the higher the work function of the pixel electrode is, the easier hole injection into the EL layer 103 is. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage.

In the case where the conductive layer 151 has high reflectance for visible light, the visible light reflectance of the conductive layer 151 is preferably higher than or equal to 40% and lower than or equal to 100%, or higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

Thus, in the display device 600A of this embodiment, an insulating layer 156 (insulating layers 156R, 156G, and 156B) is formed on the side surfaces of the conductive layer 151 and the conductive layer 152. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display device 600A to be manufactured by a high-yield method and to be accordingly inexpensive. In addition, generation of a defect in the display device 600A can be inhibited, which makes the display device 600A highly reliable. In this specification and the like, description common to the insulating layer 156R, the insulating layer 156G, and the insulating layer 156B is sometimes made using the collective term “insulating layer 156”. A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, an indium tin oxide containing silicon can be suitably used for the conductive layer 152 because of having a work function of higher than or equal to 4.0 eV, for example.

The conductive layer 151 is electrically connected to one of the source and the drain of the corresponding transistor 310 through a plug 256 embedded in the insulating layer 243, the insulating layer 255, the insulating layer 174, and the insulating layer 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

A sacrificial layer 158R is provided over the EL layer 103R of the light-emitting device 130R. A sacrificial layer 158G is provided over the EL layer 103G of the light-emitting device 130G. A sacrificial layer 158B is provided over the EL layer 103B of the light-emitting device 130B. Providing the sacrificial layer 158R, the sacrificial layer 158G, and the sacrificial layer 158B can reduce damage to the EL layer in forming the EL layer by a lithography method in the manufacturing process of the light-emitting device. Specifically, a sacrificial film to be the sacrificial layer is formed over a film to be the EL layer and then the EL layer and the sacrificial layer are formed by a lithography method, whereby the deterioration of the EL layer can be inhibited. After that, it is preferable to further remove part of the sacrificial layer to form the sacrificial layer 158R, the sacrificial layer 158G, or the sacrificial layer 158B so that the top surface of the EL layer is exposed.

As a film to be the sacrificial layer 158R, the sacrificial layer 158G, and the sacrificial layer 158B, one or more of a metal film, an alloy film, a metal oxide film, a semiconductor film, an organic insulating film, and an inorganic insulating film, for example, can be used.

A protective layer 135 is provided over the light-emitting device 130. A substrate 120 is bonded onto the protective layer 135 with a resin layer 122. The substrate 120 corresponds to the substrate 292 in FIG. 2A.

FIG. 3B illustrates a modification example of the display device 600A illustrated in FIG. 3A. The display device illustrated in FIG. 3B includes a coloring layer 136R, a coloring layer 136G, and a coloring layer 136B, and each of the light-emitting devices 130 includes a region overlapping with one of the coloring layer 136R, the coloring layer 136G, and the coloring layer 136B. In the display device illustrated in FIG. 3B, the light-emitting device 130 can emit white light, for example. For example, the coloring layer 136R, the coloring layer 136G, and the coloring layer 136B can transmit red light, green light, and blue light, respectively.

### [Display Device 600B]

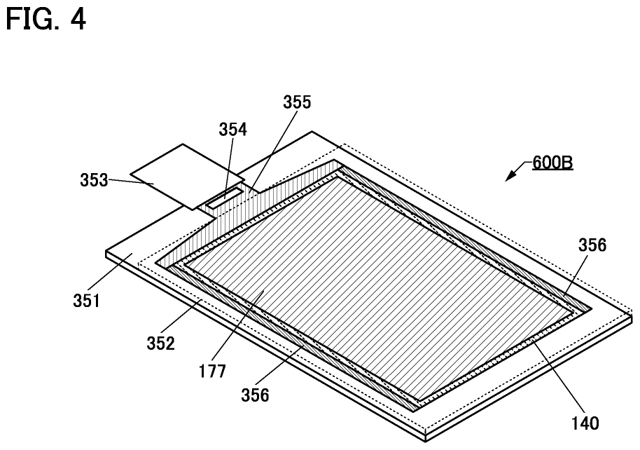

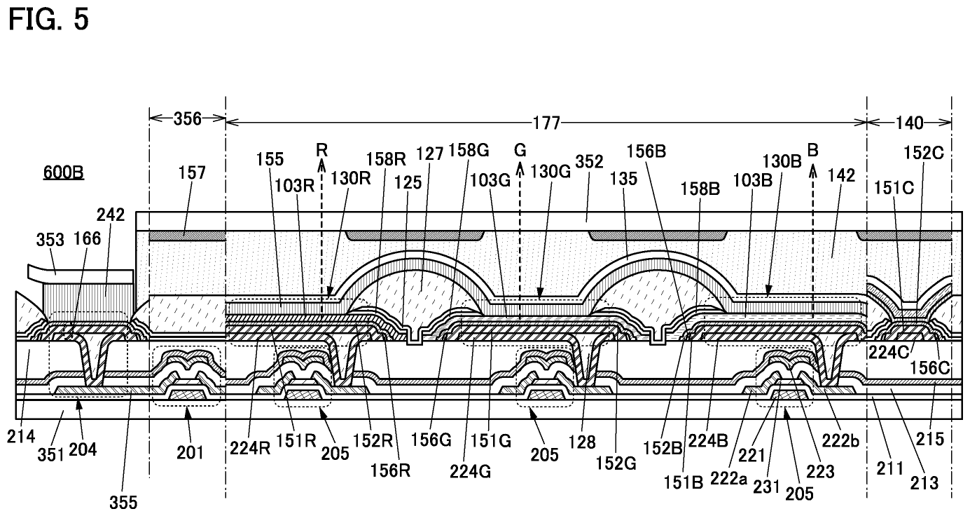

FIG. 4 is a perspective view of a display device 600B, and FIG. 5 is a cross-sectional view of the display device 600B.

In the display device 600B, a substrate 352 and a substrate 351 are bonded to each other. In FIG. 4, the substrate 352 is denoted by a dashed line.

The display device 600B includes the pixel portion 177, a connection portion 140, a circuit 356, a wiring 355, and the like. FIG. 4 shows an example in which an integrated circuit (IC) 354 and an FPC 353 are mounted on the display device 600B. Thus, the structure illustrated in FIG. 4 can be regarded as a display module including the display device 600B, the IC, and the FPC. Here, a display device in which a substrate is equipped with a connector such as an FPC or mounted with an IC is referred to as a display module.

The connection portion 140 is provided outside the pixel portion 177. The connection portion 140 can be provided along one side or a plurality of sides of the pixel portion 177. The number of the connection portions 140 may be one or more. FIG. 4 illustrates an example in which the connection portion 140 is provided to surround the four sides of the pixel portion 177. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from an IC 354.

FIG. 4 illustrates an example in which the IC 354 is provided for the substrate 351 by a chip on glass (COG) method, a chip on film (COF) method, or the like. An IC including a scan line driver circuit, a signal line driver circuit, or the like can be used as the IC 354, for example. Note that the display device 600B and the display module are not necessarily provided with an IC. Alternatively, the IC may be mounted on the FPC by a COF method, for example.

FIG. 5 shows an example of cross sections of part of a region including the FPC 353, part of the circuit 356, part of the pixel portion 177, part of the connection portion 140, and part of a region including an end portion of the display device 600B.

The display device 600B shown in FIG. 5 includes a transistor 201, a transistor 205, the light-emitting device 130R that emits red light, the light-emitting device 130G that emits green light, the light-emitting device 130B that emits blue light, and the like between the substrate 351 and the substrate 352.

The stacked-layer structure of each of the light-emitting device 130R, the light-emitting device 130G, and the light-emitting device 130B is the same as that shown in FIG. 3A except for the structure of the pixel electrode. The above embodiments can be referred to for the details of the light-emitting devices.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B. Here, the conductive layer 224R, the conductive layer 151R, and the conductive layer 152R can be collectively referred to as the pixel electrode of the light-emitting device 130R; the conductive layer 151R and the conductive layer 152R excluding the conductive layer 224R can also be referred to as the pixel electrode of the light-emitting device 130R. Similarly, the conductive layer 224G, the conductive layer 151G, and the conductive layer 152G can be collectively referred to as the pixel electrode of the light-emitting device 130G; the conductive layer 151G and the conductive layer 152G excluding the conductive layer 224G can also be referred to as the pixel electrode of the light-emitting device 130G. The conductive layer 224B, the conductive layer 151B, and the conductive layer 152B can be collectively referred to as the pixel electrode of the light-emitting device 130B; the conductive layer 151B and the conductive layer 152B excluding the conductive layer 224B can also be referred to as the pixel electrode of the light-emitting device 130B.

The conductive layer 224R is connected to a conductive layer 222b included in the transistor 205 through an opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layer 224G, the conductive layer 151G, and the conductive layer 152G, and the insulating layer 156G in the light-emitting device 130G are not described in detail because they are respectively similar to the conductive layer 224R, the conductive layer 151R, and the conductive layer 152R, and the insulating layer 156R in the light-emitting device 130R; the same applies to the conductive layer 224B, the conductive layer 151B, and the conductive layer 152B, and the insulating layer 156B in the light-emitting device 130B.

The conductive layer 224R, the conductive layer 224G, and the conductive layer 224B each have a depressed portion covering the opening provided in the insulating layer 214. A layer 128 is embedded in the depressed portion.

The layer 128 has a function of filling the depressed portions of the conductive layer 224R, the conductive layer 224G, and the conductive layer 224B to obtain planarity. Over the conductive layer 224R, the conductive layer 224G, and the conductive layer 224B and the layer 128, the conductive layer 151R, the conductive layer 151G, and the conductive layer 151B that are respectively electrically connected to the conductive layer 224R, the conductive layer 224G, and the conductive layer 224B are provided. Thus, the regions overlapping with the depressed portions of the conductive layer 224R, the conductive layer 224G, and the conductive layer 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 135 is provided over the light-emitting device 130R, the light-emitting device 130G, and the light-emitting device 130B. The protective layer 135 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 5, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided in a frame shape not to overlap with the light-emitting device. Furthermore, the space may be filled with a resin other than the frame-like adhesive layer 142.

FIG. 5 shows an example in which the connection portion 140 includes a conductive layer 224C obtained by processing the same conductive film as the conductive layer 224R, the conductive layer 224G, and the conductive layer 224B; a conductive layer 151C obtained by processing the same conductive film as the conductive layer 151R, the conductive layer 151G, and the conductive layer 151B; and a conductive layer 152C obtained by processing the same conductive film as the conductive layer 152R, the conductive layer 152G, and the conductive layer 152B. In the example illustrated in FIG. 5, an insulating layer 156C is provided to include a region overlapping with the side surface of the conductive layer 151C.

The display device 600B has a top-emission structure. Light from the light-emitting device is emitted toward the substrate 352. For the substrate 352, a material with a high visible-light-transmitting property is preferably used. The pixel electrode includes a material that reflects visible light, and a counter electrode (the common layer 155) includes a material that transmits visible light.

The transistor 201 and the transistor 205 are formed over the substrate 351. These transistors can be fabricated using the same materials in the same steps.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

A material that does not easily allow diffusion of impurities such as water and hydrogen is preferably used for at least one of the insulating layers covering the transistors. This is because such an insulating layer can function as a barrier layer. Such a structure can effectively inhibit diffusion of impurities into the transistors from the outside and improve the reliability of a display device.

An inorganic insulating film is preferably used as each of the insulating layer 211, the insulating layer 213, and the insulating layer 215. As the inorganic insulating film, a silicon nitride film, a silicon oxynitride film, a silicon oxide film, a silicon nitride oxide film, an aluminum oxide film, or an aluminum nitride film can be used, for example. A hafnium oxide film, an yttrium oxide film, a zirconium oxide film, a gallium oxide film, a tantalum oxide film, a magnesium oxide film, a lanthanum oxide film, a cerium oxide film, a neodymium oxide film, or the like may be used. Two or more of the above insulating films may also be stacked.

An organic insulating layer is suitable as the insulating layer 214 functioning as a planarization layer. Examples of materials that can be used for the organic insulating layer include an acrylic resin, a polyimide resin, an epoxy resin, a polyamide resin, a polyimide-amide resin, a siloxane resin, a benzocyclobutene-based resin, a phenol resin, and precursors of these resins. The insulating layer 214 may have a stacked-layer structure of an organic insulating layer and an inorganic insulating layer. The outermost layer of the insulating layer 214 preferably functions as an etching protective layer. This can inhibit formation of a depressed portion in the insulating layer 214 at the time of processing of the conductive layer 224R, the conductive layer 151R, the conductive layer 152R, or the like. Alternatively, a depressed portion may be provided in the insulating layer 214 at the time of processing of the conductive layer 224R, the conductive layer 151R, the conductive layer 152R, or the like.

Each of the transistor 201 and the transistor 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate. Here, a plurality of layers obtained by processing the same conductive film are shown with the same hatching pattern. The insulating layer 211 is positioned between the conductive layer 221 and the semiconductor layer 231. The insulating layer 213 is positioned between the conductive layer 223 and the semiconductor layer 231.

There is no particular limitation on the structure of the transistors included in the display device of this embodiment. For example, a planar transistor, a fin-type transistor, a staggered transistor, an inverted staggered transistor, or a vertical transistor can be used. A top-gate transistor or a bottom-gate transistor can be used. Alternatively, gates may be provided above and below a semiconductor layer where a channel is formed.

The structure in which the semiconductor layer where a channel is formed is provided between two gates is employed for each of the transistor 201 and the transistor 205. The two gates may be connected to each other and supplied with the same signal to drive the transistor. Alternatively, the threshold voltage of the transistor may be controlled by applying a potential for controlling the threshold voltage to one of the two gates and a potential for driving to the other of the two gates.

There is no particular limitation on the crystallinity of a semiconductor material used for the transistors, and either an amorphous semiconductor or a semiconductor having crystallinity (a microcrystalline semiconductor, a polycrystalline semiconductor, a single crystal semiconductor, or a semiconductor partly including crystal regions) can be used.

The semiconductor layer of the transistor preferably includes an oxide semiconductor. That is, a transistor including an oxide semiconductor in its channel formation region (hereinafter referred to as an OS transistor) is preferably used in the display device of this embodiment.

Alternatively, a transistor including silicon in its channel formation region (a Si transistor) may be used. Examples of silicon include single crystal silicon, polycrystalline silicon, and amorphous silicon. In particular, a transistor including low-temperature polysilicon (LTPS) in its semiconductor layer (hereinafter also referred to as an LTPS transistor) can be used. The LTPS transistor has high field-effect mobility and excellent frequency characteristics.

An OS transistor has much higher field-effect mobility than a transistor including amorphous silicon. In addition, the OS transistor has an extremely low leakage current between a source and a drain in an off state, and charge accumulated in a capacitor that is connected in series to the transistor can be held for a long period. Furthermore, the use of an OS transistor can reduce the power consumption of the display device.

The semiconductor layer in the OS transistor is preferably an oxide containing indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin. Alternatively, the semiconductor layer in the OS transistor is preferably an oxide containing indium (In) (also referred to as IO).

The transistors included in the circuit 356 and the transistors included in the pixel portion 177 may have the same structure or different structures. One structure or two or more kinds of structures may be employed for a plurality of transistors included in the circuit 356. Similarly, one structure or two or more kinds of structures may be employed for a plurality of transistors included in the pixel portion 177.

All transistors included in the pixel portion 177 may be OS transistors, or all transistors included in the pixel portion 177 may be Si transistors. Alternatively, some of the transistors included in the pixel portion 177 may be OS transistors and the others may be Si transistors.

For example, when both an LTPS transistor and an OS transistor are used in the pixel portion 177, the display device can have low power consumption and high driving capability. Note that a structure in which an LTPS transistor and an OS transistor are used in combination is referred to as LTPO in some cases. For example, it is preferable that an OS transistor be used as a transistor functioning as a switch for controlling electrical continuity between wirings and an LTPS transistor be used as a transistor for controlling a current.

For example, one transistor included in the pixel portion 177 functions as a transistor for controlling a current flowing through the light-emitting device and can be referred to as a driving transistor. One of a source and a drain of the driving transistor is electrically connected to the pixel electrode of the light-emitting device. An LTPS transistor is preferably used as the driving transistor. In that case, the amount of current flowing through the light-emitting device can be increased in the pixel circuit.

Another transistor included in the pixel portion 177 functions as a switch for controlling selection or non-selection of a pixel and can also be referred to as a selection transistor. A gate of the selection transistor is electrically connected to a gate line, and one of a source and a drain thereof is electrically connected to a source line (signal line). An OS transistor is preferably used as the selection transistor. In that case, the gray level of the pixel can be maintained even with an extremely low frame frequency (e.g., lower than or equal to 1 fps); thus, power consumption can be reduced by stopping the driver in displaying a still image.

As described above, the display device of one embodiment of the present invention can have all of a high aperture ratio, high resolution, high display quality, and low power consumption.

Note that the display device of one embodiment of the present invention has a structure including the OS transistor and the light-emitting device having a metal maskless (MML) structure. This structure can significantly reduce a leakage current that would flow through a transistor and a leakage current that would flow between adjacent light-emitting devices (sometimes referred to as a horizontal leakage current or a lateral leakage current). Displaying images on the display device having this structure can bring one or more of image crispness, image sharpness, high color saturation, and a high contrast ratio to the viewer. When a leakage current that would flow through the transistor and a lateral leakage current that would flow between the light-emitting devices are extremely low, leakage of light at the time of black display (black-level degradation) or the like can be minimized.

In particular, in the case where a light-emitting device having an MML structure employs a side-by-side (SBS) structure, which is the above-described structure for separately forming or coloring light-emitting layers, a layer provided between light-emitting devices (for example, also referred to as an organic layer or a common layer which is shared by the light-emitting devices) is disconnected; accordingly, side leakage can be prevented or be made extremely low.

A connection portion 204 is provided in a region of the substrate 351 not overlapping with the substrate 352. In the connection portion 204, the wiring 355 is electrically connected to the FPC 353 through a conductive layer 166 and a connection layer 242. As an example, the conductive layer 166 has a stacked-layer structure of a conductive film obtained by processing the same conductive film as the conductive layer 224R, the conductive layer 224G, and the conductive layer 224B; a conductive film obtained by processing the same conductive film as the conductive layer 151R, the conductive layer 151G, and the conductive layer 151B; and a conductive film obtained by processing the same conductive film as the conductive layer 152R, the conductive layer 152G, and the conductive layer 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrate 351 and the substrate 352.

A material that can be used for the resin layer 122 can be used for the adhesive layer 142.

As the connection layer 242, an anisotropic conductive film (ACF), an anisotropic conductive paste (ACP), or the like can be used.

Although the top-emission display device is described in this embodiment, the display device of one embodiment of the present invention may have a bottom-emission structure.

This embodiment can be combined as appropriate with the other embodiments. In this specification, in the case where a plurality of structure examples are shown in one embodiment, the structure examples can be combined as appropriate.

### Embodiment 4

In this embodiment, electronic appliances of embodiments of the present invention will be described.

Electronic appliances of this embodiment include the display device of one embodiment of the present invention in their display portions. The display device of one embodiment of the present invention is highly reliable and can be easily increased in resolution and definition. Thus, the display device of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the display device of one embodiment of the present invention can have high resolution, and thus can be suitably used for an electronic appliance having a relatively small display portion. Examples of such an electronic appliance include watch-type and bracelet-type information terminals (wearable devices) and wearable devices capable of being worn on a head, such as a VR device like a head-mounted display, a glasses-type AR device, and an MR device.

The definition of the display device of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, definition of 4K, 8K, or higher is preferable. The pixel density (resolution) of the display device of one embodiment of the present invention is preferably higher than or equal to 100 ppi, further preferably higher than or equal to 300 ppi, still further preferably higher than or equal to 500 ppi, yet still further preferably higher than or equal to 1000 ppi, yet still further preferably higher than or equal to 2000 ppi, yet still further preferably higher than or equal to 3000 ppi, yet still further preferably higher than or equal to 5000 ppi, yet still further preferably higher than or equal to 7000 ppi. With such a display device having high definition and/or high resolution, the electronic appliance can provide higher realistic sensation, sense of depth, and the like. There is no particular limitation on the screen ratio (aspect ratio) of the display device of one embodiment of the present invention. For example, the display device is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance in this embodiment can have a variety of functions. For example, the electronic appliance in this embodiment can have a function of displaying a variety of information (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

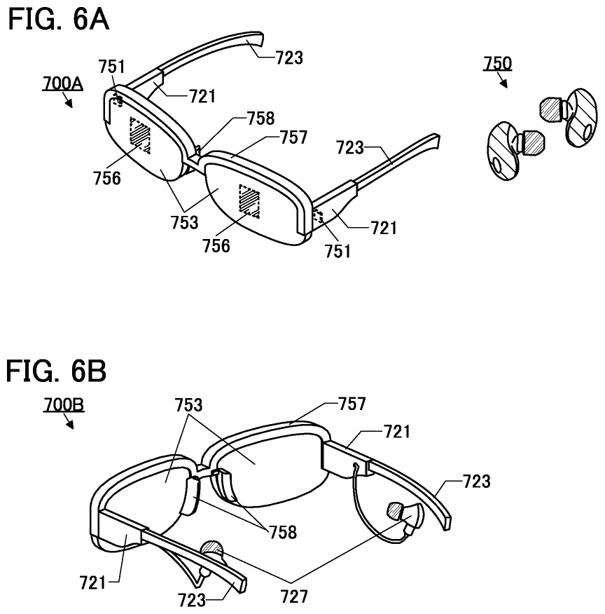

Examples of a wearable device capable of being worn on a head will be described with reference to FIGS. 6A and 6B. These wearable devices have at least one of a function of displaying AR contents, a function of displaying VR contents, a function of displaying SR contents, and a function of displaying MR contents. The electronic appliance having a function of displaying contents of at least one of AR, VR, SR, MR, and the like enables the user to feel a higher level of immersion.

An electronic appliance 700A illustrated in FIG. 6A and an electronic appliance 700B illustrated in FIG. 6B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display device of one embodiment of the present invention can be used for the display panels 751. Thus, a highly reliable electronic appliance is obtained.

The electronic appliance 700A and the electronic appliance 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic appliance 700A and the electronic appliance 700B are electronic appliances capable of performing AR display.

In the electronic appliance 700A and the electronic appliance 700B, a camera capable of capturing images of the front side may be provided as the image capturing portion. Furthermore, when the electronic appliance 700A and the electronic appliance 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliance 700A and the electronic appliance 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a moving image can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

The electronic appliance of one embodiment of the present invention may have a function of performing wireless communication with earphones 750. The earphones 750 include a communication portion (not illustrated) and have a wireless communication function. The earphones 750 can receive information (e.g., audio data) from the electronic appliance with the wireless communication function. For example, the electronic appliance 700A in FIG. 6A has a function of transmitting information to the earphones 750 with the wireless communication function.

The electronic appliance may include an earphone portion. The electronic appliance 700B in FIG. 6B includes earphone portions 727. For example, the earphone portion 727 can be connected to the control portion by wire. Part of a wiring that connects the earphone portion 727 and the control portion may be positioned inside the housing 721 or the wearing portion 723.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of a headset by including the audio input mechanism.

Note that the electronic appliance of one embodiment of the present invention can be suitably applied to a goggles-type structure without being limited to the glasses-type structure like the electronic appliance 700A and the electronic appliance 700B.

The electronic appliance of one embodiment of the present invention can transmit information to earphones by wire or wirelessly.

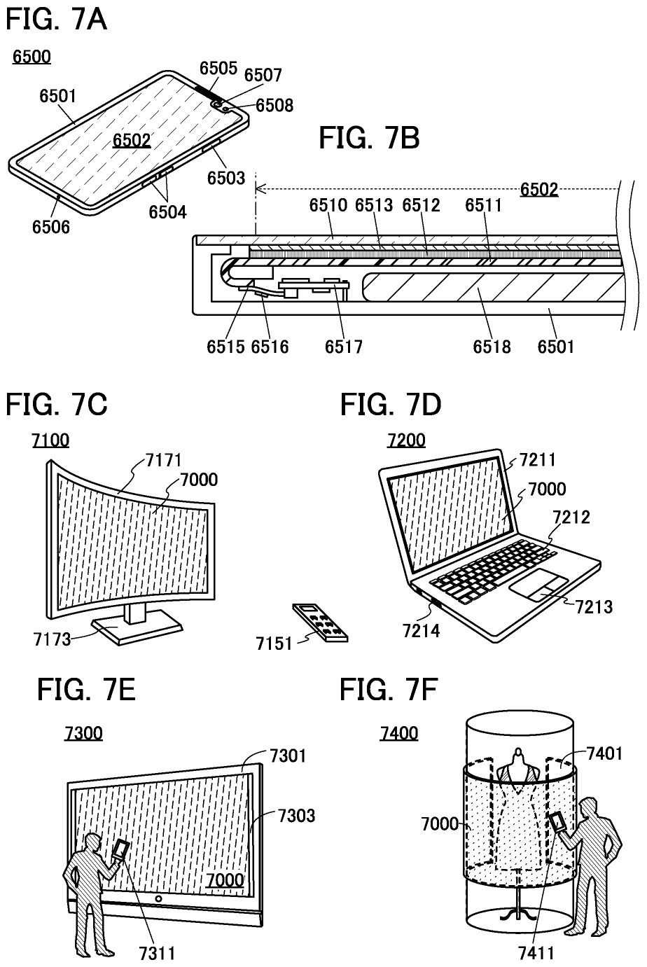

An electronic appliance 6500 illustrated in FIG. 7A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The display device of one embodiment of the present invention can be used in the display portion 6502. Thus, a highly reliable electronic appliance is obtained.

FIG. 7B is a schematic cross-sectional view including an end portion of the housing 6501 closer to the microphone 6506.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not shown).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display device of one embodiment of the present invention can be used in the display panel 6511. Thus, an extremely lightweight electronic appliance can be achieved. Since the display panel 6511 is extremely thin, the battery 6518 with high capacity can be mounted without an increase in the thickness of the electronic appliance. Moreover, part of the display panel 6511 is folded back so that a connection portion with the FPC 6515 is provided on the back side of the pixel portion, whereby an electronic appliance with a narrow bezel can be achieved.

FIG. 7C illustrates an example of a television device. In a television device 7100, a display portion 7000 is incorporated in a housing 7171. Here, the housing 7171 is supported by a stand 7173.

The display device of one embodiment of the present invention can be used in the display portion 7000. Thus, a highly reliable electronic appliance is obtained.

Operation of the television device 7100 illustrated in FIG. 7C can be performed with an operation switch provided in the housing 7171 and a separate remote control 7151. Alternatively, the display portion 7000 may include a touch sensor, and the television device 7100 may be operated by touch on the display portion 7000 with a finger or the like. The remote control 7151 may be provided with a display portion for displaying information output from the remote control 7151. With operation keys or a touch panel of the remote control 7151, channels and volume can be controlled and video displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 7D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display device of one embodiment of the present invention can be used in the display portion 7000. Thus, a highly reliable electronic appliance is obtained.

FIGS. 7E and 7F illustrate examples of digital signage that can be used for store windows, showcases, and the like.

Digital signage 7300 illustrated in FIG. 7E includes a housing 7301, the display portion 7000, a speaker 7303, and the like. The digital signage 7300 can also include an LED lamp, an operation key (including a power switch or an operation switch), a connection terminal, a variety of sensors, a microphone, and the like.

FIG. 7F illustrates digital signage 7400 attached to a showcase 7401. The digital signage 7400 includes the display portion 7000 provided along a curved surface of the showcase 7401.

In FIGS. 7E and 7F, the display device of one embodiment of the present invention can be used in the display portion 7000. Thus, a highly reliable electronic appliance is obtained.

A larger area of the display portion 7000 can increase the amount of information that can be provided at a time. The larger display portion 7000 attracts more attention, so that the effectiveness of the advertisement can be increased, for example.

Specifically, in the case where the display device of one embodiment of the present invention is used for the digital signage 7300 and the digital signage 7400 shown in FIGS. 7E and 7F that display advertisements and the like, the display device being a light-transmitting panel can increase the flexibility of representation. A light-transmitting display device can be manufactured, for example, by using a wiring and a support member each of which is formed of a conductive film that transmits visible light and adjusting the distance between pixel electrodes.

The use of the tandem light-emitting device of one embodiment of the present invention in addition to the wiring and the support member each of which is formed of the conductive film that transmits visible light can increase the luminance per pixel. That is, favorable display can be performed even when the aperture ratio of the display device is decreased; thus, the light-transmitting property of the display portion of the display device can be increased. Accordingly, such a structure is suitably used in the light-transmitting display device of one embodiment of the present invention.

As illustrated in FIGS. 7E and 7F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

This embodiment can be combined as appropriate with the other embodiments. In this specification, in the case where a plurality of structure examples are shown in one embodiment, the structure examples can be combined as appropriate.

### Embodiment 5

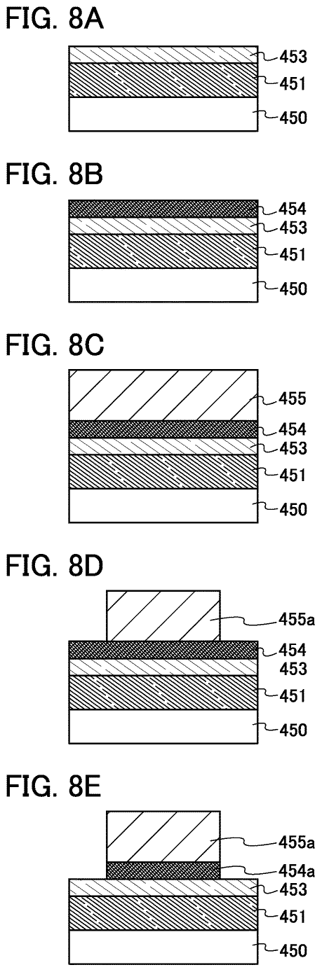

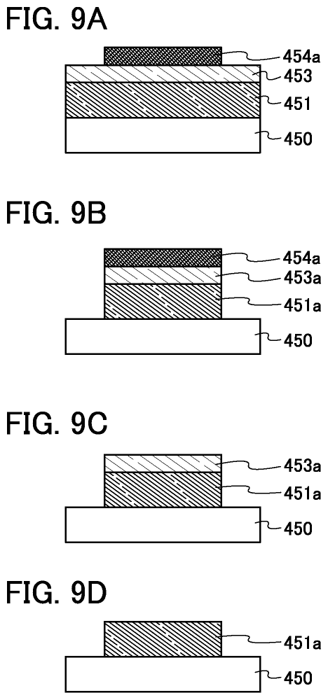

In this embodiment, a method for processing the film formed using the organic compound by a lithography method is described with reference to FIGS. 8A to 8E and FIGS. 9A to 9D.

It is necessary to solve many problems in order that the shape of the organic compound film can be processed by a lithography method. Examples of the problems include an effect of exposure to the air of the organic compound film, an effect of light irradiation when a photosensitive resin is exposed to light, an effect of developer when the exposed photosensitive resin is developed, and an effect of formation of a metal film that is sometimes formed to reduce the effect of the developer.

Here, an inorganic mask film is provided as a protective film over and in contact with an organic compound film, and then a step that would cause the above-described problems is performed. As the inorganic mask film, for example, an aluminum oxide film is used. Since an aluminum oxide film can be formed dense and is highly capable of blocking liquid and gas, the adverse effects caused by the step can be inhibited. Furthermore, the aluminum oxide film can be formed and removed by a method that is less likely to damage the organic compound film, and thus is extremely suitable as the inorganic mask film protecting the organic compound film. Note that an atomic layer deposition (ALD) method that is capable of forming a denser film and causes less damage to the organic compound film is preferably employed as a film formation method of the aluminum oxide film.

The aluminum oxide film causes less damage to the organic compound film when being formed and removed, and thus is suitably used as a protective film in processing the organic compound film by a lithography method. However, when the surface of the organic compound film is excessively exposed in a removal step of the aluminum oxide film, it is natural that the surface of the organic compound film is damaged, whereby the device characteristics might be degraded.

In one embodiment of the present invention, the organic compound represented by any of General Formula (G0) to General Formula (G7) is used for an organic compound film 451. It is preferable that the organic compound represented by any of General Formula (G0) to General Formula (G7) be used for a layer close to the outermost surface in the organic compound film 451. As described above, the organic compound represented by any of General Formula (G0) to General Formula (G7) has high T_{g} of a film and powder. Using such an organic compound for the organic compound film 451 can reduce thermal damage in particular among a variety of damage caused in steps of forming and removing an aluminum oxide film and the like, which enables the light-emitting device to have favorable characteristics.

The layer close to the outermost surface in the organic compound film 451 refers to, for example, a layer at least part of which is included in the region within 30 nm, preferably within 20 nm, further preferably within 10 nm, still further preferably within 5 nm in the depth direction from the outermost surface of the organic compound film 451. In this specification and the like, a bulk region of an organic compound film refers to, for example, an inner region located at least 40 nm away from the outermost surface of the organic compound film in the depth direction.

First, the organic compound film 451 containing the organic compound represented by any of General Formula (G0) to General Formula (G7) is formed over a base film 450 (FIG. 8A). The base film may be either an insulating film or a conductive film depending on a device manufactured later. The organic compound film 451 may be formed by a dry method such as an evaporation method or a wet method such as a spin coating method, but is preferably formed by a vacuum evaporation method.

Next, an inorganic mask film 453 is formed over the organic compound film 451 (FIG. 8A). The inorganic mask film 453 is preferably formed by a method that causes less damage to the film in contact with the organic compound film 451. An organic mask film formed using an organic compound can be formed between the organic compound film 451 and the inorganic mask film 453.

A film 454 to be a hard mask formed of a metal film or a metal compound film is preferably formed over the inorganic mask film 453 (FIG. 8B). Since damage to the organic compound film 451 can be reduced by the inorganic mask film 453, the film 454 to be a hard mask can be formed by a film formation method that causes relatively great damage to a formation surface, such as a sputtering method.

Examples of a material for the film 454 to be a hard mask include silicon, silicon nitride, silicon oxide, tungsten, titanium, molybdenum, tantalum, tantalum nitride, an alloy containing molybdenum and niobium, an alloy metal oxide containing molybdenum and tungsten, and a metal oxide such as an indium gallium zinc oxide (also denoted as In—Ga—Zn oxide or IGZO). It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

Then, a photosensitive resin is applied to the film 454 to be a hard mask, so that a resin film 455 is formed (FIG. 8C). The photosensitive resin may be either a positive type resist or a negative type resist.

Next, a photomask layer 455a is formed by performing light exposure in accordance with the photosensitivity of the resin and performing development (FIG. 8D), and the film 454 to be a hard mask is etched with the use of the photomask layer 455a, whereby a hard mask layer 454a is formed (FIG. 8E).

The film 454 to be a hard mask may be etched by either wet etching or dry etching. This etching is preferably performed under the condition where the film 454 to be a hard mask has a high selectivity with respect to the inorganic mask film 453.

After the hard mask layer 454a is formed, the photomask layer 455a is removed (FIG. 9A). Owing to the film 454 to be a hard mask and the inorganic mask film 453, the organic compound film 451 is prevented from being adversely affected, for example, being eliminated or being damaged, by treatment in forming and removing the photomask layer 455a, and thus, an organic device having favorable characteristics can be manufactured.

After that, etching is performed with the use of the film 454 to be a hard mask as a mask, whereby an organic compound layer 451a and an inorganic mask layer 453a are formed (FIG. 9B). This etching may be either wet etching or dry etching, but dry etching is preferable.

Although not illustrated, an inorganic insulating layer covering the top surface and the side surface of the organic compound layer 451a may be formed after forming the organic compound layer 451a. The inorganic insulating layer can be formed by a method similar to that for the inorganic mask layer 453a. The formation of the inorganic insulating layer can protect the side surface of the organic compound layer 451a. Furthermore, after forming the inorganic insulating layer, an insulating layer may be formed over the inorganic insulating layer in a region not overlapping with the organic compound layer 451a. In FIGS. 3A and 3B, the inorganic insulating layer and the insulating layer correspond to the inorganic insulating layer 125 and the insulating layer 127, respectively.

After that, the hard mask layer 454a is removed (FIG. 9C). The hard mask layer 454a is removed by etching. Either wet etching or dry etching can be performed, but dry etching is preferable. This etching is preferably performed under the condition where the hard mask layer 454a has a high selectivity with respect to the inorganic mask layer 453a.

Lastly, the inorganic mask layer 453a is removed by being treated with water or a liquid containing water as a solvent, so that the surface of the organic compound layer 451a is exposed (FIG. 9D). The inorganic mask layer 453a can be removed by etching. Although either wet etching or dry etching can be performed, wet etching using an alkaline solution or an acid solution is preferable, and wet etching using an alkaline solution is further preferable. In the case where the inorganic insulating layer covering the top surface and the side surface of the organic compound layer 451a is formed, at least part of the inorganic insulating film is removed by the step of removing the inorganic mask layer 453a, so that the surface of the organic compound layer 451a can be exposed.

Heat treatment is preferably performed after the surface of the organic compound layer 451a is exposed. By the heat treatment, water contained in the organic compound layer 451a can be removed. When the temperature of the heat treatment is too low, it is difficult to sufficiently remove water contained in the organic compound layer 451a. Thus, the heat treatment is preferably performed at a substrate temperature higher than or equal to 100° C.

Note that when the temperature of the heat treatment is too high, the organic compound layer 451a might deteriorate. Thus, the temperature of the heat treatment is preferably lower than the T_{g} of a film and powder of the organic compound contained in the organic compound layer 451a; specifically, the heat treatment is preferably performed at a substrate temperature higher than or equal to 100° C. and lower than or equal to 130° C., further preferably higher than or equal to 100° C. and lower than or equal to 120° C., yet still further preferably higher than or equal to 100° C. and lower than or equal to 110° C.

For a layer close to the outermost surface in the organic compound layer 451a, it is preferable to use an organic compound whose T_{g} of one or both of a film and powder is at least 30° C. higher than the temperature of the heat treatment, preferably at least 40° C. higher, and further preferably at least 50° C. higher. This is because the T_{g} in the region within 30 nm in the depth direction from the outermost surface of the organic compound layer decreases relatively easily compared to that in the bulk region. Furthermore, this is because the T_{g} in the vicinity of the outermost surface of the organic compound layer decreases approximately 30° C. from the T_{g} in the bulk region (see Non-Patent Document 5).

Thus, for the layer close to the outermost surface in the organic compound layer 451a, it is preferable to use an organic compound whose T_{g} of one or both of a film and powder is higher than or equal to 130° C., preferably higher than or equal to 140° C., further preferably higher than or equal to 150° C., still further preferably higher than or equal to 160° C., and yet still further preferably higher than or equal to 170° C. The organic compound represented by any of General Formula (G0) to General Formula (G7) has high T_{g} of a film and powder, and thus is preferably used for the layer close to the outermost surface in the organic compound layer 451a.

The heating atmosphere may be an air atmosphere or an inert gas atmosphere. Although the heating atmosphere may be an atmospheric-pressure atmosphere or a reduced-pressure atmosphere, a reduced-pressure atmosphere is preferably employed to prevent re-adsorption of water released from the organic compound layer 451a.

The organic compound layer 451a processed through the above steps has less processing damage. Thus, a light-emitting device in which the organic compound layer formed through the above steps is used can have favorable characteristics.

The structure described in this embodiment can be used in combination with any of the structures described in other embodiments as appropriate.

### Example 1

### Synthesis Example 1

This synthesis example describes a method for synthesizing the organic compound of one embodiment of the present invention, 2-(2′,7′-di-tert-butyl-9,9′-spirobi[9H-fluoren]-2-yl)-4-phenyl-6-[4-(triphenylsilyl)phenyl]-1,3,5-triazine (abbreviation: tBu-PSi-SFTzn) (Structural Formula (128)), and the physical properties thereof.

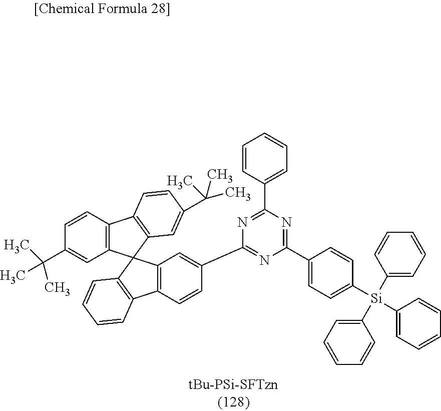 [SMILES: CC(C)(C)c1ccc2c(c1)C1(c3ccccc3-c3ccc(-c4nc(-c5ccccc5)nc(-c5ccc([Si](c6ccccc6)(c6ccccc6)c6ccccc6)cc5)n4)cc31)c1cc(C(C)(C)C)ccc1-2]

Into a 200-mL three-neck flask, 1.4 g (2.6 mmol) of 2-chloro-4-phenyl-6-[4-(triphenylsilyl)phenyl]-1,3,5-triazine, 2.2 g (4.0 mmol) of 2-(2′,7′-di-tert-butyl-9,9′-spirobi[9H-fluoren]-2-yl)-4,4,5,5-tetramethyl-1,3,2-dioxaborolane, 16 mg (53 μmol) of tris(2-methylphenyl)phosphine, and 1.1 g (5.3 mmol) of tripotassium phosphate were put. To the mixture, 10 mL of toluene, 5 mL of 1,4-dioxane, and 5 mL of water were added. The mixture was degassed by being stirred while the pressure was reduced. To the mixture, 6 mg (26 μmol) of palladium(II) acetate was added and the mixture was stirred at 100° C. for 7 hours under a nitrogen stream. After the stirring, an aqueous layer of the mixture was subjected to extraction with toluene. The extracted solution and the organic layer were combined and washed with water and a saturated saline solution. The organic layer was dried with magnesium sulfate, and then the mixture was gravity filtered. The obtained filtrate was concentrated to give an oily substance. The oily substance was purified by silica gel column chromatography (developing solvent: toluene:hexane=1:3 and then toluene:hexane=3:7). The obtained solid was recrystallized with ethyl acetate/ethanol to give 2.1 g of a white solid in a yield of 88%. Then, 2.1 g of the obtained white solid was purified by a train sublimation method in which the solid was heated at 320° C. under a pressure of 3.4 Pa with an argon flow rate of 15 mL/min. After the sublimation purification, 2.0 g of a white solid was obtained at a collection rate of 92%. The synthesis scheme is shown in Formula (a-1).

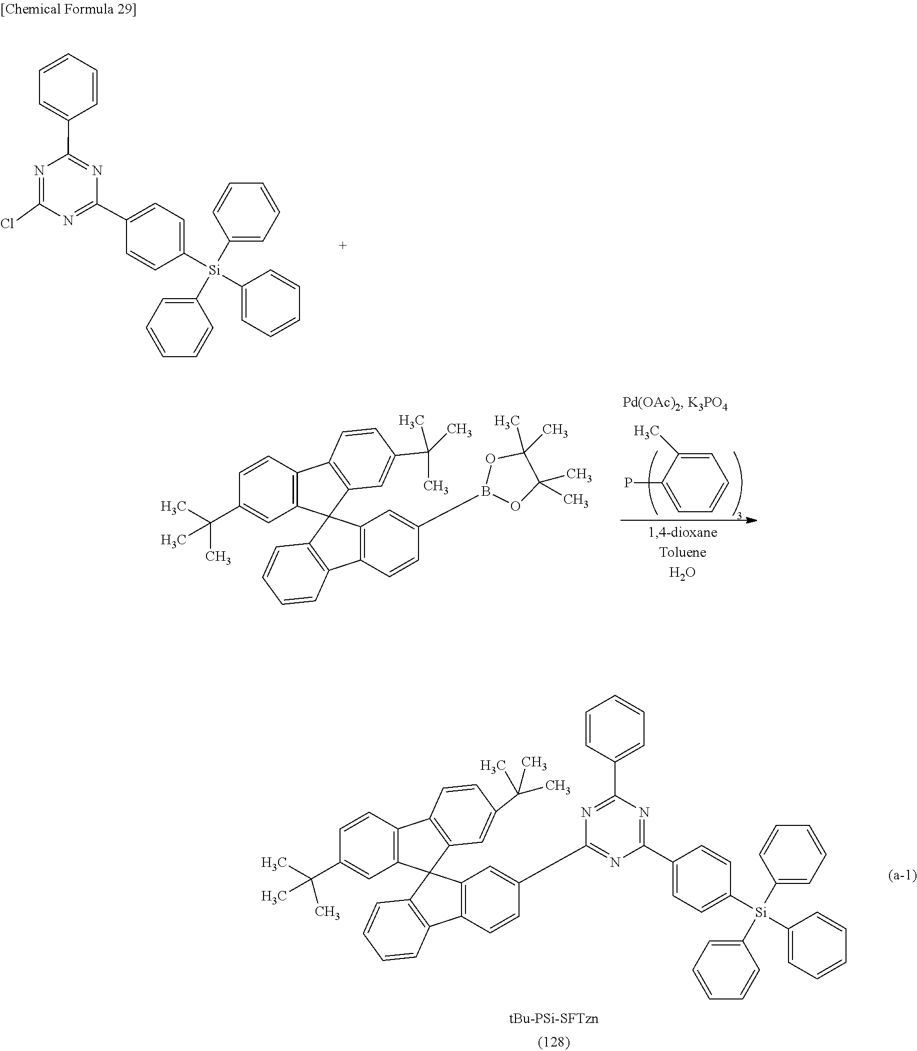

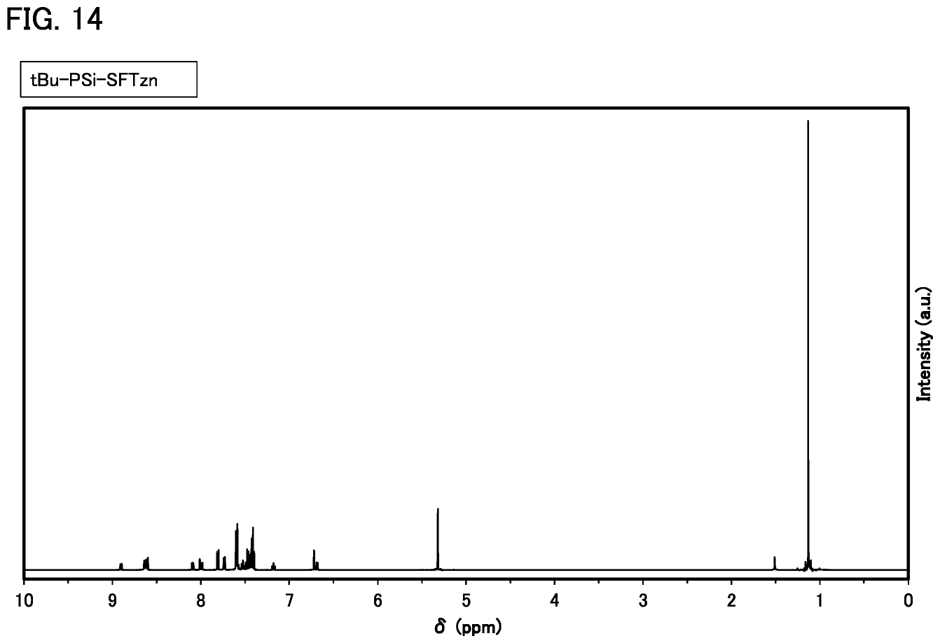

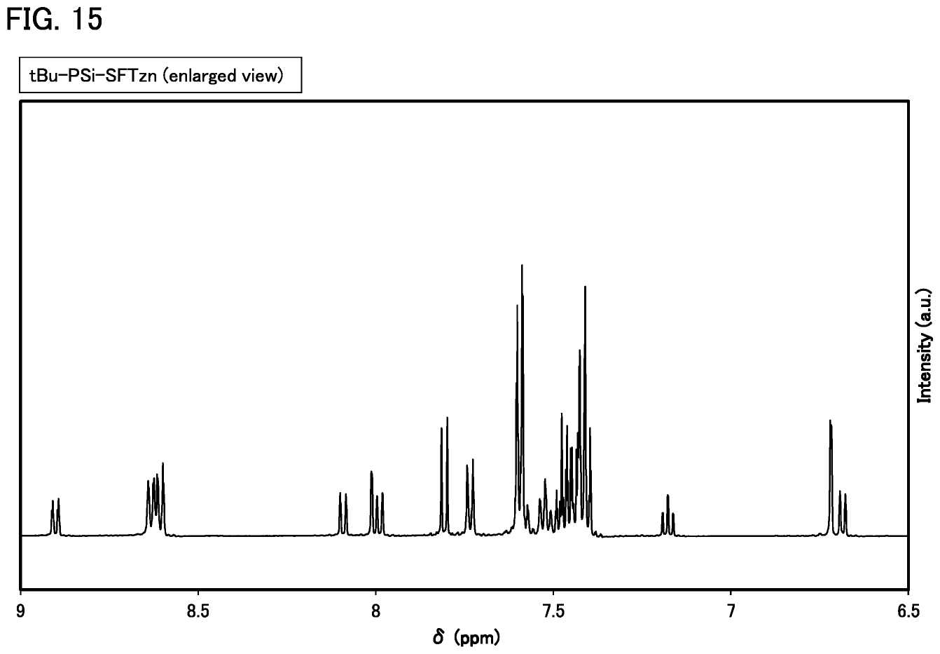

Analysis results by nuclear magnetic resonance (¹H-NMR) spectroscopy of the obtained white solid are shown below. FIG. 14 is a ¹H-NMR chart, and FIG. 15 is an enlarged chart of the range of 6.5 ppm to 9.0 ppm in FIG. 14. These results reveal that the organic compound of one embodiment of the present invention, tBu-PSi-SFTzn (Structural Formula (128)), was obtained in this synthesis example.

¹H-NMR (CD₂Cl₂, 500 MHz): δ=1.13 (s, 18H), 6.69 (d, J₁=7.5 Hz, 1H), 6.71-6.72 (m, 2H), 7.18 (td, J₁=7.5 Hz, J₂=1.0 Hz, 1H), 7.39-7.60 (m, 21H), 7.72-7.74 (m, 2H), 7.81 (d, J₁=8.5 Hz, 2H), 7.99 (d, J₁=7.5 Hz, 1H), 8.01 (d, J₁=1.0 Hz, 1H), 8.09 (d, J₁=8.0 Hz, 1H), 8.59-8.64 (m, 4H), 8.90 (dd, J₁=8.0 Hz, J₂=1.5 Hz, 1H).

### <Measurement of Emission and Absorption Spectra>

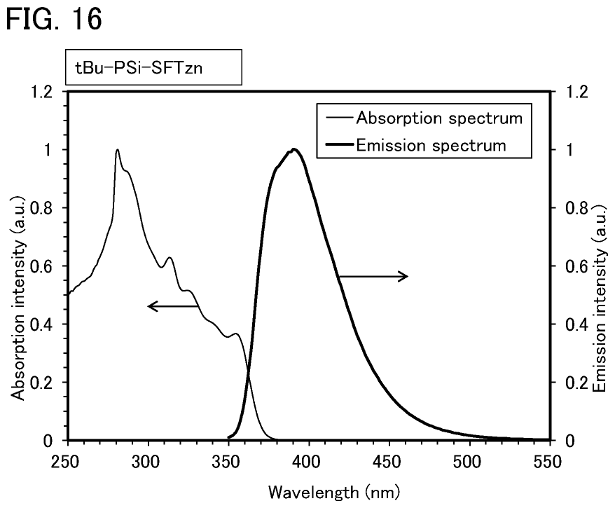

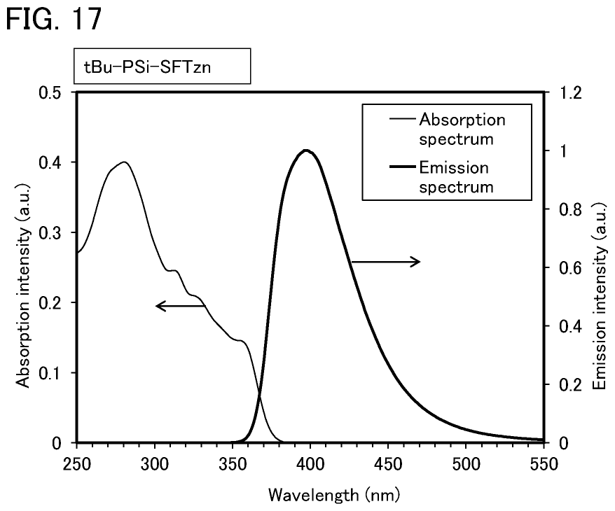

FIG. 16 shows the measurement results of emission and absorption spectra of tBu-PSi-SFTzn in a toluene solution. FIG. 17 shows the emission and absorption spectra of a thin film of tBu-PSi-SFTzn. The thin film was formed over a quartz substrate by a vacuum evaporation method. The absorption spectrum of the toluene solution was measured with an ultraviolet-visible light spectrophotometer (V-770DS, manufactured by JASCO Corporation), and the spectrum of toluene alone in a quartz cell was subtracted. The absorption spectrum of the thin film was measured with a spectrophotometer (U4100 Spectrophotometer, manufactured by Hitachi High-Technologies Corporation). The emission spectra were measured with a fluorescence spectrophotometer (FP-8600, manufactured by JASCO Corporation).

As shown in FIG. 16, tBu-PSi-SFTzn in the toluene solution has absorption spectrum peaks at wavelengths of 354 nm, 326 nm, 313 nm, and 288 nm and an emission spectrum peak at a wavelength of 391 nm (excitation wavelength: 340 nm). As shown in FIG. 17, the thin film of tBu-PSi-SFTzn has absorption spectrum peaks at wavelengths of 358 nm, 327 nm, 313 nm, 282 nm, and 269 nm and an emission spectrum peak at a wavelength of 397 nm (excitation wavelength: 330 nm).

### <Measurement of Refractive Index>

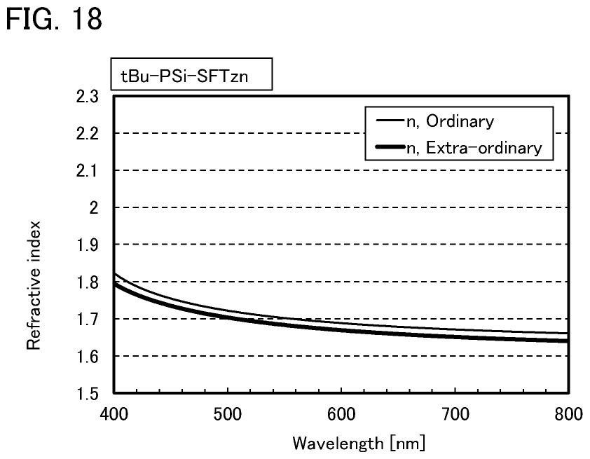

Next, the refractive index of a film formed using tBu-PSi-SFTzn was measured with a spectroscopic ellipsometer (M-2000U, manufactured by J. A. Woollam Japan Corp.). The film used for the measurement was formed to a thickness of approximately 50 nm with the material over a quartz substrate by a vacuum evaporation method. Note that a refractive index for an ordinary ray, n, Ordinary, and a refractive index for an extraordinary ray, n, Extra-ordinary, are shown in the graphs. FIG. 18 shows the measurement results. FIG. 18 shows that the film of tBu-PSi-SFTzn has a low refractive index: the ordinary refractive index in the entire blue emission region (the wavelength range of 455 nm to 465 nm) is in the range of 1.50 to 1.75 and the ordinary refractive index at a wavelength of 633 nm is 1.68.

### <Measurement of GSP_Slope>

Next, by the method similar to that described in Embodiment 1, the GSP_slope of an evaporated film formed using tBu-PSi-SFTzn was measured. The measurement result shows that the evaporated film of tBu-PSi-SFTzn has a small GSP_slope: the GSP_slope is 14.4 mV/nm.

### <Measurement of T_{g}>

Next, T_{g} was measured. The T_{g} was measured with a differential scanning calorimeter (DSC8500, manufactured by PerkinElmer Japan Co., Ltd.) in the following manner: powder of tBu-PSi-SFTzn put on an aluminum cell was heated until it had melted, cooled to −10° C., and then heated at a temperature rising rate of 40° C./min. The measurement result shows that the T_{g} of the sample is 176° C. Thus, it was found that the organic compound could give a solid having high T_{g}, and thus the evaporated film of the organic compound can also be expected to have high T_{g}.

### Example 2

### Synthesis Example 2

This synthesis example describes a method for synthesizing the organic compound of one embodiment of the present invention, 2-(2′,7′-di-tert-butyl-9,9′-spirobi[9H-fluoren]-2-yl)-4-phenyl-6-[3-(triphenylsilyl)phenyl]-1,3,5-triazine (abbreviation: tBu-mPSi-SFTzn) (Structural Formula (129)), and the physical properties thereof.

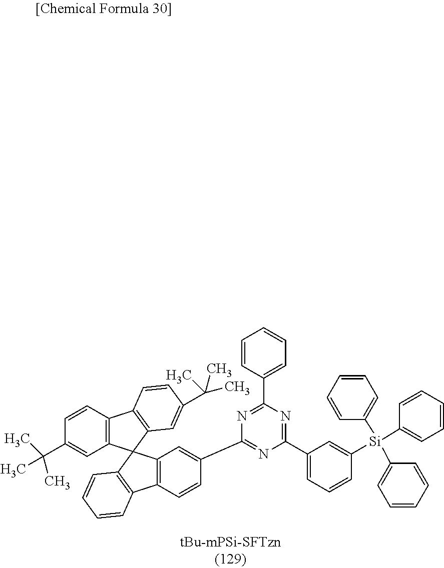 [SMILES: CC(C)(C)c1ccc2c(c1)C1(c3ccccc3-c3ccc(-c4nc(-c5ccccc5)nc(-c5cccc([Si](c6ccccc6)(c6ccccc6)c6ccccc6)c5)n4)cc31)c1cc(C(C)(C)C)ccc1-2]

Into a 200-mL three-neck flask, 2.3 g (4.4 mmol) of 2-chloro-4-phenyl-6-[3-(triphenylsilyl)phenyl]-1,3,5-triazine, 3.6 g (6.5 mmol) of 2-(2′,7′-di-tert-butyl-9,9′-spirobi[9H-fluoren]-2-yl)-4,4,5,5-tetramethyl-1,3,2-dioxaborolane, 27 mg (87 μmol) of tris(2-methylphenyl)phosphine, and 1.9 g (8.7 mmol) of tripotassium phosphate were put. To the mixture, 20 mL of toluene, 10 mL of 1,4-dioxane, and 5 mL of water were added. The mixture was degassed by being stirred while the pressure was reduced. To the mixture, 10 mg (44 μmol) of palladium(II) acetate was added and the mixture was stirred at 100° C. for 19 hours under a nitrogen stream. After the stirring, an aqueous layer of the mixture was subjected to extraction with toluene. The extracted solution and the organic layer were combined and washed with water and a saturated saline solution. The organic layer was dried with magnesium sulfate, and then the mixture was gravity filtered. The obtained filtrate was concentrated to give an oily substance. The oily substance was purified by silica gel column chromatography (developing solvent: toluene:hexane=1:3 and then toluene:hexane=3:7). The obtained solid was recrystallized with ethyl acetate/ethanol to give 2.4 g of a white solid in a yield of 61%. Then, 1.9 g of the obtained white solid was purified by a train sublimation method in which the solid was heated at 300° C. under a pressure of 3.1 Pa with an argon flow rate of 15 mL/min. After the sublimation purification, 1.7 g of a white solid was obtained at a collection rate of 92%. The synthesis scheme is shown in Formula (b-1).

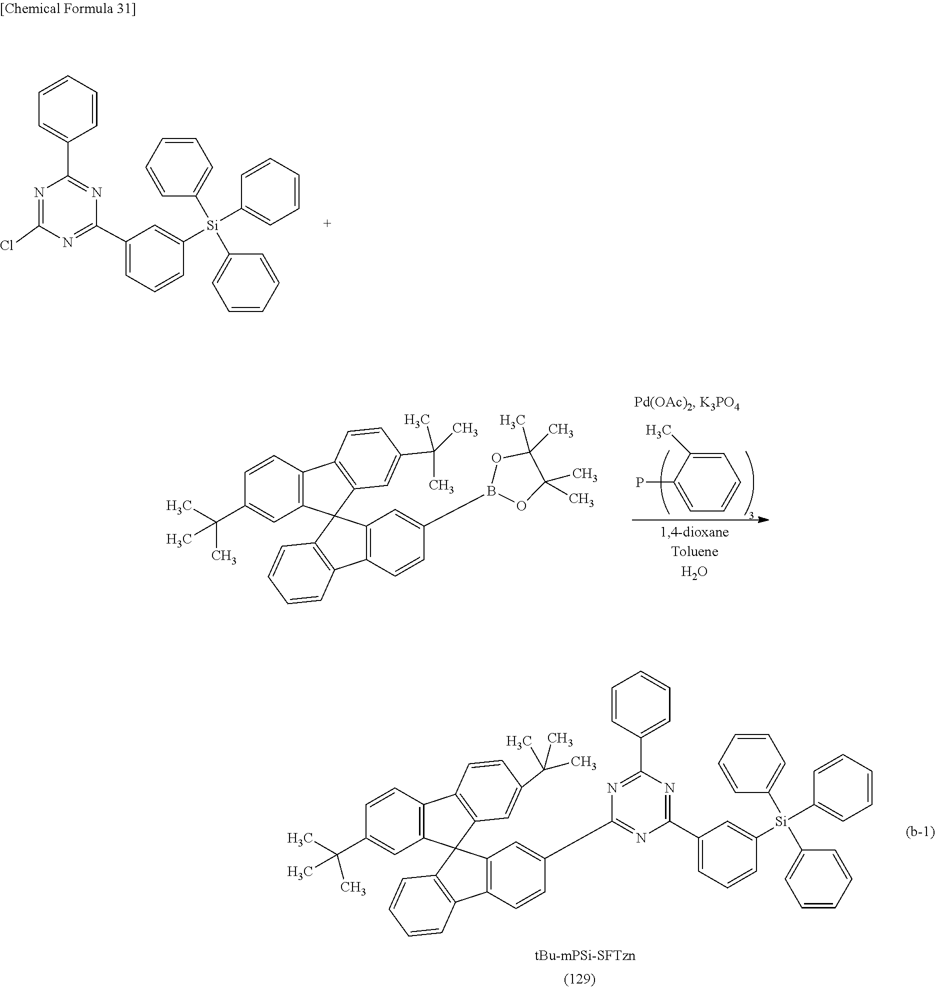

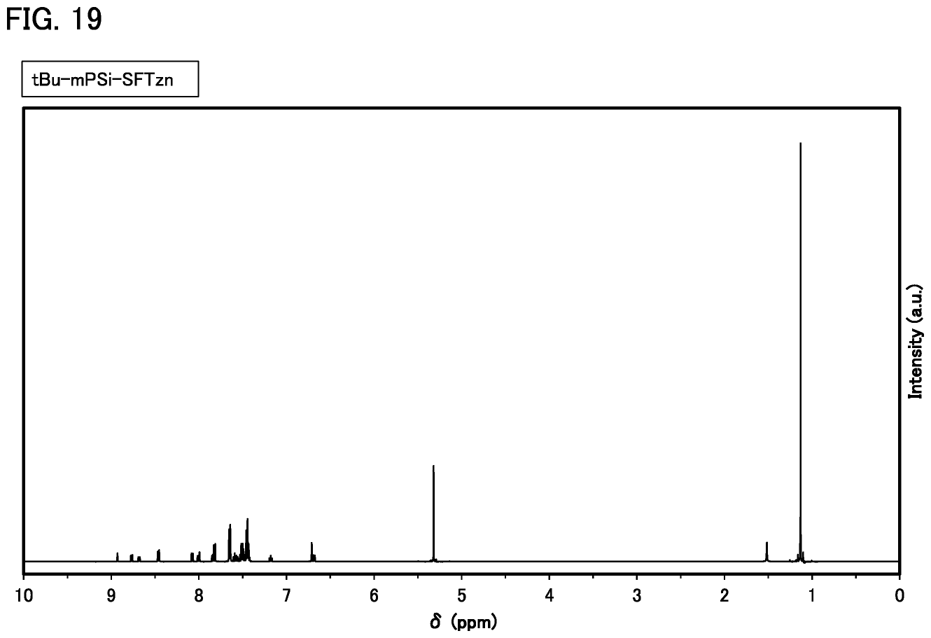

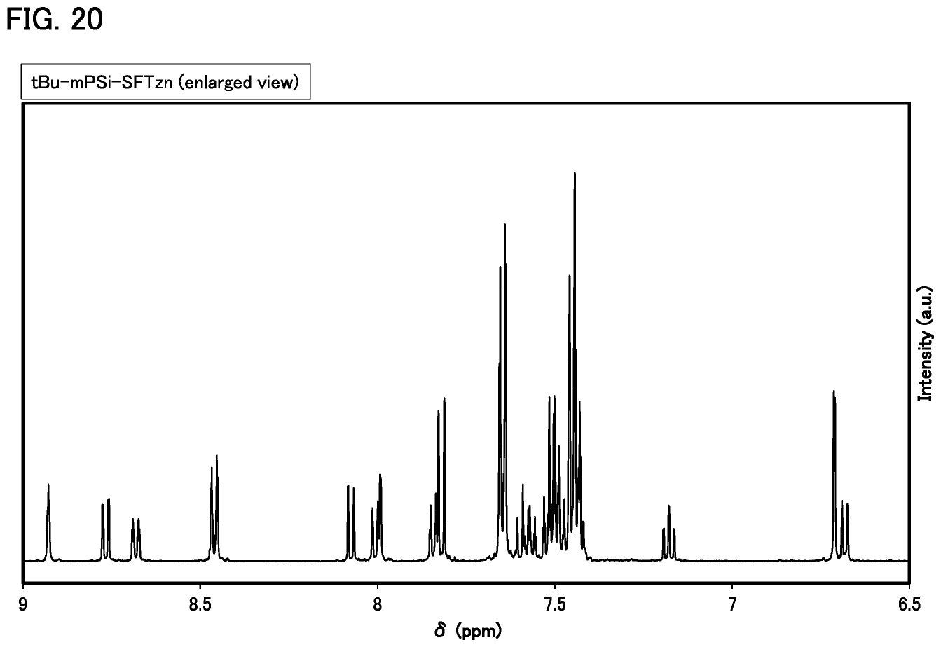

Analysis results by nuclear magnetic resonance (¹H-NMR) spectroscopy of the obtained white solid are shown below. FIG. 19 is a ¹H-NMR chart, and FIG. 20 is an enlarged chart of the range of 6.5 ppm to 9.0 ppm in FIG. 19. These results reveal that the organic compound of one embodiment of the present invention, tBu-mPSi-SFTzn (Structural Formula (129)), was obtained in this synthesis example.

¹H NMR (CD₂Cl₂, 500 MHz): δ=1.13 (s, 18H), 6.68 (d, J₁=7.5 Hz, 1H), 6.71 (d, J₁=1.5 Hz, 2H), 7.18 (td, J₁=7.5 Hz, J₂=1.0 Hz, 1H), 7.41-7.66 (m, 22H), 7.81-7.85 (m, 3H), 7.99-8.01 (m, 2H), 8.07 (dd, J₁=8.0 Hz, J₂=0.5 Hz, 1H), 8.45-8.47 (m, 2H), 8.68 (dt, J₁=8.5 Hz, J₂=1.0 Hz, 1H), 8.77 (dd, J₁=8.0 Hz, J₂=1.5 Hz, 1H), 8.93 (t, J₁=1.0 Hz, 1H).

### <Measurement of Emission and Absorption Spectra>

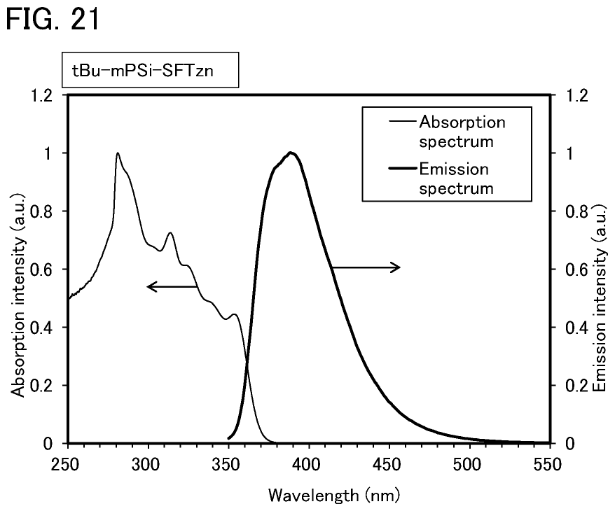

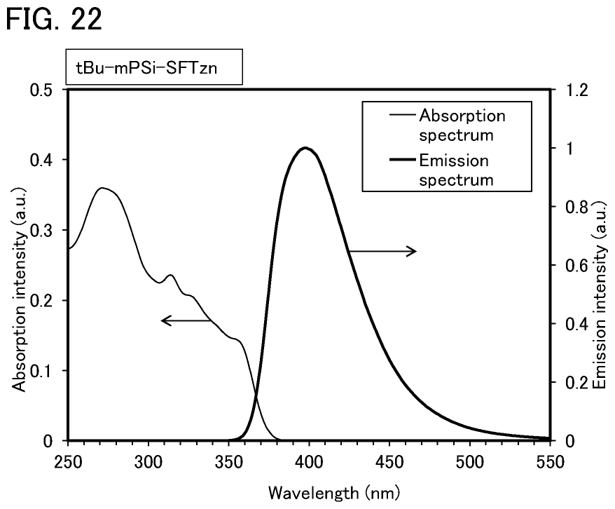

FIG. 21 shows the measurement results of emission and absorption spectra of tBu-mPSi-SFTzn in a toluene solution. FIG. 22 shows the emission and absorption spectra of a thin film of tBu-mPSi-SFTzn. The emission and absorption spectra were measured by the method similar to that described in Example 1.

As shown in FIG. 21, tBu-mPSi-SFTzn in the toluene solution has absorption spectrum peaks at wavelengths of 354 nm, 340 nm, 325 nm, 313 nm, and 288 nm and emission spectrum peaks at wavelengths of 378 nm and 388 nm (excitation wavelength: 340 nm). As shown in FIG. 22, the thin film of tBu-mPSi-SFTzn has absorption spectrum peaks at wavelengths of 355 nm, 326 nm, 312 nm, 280 nm, and 268 nm and an emission spectrum peak at a wavelength of 398 nm (excitation wavelength: 330 nm).

### <Measurement of Refractive Index>

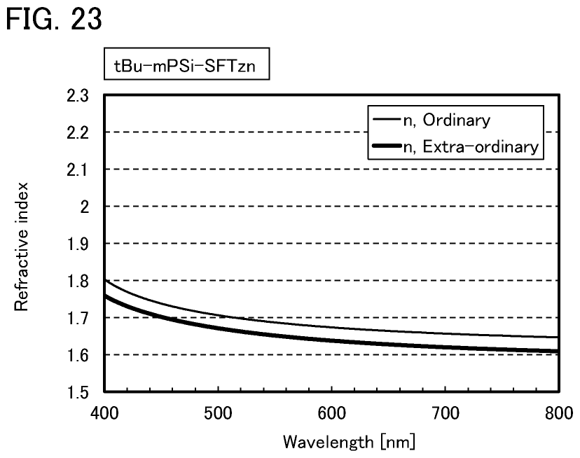

Next, by the method similar to that described in Example 1, the refractive index of a film formed using tBu-mPSi-SFTzn was measured. FIG. 23 shows the measurement results. FIG. 23 shows that the film of tBu-mPSi-SFTzn has a low refractive index: the ordinary refractive index in the entire blue emission region (the wavelength range of 455 nm to 465 nm) is in the range of 1.50 to 1.75 and the ordinary refractive index at a wavelength of 633 nm is 1.67.

### <Measurement of GSP_Slope>

Next, by the method similar to that described in Embodiment 1, the GSP_slope of an evaporated film formed using tBu-mPSi-SFTzn was measured. The measurement result shows that the evaporated film of tBu-mPSi-SFTzn has a small GSP_slope: the GSP_slope is 15.5 mV/nm.

### <Measurement of T_{g}>

Next, by the method similar to that described in Example 1, T_{g} was measured. The measurement result shows that the T_{g} of the sample formed using tBu-mPSi-SFTzn is 157° C. Thus, it was found that the organic compound could give a solid having high T_{g}, and thus the evaporated film of the organic compound can also be expected to have high T_{g}.

### Example 3

This example describes the fabrication of the device 1 (the light-emitting device 1) in which an evaporated film of tBu-PSi-SFTzn (Structural Formula (128)), which is the organic compound of one embodiment of the present invention described in Example 1, was used for the electron-transport layer, the device 2 (the light-emitting device 2) in which an evaporated film of tBu-mPSi-SFTzn (Structural Formula (129)), which is the organic compound of one embodiment of the present invention described in Example 2, was used for the electron-transport layer, and the comparative device 3 (the comparative light-emitting device 3) and the comparative device 4 (the comparative light-emitting device 4) in each of which an evaporated film of a comparative organic compound was used for the electron-transport layer, and measurement results of these devices' characteristics. These light-emitting devices are top-emission light-emitting devices that emit green phosphorescent light. Structural formulae of organic compounds used for the light-emitting devices are shown below.

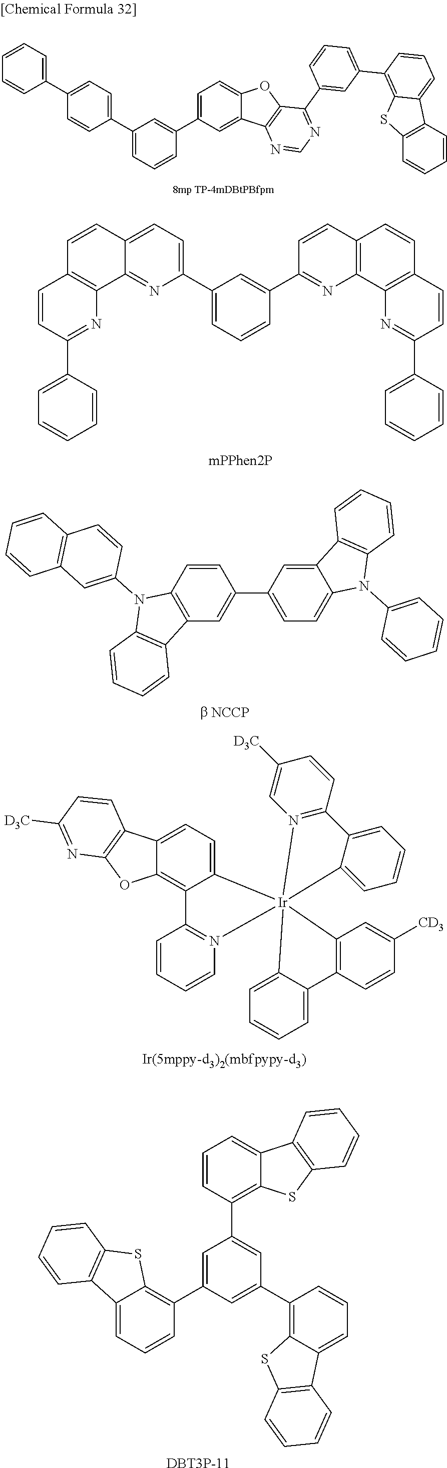

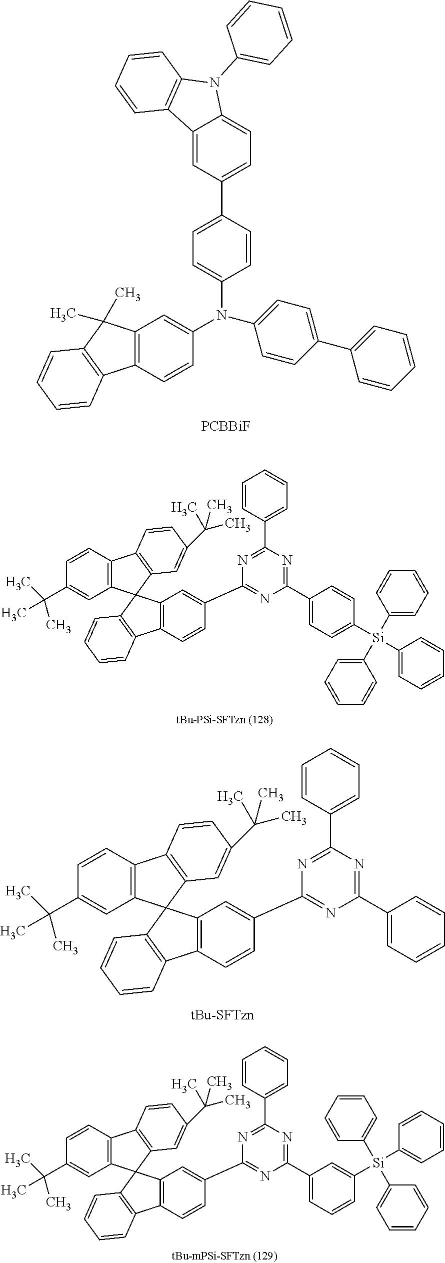

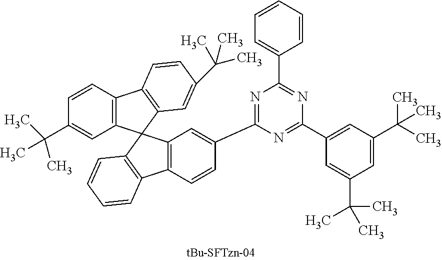 [SMILES: CC(C)(C)c1cc(-c2nc(-c3ccccc3)nc(-c3ccc4c(c3)C3(c5ccccc5-4)c4cc(C(C)(C)C)ccc4-c4ccc(C(C)(C)C)cc43)n2)cc(C(C)(C)C)c1]

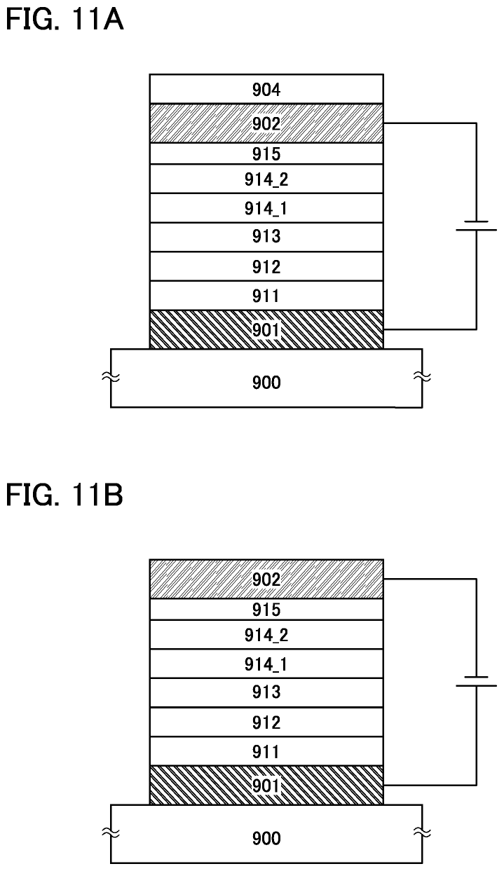

As illustrated in FIG. 11A, the light-emitting devices each have a structure in which a hole-injection layer 911, a hole-transport layer 912, a light-emitting layer 913, electron-transport layers (a first electron-transport layer 914_1 and a second electron-transport layer 9142), and an electron-injection layer 915 are stacked in this order over a first electrode 901 formed over a glass substrate 900, and a second electrode 902 and a cap layer 904 are stacked over the electron-injection layer 915.

### <Fabrication Method of Device 1>

An alloy containing silver (Ag), palladium (Pd), and copper (Cu) (abbreviation: APC) was deposited to a thickness of 100 nm as a reflective electrode over the glass substrate 900 by a sputtering method, and then, indium tin oxide containing silicon oxide (ITSO) was deposited by a sputtering method to a thickness of 50 nm, so that the first electrode 901 as a transparent electrode was formed. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁴ Pa, and vacuum baking was performed at 170° C. for 30 minutes in a heating chamber of the vacuum evaporation apparatus. After that, natural cooling was performed.

Then, the substrate provided with the first electrode 901 was fixed to a substrate holder provided in the vacuum evaporation apparatus such that the surface on which the first electrode 901 was formed faced downward. Over the first electrode 901, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were deposited by co-evaporation to a thickness of 10 nm at the weight ratio of 1:0.03 (PCBBiF:OCHD-003), whereby the hole-injection layer 911 was formed.

Then, over the hole-injection layer 911, PCBBiF was deposited by evaporation to a thickness of 95 nm, whereby the hole-transport layer 912 was formed.

Then, over the hole-transport layer 912, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-3,3′-bi-9H-carbazole (abbreviation: PNCCP), and [2-d₃-methyl-8-(2-pyridinyl-κN)benzofuro[2,3-b]pyridine-κC]bis[2-(5-d₃-methyl-2-pyridinyl-κN2)phenyl-κC]iridium(III) (abbreviation: Ir(5mppy-d₃)₂(mbfpypy-d₃)) were deposited by co-evaporation to a thickness of 40 nm at the weight ratio of 0.5:0.5:0.1 (8mpTP-4mDBtPBfpm:PNCCP:Ir(5mppy-d₃)₂(mbfpypy-d₃)), whereby the light-emitting layer 913 was formed.

Next, over the light-emitting layer 913, tBu-PSi-SFTzn, which is the organic compound of one embodiment of the present invention, was deposited by evaporation to a thickness of 35 nm, whereby the first electron-transport layer 914_1 was formed. Then, 2,2′-(1,3-phenylene)bis(9-phenyl-1,10-phenanthroline) (abbreviation: mPPhen2P) was deposited by evaporation to a thickness of 5 nm, whereby the second electron-transport layer 914_2 was formed.

Then, over the second electron-transport layer 9142, lithium fluoride (LiF) and ytterbium (Yb) were deposited by co-evaporation to a thickness of 1.5 nm at the volume ratio of 2:1 (=LiF:Yb), whereby the electron-injection layer 915 was formed.

Then, over the electron-injection layer 915, silver (Ag) and magnesium (Mg) were deposited by co-evaporation to a thickness of 15 nm at the volume ratio of 1:0.1, whereby the second electrode 902 was formed.

The second electrode is a transflective electrode having a function of reflecting light and a function of transmitting light; thus, the light-emitting device of this example is a top-emission light-emitting device in which light is extracted through the second electrode. Over the second electrode, 4,4′,4″-(benzene-1,3,5-triyl)tri(dibenzothiophene) (abbreviation: DBT3P-II) was deposited by evaporation to a thickness of 70 nm as the cap layer to improve extraction efficiency.

### <Fabrication Method of Device 2>

The device 2 is different from the device 1 in that tBu-PSi-SFTzn, which is the organic compound of one embodiment of the present invention used for the first electron-transport layer 914_1 of the device 1, was replaced with tBu-mPSi-SFTzn, which is an organic compound of another embodiment of the present invention. Other components were fabricated in a manner similar to that for the device 1.

### <Fabrication Method of Comparative Device 3>

The comparative device 3 is different from the device 1 in that tBu-PSi-SFTzn, which is the organic compound of one embodiment of the present invention used for the first electron-transport layer 914_1 of the device 1, was replaced with 2,4-diphenyl-6-(2′,7′-di-tert-butyl-9,9′-spirobi[9H-fluoren]-2-yl)-1,3,5-triazine (abbreviation: tBu-SFTzn), which is a comparative organic compound. Other components were fabricated in a manner similar to that for the device 1.

### <Fabrication Method of Comparative Device 4>

The comparative device 4 is different from the device 1 in that tBu-PSi-SFTzn, which is the organic compound of one embodiment of the present invention used for the first electron-transport layer 914_1 of the device 1, was replaced with 2-phenyl-4-(3,5-di-tert-butylphenyl)-6-(2′,7′-di-tert-butyl-9,9′-spirobi[9H-fluoren]-2-yl)-1,3,5-triazine (abbreviation: tBu-SFTzn-04), which is a comparative organic compound. Other components were fabricated in a manner similar to that for the device 1.

The device structures of the light-emitting devices are listed in Table 3.

TABLE 3

| | | Film | | | Comparative | Comparative |
|---|---|---|---|---|---|---|
| | | thickness | Device 1 | Device 2 | device 3 | device 4 |
| Second electrode | 2 | 70 nm | DBT3P-II | | | |
| | 1 | 15 nm | Ag:Mg (1:0.1) | | | |
| Electron-injection layer | | 1.5 nm | LiF:Yb (2:1) | | | |
| Electron-transport layer | 2 | 5 nm | mPPhen2P | | | |
| | 1 | 35 nm | tBu-PSi-SFTzn | tBu-mPSi-SFTzn | tBu-SFTzn | tBu-SFTzn-04 |

| Light-emitting layer | | 40 nm | 8mpTP-4mDBtPBfpm:βNCCP:Ir(5mppy-d₃)₂(mbfpypy-d₃) |
|---|---|---|---|
| | | | (0.5:0.5:0.1) |
| Hole-transport layer | | 95 nm | PCBBiF |
| Hole-injection layer | | 10 nm | PCBBiF:OCHD-003 (1:0.03) |
| First electrode | 2 | 50 nm | ITSO |
| | 1 | 100 nm | APC |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for 1 hour were performed at the time of sealing). Then, the characteristics of the light-emitting devices were measured.

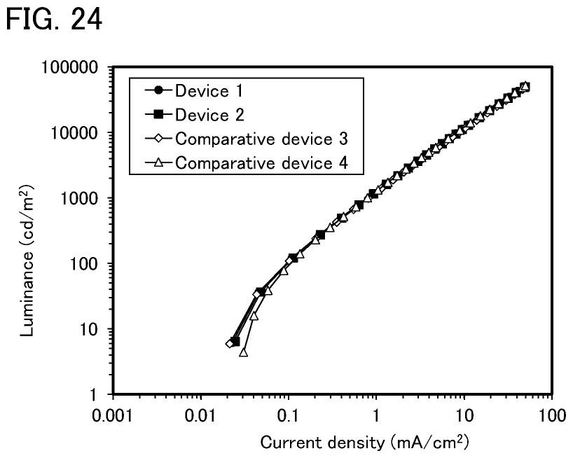

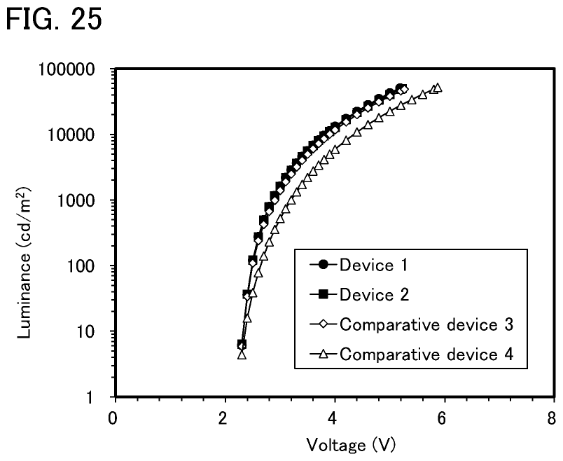

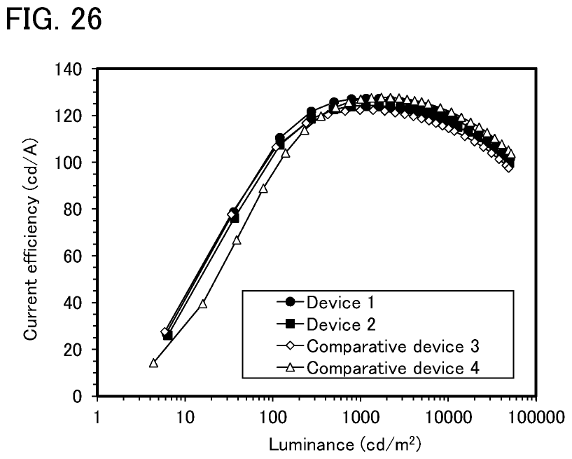

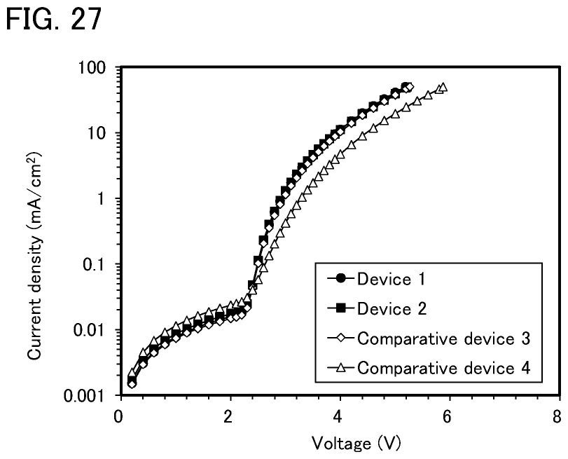

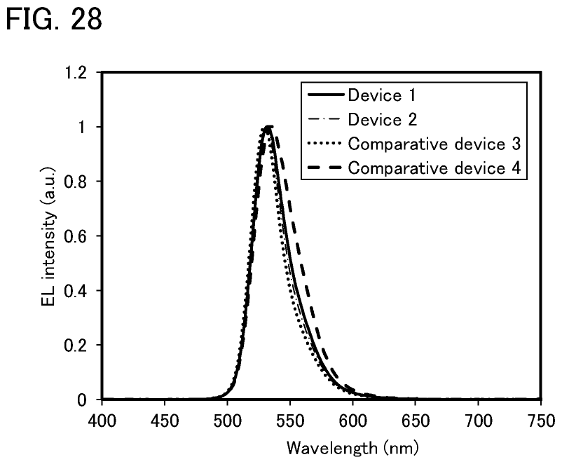

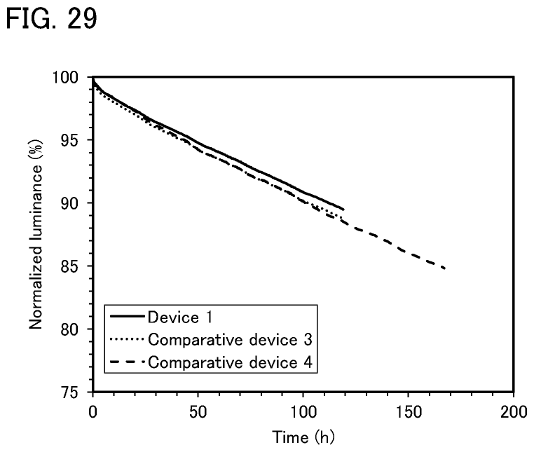

FIG. 24 shows luminance-current density characteristics of the device 1, the device 2, the comparative device 3, and the comparative device 4. FIG. 25 shows luminance-voltage characteristics thereof. FIG. 26 shows current efficiency-luminance characteristics thereof. FIG. 27 shows current density-voltage characteristics thereof. FIG. 28 shows electroluminescence spectra thereof. FIG. 29 shows luminance changes over driving time when the device 1, the comparative device 3, and the comparative device 4 are driven at a constant current of 2 mA (50 mA/cm²).

Table 4 shows the main characteristics of the light-emitting devices at a luminance of approximately 1000 cd/m². Note that the luminance, CIE chromaticity, and emission spectra were measured with a spectroradiometer (SR-UL1R, manufactured by TOPCON TECHNOHOUSE CORPORATION).

TABLE 4

| | | | Current | | | | Current |
|---|---|---|---|---|---|---|---|
| | Voltage | Current | density | Chromaticity | Chromaticity | Luminance | efficiency |
| | (V) | (mA) | (mA/cm²) | x | y | (cd/m²) | (cd/A) |
| Device 1 | 2.90 | 0.0365 | 0.912 | 0.257 | 0.711 | 1160 | 127 |
| Device 2 | 2.90 | 0.0371 | 0.928 | 0.249 | 0.716 | 1153 | 124 |
| Comparative device 3 | 2.90 | 0.0322 | 0.806 | 0.241 | 0.722 | 985 | 122 |
| Comparative device 4 | 3.20 | 0.0316 | 0.789 | 0.280 | 0.694 | 1002 | 127 |

From FIG. 24, FIG. 25, FIG. 26, FIG. 27, FIG. 28 and Table 4, the device 1 (the light-emitting device 1) and the device 2 (the light-emitting device 2) in each of which the organic compound of one embodiment of the present invention was used were found to be light-emitting devices having favorable characteristics that emit green light with a peak wavelength of 533 nm derived from Ir(5mppy-d₃)₂(mbfpypy-d₃) and having long lifetimes.

FIG. 25 and FIG. 27 show that the devices 1 and 2 each have a driving voltage that is equivalent to that of the comparative device 3 and is lower than that of the comparative device 4. FIG. 29 shows that the luminance change over driving time of the device 1 when the device 1 is driven at a constant current is smaller than those of the comparative devices 3 and 4 when the comparative devices 3 and 4 are each driven at a constant current, and thus the device 1 has a long lifetime.

Here, Table 5 shows ordinary refractive indices (no) and GSP_slopes of evaporated films and T_{g} of powder of main organic compounds used in the light-emitting devices. In Table 5, the ordinary refractive index at 533 nm that is a peak wavelength of the devices 1 and 2 is shown as the ordinary refractive index. The ordinary refractive indices of the evaporated films of the organic compounds were measured by the method similar to that described in Example 1. In Table 5, the GSP_slopes of the evaporated films of the organic compounds were measured by the method similar to that described in Embodiment 1. In Table 5, the T_{g} of powder of the organic compounds were measured by the method similar to that described in Example 1.

TABLE 5

| | nₒ | GSP_slope | T_{g} |
|---|---|---|---|
| | (@533 nm) | (mV/nm) | (°C) |
| tBu-PSi-SFTzn | 1.71 | 14.4 | 176 |
| tBu-mPSi-SFTzn | 1.69 | 15.5 | 157 |
| tBu-SFTzn | 1.68 | 16.8 | 146 |
| tBu-SFTzn-04 | 1.66 | 27.7 | 154 |

As shown in Table 5, tBu-PSi-SFTzn and tBu-mPSi-SFTzn, which are the organic compounds of one embodiment of the present invention, have as low refractive indices of films as tBu-SFTzn and tBu-SFTzn-04, which are comparative organic compounds. In addition, tBu-PSi-SFTzn and tBu-mPSi-SFTzn have smaller GSP_slopes of evaporated films and higher T_{g} than tBu-SFTzn and tBu-SFTzn-04.

As described above, it was found that the devices 1 and 2 formed using tBu-PSi-SFTzn and tBu-mPSi-SFTzn, respectively, each had a driving voltage that was equivalent to that of the comparative device 3 formed using tBu-SFTzn and was lower than that of the comparative device 4 formed using tBu-SFTzn-04. Thus, it can be said that the electron-transport properties of the layers formed using tBu-PSi-SFTzn and tBu-mPSi-SFTzn are equivalent to that of the layer formed using tBu-SFTzn, and the electron-transport property of the layer formed using tBu-SFTzn-04 is lower than those of these layers.

Note that tBu-PSi-SFTzn, tBu-mPSi-SFTzn, and tBu-SFTzn-04 each have the structure of tBu-SFTzn as a mother skeleton, and substituents bonded to the mother skeletons are different from each other. That is, tBu-PSi-SFTzn, tBu-mPSi-SFTzn, and tBu-SFTzn-04 are different from tBu-SFTzn in the following: tBu-PSi-SFTzn and tBu-mPSi-SFTzn each have a structure where a triphenylsilyl group is bonded to the mother skeleton, and tBu-SFTzn-04 has a structure where two tert-butyl groups are bonded to the mother skeleton. Since the interaction between each of a tert-butyl group and a triphenylsilyl group and the LUMO which determines an electron-transport property is extremely small, the electron-transport property of an organic compound that has the structure of tBu-SFTzn as a mother skeleton and any of the tert-butyl group and the triphenylsilyl group introduced into the vicinity of a triazine ring is lower than that of tBu-SFTzn in some cases. As shown in FIG. 25 and FIG. 27, the comparative device 4 formed using tBu-SFTzn-04 actually had a driving voltage higher than that of the comparative device 3 formed using tBu-SFTzn. Meanwhile, the devices 1 and 2 formed using tBu-PSi-SFTzn and tBu-mPSi-SFTzn, respectively, in which triphenylsilyl groups were bonded had driving voltages equivalent to that of the comparative device 3 formed using tBu-SFTzn. Thus, tBu-PSi-SFTzn and tBu-mPSi-SFTzn were found to have electron-transport properties equivalent to that of tBu-SFTzn, although having triphenylsilyl groups in the vicinity of triazine rings. This result was obtained because, even when the electron transport was impeded by three benzene rings included in the triphenylsilyl group, the electron-transport property was compensated for by hyperconjugation between an aryl group in the mother skeleton and a bond between a silicon atom and a benzene ring so that the electron-transport property was improved.

Furthermore, the T_{g} of powder of tBu-PSi-SFTzn and tBu-mPSi-SFTzn was higher than that of tBu-SFTzn and tBu-SFTzn-04, which are comparative organic compounds. This indicates that in fabricating a light-emitting device employing a step of processing an organic compound layer by a photolithography method, the sample can be heated at a higher temperature. As a result, a large amount of moisture and impurities attached in the process can be removed. Accordingly, the light-emitting device formed using the organic compound can have a longer lifetime.

The above results show that the use of the organic compound of one embodiment of the present invention enables fabrication of a light-emitting device with a low driving voltage and a long lifetime.

### Example 4

This example describes the fabrication of the device 5 (the light-emitting device 5) in which an evaporated film of tBu-PSi-SFTzn (Structural Formula (128)), which is the organic compound of one embodiment of the present invention described in Example 1, was used for the electron-transport layer, the device 6 (the light-emitting device 6) in which an evaporated film of tBu-mPSi-SFTzn (Structural Formula (129)), which is the organic compound of one embodiment of the present invention described in Example 2, was used for the electron-transport layer, and the comparative device 7 (the comparative light-emitting device 7) in which an evaporated film of a comparative organic compound was used for the electron-transport layer, and measurement results of these devices' characteristics. These light-emitting devices are top-emission light-emitting devices that emit green phosphorescent light. These light-emitting devices were fabricated through a step of processing the organic compound layer by a photolithography method. Since some structural formulae of organic compounds used in these light-emitting devices are the same as those described in Example 3, the description thereof is omitted here.

As illustrated in FIG. 11A, the light-emitting devices each have a structure in which the hole-injection layer 911, the hole-transport layer 912, the light-emitting layer 913, electron-transport layers (the first electron-transport layer 9141 and the second electron-transport layer 914_2), and the electron-injection layer 915 are stacked in this order over the first electrode 901 formed over the glass substrate 900, and the second electrode 902 and the cap layer 904 are stacked over the electron-injection layer 915.

### <Fabrication Method of Device 5>

The device 5 was fabricated in the following manner: after the fabrication of the second electron-transport layer 9142 in the fabrication method of the device 1 in Example 3, the hole-injection layer 911, the hole-transport layer 912, the light-emitting layer 913, the first electron-transport layer 914_1, and the second electron-transport layer 914_2 were processed by a photolithography method, and then the electron-injection layer 915, the second electrode 902, and the cap layer 904 were stacked. Other components were fabricated in a manner similar to that for the device 1.

Described is a method for processing the hole-injection layer 911, the hole-transport layer 912, the light-emitting layer 913, the first electron-transport layer 914_1, and the second electron-transport layer 914_2 by a photolithography method.

After the second electron-transport layer 9142 was formed, the substrate was taken out from the vacuum evaporation apparatus and exposed to the air. Then, aluminum oxide was deposited to a thickness of 30 nm by an ALD method using trimethylaluminum (abbreviation: TMA) as a precursor and water vapor as an oxidizer, whereby a first sacrificial layer was formed.

Next, molybdenum was deposited to a thickness of 50 nm over the first sacrificial layer by a sputtering method, whereby a second sacrificial layer was formed.

Next, a resist was formed using a photoresist over the second sacrificial layer, and processing was performed such that an end portion of a slit having a width of 3 μm was located 3.5 μm outward from the end surface of the first electrode 901.

Specifically, the second sacrificial layer was processed using an etching gas containing carbon tetrafluoride (CF₄), oxygen (O₂), and helium (He) at a flow rate ratio of CF₄:O₂:He=100:67:333, then using an etching gas containing oxygen (O₂) with the use of the resists as masks. Next, the first sacrificial layer was processed using an etching gas containing fluoroform (CHF₃) and helium (He) at a flow rate ratio of CHF₃:He=1:49. After that, the hole-injection layer 911, the hole-transport layer 912, the light-emitting layer 913, the first electron-transport layer 9141, and the second electron-transport layer 914_2 were processed using an etching gas containing oxygen (O₂).

After the layers were processed, the resist was removed using a basic chemical solution containing water as a solvent. After that, the second sacrificial layer was removed using an etching gas containing sulfur hexafluoride (SF₆) and oxygen (O₂), then using an etching gas containing oxygen (O₂). Next, the first sacrificial layer was removed using an acid aqueous solution containing hydrofluoric acid, so that the top surface of the second electron-transport layer was exposed. Then, the substrate was transferred into a vacuum evaporation apparatus where the pressure was reduced to approximately 1×10⁻⁴ Pa, and heat treatment was performed at 100° C. for 1 hour in a heating chamber of the vacuum evaporation apparatus.

The above is the description of the processing by a photolithography method and the heat treatment. As described above, in the fabrication the device 5, treatment using water or a chemical solution containing water as a solvent was performed in the processing by the photolithography method and the heat treatment.

### <Fabrication Method of Device 6>

The device 6 is different from the device 5 in that tBu-PSi-SFTzn, which is the organic compound of one embodiment of the present invention used for the first electron-transport layer 914_1 of the device 5, was replaced with tBu-mPSi-SFTzn, which is an organic compound of another embodiment of the present invention. Other components were fabricated in a manner similar to that for the device 5.

### <Fabrication Method of Comparative Device 7>

The comparative device 7 is different from the device 5 in that tBu-PSi-SFTzn, which is the organic compound of one embodiment of the present invention used for the first electron-transport layer 914_1 of the device 5, was replaced with tBu-SFTzn-04, which is a comparative organic compound. Other components were fabricated in a manner similar to that for the device 5.

The device structures of the light-emitting devices are listed in Table 6.

TABLE 6

| | | Film | | | | Comparative |
|---|---|---|---|---|---|---|
| | | thickness | | Device 5 | Device 6 | device 7 |
| Second electrode | 2 | 70 | nm | DBT3P-II | | |
| | 1 | 15 | nm | Ag:Mg (1:0.1) | | |
| Electron-injection layer | | 1.5 | nm | LiF:Yb (2:1) | | |
| | | | | Processing by photolithography method and heat treatment | | |
| Electron-transport | 2 | 5 | nm | mPPhen2P | | |
| layer | 1 | 35 | nm | tBu-PSi-SFTzn | tBu-mPSi-SFTzn | tBu-SFTzn-04 |

| Light-emitting layer | | 40 | nm | 8mpTP-4mDBtPBfpm:βNCCP:Ir(5mppy-d₃)₂(mbfpypy-d₃) |
|---|---|---|---|---|
| | | | | (0.5:0.5:0.1) |
| Hole-transport layer | | 95 | nm | PCBBiF |
| Hole-injection layer | | 10 | nm | PCBBiF:OCHD-003 (1:0.03) |
| First electrode | 2 | 50 | nm | ITSO |
| | 1 | 100 | nm | APC |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for 1 hour were performed at the time of sealing). Then, the characteristics of the light-emitting devices were measured.

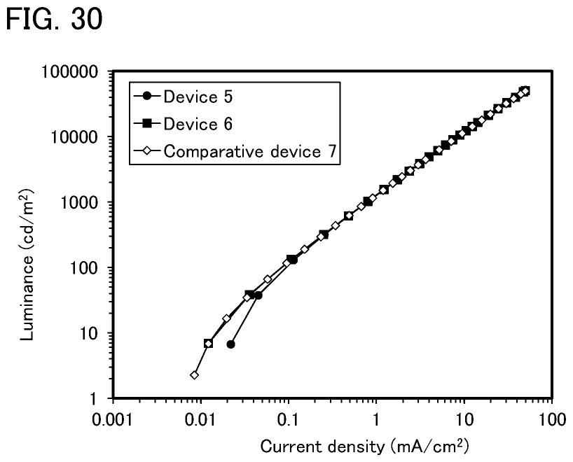

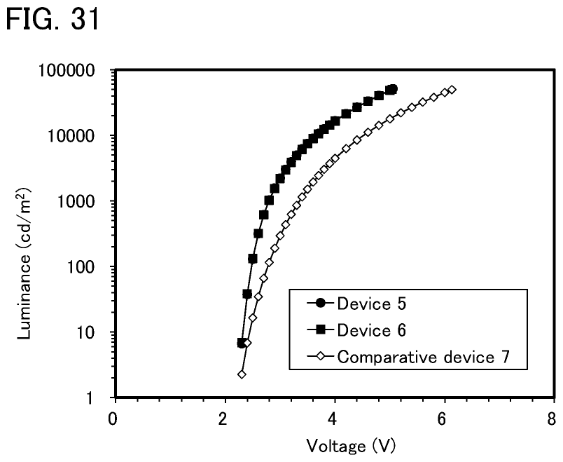

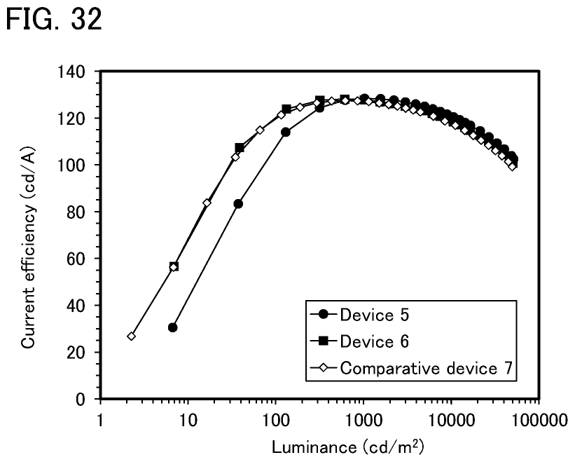

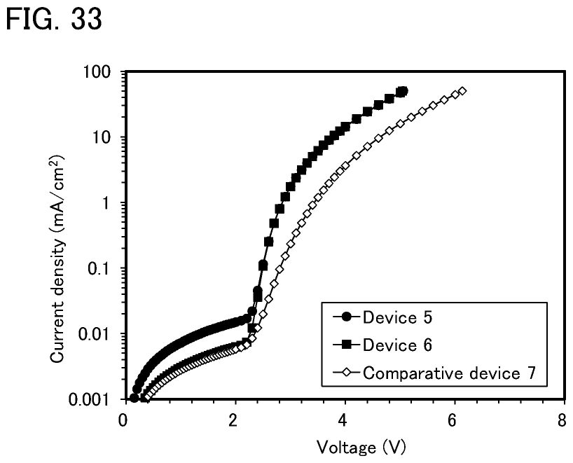

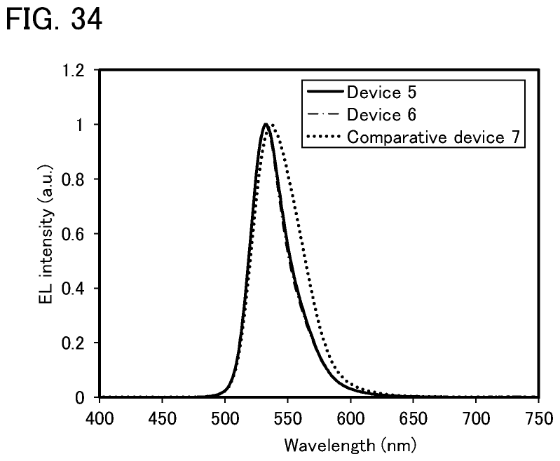

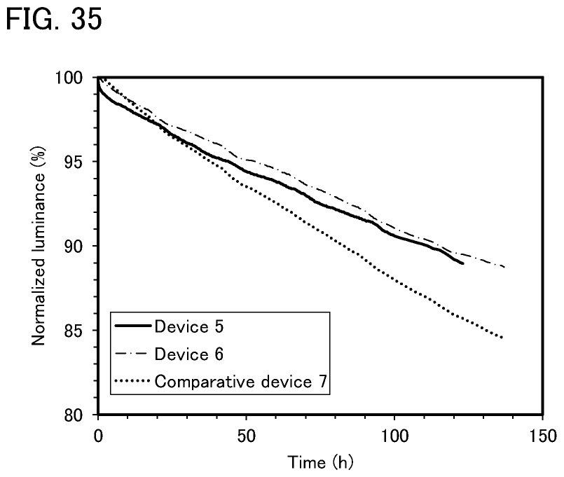

FIG. 30 shows luminance-current density characteristics of the light-emitting devices. FIG. 31 shows luminance-voltage characteristics thereof. FIG. 32 shows current efficiency-luminance characteristics thereof. FIG. 33 shows current density-voltage characteristics thereof. FIG. 34 shows electroluminescence spectra thereof. FIG. 35 shows luminance changes over driving time when the light-emitting devices are driven at a constant current of 2 mA (50 mA/cm²).

Table 7 shows the main characteristics of the light-emitting devices at a luminance of approximately 1000 cd/m². Note that the luminance, CIE chromaticity, and emission spectra were measured with a spectroradiometer (SR-UL1R, manufactured by TOPCON TECHNOHOUSE CORPORATION).

TABLE 7

| | | | Current | | | | Current |
|---|---|---|---|---|---|---|---|
| | Voltage | Current | density | Chromaticity | Chromaticity | Luminance | efficiency |
| | (V) | (mA) | (mA/cm²) | x | y | (cd/m²) | (cd/A) |
| Device 5 | 2.80 | 0.0321 | 0.802 | 0.265 | 0.705 | 1029 | 128 |
| Device 6 | 2.80 | 0.0320 | 0.799 | 0.260 | 0.708 | 1021 | 128 |
| Comparative device 7 | 3.30 | 0.0269 | 0.671 | 0.297 | 0.680 | 855 | 127 |

Upon observing the light-emitting regions of the devices in driving states, it was confirmed that each of the devices 5 and 6 had no uneven light emission in the light-emitting region and exhibited a uniform image. Meanwhile, the comparative device 7 had uneven light emission intensity in the light-emitting region and exhibited an uneven image. The comparative device 4, which has the same structure as the comparative device 7, had no such unevenness. Therefore, the uneven light emission in the comparative device 7 is caused in a step of processing an organic compound layer by a photolithography method.

From FIG. 30, FIG. 31, FIG. 32, FIG. 33, FIG. 34, FIG. 35, and Table 7, the device 5 (the light-emitting device 5) and the device 6 (the light-emitting device 6) in each of which the organic compound of one embodiment of the present invention was used were found to be light-emitting devices having favorable characteristics that emit green light derived from Ir(5mppy-d₃)₂(mbfpypy-d₃).

FIG. 31 and FIG. 33 show that the devices 5 and 6 each have a driving voltage lower than that of the comparative device 7. FIG. 35 shows that the luminance changes over driving time of the devices 5 and 6 when the devices 5 and 6 are each driven at a constant current are smaller than that of the comparative device 7 when the comparative device 7 is driven at a constant current, and thus the devices 5 and 6 have long lifetimes.

As shown in Table 5 in Example 3, tBu-PSi-SFTzn and tBu-mPSi-SFTzn, which are the organic compounds of one embodiment of the present invention, have as low refractive indices of films as tBu-SFTzn-04, which is a comparative organic compound. In addition, tBu-PSi-SFTzn and tBu-mPSi-SFTzn have smaller GSP_slopes of evaporated films and higher T_{g} of powder than tBu-SFTzn-04, which is a comparative organic compound.

As described above, it was found that the devices 5 and 6 formed using tBu-PSi-SFTzn and tBu-mPSi-SFTzn, respectively, each had a driving voltage lower than that of the comparative device 7 formed using tBu-SFTzn-04. This difference in driving voltages between each of the devices 5 and 6 and the comparative device 7 is larger than that between each of the devices 1 and 2 and the comparative device 4, which were also formed using tBu-PSi-SFTzn, tBu-mPSi-SFTzn, and tBu-SFTzn-04, respectively, and were fabricated without using a photolithography method. Such a difference in driving voltages relates to the unevenness in the light-emitting region of the comparative device 7 described above. That is, tBu-PSi-SFTzn and tBu-mPSi-SFTzn were found to be suitable for a step of processing the organic compound layer by a photolithography method. This is because the T_{g} of the films formed using tBu-PSi-SFTzn and the tBu-mPSi-SFTzn being higher than that of the film formed using tBu-SFTzn-04 affected resistance to heating process to differ.

As described above, the luminance changes over driving time of the devices 5 and 6 formed using tBu-PSi-SFTzn and tBu-mPSi-SFTzn, respectively when the devices 5 and 6 were each driven at a constant current were smaller than that of the comparative device 7 formed using tBu-SFTzn-04 when the comparative device 7 was driven at a constant current. This result can be explained by the T_{g} of powder of tBu-PSi-SFTzn and tBu-mPSi-SFTzn being higher than that of tBu-SFTzn-04. In other words, when fabricated using organic compounds having high T_{g} of powder, the device 5 and the device 6 were able to be highly resistant to processing by a photolithography method and heat treatment and thus had increased reliability.

The results in Example 3 and Example 4 show that the use of the organic compound of one embodiment of the present invention enables fabrication of a light-emitting device with a low driving voltage and a long lifetime. In addition, the use of the organic compound of one embodiment of the present invention for a light-emitting device fabricated through a step of processing its organic compound layer by a photolithography method enables the light-emitting device to have no defect in light emission display and a longer lifetime.

### Example 5

This example describes the fabrication of the device 8 (the light-emitting device 8) in which a co-evaporated film of tBu-PSi-SFTzn (Structural Formula (128)), which is the organic compound of one embodiment of the present invention described in Example 1, and 8-quinolinolato-lithium (abbreviation: Liq) was used for the electron-transport layer, the device 9 (the light-emitting device 9) in which a co-evaporated film of tBu-mPSi-SFTzn (Structural Formula (129)), which is the organic compound of one embodiment of the present invention described in Example 2, and Liq was used for the electron-transport layer, and the comparative device 10 (the comparative light-emitting device 10) to the comparative device 12 (the comparative light-emitting device 12) in each of which a co-evaporated film of a comparative organic compound and Liq was used for the electron-transport layer, and measurement results of these devices' characteristics. These light-emitting devices are bottom-emission light-emitting devices that emit green phosphorescent light. Structural formulae of organic compounds used for the light-emitting devices are shown below.

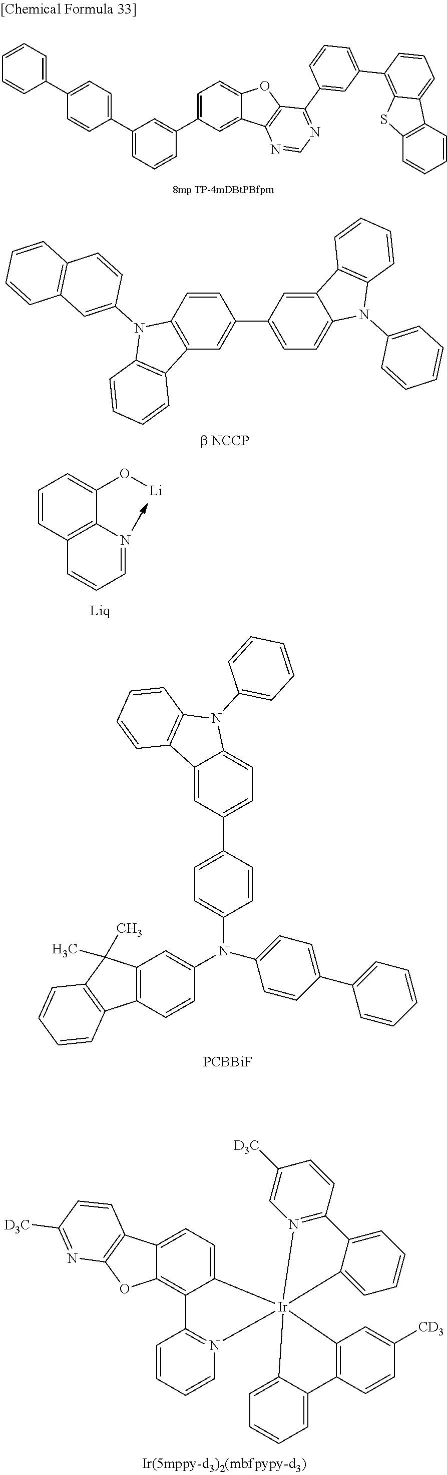

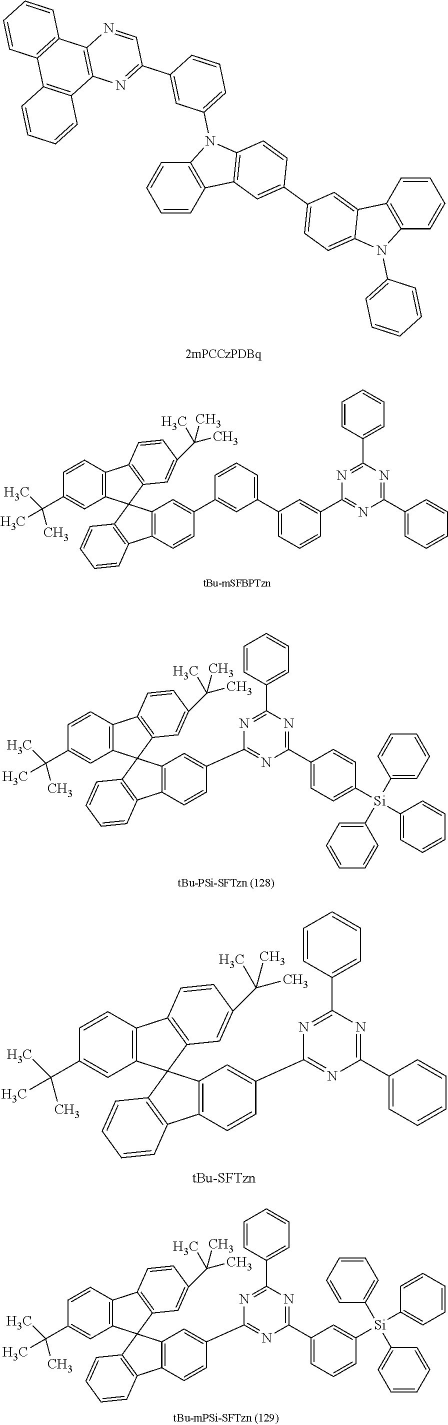

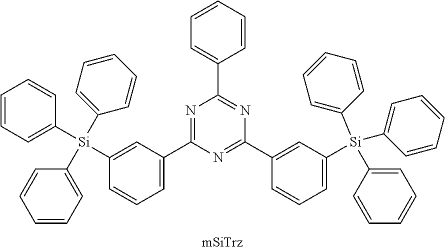 [SMILES: c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1]

As illustrated in FIG. 11B, the light-emitting devices each have a structure in which the hole-injection layer 911, the hole-transport layer 912, the light-emitting layer 913, electron-transport layers (the first electron-transport layer 9141 and the second electron-transport layer 914_2), and the electron-injection layer 915 are stacked in this order over the first electrode 901 formed over the glass substrate 900, and the second electrode 902 is stacked over the electron-injection layer 915.

### <Fabrication Method of Device 8>

Indium tin oxide containing silicon oxide (ITSO) was deposited by a sputtering method over the glass substrate 900 to a thickness of 55 nm, so that the first electrode 901 as a transparent electrode was formed. The electrode area was set to 4 mm² (2 mm×2 mm).

Next, as in the fabrication method of the device 1 in Example 3, pretreatment for fabricating the light-emitting device over a substrate and vacuum baking were performed. After that, natural cooling was performed.

Then, after forming the hole-injection layer 911 as in the fabrication method of the device 1, PCBBiF was deposited by evaporation to a thickness of 60 nm over the hole-injection layer 911, whereby the hole-transport layer 912 was formed. Then, as in the fabrication method of the device 1, the light-emitting layer 913 was formed over the hole-transport layer 912.

Next, over the light-emitting layer 913, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 20 nm, whereby the first electron-transport layer 914_1 was formed. Then, tBu-PSi-SFTzn, which is the organic compound of one embodiment of the present invention, and Liq were deposited by co-evaporation to a thickness of 20 nm of at the weight ratio of 1:1 (tBu-PSi-SFTzn: Liq), whereby the second electron-transport layer 914_2 was formed.

Then, over the second electron-transport layer 9142, lithium fluoride (LiF) was deposited by evaporation to a thickness of 1 nm, whereby the electron-injection layer 915 was formed.

Next, over the electron-injection layer 915, aluminum (Al) was deposited by evaporation to a thickness of 100 nm, whereby the second electrode 902 was formed.

### <Fabrication Method of Device 9>

The device 9 is different from the device 8 in that tBu-PSi-SFTzn, which is the organic compound of one embodiment of the present invention used in the second electron-transport layer 914_2 of the device 8, was replaced with tBu-mPSi-SFTzn, which is an organic compound of another embodiment of the present invention. Other components were fabricated in a manner similar to that for the device 8.

### <Fabrication Method of Comparative Device 10>

The comparative device 10 is different from the device 8 in that tBu-PSi-SFTzn, which is the organic compound of one embodiment of the present invention used for the first electron-transport layer 914_1 of the device 8, was replaced with tBu-SFTzn, which is a comparative organic compound. Other components were fabricated in a manner similar to that for the device 8.

### <Fabrication Method of Comparative Device 11>

The comparative device 11 is different from the device 8 in that tBu-PSi-SFTzn, which is the organic compound of one embodiment of the present invention used for the first electron-transport layer 914_1 of the device 8, was replaced with 2-[3′-(2′,7′-di-tert-butyl-9,9′-spirobi[9H-fluoren]-2-yl)biphenyl-3-yl]-4,6-diphenyl-1,3,5-triazine (abbreviation: tBu-mSFBPTzn), which is a comparative organic compound. Other components were fabricated in a manner similar to that for the device 8.

### <Fabrication Method of Comparative Device 12>

The comparative device 12 is different from the device 8 in that tBu-PSi-SFTzn, which is the organic compound of one embodiment of the present invention used for the first electron-transport layer 914_1 of the device 8, was replaced with 2-phenyl-4,6-bis[3-(triphenylsilyl)phenyl]-1,3,5-triazine (abbreviation: mSiTrz), which is a comparative organic compound. Other components were fabricated in a manner similar to that for the device 8.

The device structures of the light-emitting devices are listed in the table below.

TABLE 8

| | | Film | | | | | |
|---|---|---|---|---|---|---|---|
| | | thickness | | | Comparative | Comparative | Comparative |
| | | (nm) | Device 8 | Device 9 | device 10 | device 11 | device 12 |
| Second electrode | | 100 | Al | | | | |
| Electron-injection layer | | 1 | LiF | | | | |
| Electron-transport | 2 | 20 | tBu-PSi- | tBu-mPSi- | tBu-SFTzn:Liq | tBu-mSFBPTzn:Liq | mSiTrz:Liq |
| layer | | | SFTzn:Liq (1:1) | SFTzn:Liq (1:1) | (1:1) | (1:1) | (1:1) |

| | | |
|---|---|---|
| 1 | 20 | 2mPCCzPDBq |

| | | |
|---|---|---|
| Light-emitting layer | 40 | 8mpTP-4mDBtPBfpm:βNCCP:Ir(5mppy-d₃)₂(mbfpypy-d₃) |
| | | (0.5:0.5:0.1) |
| Hole-transport layer | 60 | PCBBiF |
| Hole-injection layer | 10 | PCBBiF:OCHD-003 (1:0.03) |
| First electrode | 55 | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for 1 hour were performed at the time of sealing). Then, the characteristics of the light-emitting devices were measured.

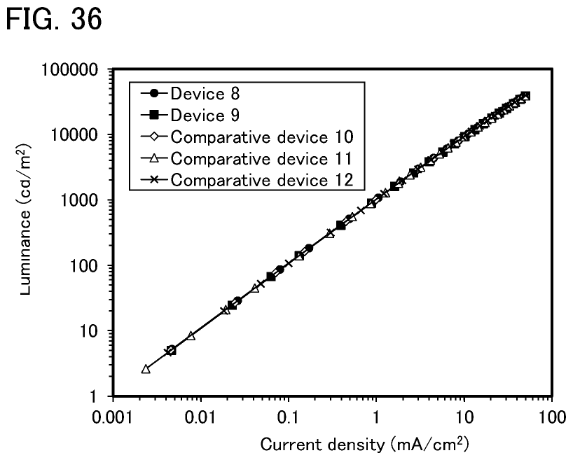

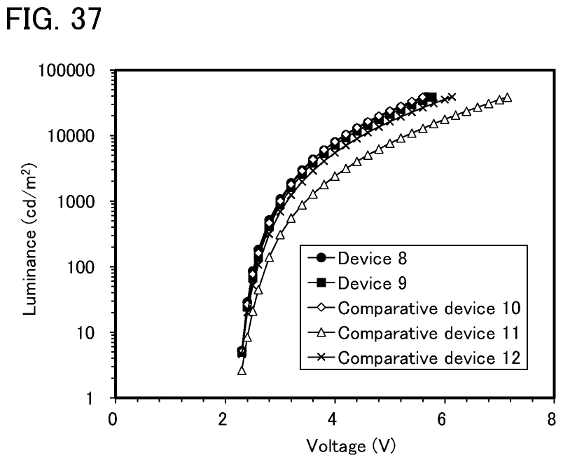

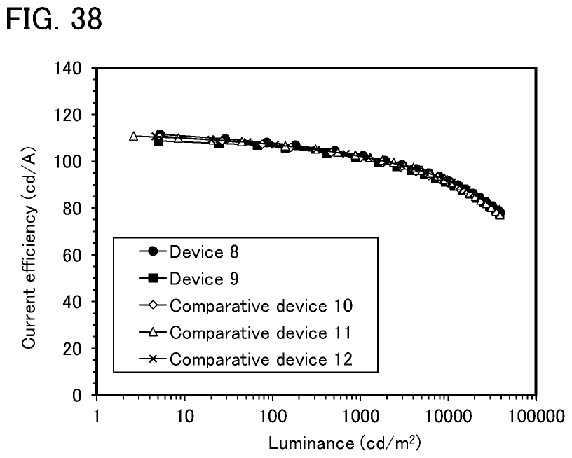

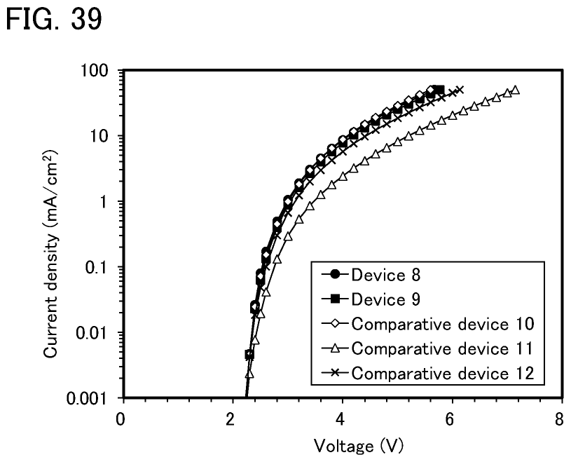

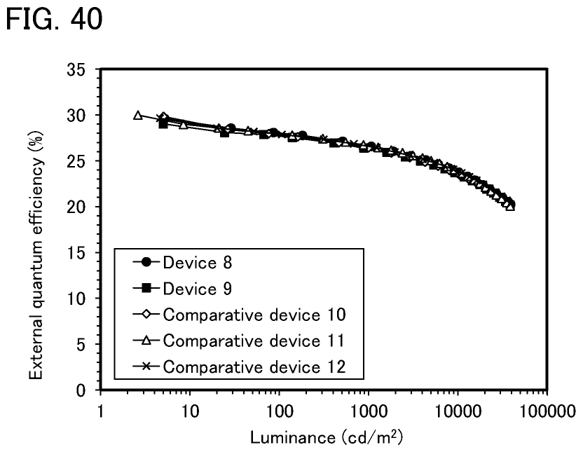

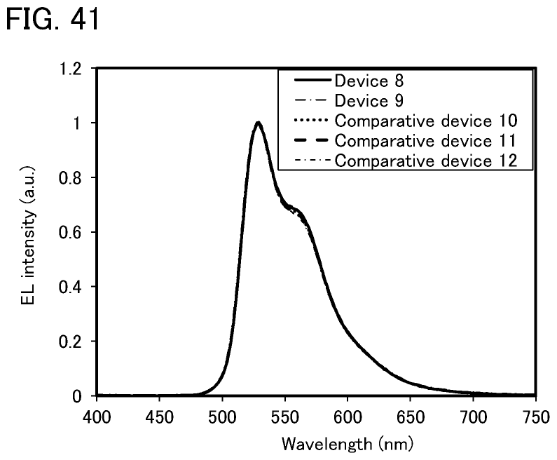

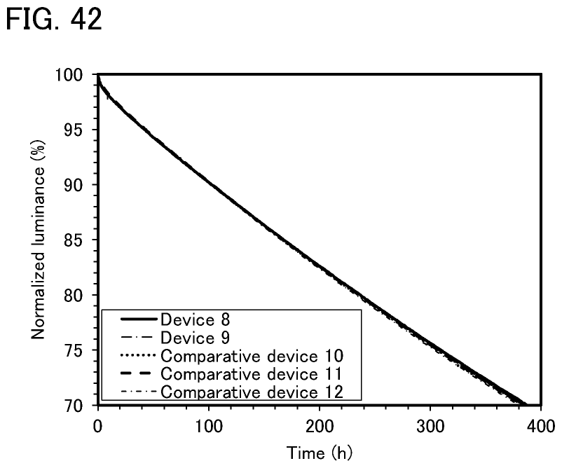

FIG. 36 shows luminance-current density characteristics of the light-emitting devices. FIG. 37 shows luminance-voltage characteristics thereof. FIG. 38 shows current efficiency-luminance characteristics thereof. FIG. 39 shows current density-voltage characteristics thereof. FIG. 40 shows external quantum efficiency-luminance characteristics thereof. FIG. 41 shows electroluminescence spectra thereof. FIG. 42 shows luminance changes over driving time when the light-emitting devices are driven at a constant current of 2 mA (50 mA/cm²).

Table 9 shows the main characteristics of the light-emitting devices at a luminance of approximately 1000 cd/m². Note that the luminance, CIE chromaticity, and emission spectra were measured with a spectroradiometer (SR-UL1R, manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 9

| | | | Current | | | | Current | External |
|---|---|---|---|---|---|---|---|---|
| | Voltage | Current | density | Chromaticity | Chromaticity | Luminance | efficiency | quantum |
| | (V) | (mA) | (mA/cm²) | x | y | (cd/m²) | (cd/A) | efficiency (%) |
| Device 8 | 3.00 | 0.0420 | 1.05 | 0.353 | 0.621 | 1074 | 102 | 26.6 |
| Device 9 | 3.00 | 0.0350 | 0.875 | 0.355 | 0.619 | 888 | 101 | 26.4 |
| Comparative device 10 | 3.00 | 0.0394 | 0.985 | 0.353 | 0.621 | 999 | 101 | 26.4 |
| Comparative device 11 | 3.40 | 0.0341 | 0.853 | 0.353 | 0.621 | 875 | 103 | 26.7 |
| Comparative device 12 | 3.20 | 0.0487 | 1.22 | 0.351 | 0.622 | 1236 | 101 | 26.4 |

From FIG. 36, FIG. 37, FIG. 38, FIG. 39, FIG. 40, FIG. 41, FIG. 42, and Table 9, the device 8 (the light-emitting device 8) and the device 9 (the light-emitting device 9) in each of which the organic compound of one embodiment of the present invention was used were found to be light-emitting devices having favorable characteristics that emit green light with a peak wavelength of approximately 533 nm derived from Ir(5mppy-d₃)₂(mbfpypy-d₃) and having long lifetimes.

FIG. 37 and FIG. 39 show that the devices 8 and 9 each have a driving voltage that is lower than those of the comparative devices 11 and 12 and is equivalent to that of the comparative device 10.

Here, Table 10 shows ordinary refractive indices (no) and GSP_slopes of evaporated films and T_{g} of powder of main organic compounds used in the light-emitting devices. In Table 10, the ordinary refractive index at 533 nm that is a peak wavelength of the devices 8 and 9 is shown as the ordinary refractive index. The ordinary refractive indices of the evaporated films of the organic compounds were measured by the method similar to that described in Example 1. In Table 10, the GSP_slopes of the evaporated films of the organic compounds were measured by the method similar to that described in Embodiment 1. In Table 10, the T_{g} of powder of the organic compounds were measured by the method similar to that described in Example 1.

TABLE 10

| | nₒ | GSP_slope | T_{g} |
|---|---|---|---|
| | (@533 nm) | (mV/nm) | (°C) |
| tBu-PSi-SFTzn | 1.71 | 14.4 | 176 |
| tBu-mPSi-SFTzn | 1.69 | 15.5 | 157 |
| tBu-SFTzn | 1.68 | 16.8 | 146 |
| tBu-mSFBPTzn | 1.72 | 16.5 | 161 |
| mSiTrz | 1.70 | 10.2 | 108 |

As shown in Table 10, tBu-PSi-SFTzn and tBu-mPSi-SFTzn, which are the organic compounds of one embodiment of the present invention, have as low refractive indices of films as tBu-SFTzn, tBu-mSFBPTzn, and mSiTrz, which are comparative organic compounds. In addition, tBu-PSi-SFTzn and tBu-mPSi-SFTzn have smaller GSP_slopes of evaporated films and higher T_{g} than tBu-SFTzn, tBu-mSFBPTzn, and mSiTrz.

As described above, the devices 8 and 9 each had a driving voltage that was lower than the comparative device 11 and was equivalent to that of the device 10. This shows that also in these light-emitting devices, bonding of the triphenylsilyl group to a vicinity of the triazine ring does not affect the electron-transport property, as in Example 3.

In addition, as described above, the devices 8 and 9 each had a driving voltage higher than that of the comparative device 12. Unlike tBu-PSi-SFTzn and tBu-mPSi-SFTzn, which are the organic compounds of one embodiment of the present invention, mSiTrz, which is the organic compound used for the comparative device 12, includes no 9,9′-spirobifluorenyl group. These results show that it is important to include both a 9,9′-spirobifluorenyl group and a triphenylsilyl group to improve the characteristics of the light-emitting device, as in the organic compound of one embodiment of the present invention.

The above results show that the use of the organic compound of one embodiment of the present invention enables fabrication of a light-emitting device with a low driving voltage and a long lifetime.

### Example 6

### Synthesis Example 3

This synthesis example describes a method for synthesizing the organic compound of one embodiment of the present invention, 2-(2′,7′-di-tert-butyl-9,9′-spirobi[9H-fluoren]-2-yl)-4-{3-[(3,5-di-tert-butylphenyl)diphenylsilyl]phenyl}-6-phenyl-1,3,5-triazine (abbreviation: tBu-mPSi-SFTzn-02) (Structural Formula (132)), and the physical properties thereof.

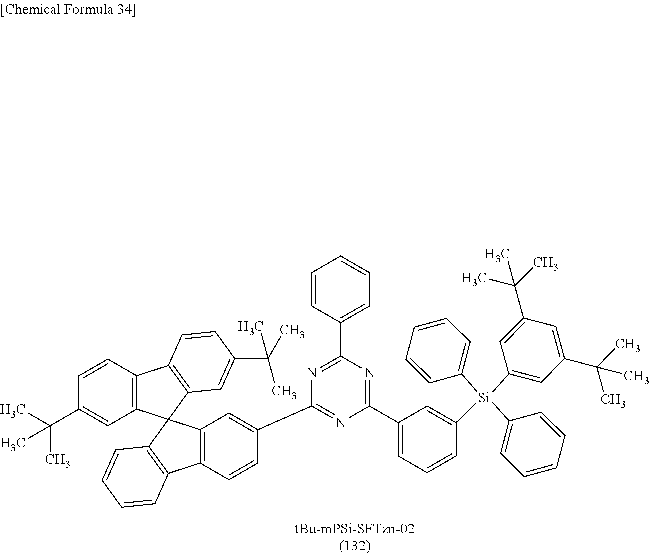 [SMILES: CC(C)(C)c1cc(C(C)(C)C)cc([Si](c2ccccc2)(c2ccccc2)c2cccc(-c3nc(-c4ccccc4)nc(-c4ccc5c(c4)C4(c6ccccc6-5)c5cc(C(C)(C)C)ccc5-c5ccc(C(C)(C)C)cc54)n3)c2)c1]

### Step 1: Synthesis of 1-(methoxydiphenylsilyl)-3,5-di-tert-butylbenzene (intermediate 1)

Into a 300-mL three-neck flask, 10 g (37 mmol) of 1-bromo-3,5-di-tert-butylbenzene was put, and the air in the flask was replaced with nitrogen. To the flask, 185 mL of tetrahydrofuran (abbreviation: THF) was added, and the solution was cooled down to −80° C. To this solution, 23 mL (37 mmol) of n-butyllithium (1.6 mol/L of hexane solution) was dropped with a syringe, and the mixture was stirred for 4 hours. After the stirring, 7.7 mL (37 mmol) of dimethoxydiphenylsilane was dropped, and the mixture was stirred overnight while it was brought to room temperature. After the stirring, approximately 100 mL of 1 mol/L hydrochloric acid was added to the solution, and the mixture was stirred. After the stirring, an aqueous layer of the mixture was subjected to extraction with ethyl acetate. The extracted solution and the organic layer were combined and washed with water and a saturated saline solution. The organic layer was dried with magnesium sulfate, and then the mixture was gravity filtered. The obtained filtrate was concentrated to give a solid. The obtained solid was purified by silica gel column chromatography (developing solvent: toluene:hexane=1:3 and then toluene:hexane=1:1). The obtained solid was purified by high performance liquid column chromatography. The high performance liquid column chromatography was performed using chloroform as a developing solvent. The obtained fraction was concentrated to give 11 g of a target white solid in a yield of 71%. The synthesis scheme of Step 1 is shown in Formula (c-1).

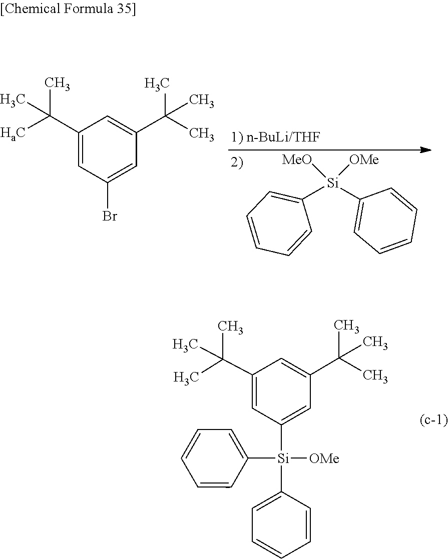

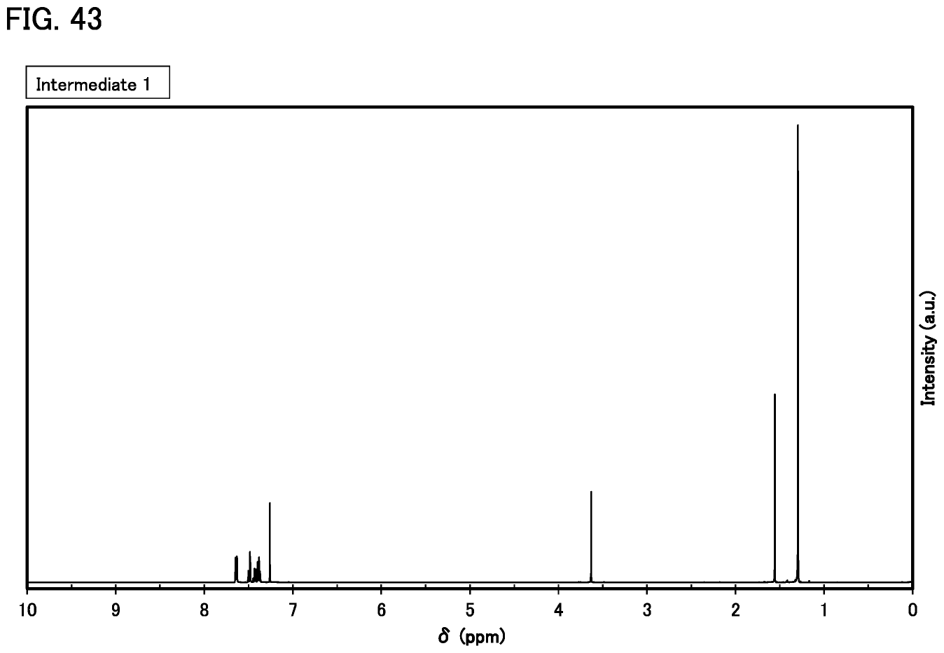

Analysis results by nuclear magnetic resonance (¹H-NMR) spectroscopy of the white solid obtained in the Step 1 are shown below. FIG. 43 is a ¹H-NMR chart. These results reveal that 1-(methoxydiphenylsilyl)-3,5-di-tert-butylbenzene (the intermediate 1) was obtained.

¹H NMR (CDCl₃, 500 MHz): δ=1.30 (s, 18H), 3.63 (s, 3H), 7.37-7.45 (m, 6H), 7.48 (d, J₁=2.0 Hz, 2H), 7.50 (t, J₁=2.0 Hz, 1H), 7.62-7.65 (m, 4H).

### Step 2: Synthesis of 1-bromo-3-[(3,5-di-tert-butylphenyl)diphenylsilyl]benzene (intermediate 2)

Into a 300-mL three-neck flask, 1.1 mL (9.5 mmol) of 1,3-dibromobenzene was put, and the air in the flask was replaced with nitrogen. To the flask, 50 mL of THF was added, and the solution was cooled down to −80° C. To this solution, 5.9 mL (9.5 mmol) of n-butyllithium (1.6 mol/L of hexane solution) was dropped with a syringe, and the mixture was stirred for 1.5 hours. After the stirring, 3.8 g (9.5 mmol) of 1-(methoxydiphenylsilyl)-3,5-di-tert-butylbenzene in a THF solution was dropped, and the mixture was stirred overnight while it was brought to room temperature. After the stirring, water was added to the mixture. An aqueous layer of the mixture was subjected to extraction with ethyl acetate. The extracted solution and the organic layer were combined and washed with water and a saturated saline solution. The organic layer was dried with magnesium sulfate, and then the mixture was gravity filtered. The obtained filtrate was concentrated to give a solid. The obtained solid was purified by silica gel column chromatography (developing solvent: toluene:hexane=1:3). The obtained solid was purified by high performance liquid column chromatography. The high performance liquid column chromatography was performed using chloroform as a developing solvent. The obtained fraction was concentrated to give 2.0 g of a target white solid in a yield of 40%. The synthesis scheme of Step 2 is shown in Formula (c-2).

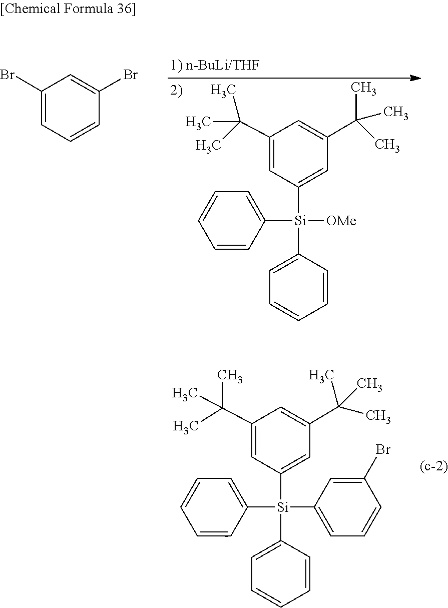 [SMILES: Brc1cccc(Br)c1 ; CO[Si](c1ccccc1)(c1cc(C(C)(C)C)cc(C(C)(C)C)c1)c1ccccc1C ; Cc1ccccc1[Si](c1ccccc1)(c1cccc(Br)c1)c1cc(C(C)(C)C)cc(C(C)(C)C)c1]

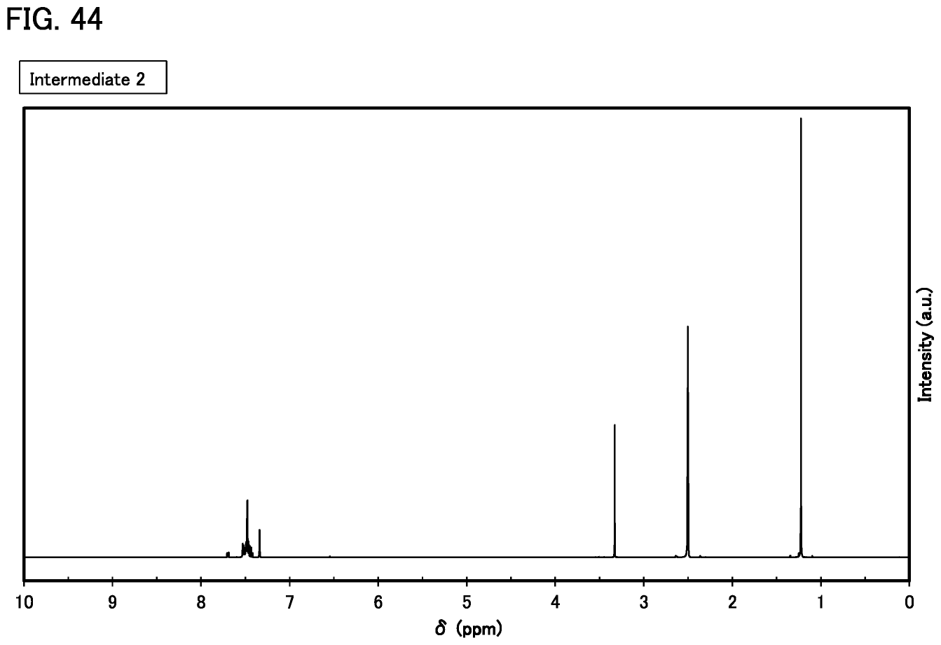

Analysis results by nuclear magnetic resonance (¹H-NMR) spectroscopy of the white solid obtained in the Step 2 are shown below. FIG. 44 is a ¹H-NMR chart. These results reveal that 1-bromo-3-[(3,5-di-tert-butylphenyl)diphenylsilyl]benzene (the intermediate 2) was obtained.

¹H NMR (DMSO-d₆, 500 MHz): δ=1.22 (s, 18H), 7.34 (d, J₁=2.0 Hz, 2H), 7.42-7.55 (m, 14H), 7.70 (ddd, J₁=7.5 Hz, J₂=2.0 Hz, J₃=1.0 Hz, 1H).

### Step 3: Synthesis of 2-{3-[(3,5-di-tert-butylphenyl)diphenylsilyl]phenyl}-4,4,5,5-tetramethyl-1,3,2-dioxaborane (intermediate 3)

Into a 200-mL three-neck flask, 2.0 g (3.8 mmol) of 1-bromo-3-[(3,5-di-tert-butylphenyl)diphenylsilyl]benzene, 1.4 g (5.6 mmol) of bis(pinacolato)diboron, and 1.1 g (11 mmol) of potassium acetate were put, and the air in the flask was replaced with nitrogen. To the flask, 20 mL of N,N-dimethylformamide was added, and the mixture was degassed while the pressure in the flask was reduced. After the degassing, 61 mg (75 μmol) of [1,1′-bis(diphenylphosphino)ferrocene]palladium(II) dichloride was added and the mixture was stirred at 100° C. for 6 hours. After the stirring, water was added to the mixture. The precipitated solid was collected by filtration. This solid was purified by silica gel column chromatography (developing solvent: toluene:hexane=1:3 and then toluene:hexane=2:3) to give 1.4 g of a white solid in a yield of 66%. The synthesis scheme of Step 3 is shown in Formula (c-3).

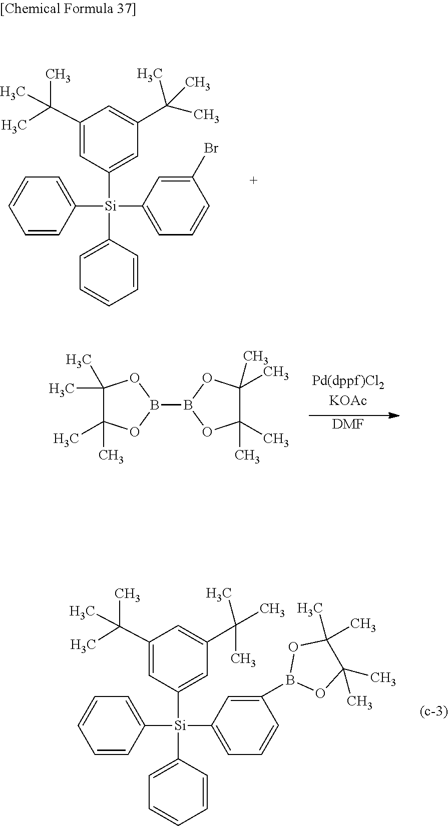

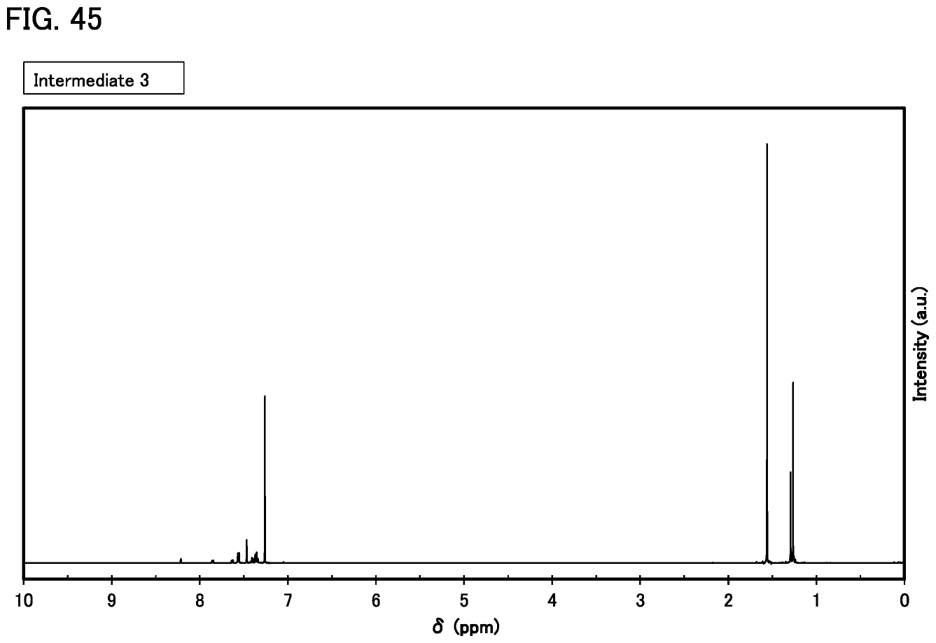

Analysis results by nuclear magnetic resonance (¹H-NMR) spectroscopy of the white solid obtained in the Step 3 are shown below. FIG. 45 is a ¹H-NMR chart. These results reveal that 2-{3-[(3,5-di-tert-butylphenyl)diphenylsilyl]phenyl}-4,4,5,5-tetramethyl-1,3,2-dioxaborane (the intermediate 3) was obtained.

¹H NMR (CDCl₃, 500 MHz): δ=1.27 (s, 18H), 1.29 (s, 12H), 7.34-7.47 (m, 10H), 7.18 (dd, J₁=7.0 Hz, J₂=1.0 Hz, 4H), 7.63 (dt, J₁=7.0 Hz, J₂=1.5 Hz, 1H), 7.85 (dt, J₁=7.0 Hz, J₂=1.5 Hz, 1H), 8.22 (s, 1H).

### Step 4: Synthesis of tBu-mPSi-SFTzn-02

Into a 200-mL three-neck flask, 1.0 g (1.7 mmol) of 2-chloro-4-(2′,7′-di-tert-butyl-9,9′-spirobi[9H-fluoren]-2-yl)-6-phenyl-1,3,5-triazine, 1.4 g (2.5 mmol) of 2-{3-[(3,5-di-tert-butylphenyl)diphenylsilyl]phenyl}-4,4,5,5-tetramethyl-1,3,2-dioxaborane, 10 mg (33 μmol) of tris(2-methylphenyl)phosphine, and 0.70 g (3.3 mmol) of tripotassium phosphate were put. To the mixture, 10 mL of toluene, 5 mL of 1,4-dioxane, and 5 mL of water were added. The mixture was degassed by being stirred while the pressure was reduced. To the mixture, 4.0 mg (17 μmol) of palladium(II) acetate was added and the mixture was stirred at 100° C. for 7 hours under a nitrogen stream. After the stirring, an aqueous layer of the mixture was subjected to extraction with toluene. The extracted solution and the organic layer were combined and washed with water and a saturated saline solution. The organic layer was dried with magnesium sulfate, and then the mixture was gravity filtered. The obtained filtrate was concentrated to give a solid. This solid was purified by silica gel column chromatography (developing solvent: toluene:hexane=1:3). The obtained solid was recrystallized with ethyl acetate/ethanol to give 1.4 g of a white solid in a yield of 80%. Then, 1.2 g of the obtained solid was purified by a train sublimation method in which the solid was heated at 350° C. under a pressure of 1.4 Pa with an argon flow rate of 5 mL/min. After the sublimation purification, 1.0 g of a white solid was obtained at a collection rate of 87%. The synthesis scheme of Step 4 is shown in Formula (c-4).

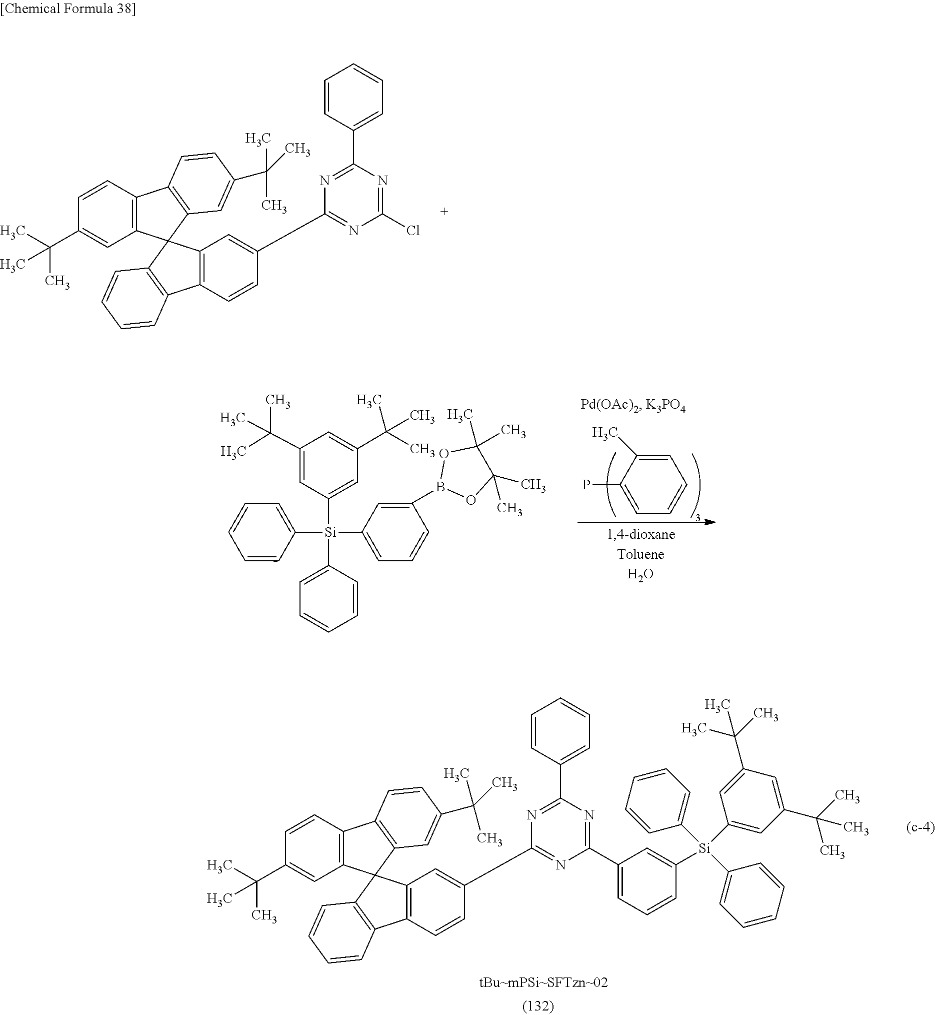

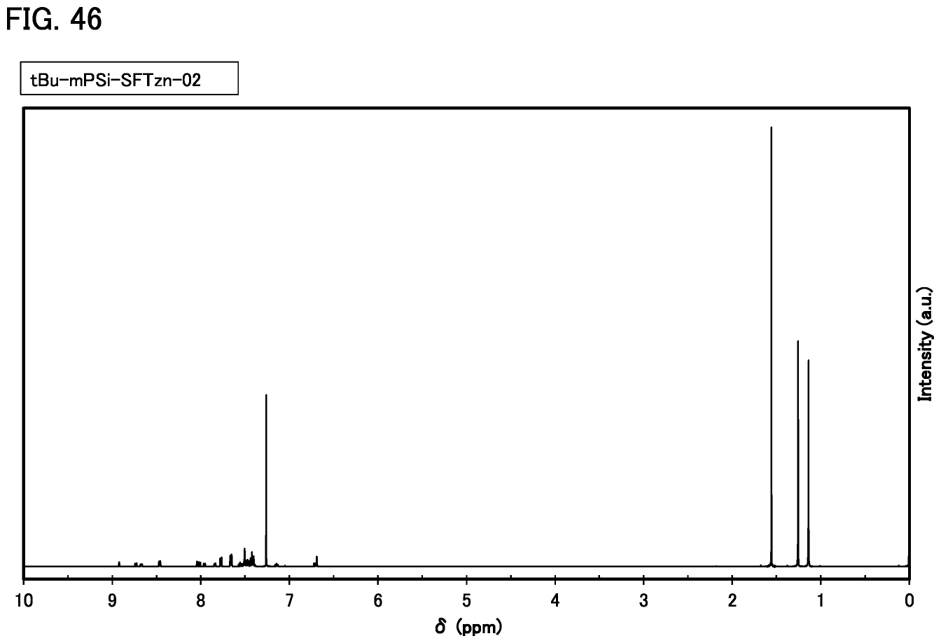

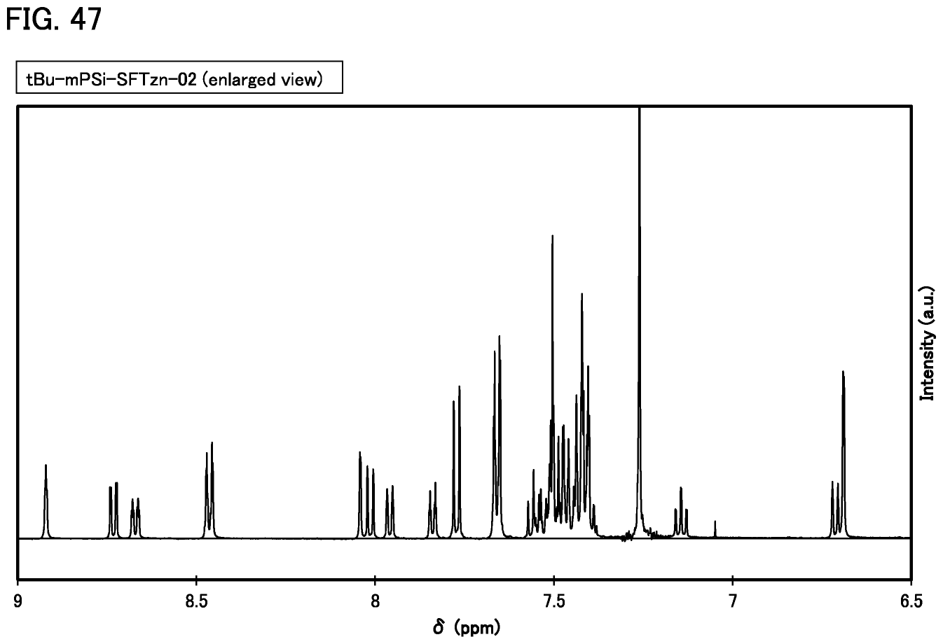

Analysis results by nuclear magnetic resonance (¹H-NMR) spectroscopy of the white solid obtained in the Step 4 are shown below. FIG. 46 is a ¹H-NMR chart, and FIG. 47 is an enlarged chart of the range of 6.5 ppm to 9 ppm in FIG. 46. These results reveal that the organic compound of one embodiment of the present invention, tBu-mPSi-SFTzn-02, was obtained.

¹H NMR (CDCl₃, 500 MHz): δ=1.14 (s, 18H), 1.26 (s, 18H), 6.69 (d, J₁=1.5 Hz, 2H), 6.71 (d, J₁=8.0 Hz, 1H), 7.14 (td, J₁=7.5 Hz, J₂=1.5 Hz, 1H), 7.38-7.57 (m, 16H), 7.66 (dd, J₁=8.0 Hz, J₂=1.5 Hz, 4H), 7.77 (d, J₁=8.0 Hz, 2H), 7.84 (dt, J₁=7.5 Hz, J₂=1.5 Hz, 1H), 7.96 (d, J₁=7.5 Hz, 1H), 8.01 (d, J₁=8.0 Hz, 1H), 8.01 (d, J₁=1.0 Hz, 1H), 8.46 (dd, J₁=8.5 Hz, J₂=1.5 Hz, 2H), 8.67 (dt, J₁=7.5 Hz, J₂=1.5 Hz, 1H), 8.73 (dd, J₁=8.0 Hz, J₂=1.5 Hz, 1H), 8.92 (t, J₁=1.5 Hz, 1H).

### <Measurement of Emission and Absorption Spectra>

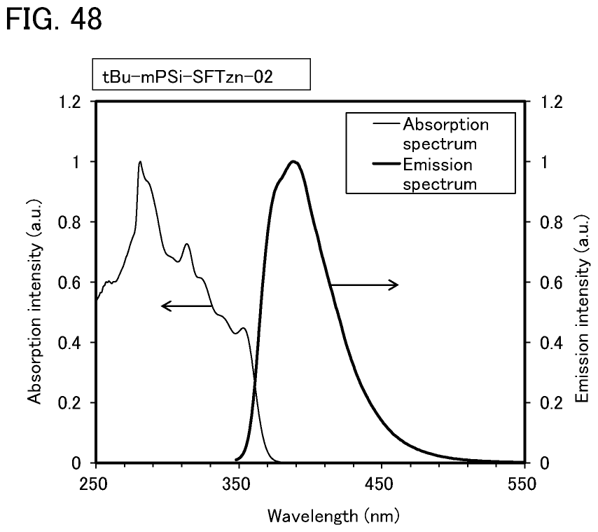

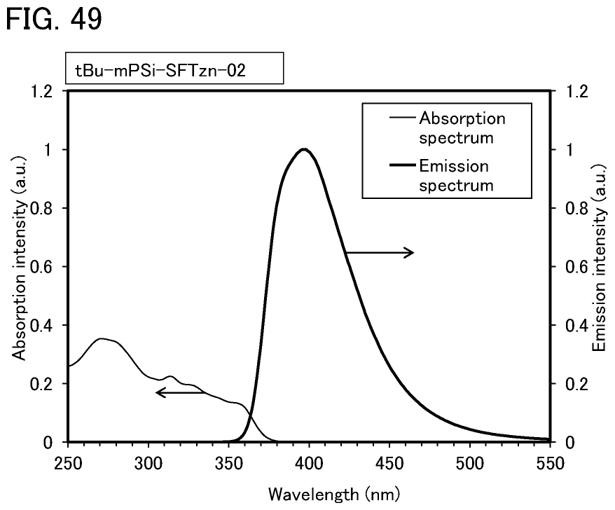

FIG. 48 shows the measurement results of emission and absorption spectra of tBu-mPSi-SFTzn-02 in a toluene solution. FIG. 49 shows the emission and absorption spectra of a thin film of tBu-mPSi-SFTzn-02. The emission and absorption spectra were measured by the method similar to that described in Example 1.

As shown in FIG. 48, tBu-mPSi-SFTzn-02 in the toluene solution has absorption spectrum peaks at wavelengths of 353 nm, 338 nm, 324 nm, 314 nm, and 287 nm and an emission spectrum peak at a wavelength of 388 nm (excitation wavelength: 338 nm). As shown in FIG. 49, the thin film of tBu-mPSi-SFTzn-02 has absorption spectrum peaks at wavelengths of 357 nm, 327 nm, 315 nm, 280 nm, and 271 nm and an emission spectrum peak at a wavelength of 396 nm (excitation wavelength: 330 nm).

### <Measurement of Refractive Index>

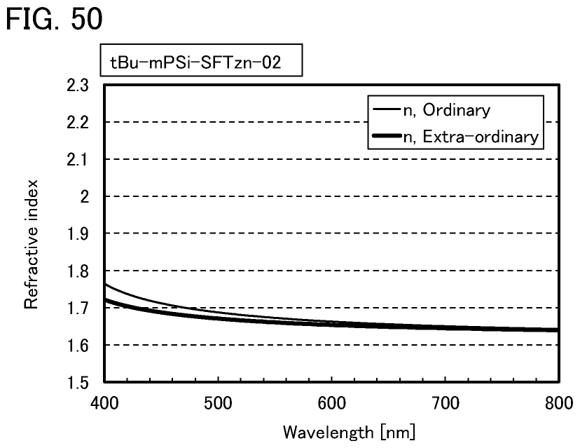

Next, by the method similar to that described in Example 1, the refractive index of a film formed using tBu-mPSi-SFTzn-02 was measured. FIG. 50 shows the measurement results. FIG. 50 shows that the film of tBu-mPSi-SFTzn-02 has a low refractive index: the ordinary refractive index in the entire blue emission region (the wavelength range of 455 nm to 465 nm) is in the range of 1.50 to 1.75 and the ordinary refractive index at a wavelength of 633 nm is 1.66.

### <Measurement of GSP_Slope>

Next, by the method similar to that described in Embodiment 1, the GSP_slope of an evaporated film formed using tBu-mPSi-SFTzn-02 was measured. The measurement result shows that the evaporated film of tBu-mPSi-SFTzn-02 has a GSP_slope of 34.6 mV/nm.

Here, tBu-mPSi-SFTzn-02 has a structure where two tert-butyl groups are introduced into a triphenylsilyl group of tBu-mPSi-SFTzn whose synthesizing method is shown in Example 2. As described in Example 2, the ordinary refractive index of an evaporated film formed using tBu-mPSi-SFTzn was 1.67 at a wavelength of 633 nm, and GSP_slope thereof was 15.5 mV/nm. Thus, it was found that when including two tert-butyl groups introduced into a triphenylsilyl group, tBu-mPSi-SFTzn-02 had a lower refractive index and a higher GSP_slope.

The above measurement results of the refractive index and GSP_slope indicate that in the case where each of R^{a}, R^{b}, and R^{c} in the organic compound of one embodiment of the present invention represents an aryl group having 6 to 12 carbon atoms, the refractive index of the film can be further reduced when the aryl group having 6 to 12 carbon atoms includes an alkyl group such as a tert-butyl group. Meanwhile, it is indicated that in the case where each of R^{a}, R^{b}, and R^{c} in the organic compound of one embodiment of the present invention represents an aryl group having 6 to 12 carbon atoms, a GSP_slope of the evaporated film can be further reduced in some cases when the aryl group having 6 to 12 carbon atoms includes no substituent.

### <Measurement of T_{g}>

Next, by the method similar to that described in Example 1, T_{g} was measured. The measurement result shows that the T_{g} of the sample formed using tBu-mPSi-SFTzn-02 is 142° C. Thus, it was found that the organic compound could give a solid having high T_{g}, and thus the evaporated film of the organic compound can also be expected to have high T_{g}.

### Example 7

### Synthesis Example 4

This synthesis example describes a method for synthesizing the organic compound of one embodiment of the present invention, 2-[3,5-bis(trimethylsilyl)phenyl]-4-(2′,7′-di-tert-butyl-9,9′-spirobi[9H-fluoren]-2-yl)-6-phenyl-1,3,5-triazine (abbreviation: tBu-mmTMSP-SFTzn) (Structural Formula (151)), and the physical properties thereof.

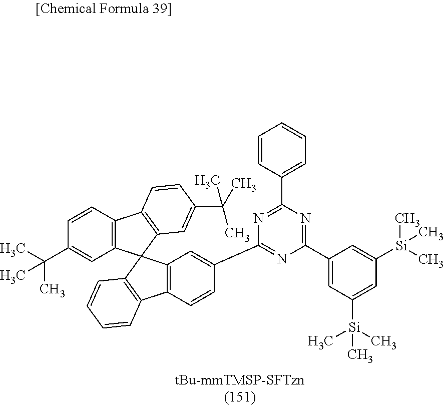 [SMILES: CC(C)(C)c1ccc2c(c1)C1(c3ccccc3-c3ccc(-c4nc(-c5ccccc5)nc(-c5cc([Si](C)(C)C)cc([Si](C)(C)C)c5)n4)cc31)c1cc(C(C)(C)C)ccc1-2 ; "tBu-mmTMSP-SFTzn(151)"]

Into a 200-mL three-neck flask, 1.9 g (3.1 mmol) of 2-chloro-4-(2′,7′-di-tert-butyl-9,9′-spirobi[9H-fluoren]-2-yl)-6-phenyl-1,3,5-triazine, 1.6 g (4.6 mmol) of 2-[3,5-bis(trimethylsilyl)phenyl]-4,4,5,5-tetramethyl-1,3,2-dioxaborolane, 19 mg (62 μmol) of tris(2-methylphenyl)phosphine, and 1.3 g (6.2 mmol) of tripotassium phosphate were put. To the mixture, 10 mL of toluene, 5 mL of 1,4-dioxane, and 5 mL of water were added. The mixture was degassed by being stirred while the pressure was reduced. To the mixture, 7.0 mg (31 μmol) of palladium(II) acetate was added and the mixture was stirred at 100° C. for 6 hours under a nitrogen stream. After the stirring, an aqueous layer of the mixture was subjected to extraction with toluene. The extracted solution and the organic layer were combined and washed with water and a saturated saline solution. The organic layer was dried with magnesium sulfate, and then the mixture was gravity filtered. The obtained filtrate was concentrated to give a solid. This solid was purified by silica gel column chromatography (developing solvent: toluene:hexane=1:4). The obtained solid was recrystallized with ethyl acetate/ethanol to give 1.9 g of a white solid in a yield of 78%. Then, 1.9 g of the obtained solid was purified by a train sublimation method in which the solid was heated at 250° C. under a pressure of 3.9 Pa with an argon flow rate of 15 mL/min. After the sublimation purification, 1.8 g of a white solid was obtained at a collection rate of 95%. The synthesis scheme is shown in Formula (d-1).

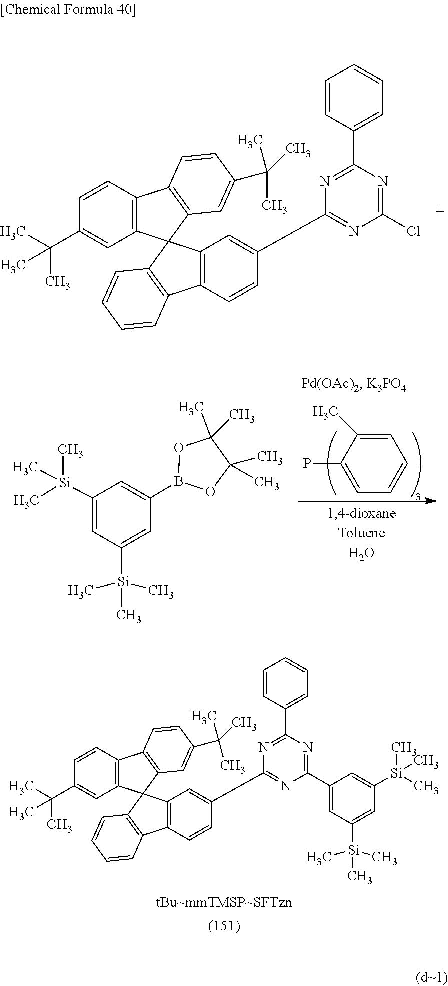

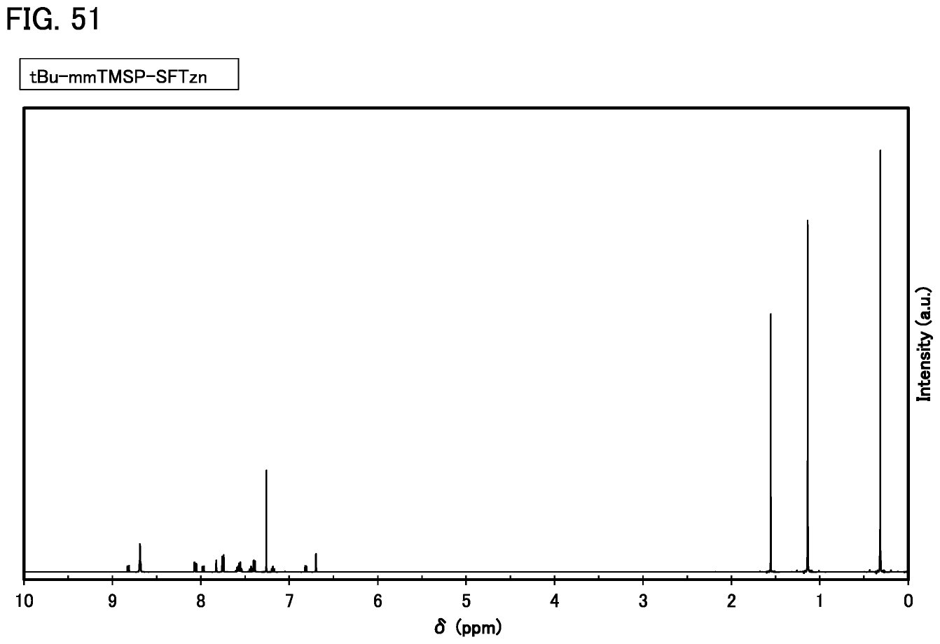

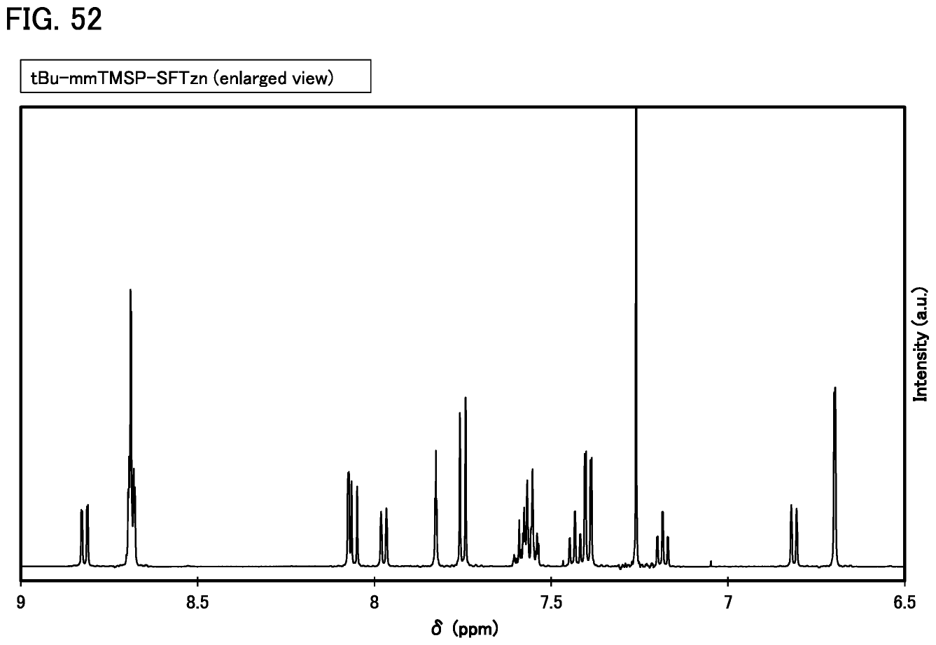

Analysis results by nuclear magnetic resonance (¹H-NMR) spectroscopy of the obtained white solid are shown below. FIG. 51 is a ¹H-NMR chart, and FIG. 52 is an enlarged chart of the range of 6.5 ppm to 9 ppm in FIG. 51. These results reveal that the organic compound of one embodiment of the present invention, tBu-mmTMSP-SFTzn, was obtained.

¹H NMR (CDCl₃, 500 MHz): δ=0.32 (s, 18H), 1.14 (s, 18H), 6.70 (d, J₁=1.5 Hz, 2H), 6.81 (d, J₁=7.5 Hz, 1H), 7.18 (td, J₁=7.5 Hz, J₂=1.0 Hz, 1H), 7.40 (dd, J₁=8.0 Hz, J₂=2.0 Hz, 2H), 7.43 (td, J₁=7.5 Hz, J₂=1.0 Hz, 1H), 7.54-7.61 (m, 3H), 7.75 (d, J₁=8.0 Hz, 2H), 7.83 (t, J₁=1.0 Hz, 1H), 7.97 (d, J₁=7.5 Hz, 1H), 8.05-8.07 (m, 2H), 8.68-8.70 (m, 4H), 8.82 (dd, J₁=8.0 Hz, J₂=1.5 Hz, 1H).

### <Measurement of Emission and Absorption Spectra>

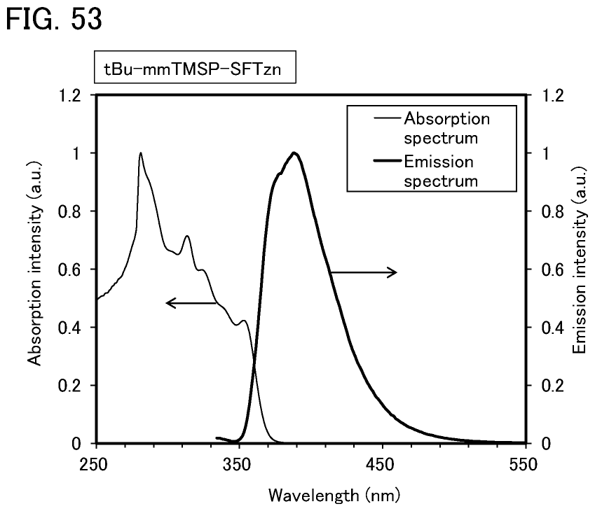

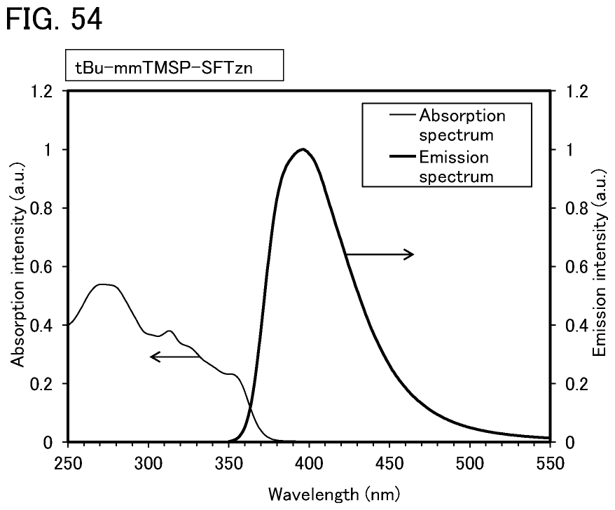

FIG. 53 shows the measurement results of emission and absorption spectra of tBu-mmTMSP-SFTzn in a toluene solution. FIG. 54 shows the emission and absorption spectra of a thin film of tBu-mmTMSP-SFTzn. The emission and absorption spectra were measured by the method similar to that described in Example 1.

As shown in FIG. 53, tBu-mmTMSP-SFTzn in the toluene solution has absorption spectrum peaks at wavelengths of 353 nm, 324 nm, and 313 nm and emission spectrum peaks at wavelengths of 377 nm and 388 nm (excitation wavelength: 324 nm). As shown in FIG. 54, the thin film of tBu-mmTMSP-SFTzn has absorption spectrum peaks at wavelengths of 354 nm, 313 nm, and 272 nm and an emission spectrum peak at a wavelength of 396 nm (excitation wavelength: 325 nm).

### <Measurement of Refractive Index>

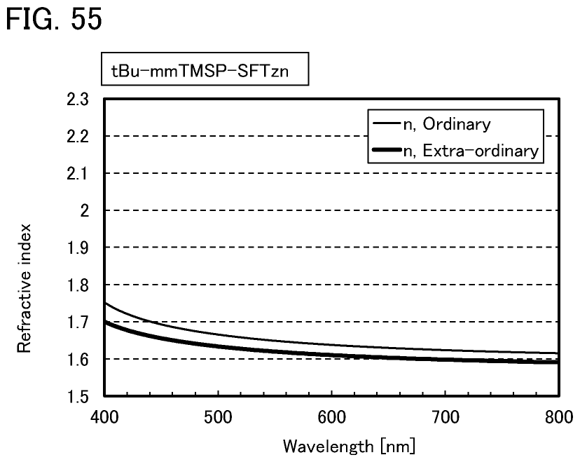

Next, by the method similar to that described in Example 1, the refractive index of a film formed using tBu-mmTMSP-SFTzn was measured. FIG. 55 shows the measurement results. FIG. 55 shows that the film of tBu-mmTMSP-SFTzn has a low refractive index: the ordinary refractive index in the entire blue emission region (the wavelength range of 455 nm to 465 nm) is in the range of 1.50 to 1.75 and the ordinary refractive index at a wavelength of 633 nm is 1.63.

### <Measurement of GSP_Slope>

Next, by the method similar to that described in Embodiment 1, the GSP_slope of an evaporated film formed using tBu-mmTMSP-SFTzn was measured. The measurement result shows that the evaporated film of tBu-mmTMSP-SFTzn has a small GSP_slope: the GSP_slope is 19.4 mV/nm.

Here, it was found that the GSP_slope of the evaporated film of the comparative organic compound tBu-SFTzn-04, which has a structure where two trimethylsilyl groups included in tBu-mmTMSP-SFTzn are replaced with tert-butyl groups, was 27.7 mV/nm; thus, GSP_slope of an evaporated film of tBu-mmTMSP-SFTzn was smaller than that of the comparative organic compound tBu-SFTzn-04. This indicates that the GSP_slope of the evaporated film of the organic compound of one embodiment of the present invention in which a trisubstituted silyl group is introduced can be smaller than that of the organic compound in which a trisubstituted methyl group is introduced. Structural formulae of tBu-mmTMSP-SFTzn and tBu-SFTzn-04 are shown below.

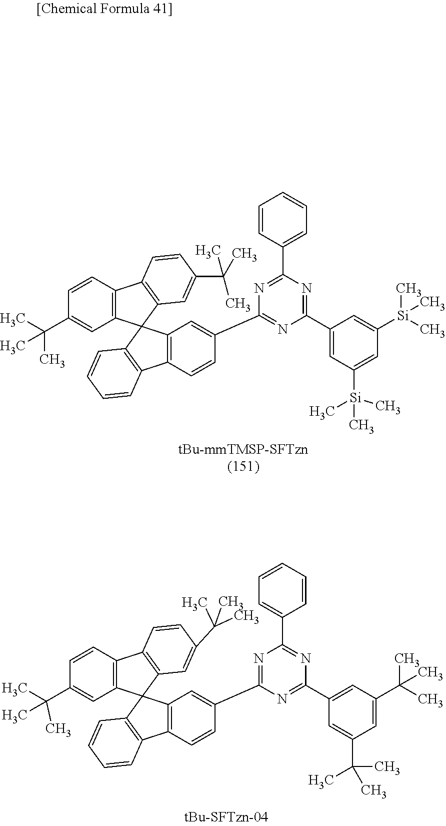

### <Measurement of T_{g}>

Next, by the method similar to that described in Example 1, T_{g} was measured. The measurement result shows that the T_{g} of the sample formed using tBu-mmTMSP-SFTzn is 146° C. Thus, it was found that the organic compound could give a solid having high T_{g}, and thus the evaporated film of the organic compound can also be expected to have high T_{g}.

### Example 8

### Synthesis Example 5

This synthesis example describes a method for synthesizing the organic compound of one embodiment of the present invention, 2-(2′,7′-di-tert-butyl-9,9′-spirobi[9H-fluoren]-2-yl)-4-phenyl-6-[4-(triphenylmethyl)phenyl]-1,3,5-triazine (abbreviation: tBu-TrP-SFTzn) (Structural Formula (122)), and the physical properties thereof.

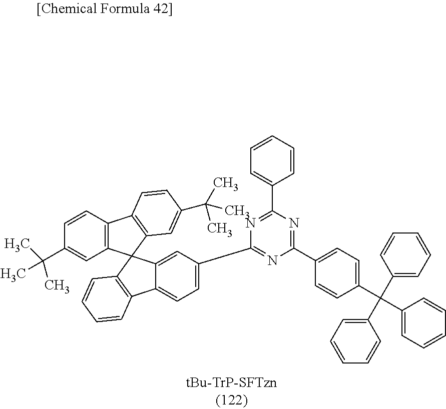 [SMILES: CC(C)(C)c1ccc2c(c1)C1(c3ccccc3-c3ccc(-c4nc(-c5ccccc5)nc(-c5ccc(C(c6ccccc6)(c6ccccc6)c6ccccc6)cc5)n4)cc31)c1cc(C(C)(C)C)ccc1-2]

Into a 200-mL three-neck flask, 2.5 g (4.0 mmol) of 2-chloro-4-(2′,7′-di-tert-butyl-9,9′-spirobi[9H-fluoren]-2-yl)-6-phenyl-1,3,5-triazine, 2.0 g (4.5 mmol) of 2-[4-(triphenylmethyl)phenyl]-4,4,5,5-tetramethyl-1,3,2-dioxaborolane, 1.1 g (8.1 mmol) of potassium carbonate were put. To the mixture, 13 mL of toluene, 6 mL of 1,4-dioxane, and 4 mL of water were added. The mixture was degassed by being stirred while the pressure was reduced. To the mixture, 9.1 mg (40 μmol) of palladium(II) acetate and 25 mg (81 μmol) of tris(2-methylphenyl)phosphine were added and the mixture was stirred at 120° C. for 14 hours under a nitrogen stream. After the stirring, water was added to the mixture to separate the mixture into an organic layer and an aqueous layer. The aqueous layer was subjected to extraction with toluene. The extracted solution and the organic layer were combined. The mixture was dried with magnesium sulfate, and then was gravity filtered. The obtained filtrate was concentrated to give a solid. This solid was purified by silica gel column chromatography (developing solvent: toluene:hexane=1:3 and then toluene:hexane=1:2). After that, further purification was performed by high performance liquid chromatography using chloroform as a mobile phase. The obtained solid was recrystallized with toluene/ethanol to give 2.0 g of a white solid in a yield of 55%. Then, 2.0 g of the obtained solid was purified by a train sublimation method in which the solid was heated at 325° C. under a pressure of 5.3 Pa with an argon flow rate of 10 mL/min. After the sublimation purification, 1.7 g of a white solid was obtained at a collection rate of 87%. The synthesis scheme is shown in Formula (e-1).

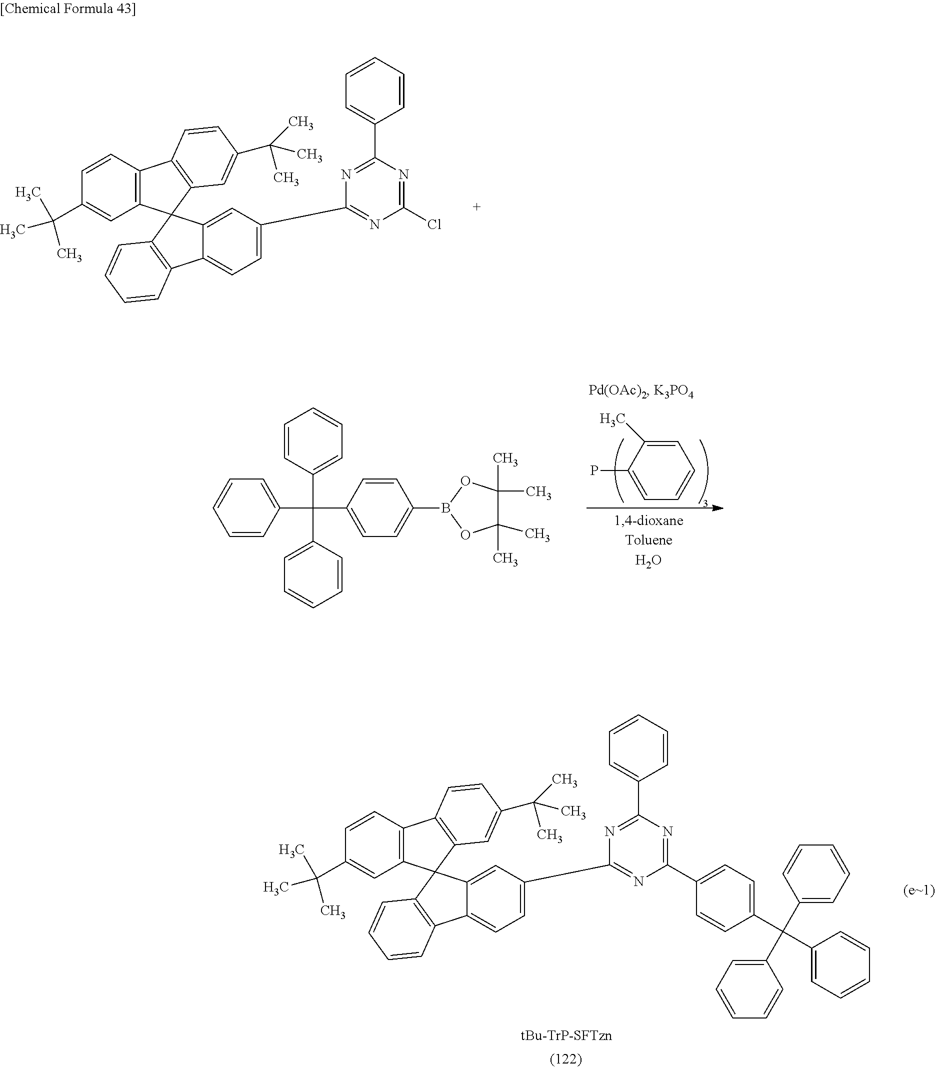

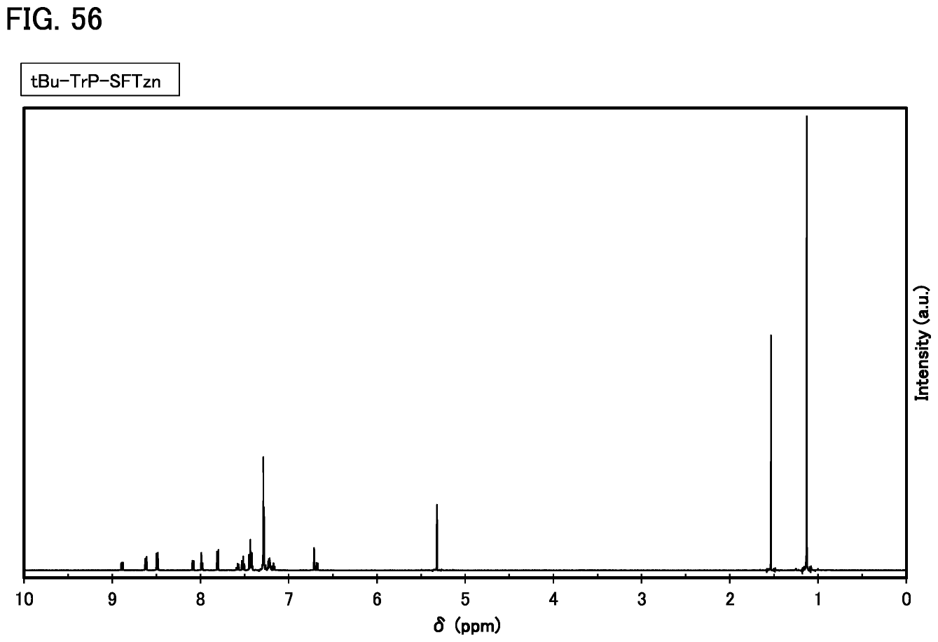

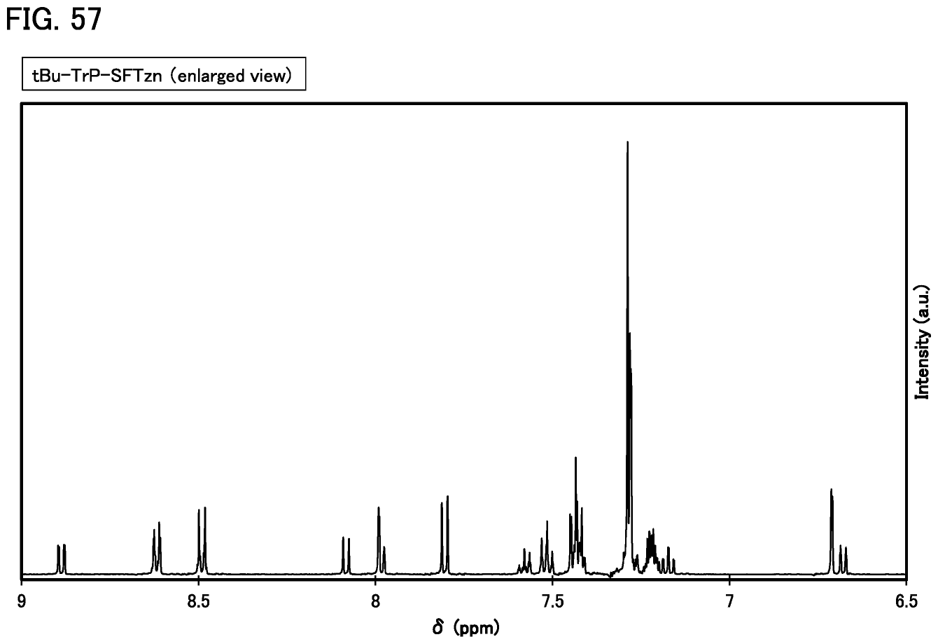

Analysis results by nuclear magnetic resonance (¹H-NMR) spectroscopy of the obtained white solid are shown below. FIG. 56 is a ¹H-NMR chart, and FIG. 57 is an enlarged chart of the range of 6.5 ppm to 9 ppm in FIG. 56. These results reveal that the organic compound of one embodiment of the present invention, tBu-TrP-SFTzn, was obtained.

¹H NMR (CD₂Cl₂, 500 MHz): δ=1.13 (s, 18H), 6.68 (d, J₁=7.6 Hz, 1H), 6.71 (d, J₁=1.4 Hz, 2H), 7.17 (td, J₁=7.5 Hz, J₂=1.1 Hz, 1H), 7.20-7.24 (m, 3H), 7.26-7.30 (m, 12H), 7.41-7.46 (m, 5H), 7.50-7.53 (m, 2H), 7.58 (tt, J₁=7.3 Hz, J₂=1.6 Hz, 1H), 7.80 (d, J₁=8.2 Hz, 2H), 7.98-7.99 (m, 2H), 8.08 (d, J₁=8.2 Hz, 1H), 8.48-8.50 (m, 2H), 8.61-8.63 (m, 2H), 8.89 (dd, J₁=8.1 Hz, J₂=1.5 Hz, 1H).

### <Measurement of Emission and Absorption Spectra>

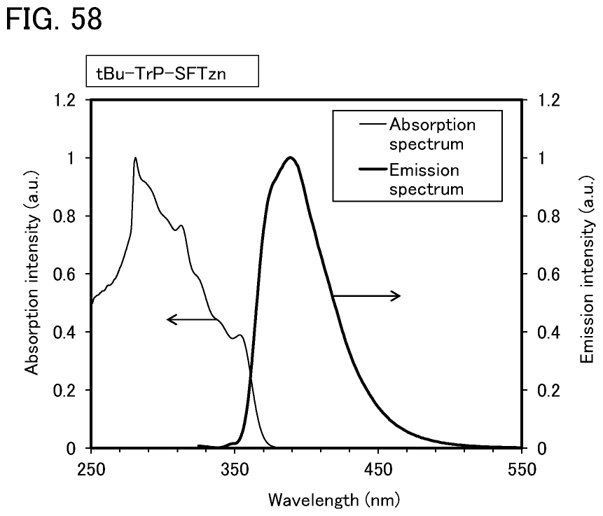

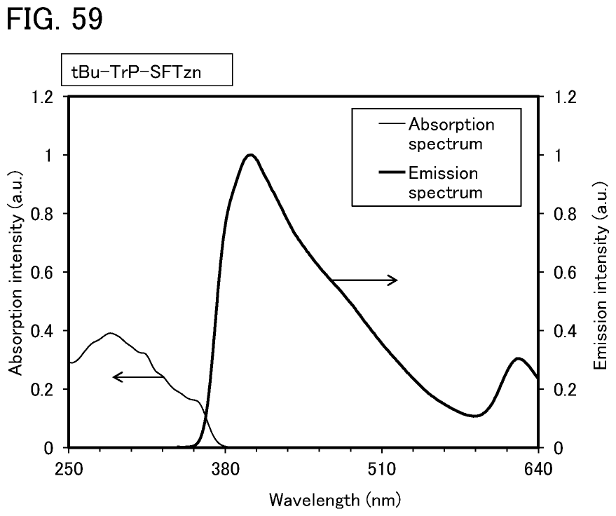

FIG. 58 shows the measurement results of emission and absorption spectra of tBu-TrP-SFTzn in a toluene solution. FIG. 59 shows the emission and absorption spectra of a thin film of tBu-TrP-SFTzn. The emission and absorption spectra were measured by the method similar to that described in Example 1.

As shown in FIG. 58, tBu-TrP-SFTzn in the toluene solution has absorption spectrum peaks at wavelengths of 354 nm and 313 nm and an emission spectrum peak at a wavelength of 389 nm (excitation wavelength: 315 nm). As shown in FIG. 59, the thin film of tBu-TrP-SFTzn has absorption spectrum peaks at wavelengths of 356 nm, 312 nm, and 286 nm and emission spectrum peaks at wavelengths of 402 nm and 624 nm (excitation wavelength: 328 nm).

### <Measurement of Refractive Index>

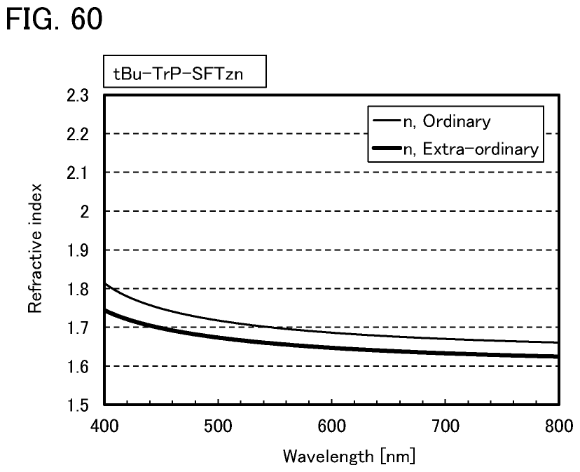

Next, by the method similar to that described in Example 1, the refractive index of a film formed using tBu-TrP-SFTzn was measured. FIG. 60 shows the measurement results. FIG. 60 shows that the film of tBu-TrP-SFTzn has a low refractive index: the ordinary refractive index in the entire blue emission region (the wavelength range of 455 nm to 465 nm) is in the range of 1.50 to 1.75 and the ordinary refractive index at a wavelength of 633 nm is 1.71.

### <Measurement of GSP_Slope>

Next, by the method similar to that described in Embodiment 1, the GSP_slope of an evaporated film formed using tBu-TrP-SFTzn was measured. The measurement result shows that the evaporated film of tBu-TrP-SFTzn has a small GSP_slope: the GSP_slope is 15.4 mV/nm.

### <Measurement of T_{g}>

Next, by the method similar to that described in Example 1, T_{g} was measured. The measurement result shows that the T_{g} of the sample formed using tBu-TrP-SFTzn is 187° C. Thus, it was found that the organic compound could give a solid having high T_{g}, and thus the evaporated film of the organic compound can also be expected to have high T_{g}.

### Example 9

This example describes the fabrication of the device 13 (the light-emitting device 13) in which tBu-mPSi-SFTzn (Structural Formula (129)), which is the organic compound of one embodiment of the present invention described in Example 2, was used for the electron-transport layer, the device 14 (the light-emitting device 14) in which tBu-mPSi-SFTzn-02 (Structural Formula (132)), which is the organic compound of one embodiment of the present invention described in Example 6, was used for the electron-transport layer, and the comparative device 15 (the comparative light-emitting device 15) in which a comparative organic compound was used for the electron-transport layer, and measurement results of these devices' characteristics. These light-emitting devices are bottom-emission light-emitting devices that emit green phosphorescent light. Structural formulae of organic compounds used for the light-emitting devices are shown below.

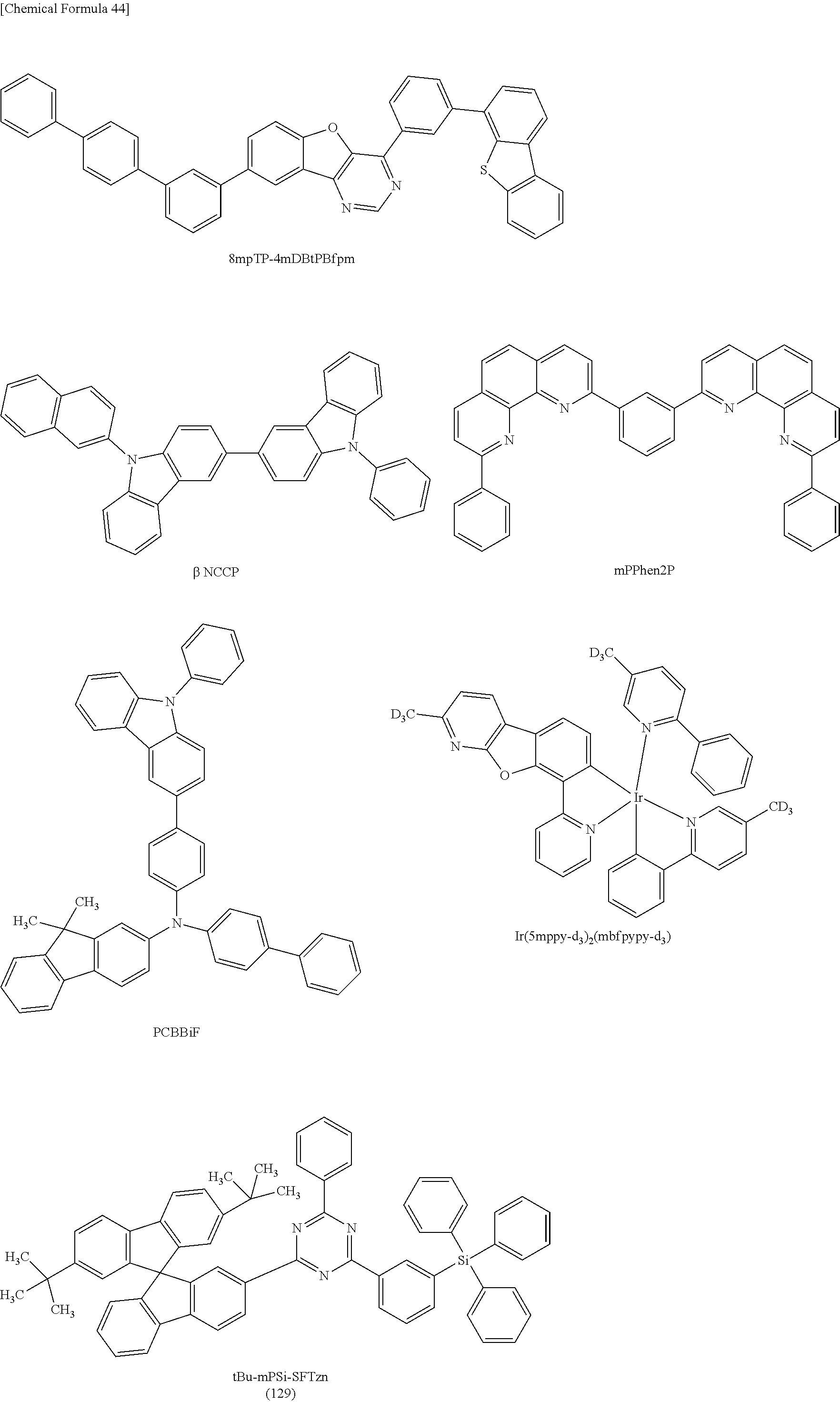

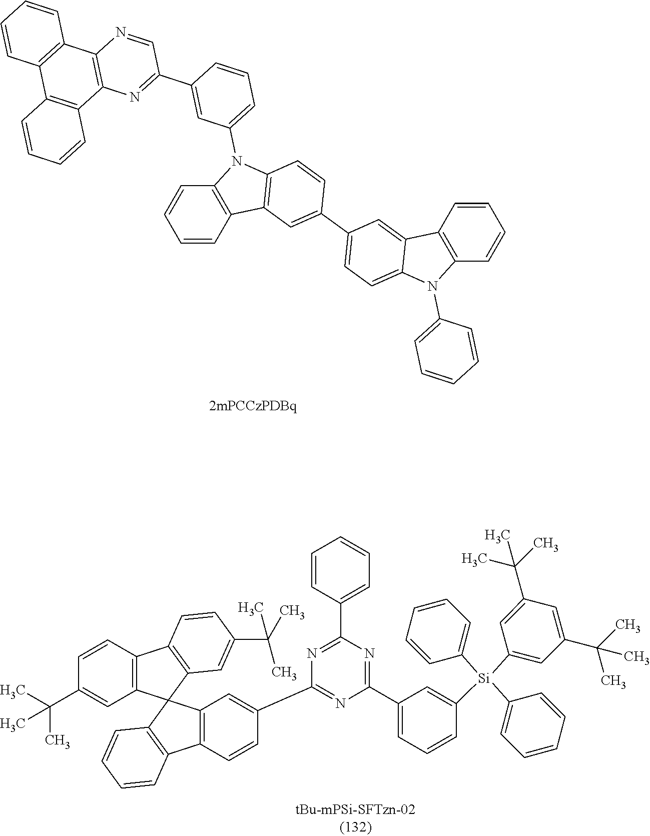 [SMILES: c1ccc(-n2c3ccccc3c3cc(-c4ccc5c(c4)c4ccccc4n5-c4cccc(-c5cnc6c7ccccc7c7ccccc7c6n5)c4)ccc32)cc1 ; CC(C)(C)c1cc(C(C)(C)C)cc([Si](c2ccccc2)(c2ccccc2)c2cccc(-c3nc(-c4ccccc4)nc(-c4ccc5c(c4)C4(c6ccccc6-5)c5cc(C(C)(C)C)ccc5-c5ccc(C(C)(C)C)cc54)n3)c2)c1]

As illustrated in FIG. 11B, the light-emitting devices each have a structure in which the hole-injection layer 911, the hole-transport layer 912, the light-emitting layer 913, electron-transport layers (the first electron-transport layer 9141 and the second electron-transport layer 914_2), and the electron-injection layer 915 are stacked in this order over the first electrode 901 formed over the glass substrate 900, and the second electrode 902 is stacked over the electron-injection layer 915.

### <Fabrication Method of Device 13>

ITSO was deposited by a sputtering method over the glass substrate 900 to a thickness of 70 nm, so that the first electrode 901 as a transparent electrode was formed. The electrode area was set to 4 mm² (2 mm×2 mm).

Next, as in the fabrication method of the device 1 in Example 3, pretreatment for fabricating the light-emitting device over a substrate and vacuum baking were performed. After that, natural cooling was performed.

Then, after forming the hole-injection layer 911 as in the fabrication method of the device 1, PCBBiF was deposited by evaporation to a thickness of 40 nm over the hole-injection layer 911, whereby the hole-transport layer 912 was formed. Then, as in the fabrication method of the device 1, the light-emitting layer 913 was formed over the hole-transport layer 912.

Next, over the light-emitting layer 913, tBu-mPSi-SFTzn, which is the organic compound of one embodiment of the present invention, was deposited by evaporation to a thickness of 20 nm, whereby the first electron-transport layer 914_1 was formed. Then, mPPhen2P was deposited by evaporation to a thickness of 20 nm, whereby the second electron-transport layer 914_2 was formed.

Then, as in the fabrication method of the device 8 in Example 5, the electron-injection layer 915 and the second electrode 902 were formed.

### <Fabrication Method of Device 14>

The device 14 is different from the device 13 in that tBu-mPSi-SFTzn, which is the organic compound of one embodiment of the present invention used for the first electron-transport layer 914_1 of the device 13, was replaced with tBu-mPSi-SFTzn-02, which is an organic compound of another embodiment of the present invention. Other components were fabricated in a manner similar to that for the device 13.

### <Fabrication Method of Comparative Device 15>

The comparative device 15 is different from the device 13 in that tBu-mPSi-SFTzn, which is the organic compound of one embodiment of the present invention used for the first electron-transport layer 914_1 of the device 13, was replaced with 2mPCCzPDBq, which is a comparative organic compound. Other components were fabricated in a manner similar to that for the device 13.

The device structures of the light-emitting devices are listed in Table 11.

TABLE 11

| | | Film | | | Comparative |
|---|---|---|---|---|---|
| | | thickness | Device 13 | Device 14 | device 15 |
| Second electrode | | 100 nm | Al | | |
| Electron-injection layer | | 1 nm | LiF | | |
| Electron-transport | 2 | 20 nm | mPPhen2P | | |
| layer | 1 | 20 nm | tBu-mPSi-SFTzn | tBu-mPSi-SFTzn-02 | 2mPCCzPDBq |

| Light-emitting layer | 40 nm | 8mpTP-4mDBtPBfpm:βNCCP:Ir(5mppy-d₃)₂(mbfpypy-d₃) |
|---|---|---|
| | | (0.5:0.5:0.1) |
| Hole-transport layer | 40 nm | PCBBiF |
| Hole-injection layer | 10 nm | PCBBiF:OCHD-003 (1:0.03) |
| First electrode | 70 nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for 1 hour were performed at the time of sealing). Then, the characteristics of the light-emitting devices were measured.

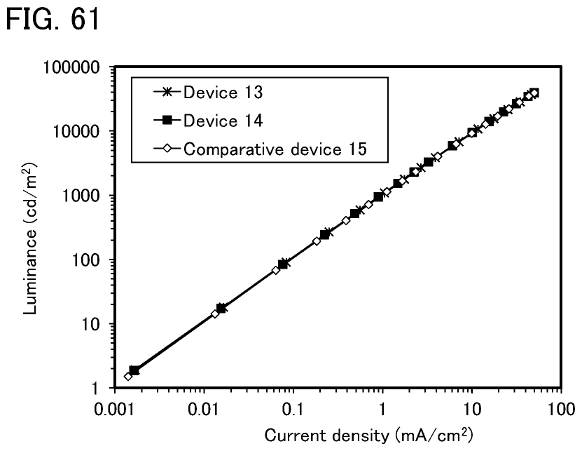

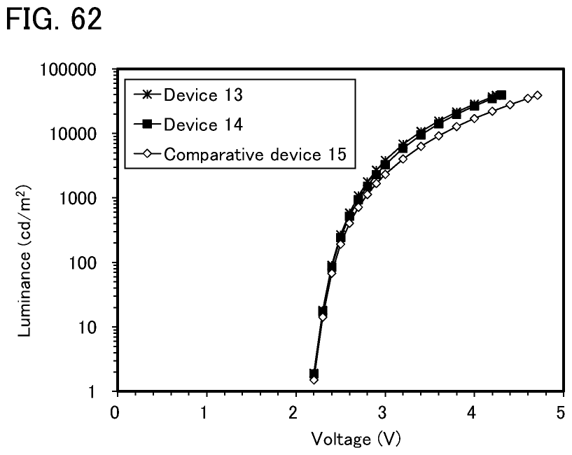

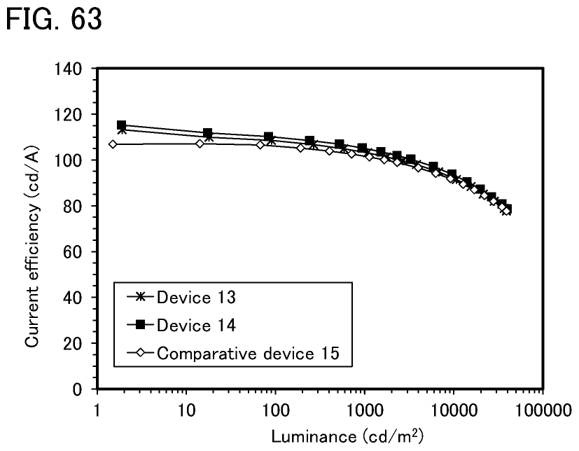

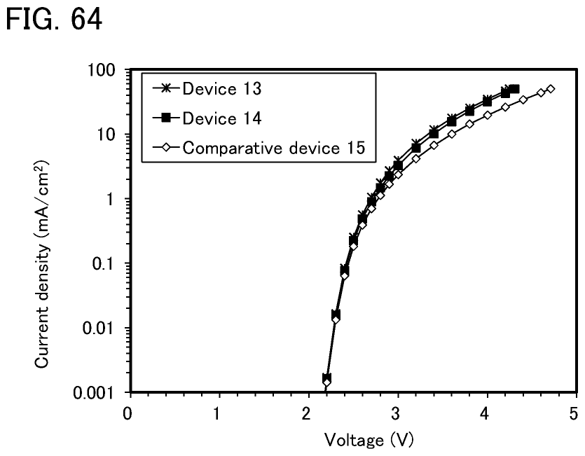

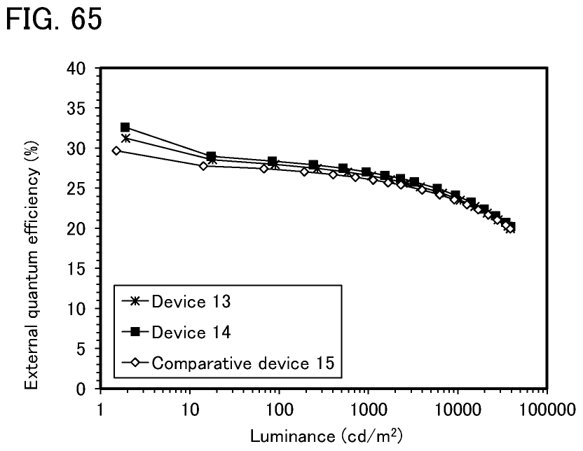

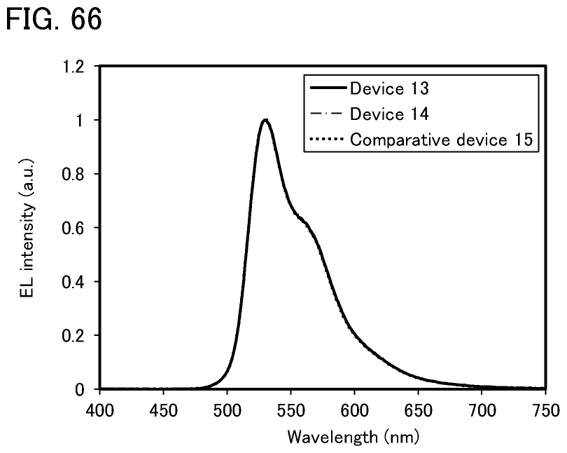

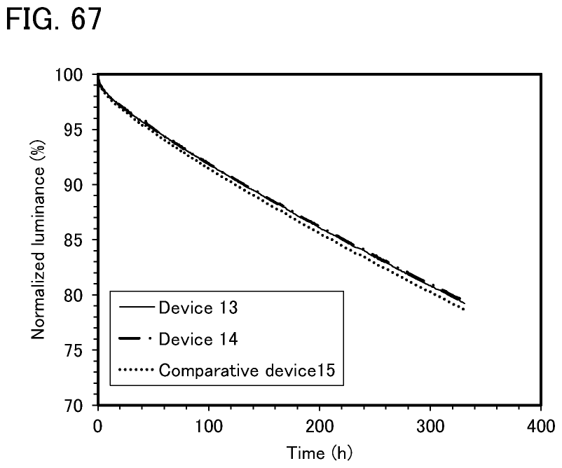

FIG. 61 shows luminance-current density characteristics of the light-emitting devices. FIG. 62 shows luminance-voltage characteristics thereof. FIG. 63 shows current efficiency-luminance characteristics thereof. FIG. 64 shows current density-voltage characteristics thereof. FIG. 65 shows external quantum efficiency-luminance characteristics thereof. FIG. 66 shows electroluminescence spectra thereof. FIG. 67 shows luminance changes over driving time when the light-emitting devices are driven at a constant current of 2 mA (50 mA/cm²).

Table 12 shows the main characteristics of the light-emitting devices at a luminance of approximately 1000 cd/m². Note that the luminance, CIE chromaticity, and emission spectra were measured with a spectroradiometer (SR-UL1R, manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 12

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Device 13 | 2.70 | 0.0418 | 1.05 | 0.347 | 0.627 | 1078 | 103 | 26.5 |
| Device 14 | 2.70 | 0.0357 | 0.892 | 0.346 | 0.628 | 939 | 105 | 27.0 |
| Comparative device 15 | 2.80 | 0.0446 | 1.12 | 0.346 | 0.629 | 1130 | 101 | 26.0 |

From FIG. 61, FIG. 62, FIG. 63, FIG. 64, FIG. 65, FIG. 66, FIG. 67, and Table 12, the device 13 (the light-emitting device 13) and the device 14 (the light-emitting device 14) in each of which the organic compound of one embodiment of the present invention was used were found to be light-emitting devices having favorable characteristics that emit green light with a peak wavelength of approximately 533 nm derived from Ir(5mppy-d₃)₂(mbfpypy-d₃) and having long lifetimes.

FIG. 62 and FIG. 64 show that the devices 13 and 14 each have a driving voltage lower than that of the comparative device 15. FIG. 63 and FIG. 65 show that the devices 13 and 14 each have current efficiency and external quantum efficiency that are higher than those of the comparative device 15. The device 14 has current efficiency and external quantum efficiency that are higher than those of the device 13. FIG. 67 shows that the device 13, the device 14, and the comparative device 15 have equivalent lifetimes.

Here, Table 13 shows ordinary refractive indices (no) of evaporated films of main organic compounds used in the light-emitting devices. In Table 13, the ordinary refractive index at 533 nm that is a peak wavelength of the devices 13 and 14 is shown as the ordinary refractive index. The ordinary refractive indices of the evaporated films of the organic compounds were measured by the method similar to that described in Example 1.

TABLE 13

| | nₒ (@533 nm) |
|---|---|
| tBu-mPSi-SFTzn | 1.69 |
| tBu-mPSi-SFTzn-02 | 1.68 |
| 2mPCCzPDBq | 1.92 |

As shown in Table 13, tBu-mPSi-SFTzn and tBu-mPSi-SFTzn-02, which are the organic compounds of one embodiment of the present invention, have refractive indices of films lower than that of 2mPCCzPDBq, which is a comparative organic compound. Furthermore, tBu-mPSi-SFTzn-02 has a refractive index of a film lower than that of tBu-mPSi-SFTzn.

As described above, each of the devices 13 and 14 formed using the organic compound of one embodiment of the present invention had current efficiency and external quantum efficiency that were higher than those of the comparative device 15 formed using the comparative organic compound. This is because, as shown in Table 13, tBu-mPSi-SFTzn and tBu-mPSi-SFTzn-02, which are the organic compounds of one embodiment of the present invention, each have a refractive index of a film lower than that of 2mPCCzPDBq, which is a comparative organic compound, and thus the devices 13 and 14 each have light extraction efficiency higher than that of the comparative device 15.

As described above, the device 14 formed using tBu-mPSi-SFTzn-02 had current efficiency and external quantum efficiency that were higher than those of the device 13 formed using tBu-mPSi-SFTzn. This is because, as shown in Table 13, tBu-mPSi-SFTzn-02 has a refractive index of a film lower than that of tBu-mPSi-SFTzn, and thus the device 14 has light extraction efficiency higher than that of the device 13.

As described above, the device 13 including tBu-mPSi-SFTzn, which is the organic compound of one embodiment of the present invention, for the first electron-transport layer 914_1 and the device 14 including tBu-mPSi-SFTzn-02, which is the organic compound of one embodiment of the present invention, had driving voltages lower than that of the comparative device 15 including 2mPCCzPDBq, which is the comparative organic compound, for the first electron-transport layer 914_1. This indicates that the layers formed using tBu-mPSi-SFTzn and tBu-mPSi-SFTzn-02, respectively, have electron-transport properties higher than that of the layer formed using 2mPCCzPDBq.

The above results show that the use of the organic compound of one embodiment of the present invention enables fabrication of a light-emitting device with high emission efficiency, a low driving voltage, and a long lifetime.

### Example 10

This example describes the fabrication of the device 16 (the light-emitting device 16) in which tBu-mmTMSP-SFTzn (Structural Formula (151)), which is the organic compound of one embodiment of the present invention described in Example 7, was used for the electron-transport layer and the comparative device 17 (the comparative light-emitting device 17) and the comparative device 18 (the comparative light-emitting device 18) in each of which a comparative organic compound was used for the electron-transport layer, and measurement results of these devices' characteristics. These light-emitting devices are bottom-emission light-emitting devices that emit green phosphorescent light. Structural formulae of organic compounds used for the light-emitting devices are shown below.

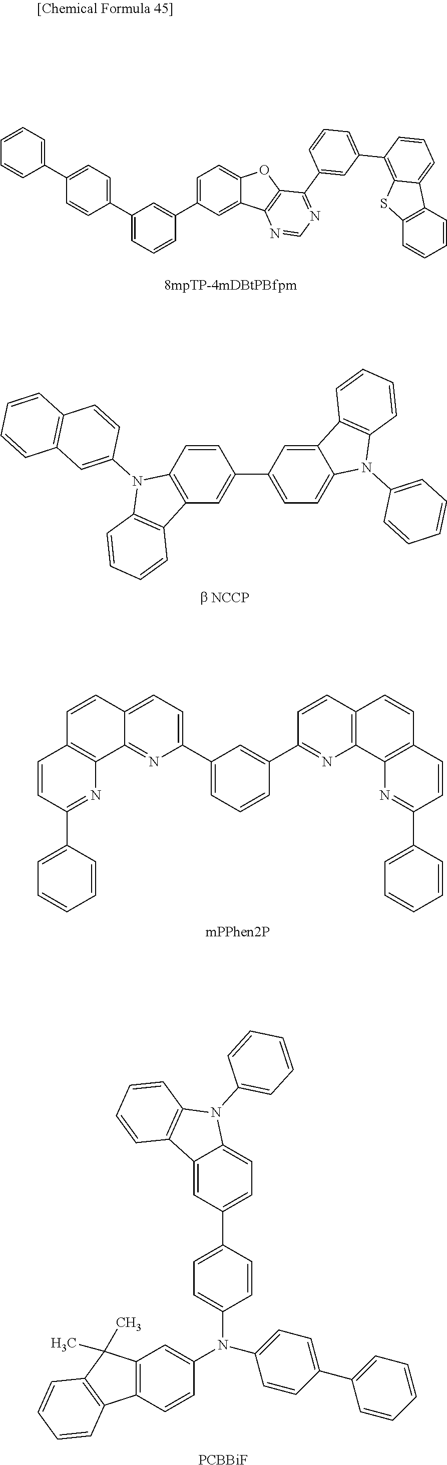

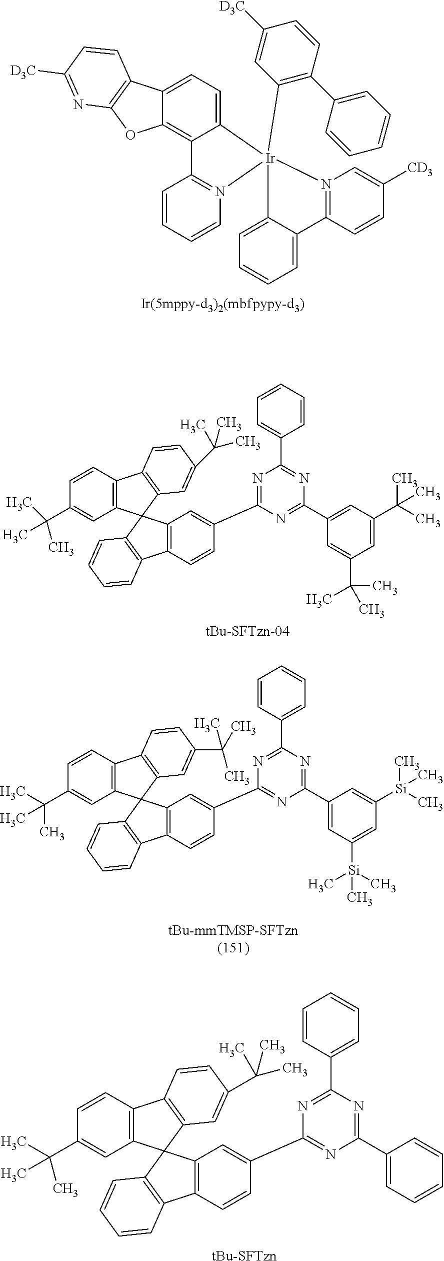

As illustrated in FIG. 11B, the light-emitting devices each have a structure in which the hole-injection layer 911, the hole-transport layer 912, the light-emitting layer 913, electron-transport layers (the first electron-transport layer 9141 and the second electron-transport layer 914_2), and the electron-injection layer 915 are stacked in this order over the first electrode 901 formed over the glass substrate 900, and the second electrode 902 is stacked over the electron-injection layer 915.

### <Fabrication Method of Device 16>

The device 16 is different from the device 13 in that tBu-mPSi-SFTzn, which is the organic compound of one embodiment of the present invention used for the first electron-transport layer 914_1 of the device 13 in Example 7, was replaced with tBu-mmTMSP-SFTzn, which is an organic compound of another embodiment of the present invention. Other components were fabricated in a manner similar to that for the device 13.

### <Fabrication Method of Comparative Device 17>

The comparative device 17 is different from the device 13 in that tBu-mPSi-SFTzn, which is the organic compound of one embodiment of the present invention used for the first electron-transport layer 914_1 of the device 13, was replaced with tBu-SFTzn, which is a comparative organic compound. Other components were fabricated in a manner similar to that for the device 13.

### <Fabrication Method of Comparative Device 18>

The comparative device 18 is different from the device 13 in that tBu-mPSi-SFTzn, which is the organic compound of one embodiment of the present invention used for the first electron-transport layer 914_1 of the device 13, was replaced with tBu-SFTzn-04, which is a comparative organic compound. Other components were fabricated in a manner similar to that for the device 13.

The device structures of the light-emitting devices are listed in Table 14.

TABLE 14

| | | Film | | Comparative | Comparative |
|---|---|---|---|---|---|
| | | thickness | Device 16 | device 17 | device 18 |
| Second electrode | | 100 nm | Al | | |
| Electron-injection layer | | 1 nm | LiF | | |
| Electron-transport | 2 | 20 nm | mPPhen2P | | |
| layer | 1 | 20 nm | tBu-mmTMSP-SFTzn | tBu-SFTzn | tBu-SFTzn-04 |

| Light-emitting layer | 40 nm | 8mpTP-4mDBtPBfpm:βNCCP:Ir(5mppy-d₃)₂(mbfpypy-d₃) |
|---|---|---|
| | | (0.5:0.5:0.1) |
| Hole-transport layer | 40 nm | PCBBiF |
| Hole-injection layer | 10 nm | PCBBiF:OCHD-003 (1:0.03) |
| First electrode | 70 nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for 1 hour were performed at the time of sealing). Then, the characteristics of the light-emitting devices were measured.

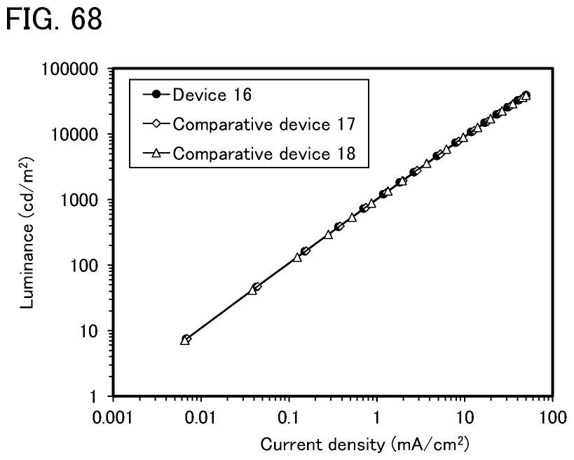

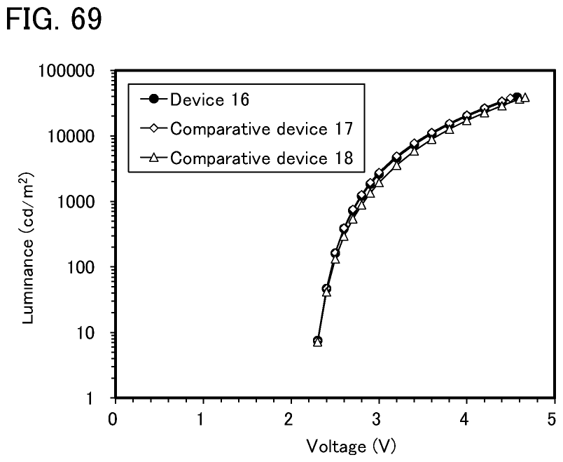

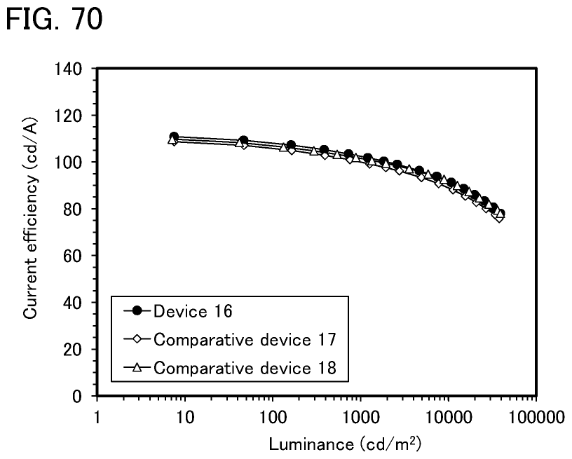

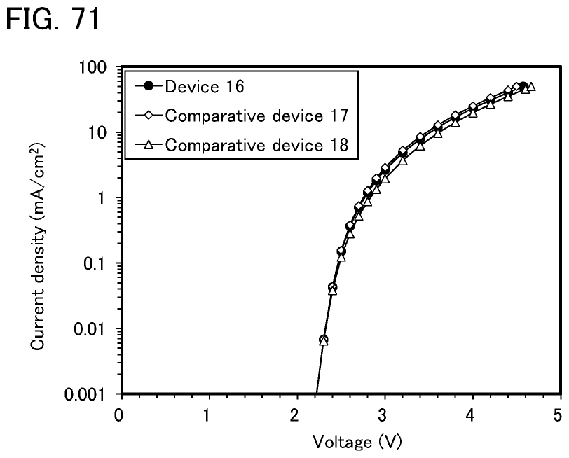

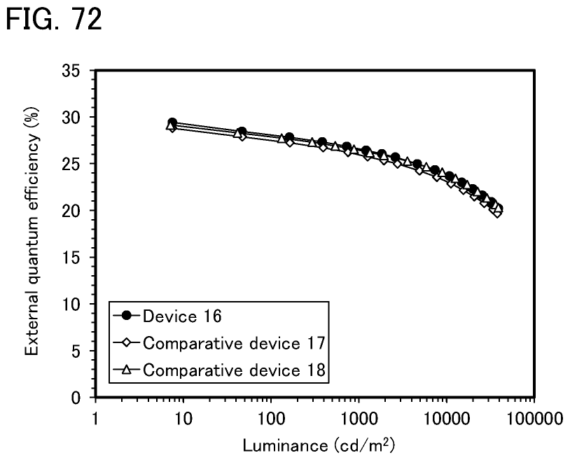

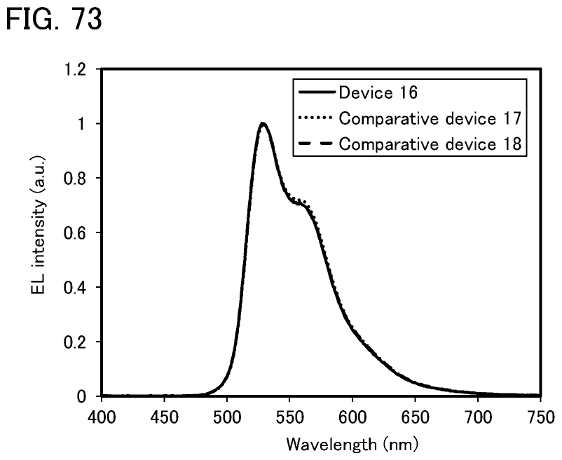

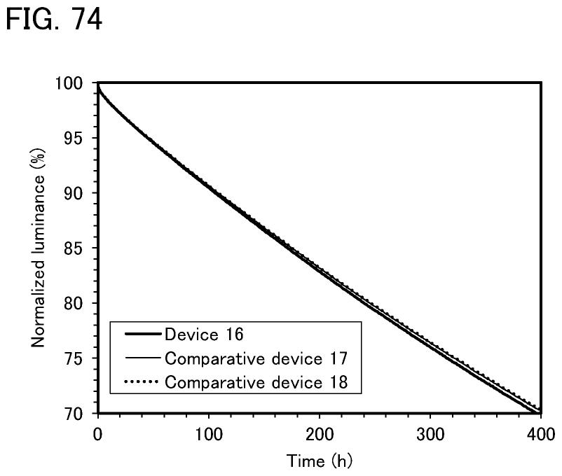

FIG. 68 shows luminance-current density characteristics of the light-emitting devices. FIG. 69 shows luminance-voltage characteristics thereof. FIG. 70 shows current efficiency-luminance characteristics thereof. FIG. 71 shows current density-voltage characteristics thereof. FIG. 72 shows external quantum efficiency-luminance characteristics thereof. FIG. 73 shows electroluminescence spectra thereof. FIG. 74 shows luminance changes over driving time when the light-emitting devices are driven at a constant current of 2 mA (50 mA/cm²).

Table 15 shows the main characteristics of the light-emitting devices at a luminance of approximately 1000 cd/m². Note that the luminance, CIE chromaticity, and emission spectra were measured with a spectroradiometer (SR-UL1R, manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 15

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Device 16 | 2.80 | 0.0475 | 1.19 | 0.356 | 0.619 | 1208 | 102 | 26.4 |
| Comparative device 17 | 2.70 | 0.0300 | 0.749 | 0.359 | 0.616 | 756 | 101 | 26.2 |
| Comparative device 18 | 2.80 | 0.0347 | 0.867 | 0.358 | 0.617 | 884 | 102 | 26.5 |

From FIG. 68, FIG. 69, FIG. 70, FIG. 71, FIG. 72, FIG. 73, FIG. 74, and Table 15, the device 16 (the light-emitting device 16) in which the organic compound of one embodiment of the present invention was used was found to be a light-emitting device having favorable characteristics that emits green light with a peak wavelength of approximately 533 nm derived from Ir(5mppy-d₃)₂(mbfpypy-d₃) and having a long lifetime.

FIG. 69 and FIG. 71 show that the device 16 has a driving voltage lower than that of the comparative device 18. FIG. 70 and FIG. 72 show that the device 16 has current efficiency and external quantum efficiency that are lower than those of the comparative device 17. FIG. 74 shows that the device 16 has a luminance change over driving time equivalent to those of the comparative devices 17 and 18.

Here, Table 16 shows ordinary refractive indices (no) and GSP_slopes of evaporated films and T_{g} of powder of main organic compounds used in the light-emitting devices. In Table 16, the ordinary refractive index at 533 nm that is a peak wavelength of the device 16 is shown as the ordinary refractive index. The ordinary refractive indices of the evaporated films of the organic compounds were measured by the method similar to that described in Example 1. In Table 16, the GSP_slopes of the evaporated films of the organic compounds were measured by the method similar to that described in Embodiment 1. In Table 16, the T_{g} of powder of the organic compounds were measured by the method similar to that described in Example 1.

TABLE 16

| | nₒ | GSP_slope | T_{g} |
|---|---|---|---|
| | (@533 nm) | (mV/nm) | (°C) |
| tBu-mmTMSP-SFTzn | 1.65 | 19.4 | 146 |
| tBu-SFTzn | 1.68 | 16.8 | 146 |
| tBu-SFTzn-04 | 1.66 | 27.7 | 154 |

As shown in Table 16, tBu-mmTMSP-SFTzn, which is the organic compound of one embodiment of the present invention, has a refractive index of a film lower than those of tBu-SFTzn and tBu-SFTzn-04, which are comparative organic compounds. In addition, tBu-mmTMSP-SFTzn has a GSP_slope of an evaporated film smaller than that of tBu-SFTzn-04. In addition, tBu-mmTMSP-SFTzn has T_{g} of powder equivalent to that of tBu-SFTzn.

As described above, the device 16 had current efficiency and external quantum efficiency higher than those of the comparative device 17. This is because, as shown in Table 16, tBu-mmTMSP-SFTzn, which is the organic compound of one embodiment of the present invention used for the device 16, has a refractive index of a film lower than that of tBu-SFTzn, which is the comparative organic compound used for the comparative device 17, and thus the device 16 has light extraction efficiency higher than that of the comparative device 17. Furthermore, as shown in Table 16, tBu-mmTMSP-SFTzn has as high T_{g} of powder as tBu-SFTzn. Thus, it can be said that the light-emitting device formed using tBu-mmTMSP-SFTzn has as high resistance to processing by a photolithography method and heat treatment as the light-emitting device formed using tBu-SFTzn and has efficiency higher than that of the light-emitting device formed using tBu-SFTzn.

As described above, the device 16 formed using tBu-mmTMSP-SFTzn, which is the organic compound of one embodiment of the present invention, for the first electron-transport layer 914_1 had a driving voltage lower than that of the comparative device 18 formed using tBu-SFTzn-04, which is the comparative organic compound, for the first electron-transport layer 914_1. This indicates that the layer formed using tBu-mmTMSP-SFTzn has an electron-transport property higher than that of the layer formed using tBu-SFTzn-04.

The above results show that the use of the organic compound of one embodiment of the present invention enables fabrication of a light-emitting device with high emission efficiency, a low driving voltage, and a long lifetime.

### Example 11

This example describes the fabrication of the device 19 (the light-emitting device 19) in which tBu-TrP-SFTzn (Structural Formula (122)), which is the organic compound of one embodiment of the present invention described in Example 8, was used for the electron-transport layer and the comparative device 20 (the comparative light-emitting device 20) and the comparative device 21 (the comparative light-emitting device 21) in each of which a comparative organic compound was used for the electron-transport layer, and measurement results of these devices' characteristics. These light-emitting devices are bottom-emission light-emitting devices that emit green phosphorescent light. Structural formulae of organic compounds used for the light-emitting devices are shown below.

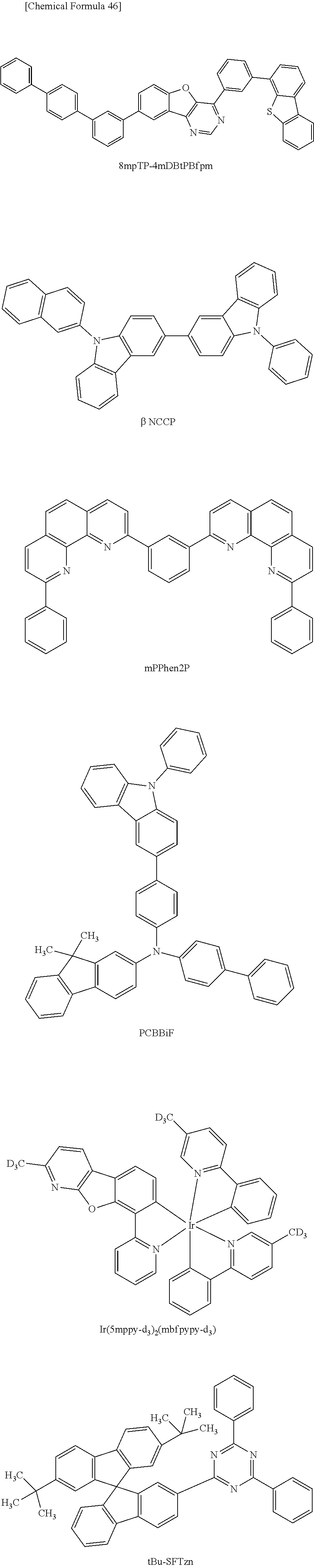

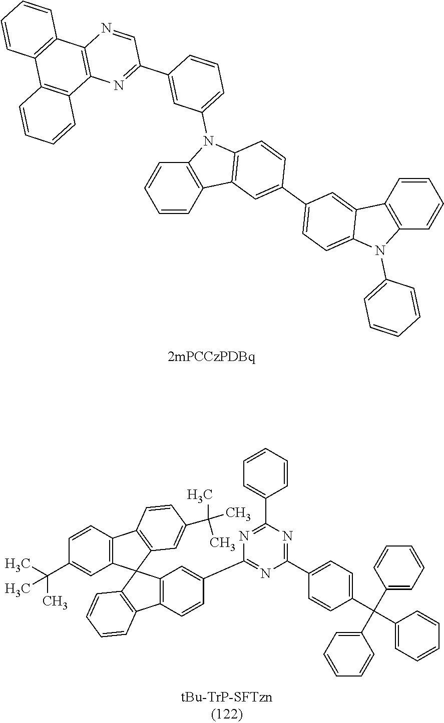 [SMILES: c1ccc(-n2c3ccccc3c3cc(-c4ccc5c(c4)c4ccccc4n5-c4cccc(-c5cnc6c7ccccc7c7ccccc7c6n5)c4)ccc32)cc1 ; CC(C)(C)c1ccc2c(c1)C1(c3ccccc3-c3ccc(-c4nc(-c5ccccc5)nc(-c5ccc(C(c6ccccc6)(c6ccccc6)c6ccccc6)cc5)n4)cc31)c1cc(C(C)(C)C)ccc1-2]

As illustrated in FIG. 11B, the light-emitting devices each have a structure in which the hole-injection layer 911, the hole-transport layer 912, the light-emitting layer 913, electron-transport layers (the first electron-transport layer 914_1 and the second electron-transport layer 914_2), and the electron-injection layer 915 are stacked in this order over the first electrode 901 formed over the glass substrate 900, and the second electrode 902 is stacked over the electron-injection layer 915.

### <Fabrication Method of Device 19>

The device 19 is different from the device 13 in that tBu-mPSi-SFTzn, which is the organic compound of one embodiment of the present invention used for the first electron-transport layer 914_1 of the device 13 in Example 7, was replaced with tBu-TrP-SFTzn, which is an organic compound of another embodiment of the present invention. Other components were fabricated in a manner similar to that for the device 13.

### <Fabrication Method of Comparative Device 20>

The comparative device 20 is different from the device 13 in that tBu-mPSi-SFTzn, which is the organic compound of one embodiment of the present invention used for the first electron-transport layer 914_1 of the device 13, was replaced with 2mPCCzPDBq, which is a comparative organic compound. Other components were fabricated in a manner similar to that for the device 13.

### <Fabrication Method of Comparative Device 21>

The comparative device 21 is different from the device 13 in that tBu-mPSi-SFTzn, which is the organic compound of one embodiment of the present invention used for the first electron-transport layer 914_1 of the device 13, was replaced with tBu-SFTzn, which is a comparative organic compound. Other components were fabricated in a manner similar to that for the device 13.

The device structures of the light-emitting devices are listed in Table 17.

TABLE 17

| | | Film | | Comparative | Comparative |
|---|---|---|---|---|---|
| | | thickness | Device 19 | device 20 | device 21 |
| Second electrode | | 100 nm | Al | | |
| Electron-injection layer | | 1 nm | LiF | | |
| Electron-transport | 2 | 20 nm | mPPhen2P | | |
| layer | 1 | 20 nm | tBu-TrP-SFTzn | 2mPCCzPDBq | tBu-SFTzn |

| | | |
|---|---|---|
| Light-emitting layer | 40 nm | 8mpTP-4mDBtPBfpm:βNCCP:Ir(5mppy-d₃)₂(mbfpypy-d₃) |

| | | (0.5:0.5:0.1) |
|---|---|---|
| Hole-transport layer | 40 nm | PCBBiF |
| Hole-injection layer | 10 nm | PCBBiF:OCHD-003 (1:0.03) |
| First electrode | 70 nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for 1 hour were performed at the time of sealing). Then, the characteristics of the light-emitting devices were measured.

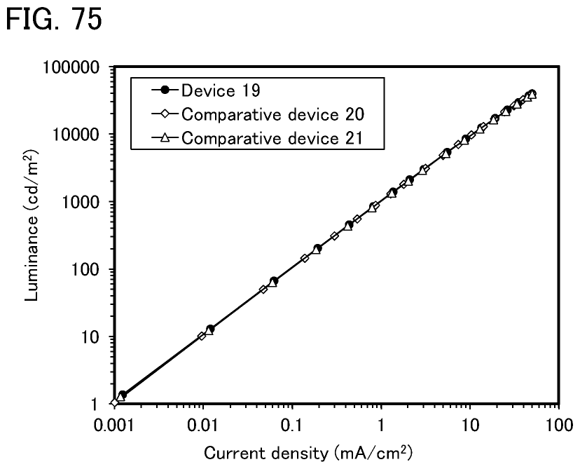

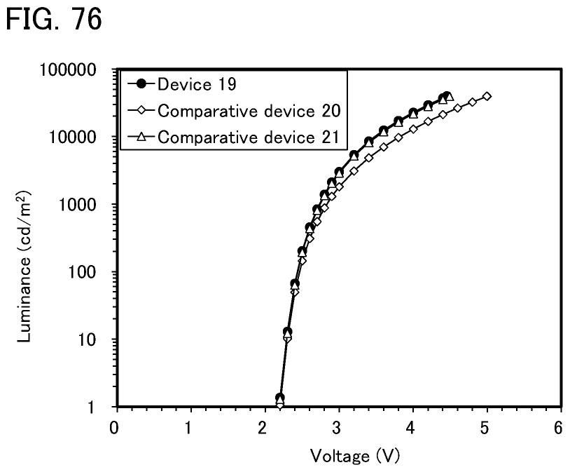

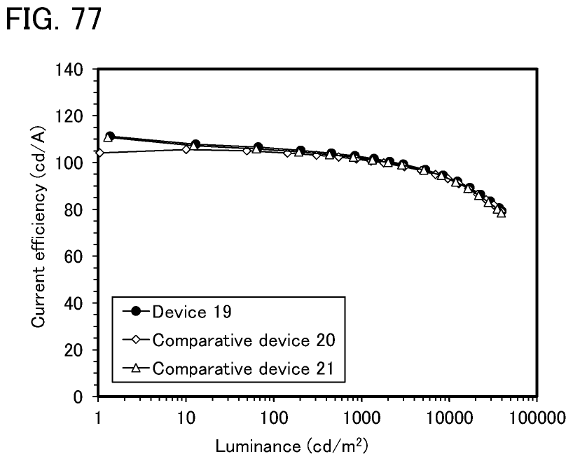

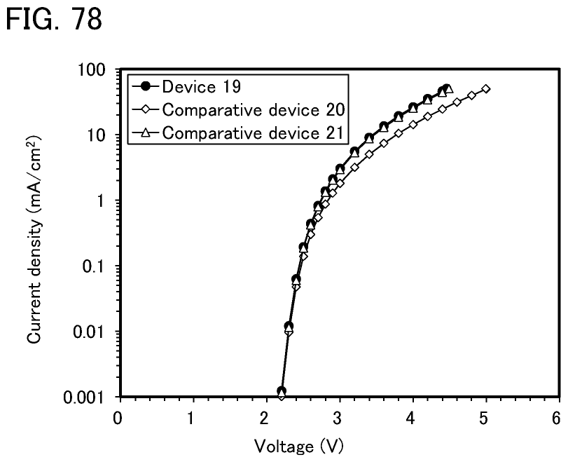

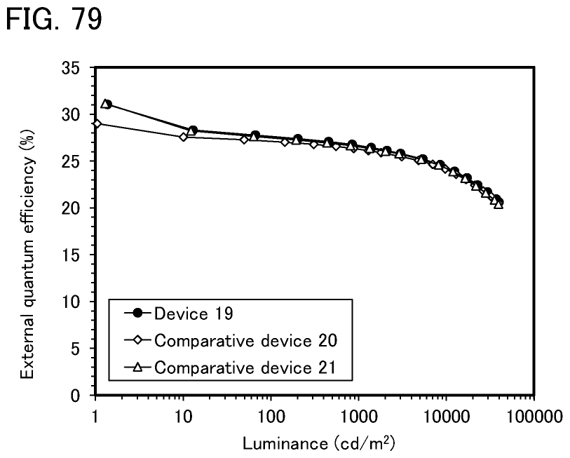

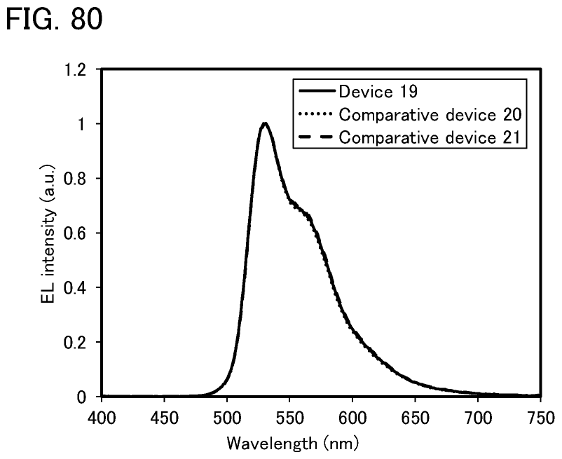

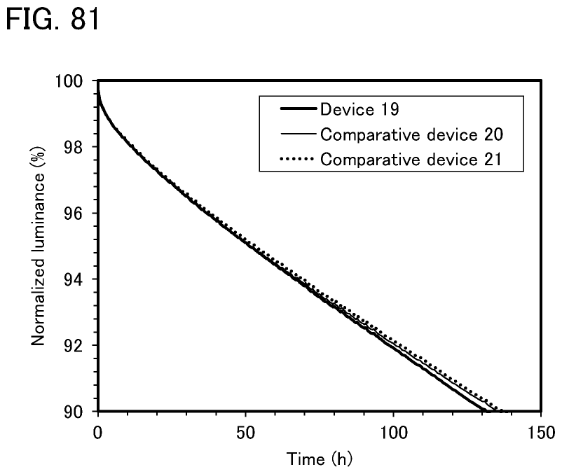

FIG. 75 shows luminance-current density characteristics of the light-emitting devices. FIG. 76 shows luminance-voltage characteristics thereof. FIG. 77 shows current efficiency-luminance characteristics thereof. FIG. 78 shows current density-voltage characteristics thereof. FIG. 79 shows external quantum efficiency-luminance characteristics thereof. FIG. 80 shows electroluminescence spectra thereof. FIG. 81 shows luminance changes over driving time when the light-emitting devices are driven at a constant current of 2 mA (50 mA/cm²).

Table 18 shows the main characteristics of the light-emitting devices at a luminance of approximately 1000 cd/m². Note that the luminance, CIE chromaticity, and emission spectra were measured with a spectroradiometer (SR-UL1R, manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 18

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Device 19 | 2.70 | 0.0328 | 0.819 | 0.359 | 0.617 | 843 | 103 | 26.7 |
| Comparative device 20 | 2.80 | 0.0346 | 0.864 | 0.357 | 0.619 | 877 | 101 | 26.3 |
| Comparative device 21 | 2.70 | 0.0316 | 0.789 | 0.361 | 0.616 | 808 | 102 | 26.7 |

From FIG. 75, FIG. 76, FIG. 77, FIG. 78, FIG. 79, FIG. 80, FIG. 81, and Table 18, the device 19 (the light-emitting device 19) in which the organic compound of one embodiment of the present invention was used was found to be a light-emitting device having favorable characteristics that emits green light with a peak wavelength of approximately 533 nm derived from Ir(5mppy-d₃)₂(mbfpypy-d₃) and having a long lifetime.

FIG. 76 and FIG. 78 show that the device 19 has a driving voltage lower than that of the comparative device 20.

Here, Table 19 shows ordinary refractive indices (no) and GSP_slopes of evaporated films and T_{g} of powder of main organic compounds used in the light-emitting devices. In Table 19, the ordinary refractive index at 533 nm that is a peak wavelength of the device 19 is shown as the ordinary refractive index. The ordinary refractive indices of the evaporated films of the organic compounds were measured by the method similar to that described in Example 1. In Table 19, the GSP_slopes of the evaporated films of the organic compounds were measured by the method similar to that described in Embodiment 1. In Table 19, the T_{g} of powder of the organic compounds were measured by the method similar to that described in Example 1.

TABLE 19

| | nₒ | GSP_slope | T_{g} |
|---|---|---|---|
| | (@533 nm) | (mV/nm) | (°C) |
| tBu-TrP-SFTzn | 1.71 | 15.4 | 187 |
| 2mPCCzPDBq | 1.92 | 12.4 | 159 |
| tBu-SFTzn | 1.68 | 16.8 | 146 |

As shown in Table 19, tBu-TrP-SFTzn, which is the organic compound of one embodiment of the present invention, is an organic compound whose refractive index of a film is as low as that of tBu-SFTzn, which is the comparative organic compound, and is lower than that of 2mPCCzPDBq, which is the comparative organic compound. In addition, tBu-TrP-SFTzn has a GSP_slope of an evaporated film smaller than that of tBu-SFTzn, which is the comparative organic compound. In addition, tBu-TrP-SFTzn has T_{g} of a film higher than that of tBu-SFTzn and 2mPCCzPDBq, which are the comparative organic compounds.

As described above, it was found that the device 19 formed using tBu-TrP-SFTzn for the first electron-transport layer 9141 had a driving voltage that is equivalent to that of the comparative device 21 formed using tBu-SFTzn for the first electron-transport layer 914_1 and is lower than that of the comparative device 20 formed using 2mPCCzPDBq for the first electron-transport layer 914_1. Thus, it can be said that the electron-transport property of the layer formed using tBu-TrP-SFTzn is equivalent to that of the layer formed using tBu-SFTzn, and the electron-transport property of the layer formed using 2mPCCzPDBq is lower than those of these layers.

Note that tBu-TrP-SFTzn has a structure where a triphenylmethyl group is bonded to the vicinity of a triazine ring in tBu-SFTzn (the mother skeleton). Like the tert-butyl group and the triphenylsilyl group described in Example 3, since the interaction between a triphenylmethyl group and the LUMO which determines an electron-transport property is extremely small, the electron-transport property of an organic compound that has the structure of tBu-SFTzn as a mother skeleton and the triphenylmethyl group introduced into the vicinity of a triazine ring is lower than that of tBu-SFTzn in some cases. However, as shown in FIG. 76 and FIG. 78, the device 19 formed using tBu-TrP-SFTzn had a driving voltage equivalent to that of the comparative device 21 formed using tBu-SFTzn. Thus, tBu-TrP-SFTzn was found to have an electron-transport property equivalent to that of tBu-SFTzn, although having a triphenylmethyl group in the vicinity of a triazine ring. This result was obtained because, even when the electron transport was impeded by three benzene rings included in the triphenylmethyl group, the electron-transport property was compensated for by hyperconjugation between an aryl group in the mother skeleton and a bond between a quaternary carbon atom and a benzene ring so that the electron-transport property was improved.

Furthermore, the T_{g} of powder of tBu-TrP-SFTzn was higher than that of 2mPCCzPDBq and tBu-SFTzn, which are comparative organic compounds. This indicates that in fabricating a light-emitting device employing a step of processing an organic compound layer by a photolithography method, the sample can be heated at a higher temperature. As a result, a large amount of moisture and impurities attached in the process can be removed. Accordingly, the light-emitting device formed using the organic compound can have a longer lifetime.

The above results show that the use of the organic compound of one embodiment of the present invention enables fabrication of a light-emitting device with a low driving voltage and a long lifetime.

This application is based on Japanese Patent Application Serial No. 2025-046525 filed with Japan Patent Office on Mar. 21, 2025, the entire contents of which are hereby incorporated by reference.

What is claimed is:

1. An organic compound represented by General Formula (G1): 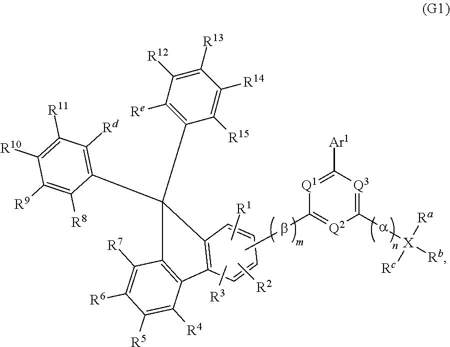
wherein:
each of Q¹, Q², and Q³ independently represents N or CH, and at least any two of Q¹, Q², and Q³ represent N;
X represents C, Si, or Ge;
each of R^{a}, R^{b}, and R^{c} independently represents a cycloalkyl group having 3 to 10 carbon atoms or a substituted or unsubstituted aryl group having 6 to 12 carbon atoms;
Ar¹ represents a substituted or unsubstituted aryl group having 6 to 30 carbon atoms;
each of α and β independently represents a substituted or unsubstituted arylene group having 6 to 30 carbon atoms;
R^{d} and R^{e} represent bonds that are bonded to each other to form a ring, or each of R^{d} and R^{e} independently represents hydrogen, an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms;
each of R¹ to R¹⁵ independently represents hydrogen, an alkyl group having 1 to 10 carbon atoms, a cycloalkyl group having 3 to 10 carbon atoms, a trisubstituted methyl group having 4 to 37 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms; and
each of n and m independently represents an integer greater than or equal to 0 and less than or equal to 3.

2. The organic compound according to claim 1,
wherein each of α and β independently represents a substituted or unsubstituted arylene group having 6 to 10 carbon atoms.

3. The organic compound according to claim 1,
wherein each of n and m independently represents an integer greater than or equal to 0 and less than or equal to 2.

4. The organic compound according to claim 1,
wherein X is Si.

5. The organic compound according to claim 1,
wherein R^{d} and R^{e} represent bonds that are bonded to each other to form a ring.

6. An organic compound represented by General Formula (G4): 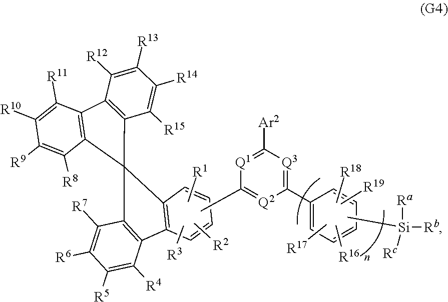
wherein:
each of Q¹, Q², and Q³ independently represents N or CH, and at least any two of Q¹, Q², and Q³ represent N;
each of R^{a}, R^{b}, and R^{c} independently represents an alkyl group having 1 to 4 carbon atoms, a cycloalkyl group having 3 to 10 carbon atoms, or a substituted or unsubstituted aryl group having 6 to 12 carbon atoms;
Ar² represents a substituted or unsubstituted aryl group having 6 to 10 carbon atoms;
each of R¹ to R¹⁹ independently represents hydrogen, an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms; and
n is an integer greater than or equal to 0 and less than or equal to 3.

7. The organic compound according to claim 6,
wherein Ar² represents a substituted or unsubstituted phenyl group.

8. The organic compound according to claim 6,
wherein n is 1.

9. The organic compound according to claim 6, wherein the organic compound is represented by General Formula (G6): 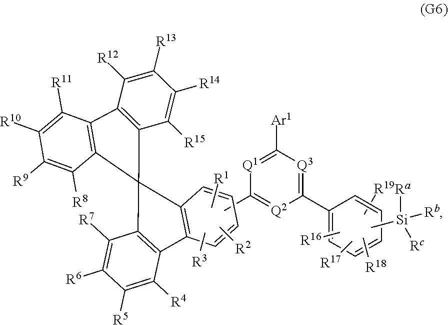
wherein:
each of Q¹, Q², and Q³ independently represents N or CH, and at least any two of Q¹, Q², and Q³ represent N;
each of R^{a}, R^{b}, and R^{c} independently represents a cycloalkyl group having 3 to 10 carbon atoms or a substituted or unsubstituted aryl group having 6 to 12 carbon atoms;
Ar¹ represents a substituted or unsubstituted aryl group having 6 to 10 carbon atoms; and
each of R¹ to R¹⁹ independently represents hydrogen, an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms.

10. The organic compound according to claim 9,
wherein each of R^{a}, R^{b}, and R^{c} independently represents the substituted or unsubstituted aryl group having 6 to 12 carbon atoms.

11. An organic compound represented by General Formula (G7): 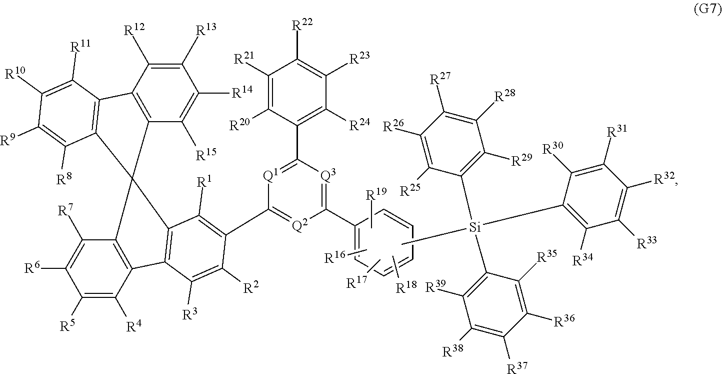
wherein:
each of Q¹, Q², and Q³ independently represents N or CH, and at least any two of Q¹, Q², and Q³ represent N;
each of R¹ to R²⁴ independently represents hydrogen, an alkyl group having 1 to 10 carbon atoms, a cycloalkyl group having 3 to 10 carbon atoms, a trisubstituted silyl group having 3 to 36 carbon atoms, a substituted or unsubstituted aryl group having 6 to 30 carbon atoms, or a substituted or unsubstituted heteroaryl group having 3 to 30 carbon atoms, and at least any one of R¹ to R¹⁵ represents an alkyl group having 1 to 10 carbon atoms; and
each of R²⁵ to R³⁹ independently represents hydrogen or an alkyl group having 1 to 10 carbon atoms.

12. The organic compound according to claim 1, wherein the organic compound is represented by any one of Structural Formula (122), Structural Formula (128), Structural Formula (129), and Structural Formula (132):

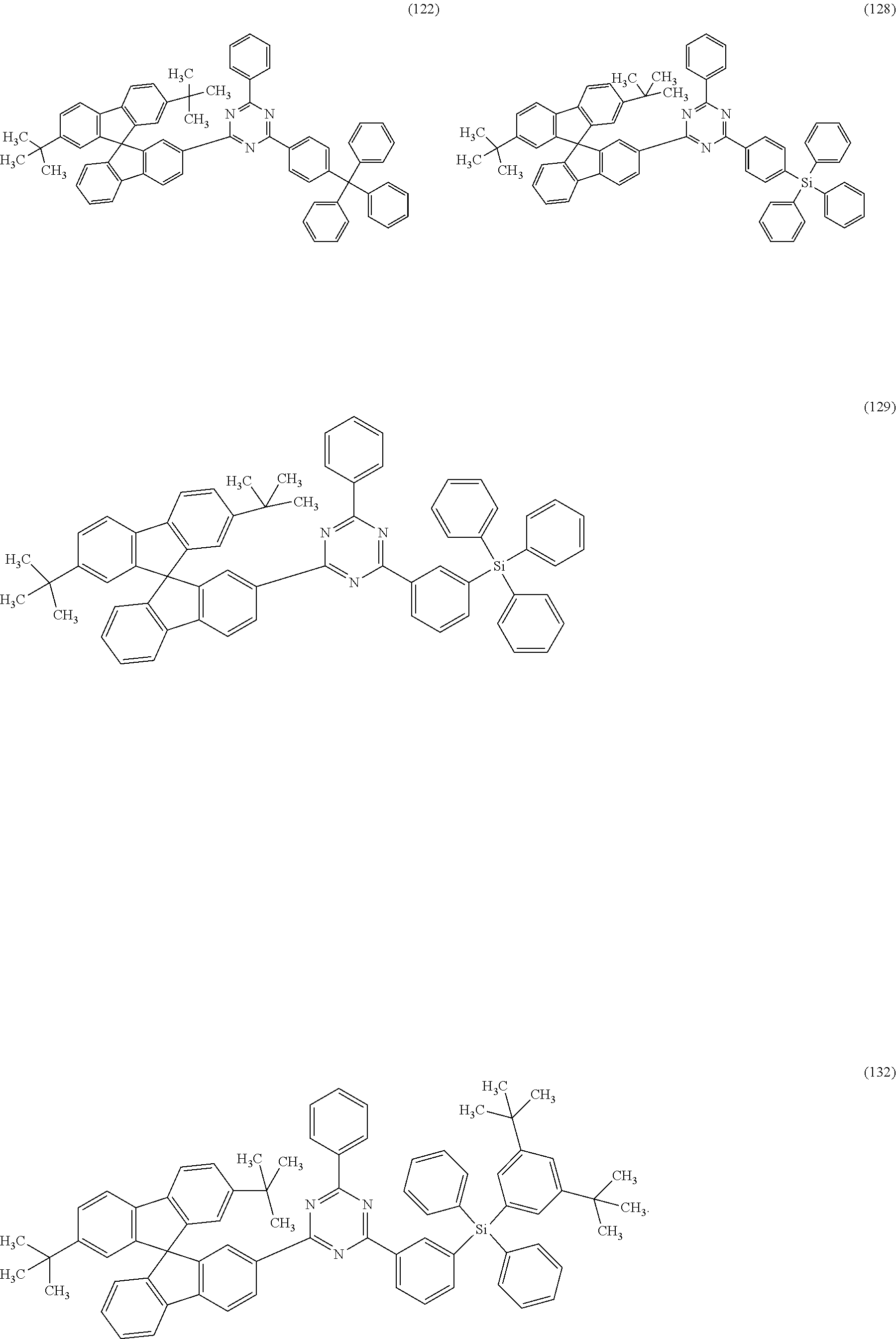

13. The organic compound according to claim 6, wherein the organic compound is represented by Structural Formula (151):

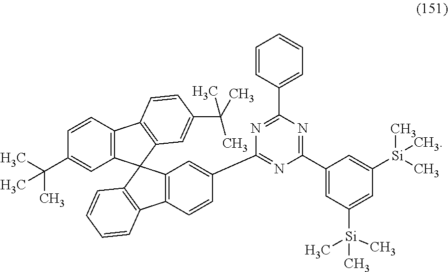 [SMILES: CC(C)(C)c1ccc2c(c1)C1(c3ccccc3-c3ccc(-c4nc(-c5ccccc5)nc(-c5cc([Si](C)(C)C)cc([Si](C)(C)C)c5)n4)cc31)c1cc(C(C)(C)C)ccc1-2]

14. A light-emitting device comprising the organic compound according to claim 1.

15. A light-emitting device comprising the organic compound according to claim 6.

16. A light-emitting device comprising the organic compound according to claim 11.
